(19) United States
(12) Patent Application Publication
(10) Pub. No.: US 2026/0028420 A1
(43) Pub. Date: Jan. 29, 2026

(54) ANTI-TUMOR ANTIBODIES

(71) Applicant: Immunome, Inc., Bothell, WA (US)

(72) Inventors: Zahra Cherry, San Carlos, CA (US); Daniel Eric Emerling, San Carlos, CA (US); Jessica Finn, San Carlos, CA (US); Shaun M. Lippow, San Carlos, CA (US); Philippe Marguet, San Carlos, CA (US); Alexander Scholz, San Carlos, CA (US); Tito Serafini, San Carlos, CA (US); Erin Wechsler, San Carlos, CA (US); Ngan Nguyen Atkins, San Carlos, CA (US); Amy Manning-Bog, San Carlos, CA (US); Sergey Boyarskiy, San Carlos, CA (US)

(21) Appl. No.: 18/854,467

(22) PCT Filed: Apr. 4, 2023
(86) PCT No.: PCT/US2023/065345

§ 371 Date: Oct. 4, 2024

Related U.S. Application Data
(60) Provisional application No. 63/362,520, filed on Apr. 5, 2022.
(60) Provisional application No. 63/374,682, filed on Sep. 6, 2022.

(51) Int. Cl. C07K 16/40; A61K 39/00; A61K 47/68; A61P 35/00; G01N 33/574

(52) U.S. Cl. CPC C07K 16/40; A61K 47/68031; A61K 47/6851; A61P 35/00; C12Y 204/01152; C12Y 204/01244; G01N 33/57492; A61K 2039/505; C07K 2317/77; C07K 2317/92; G01N 2333/91102

(57) ABSTRACT

Provided herein are antibodies that target tumors, wherein the binding of the antibody to the tumor is dependent on the expression of one or more glycosyltransferases in the tumor. These antibodies bind preferentially to tumor tissue as compared to normal tissue. Such antibodies are used in methods of inhibiting tumor cell growth.

### RELATED APPLICATIONS

This application claims priority to U.S. Provisional Application No. 63/362,520, filed on Apr. 5, 2022, and U.S. Provisional Application No. 63/374,682, filed on Sep. 6, 2022. The entire content of each provisional application is incorporated herein by reference for all purposes.

### FIELD OF CANCER THERAPEUTICS

This application relates to therapeutic antibodies for the treatment of various cancers.

### BACKGROUND

Protein glycosylation is one of the most complex and common post-translational modifications representing the enzymatic addition of carbohydrate chains called glycans. Glycan-specific antibodies can be detected early in life without immunization, as through infections and vaccinations. The expression of glycans and glycan-specific antibodies may change during the cancer progression. Glycans and glycan-specific antibodies have been suggested to serve as cancer diagnostic and prognostic markers (Tikhonov et al., Glycan-specific antibodies as potential cancer biomarkers: a focus on microarray application, Clinical Chemistry and Laboratory Medicine (CCLM) (2019) Vol. 58: Issue 10). However, since most known glycans are found in cancer patients and healthy individuals, the utility of glycans in cancer therapeutics has not been fully explored.

### BRIEF SUMMARY

In some embodiments, provided herein is an antibody that binds a tumor, wherein the binding of the antibody to the tumor is dependent on the expression of one or more glycosyltransferases in the tumor, wherein one of the one or more glycosyltransferases has N-acetyl-galactosaminyltransferase activity.

In some embodiments, one of the one or more glycosyltransferases has fucosyltransferase activity. In some embodiments, the glycosyltransferase that has N-acetyl-galactosaminyltransferase activity is selected from the group consisting of B4GALNT3 and B4GALNT4. In some embodiments, one of the one or more glycosyltransferases that has fucosyltransferase activity is selected from the group consisting of FUT3, FUT4, FUT5, FUT6, FUT7, FUT9, FUT10, and FUT11.

In some embodiments, the tumor expresses a tumor-associated glycan. In some embodiments, the tumor-associated glycan is an extracellular glycan. In some embodiments, the one or more glycosyltransferases is selected from the group consisting of B4GALNT3 and B4GALNT4. In some embodiments, the one or more glycosyltransferases is selected from the group consisting of FUT3, FUT4, FUT5, FUT6, FUT7, FUT9, FUT10, and FUT11 In some embodiments, the glycosyltransferase is FUT4. In some embodiments, the glycosyltransferase is B4GALNT3. In some embodiments, the presence of the tumor-associated glycan is dependent on the expression of B4GALNT3 and FUT4 in the tumor. In some embodiments, the antibody preferentially binds to a tumor tissue relative to a normal tissue. In some embodiments, the antibody is internalized by the tumor cells upon contacting the tumor.

In some embodiments, the antibody binds to a tumor, wherein the antibody comprises: a heavy chain variable region comprising: an HCDR1, HCDR2, and/or HCDR3 amino acid sequence listed in Table 1, or variants of the HCDR1, HCDR2, and/or HCDR3 amino acid sequence in which 1, 1, 2, 3, 4, 5, or more amino acids are substituted; and/or a light chain variable region comprising: an LCDR1, LCDR2, and/or LCDR3 amino acid sequence listed in Table 2, or variants of the LCDR1, LCDR2, and/or LCDR3 amino acid sequence in which 1, 2, 3, 4, 5, or more amino acid are substituted.

In some embodiments, the antibody comprises: wherein the antibody comprises all six CDRs of an antibody selected from the group consisting of AB-006410, AB-011110, AB-011111, AB-011366, AB-011367, AB-011368, AB-011369, AB-011370, AB-011371, AB-011372, AB-011373, AB-011374. AB-011375, AB-011376, AB-011622, AB-011623, AB-011624, AB-011625, AB-011626, AB-011627, AB-011628, AB-011781, AB-011782, AB-011783, AB-011784, AB-011785, AB-011786. AB-011787, AB-011788, AB-011789, AB-011790, AB-011791, AB-011792, AB-011793, AB-011794, AB-011795, AB-011796, AB-011797, AB-011798, AB-011799, AB-011800, AB-011801. AB-011802, AB-011803, AB-011804, AB-011805, AB-011806, AB-011807, AB-011808, AB-011809, AB-011810, AB-011811, AB-011812, AB-011813, AB-011814, AB-011815, AB-011816. AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861. AB-011862, AB-011863, AB-011864, AB-011865, AB-011866, AB-011867, AB-011868, AB-011869, AB-011870, AB-011871, AB-011872, and AB-011873.

In some embodiments, the antibody comprises a VH region comprising a VH amino acid sequence in Table 3 or an amino sequence having at least 70%, at least 75%, at least 80%, at least 85%, at least 90%, or at least 95% identity to the VH amino acid sequence in Table 3, and/or wherein the antibody comprises a VL region comprising a VL amino acid sequence in Table 3; and an amino sequence having at least 70%, at least 75%, at least 80%, at least 85%, at least 90%, or at least 95% identity to the VL amino acid sequence in Table 3.

In some embodiments, the antibody comprises both the VH and VL of an antibody selected from the group consisting of AB-006410, AB-011110, AB-011111, AB-011366, AB-011367, AB-011368, AB-011369, AB-011370, AB-011371, AB-011372, AB-011373, AB-011374, AB-011375, AB-01137, AB-011622, AB-011623, AB-011624, AB-011625, AB-011626, AB-011627, AB-011628, AB-011781. AB-011782, AB-011783, AB-011784, AB-011785, AB-011786, AB-011787, AB-011788, AB-011789, AB-011790, AB-011791, AB-011792, AB-011793, AB-011794, AB-011795, AB-011796. AB-011797, AB-011798, AB-011799, AB-011800, AB-011801, AB-011802, AB-011803, AB-011804, AB-011805, AB-011806, AB-011807, AB-011808, AB-011809, AB-011810, AB-011811. AB-011812, AB-011813, AB-011814, AB-011815, AB-011816, AB-011817, AB-011818, AB-011819, AB-011820, AB-011821, AB-011822, AB-011823, AB-011824, AB-011825, AB-011826. AB-011827, AB-011828, AB-011829, AB-011830, AB-011831, AB-011832, AB-011833, AB-011834, AB-011835, AB-011836, AB-011837, AB-011838, AB-011839, AB-011840, AB-011841. AB-011842, AB-011843, AB-011844, AB-011845, AB-011846, AB-011847, AB-011848, AB-011849, AB-011850, AB-011851, AB-011852, AB-011853, AB-011854, AB-011855, AB-011856. AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, AB-011873 and a variant thereof. In some embodiments, the antibody comprises both the VH and VL of an antibody selected from the group consisting of AB-011110, AB-011788, AB-011789, AB-011794, AB-011367, and AB-011861. This disclosure also provides an antibody that competes for binding with any of the antibody above.

Also provided herein is an immunoconjugate comprising the antibody disclosed above and a cytotoxic agent. In some embodiments, the cytotoxic agent is Auristatin. In some embodiments, the cytotoxic agent is ZymeLink™ Auristatin (ZLA).

In some embodiments, the immunoconjugate comprises Formula (I) or (II):

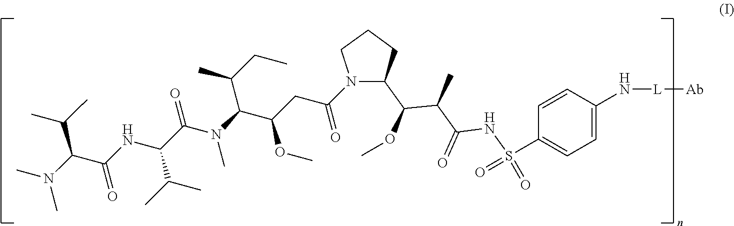

wherein: L is a cleavable linker; n is the drug-to-antibody ratio (DAR) and is an integer from 1 to 12, and Ab is an antibody disclosed herein or

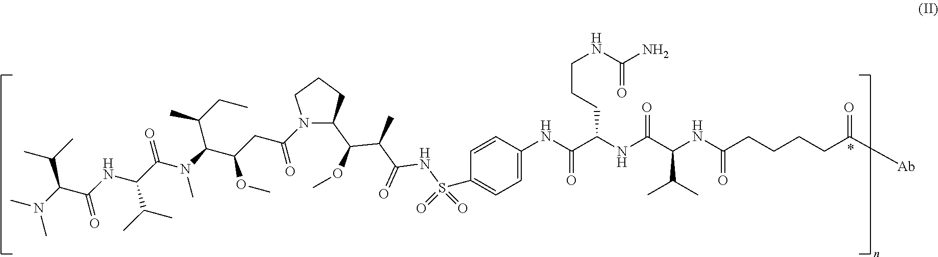

wherein: n is the drug-to-antibody ratio (DAR) and is an integer from 1 to 12, and Ab is an antibody disclosed herein.

Also provided herein is a polynucleotide encoding a polypeptide comprising a V_{H} sequence and/or a V_{L} sequence of an antibody disclosed above. Also provided herein is an expression vector comprising the polynucleotide. Also provided herein is a host cell that comprises the expression vector. Also provided herein is a pharmaceutical composition comprising an antibody of the immunoconjugate and a pharmaceutically acceptable carrier.

Also provided herein is a method of treating a cancer patient, the method comprising administering the immunoconjugate or the antibody to the patient.

Also provided herein is a method of identifying a patient having a tumor suitable for treatment with an antibody, wherein the binding of the antibody to the tumor is dependent on the expression of one or more glycotransferases in the tumor, wherein one of the one or more glycosyltransferases has N-acetyl-galactosaminyltransferase activity, wherein the method comprises: contacting a tumor sample from the patient with an antibody disclosed herein, and detecting binding of the antibody to the tumor sample, wherein detection of the binding identifies the patient having a tumor suitable for treatment with the antibody.

Also provided herein is a method of selecting an anti-tumor antibody, the method comprising (1) contacting a candidate antibody with a tumor cell (or a lysate thereof) or a control cell (or a lysate thereof), wherein the tumor cell (or a lysate thereof) comprises (i) one or more glycosyltransferases in the tumor, wherein one of the one or more glycosyltransferases has N-acetyl-galactosaminyltransferase activity, (2) detecting binding of the candidate antibody with the tumor cell (or the lysate thereof) or with the control cell (or a lysate thereof), and (3) selecting the candidate antibody as the anti-tumor antibody if the binding of the candidate antibody to the tumor cell (or the lysate thereof) is greater than the binding of the candidate antibody to the control cell (or the lysate thereof).

Also provided herein is use of an antibody or an immunoconjugate disclosed herein for a method of treating cancer.

### BRIEF DESCRIPTION OF THE DRAWINGS

FIG. 1 shows strong binding of AB-006410 to human cancer cell line LoVo but diminished binding of the same antibody to LoVo cells in which glycosyltransferases have been inhibited.

FIG. 2A-B show that binding of AB-006410 is reduced in cells engineered by CRISPR to knock-out the glycosyltransferases B4GALNT3 (A) or FUT4 (B).

FIG. 3A-D shows that binding of AB-006410 (B) and AB-011110 (D) is enhanced in A549 cells that have had their glycan profiles altered by overexpression of B4GALNT3 and FUT4 as compared to cells that have not been altered (A) and (C).

FIG. 4 shows flow cytometry analysis of the binding of AB-006410 to dissociated human colorectal carcinoma tumor cells.

FIG. 5 shows that variant AB-011111 exhibited the same reactivity profile on colorectal carcinoma tumor tissue as AB-006410.

FIG. 6A shows tumor-specific reactivity of AB-006410 to multiple human cancer types.

FIG. 6B shows tumor-specific reactivity of AB-006410, AB-011110, and AB-011628 to colorectal and pancreatic cancers.

FIG. 7 shows the ADC activity of the glycan binders on the LoVo cells as compared to Cetuxinab Fv.

FIG. 8 shows that variants AB-011110 and AB-011111 retain the ADC cytotoxicity of the parent AB-006410.

FIG. 9A shows the cytotoxicity of AB-006410-auristatin Zymelink™ ADC (AB-006410-ZLA) on LoVo cells, with cetuximab-auristatin ZymeLink™ ADC (Cetuximab-ZLA), included as a positive control and Isotype-auristatin ZymeLink™ ADC (Isotype-ZLA) included as the negative control.

FIGS. 9B and 9C show the cytotoxicity of various glycan binders as auristatin Zymelink™ ADC constructs on LoVo cells.

FIG. 10 shows tumor inhibition by AB-006410-auristatin ZymeLink™ ADC constructs (AB6410-Zyme), with cetuximab-auristatin ZymeLink™ ADC (Cetuximab-zyme) included as a positive control and Isotype-auristatin ZymeLink™ ADC (Isotype-Zyme) included as the negative control.

FIGS. 11A-D show alignments and CDR designations for AB-006410 and other various glycan binders.

FIG. 12 shows that overexpression of B4GALNT3 and FUT4 in A549 cells sensitizes these cells to AB-006410-ADC-mediated killing.

FIG. 13 shows immunofluorescence staining results of colorectal cancer tissue sections with AB-006410. B4GALNT3, and FUT4 antibodies. The upper right corner of each image labeled “AB-006410” or “B4GALNT3” shows the magnified version of the boxed content.

FIG. 14 shows flow cytometry analysis of the binding of variants of AB-006410 to CRC cell lines: LoVo, HT29, and LS174T.

FIG. 15 shows flow cytometry analysis of internalization of AB-006410 in HT55, LoVo, and NUGC4 cells. The upper panels show the signal from internalized AB-006410 in the cells, and the lower panels show the signal from surface-bound AB-006410.

FIG. 16 shows the structures of glycans comprising LacdiNAc.

### DETAILED DESCRIPTION

As used herein, the singular forms “a,” “an,” and “the” include plural referents unless the content dictates otherwise. Thus, for example, reference to “an antibody” optionally includes a combination of two or more such molecules and the like.

The term “about,” as used herein, refers to the usual error range for the respective value readily known to the skilled person in this technical field; for example, ±20%, ±10%, or ±5%, are within the intended meaning of the recited value.

As used herein, the term “antibody” means an isolated or recombinant binding agent that comprises the necessary variable region sequences to specifically bind an antigenic epitope. Therefore, an “antibody” as used herein is any form of an antibody of any class or subclass or fragment thereof that exhibits the desired biological activity, e.g., binding a specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)₂, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment with two antigen combining sites and is still capable of cross-linking antigen.

As used herein, the term “targets a tumor,” or “tumor-targeting,” with respect to an antibody, refers to an antibody that binds preferentially to a tumor tissue than normal tissue. In some embodiments, the normal tissue is the tissue that is adjacent to the tumor, referred to as tumor-adjacent tissue or TAT. In some embodiments, a tumor-targeting antibody also decreases the rate of tumor growth, tumor size, invasion, and/or metastasis, via direct or indirect effects on tumor cells.

As used herein, “V-region” refers to an antibody variable region domain comprising the segments of Framework 1, CDR1, Framework 2, CDR2, and Framework 3, including CDR3 and Framework 4. The heavy chain V-region, V_{H}, is a consequence of the rearrangement of a V-gene (HV), a D-gene (HD), and a J-gene (HJ), in what is termed V(D)J recombination during B-cell differentiation. The light chain V-region, V_{L}, is a consequence of the rearrangement of a V-gene (LV) and a J-gene (LJ).

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the CDR is located. Thus, a V_{H} CDR3 (HCDR3) is in the variable domain of the heavy chain of the antibody in which it is found, whereas a V_{L} CDR3 (LCDR3) is the CDR3 from the variable domain of the light chain of the antibody in which it is located. The term “CDR” is used interchangeably with “HVR” when referring to CDR sequences.

The amino acid sequences of the CDRs and framework regions can be determined using various well-known definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, Structural repertoire of the human V_{H} segments J. Mol. Biol. 227, 799-817; Al-Lazikani et al., J. Mol. Biol. 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al., Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin et al., Methods Enzymol., 203, 121-153, (1991); Pedersen et al., Immunomethods, 1, 126, (1992); and Rees et al., In Steinberg M. J. E. (ed.), Protein Structure Prediction. Oxford University Press, Oxford, 141-172 1996). Reference to CDRs as determined by Kabat numbering is based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

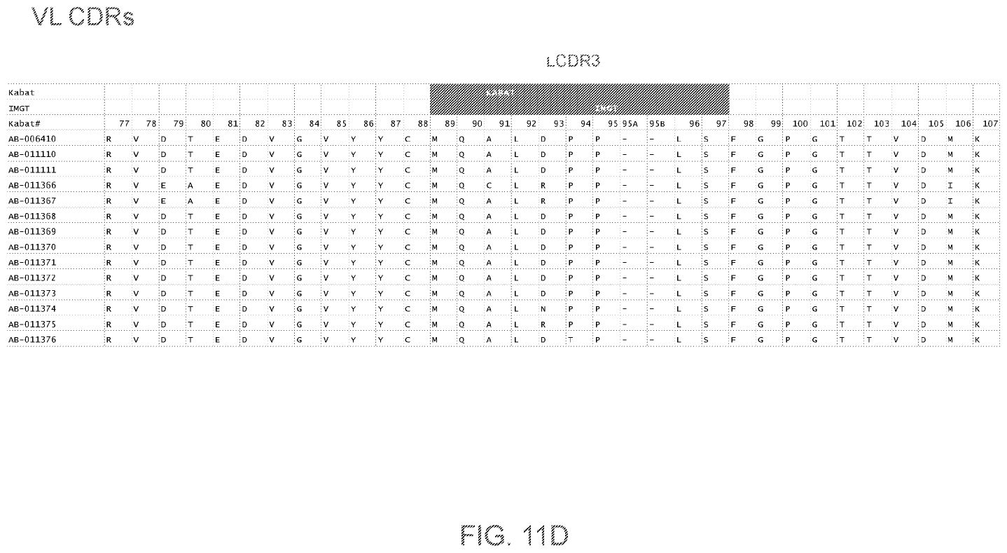

CDRs as shown in Tables 1 and 2 are defined by IMGT and Kabat. The V_{H} CDRs as listed in Table 1, are defined as follows: HCDR1 is defined by combining Kabat and IMGT; HCDR2 is defined by Kabat, and the HCDR3 is defined by IMGT. The V_{L} CDRs as listed in Table 2 are defined by Kabat. FIG. 11A-11D show alignment of certain glycan binder V_{H} and V_{L} sequences with CDRs designated by Kabat and IMGT. The CDRs of other glycan binders provided herein can be similarly designated. As known in the art, numbering and placement of the CDRs can differ depending on the numbering system employed. It is understood that disclosure of a variable heavy and/or variable light sequence includes the disclosure of the associated CDRs, regardless of the numbering system employed.

An “Fc region” refers to the constant region of an antibody excluding the first constant region immunoglobulin domain. Thus, e.g., for human immunoglobulins, “Fc” refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fe comprises immunoglobulin domains Cγ2 and Cγ3 and the hinge between Cγ1 and Cγ2. It is understood in the art that the boundaries of the Fc region may vary, however, the human IgG heavy chain Fe region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fe region” includes naturally occurring allelic variants of the Fc region as well as modified Fe regions, e.g., that are modified to modulate effector function or other properties such as pharmacokinetics, stability or production properties of an antibody. Fe regions also include variants that do not exhibit alterations in biological function. For example, one or more amino acids can be deleted from the N-terminus or C-terminus of the Fc region of an immunoglobulin without substantial loss of biological function. Such variants can be selected according to general rules known in the art to have minimal effect on activity (see, e.g., Bowie et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibodies (see, e.g., Angal et al., Mol Immunol 30:105-108, 1993).

An “EC₅₀” as used herein refers to the half-maximal effective concentration, which is the concentration of an antibody that induces a response (signal generated in engagement assay) halfway between the baseline and maximum after a specified exposure time. In some embodiments, the “fold over EC50” is determined by dividing the EC50 of a reference antibody by the EC₅₀ of the test antibody.

The term “equilibrium dissociation constant” abbreviated (K_{D}), refers to the dissociation rate constant (k_{d}, time⁻¹) divided by the association rate constant (kₐ, time⁻¹ M⁻¹). Equilibrium dissociation constants can be measured using any method. Thus, in some embodiments antibodies of the present disclosure have a K_{D} of less than about 50 nM, typically less than about 25 nM, or less than 10 nM, e.g., less than about 5 nM or than about 1 nM and often less than about 10 nM as determined by surface plasmon resonance analysis using a biosensor system such as a Biacore® system performed at 37° C. In some embodiments, an antibody of the present disclosure has a K_{D} of less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a bivalent antibody. In the context of the present invention, an “improved” K_{D} refers to a lower K_{D}. In some embodiments, an antibody of the present disclosure has a K_{D} of less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a monovalent antibody, such as a monovalent Fab. In some embodiments, a glycan binder of the present disclosure has K_{D} less than 100 pM, e.g., or less than 75 pM, e.g., in the range of 1 to 100 pM, when measured by surface plasmon resonance analysis using a biosensor system such as a Biacore® system performed at 37° C. In some embodiments, a glycan binder of the present disclosure has K_{D} of greater than 100 pM, e.g., in the range of 100-1000 pM or 500-1000 pM when measured by surface plasmon resonance analysis using a biosensor system such as a Biacore® system performed at 37° C.

The term “monovalent molecule” refers to a molecule having one antigen-binding site, e.g., a Fab or scFv.

The term “bivalent molecule” as used herein, refers to a molecule having two antigen-binding sites. In some embodiments, a bivalent molecule of the present invention is a bivalent antibody or a bivalent fragment thereof. In some embodiments, a bivalent molecule of the present invention is a bivalent antibody. In some embodiments, a bivalent molecule of the present invention is an IgG. In general. monoclonal antibodies have a bivalent basic structure. IgG and IgE have only one bivalent unit, while IgA and IgM consist of multiple bivalent units (2 and 5, respectively) and thus have higher valencies. This bivalency increases the avidity of antibodies for antigens.

The terms “monovalent binding” or “monovalently binds to” as used herein refer to the binding of one antigen-binding site to its antigen.

The terms “bivalent binding” or “bivalently binds to” refer to the binding of both antigen-binding sites of a bivalent molecule to its antigen. In some embodiments, both antigen-binding sites of a bivalent molecule share the same antigen specificity.

The term “valency” refers to the number of different binding sites of an antibody for an antigen. A monovalent antibody comprises one binding site for an antigen. A bivalent antibody comprises two binding sites for the same antigen.

The term “avidity” in the context of antibody binding to an antigen refers to the combined binding strength of multiple binding sites of the antibody. Thus, “bivalent avidity” refers to the combined strength of two binding sites.

The terms “identical” or percent “identity,” in the context of two or more polynucleotide or polypeptide sequences, refer to two or more sequences or subsequences that are the same or have a specified percentage of amino acid residues that are the same (e.g., 100% identity) or have a specified percentage of nucleotides or amino acid residues are the same (e.g., at least 70%, at least 75%, at least 80%, 85%, 90%, 91%, 92%, 93%, 94%, 95%, 96%, 97%, 98%, 99%, or higher sequence identity; or 70%, 75%, 80%, 85%, 90%, 91%, 92%, 93%, 94%, 95%, 96%, 97%, 98%, or 99% sequence identity)) identity over a specified region, e.g., the length of the two sequences, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity include the BLAST 2.0 algorithms, which are described in Altschul et al., Nuc. Acids Res. 25:3389-3402 (1977) and Altschul et al., J. Mol. Biol. 215:403-410 (1990). Thus, for purposes of this disclosure, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. The polypeptide that is aligned to the reference sequence need not be the same length as the reference sequence.

A “conservative” substitution refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile; (vi) hydrophobic sulfur-containing amino acids Met and Cys, which are not as hydrophobic as Val, Leu, and Ile; (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid in this paragraph refers to the charge at pH 6-7.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, or combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, the substitution of one or more of the naturally occurring nucleotides with an analog, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used herein refers to a recombinant construct in which a nucleic acid sequence of interest is inserted into the vector. Certain vectors can direct the expression of nucleic acids to which they are operatively linked. Such vectors are referred to herein as “expression vectors”.

A “substitution” denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy or light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell is a recombinant host cell and includes the primary transformed cell and progeny derived therefrom without regard to the number of passages.

A polypeptide “variant” is a polypeptide that typically differs from one or more polypeptide sequences specifically disclosed herein in one or more substitutions, deletions, additions, and/or insertions.

The term “cancer cell” or “tumor cell” as used herein refers to a neoplastic cell. The term includes cells from tumors that are benign as well as malignant. Neoplastic transformation is associated with phenotypic changes of the tumor cell relative to the cell type from which it is derived. The changes can include loss of contact inhibition, morphological changes, and unregulated cell growth,

The terms “inhibiting growth of a tumor” and “inhibiting growth of a cancer” are interchangeable and refer to slowing growth and/or reducing the cancer cell burden of a patient that has cancer. “Inhibiting growth of a cancer” thus includes killing cancer cells, as well as decreasing the rate of tumor growth, tumor size, invasion, and/or metastasis by direct or indirect effects on tumor cells.

A “therapeutic agent” refers to an agent that when administered to a patient suffering from a disease, in a therapeutically effective dose, will cure, or at least partially arrest the symptoms of the disease and complications associated with the disease.

“Expression of a glycan by a cell” or a “glycan expressed by a cell” means that the glycan is present in or on that cell.

A “tumor overexpressing glycans”, or a “tumor that overexpresses glycans”, or a “cancer overexpressing glycans” or a “cancer that overexpresses glycans”, refers to a tumor or cancer that expresses specific glycans at a level that higher than the level of those glycans expressed in normal tissue (e.g., tumor adjacent tissues or TAT) or otherwise has an increased amount of those glycans as compared to normal tissue. In certain embodiments, a tumnor or cancer that overexpresses glycans expresses glycan at a level that is at least 10%, at least 20%, at least 30%, at least 40%, at least 50%, at least 80%, at least 100%, at least 200%, at least 300% higher or more than the normal tissue (e.g., tumor-adjacent tissues or TAT).

The term “tumor-associated glycan” refers to a glycan expressed by a tumnor cell. In some embodiments, the tumor-associated glycan is not expressed by normal tissue cells. In some embodiments, the tumor-associated glycan is expressed by a tumor at a level that is at least 10%, at least 20%, at least 30%, at least 40%, at least 50%, at least 80%, at least 100%, at least 200%, at least 300% higher or more as compared to normal tissue (e.g., tumor-adjacent tissues or TAT), In some embodiments, the tumor-associated glycan is expressed on the cell surface, i.e., is an extracellular glycan. A glycan or a tumor-associated glycan disclosed herein may be attached to proteins or lipids, known as glycoproteins and glycolipids.

In some aspects, the disclosure additionally provides methods of identifying subjects who are candidates for treatment with a glycan binder having tumor-targeting effects. Thus, in one embodiment, the invention provides a method of identifying a patient who can benefit from treatment with a glycan binder of the present disclosure. In one embodiment, the patient has tumor that expresses glycans. In one embodiment, the patient has tumor expressing a tumor-associated glycan. In some embodiments, the tumor sample is from a primary tumor. In alternative embodiments, the tumor sample is a metastatic lesion. Binding of antibody to tumor cells through a binding interaction with the glycans can be measured using any assay, such as immunohistochemistry or flow cytometry. In some embodiments, binding of antibody to at least 0.2%, 0.5%, or 1%, or at least 5% or 10%, or at least 20%, 30%, or 50%, of the tumor cells in a sample may be used as a selection criterion for determining a patient to be treated with a glycan binder as described herein. In other embodiments, analysis of components of the blood is used to identify a patient whose tumor cells are expressing a tumor-associated glycan.

A glycan binder disclosed herein can be used to treat several different cancers. In some embodiments, a cancer patient who can benefit from the treatment of the glycan binder has a cancer expressing glycans. In some embodiments, a cancer patient who can benefit from the treatment of the glycan binder has a tumor expressing a tumor-associated glycan. In some embodiments, the cancer is a carcinoma, a melanoma, or a sarcoma.

As used herein, the term “a glycan binder,” “an antibody that binds to glycans”, or “an anti-glycan antibody” refers to a molecule, for example, an antibody or antibody binding domain, that binds to a tumor and the binding is dependent on the activity of one or more glycosyltransferases. In some embodiments, the glycan binder binds to a tumor-associated glycan under permissible conditions (e.g., in a suitable buffer), and the detected signal resulted from the binding is at least 2 fold, at least 5 fold, at least 10 fold, at least 20 fold, at least 100 fold, at least 150 fold, or at least 200 fold above a reference level. In some embodiments, the reference level is a detected signal produced by contacting a control antibody with the glycans, or by contacting the antibody with a control protein.

A “variant” of a reference antibody refers to an antibody that typically differs from the reference antibody in one or more substitutions, deletions, additions, and/or insertions in the amino acid sequence of the heavy and/or light chain.

As used herein, the term “internalize,” or “internalization” refer to the phenomenon that an antibody molecule crosses the cell membrane and reaches the cytoplasm and/or the nucleus.

This application relates to tumor-targeting antibodies that bind to tumors and the binding is dependent on the activity one or more glycosyltransferases. In some embodiments, the glycan binder binds to a tumor-associated glycan. These antibodies are referred to glycan binders in this disclosure. The antibodies bind preferentially to tumor tissues relative to normal tissue and demonstrate cytotoxicity in various antibody-toxin or antibody-immunomodulating agent constructs towards tumor cells. Thus, these antibodies show therapeutic potential in treating cancers.

The glycan binders are also useful in detecting tumors suitable for treatment with a glycan binder in diagnostic applications.

### Glycans

Glycans are a complex group of monosaccharide or polysaccharide compounds comprised of diverse monosaccharide residues linked glycosidically. Glycosidic bonds are covalent linkages of carbohydrate moieties to another group which may or may not be another carbohydrate (e.g., C—, O—, N—). Through these glycosidic bonds, glycolipids, glycoproteins, and polysaccharides are formed. For purposes of this disclosure, the term “glycan,” refers to a polysaccharide or oligosaccharide, or the carbohydrate portion of all glycol conjugate such as glycoprotein, glycolipid, or glycopeptide, peptidoglycan, lipopolysaccharide or a proteoglycan. Glycans can be homo or heteropolymers of monosaccharide residues.

Forming glycosidic bonds typically require glycosyltransferases. Glycosyltransferases modify glycans in the ER and Golgi apparatus during their biosynthesis. Glycosyltransferases catalyze the transfer of saccharide moieties from an activated nucleotide sugar (also known as the “glycosyl donor”) to a nucleophilic glycosyl acceptor molecule, the nucleophile of which can be oxygen- carbon-, nitrogen-, or sulfur-based. Williams, GJ; Thorson, J S (2009). Natural product glycosyltransferases: properties and applications. Advances in Enzymology and Related Areas of Molecular Biology. Vol. 76. pp. 55-119. At least 200 glycosyltransferases have been identified to date. Cummings R D, Pierce J M. 2014. The challenge and promise of glycomics. Chem. Biol. 21:1-15.

### Glycosyltransferase

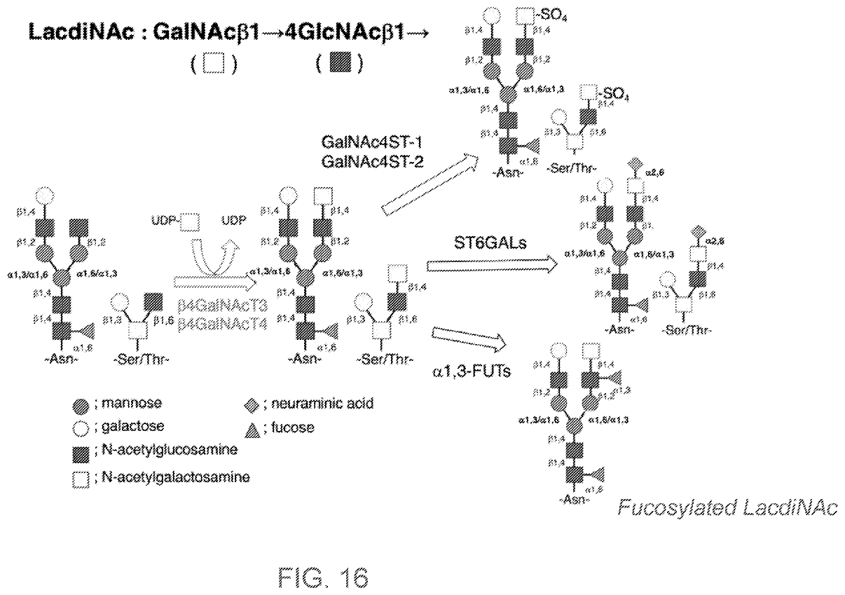

Glycosyltransferase B4GALNT3 (beta-1,4-N-acetyl-galactosaminyltransferase 3) is responsible for transfer of N-acetylgalactosamine (GalNAc) to N-acetylglucosamine-beta (GlcNAc) to form N, N′-diacetyllactosediamine with beta1,4-linkage. The product of this reaction is GalNAcbeta1,4GlcNAc (LacdiNAc). FIG. 16. 10.3390/biom12020195 See Ikehara Y et al. “Apical Golgi localization of N,N′-diacetyllactosediamine synthase, beta4GalNAc-T3, is responsible for LacdiNAc expression on gastric mucosa. Glycobiology”. 2006 September; 16(9):777-85. doi: 10.1093/glycob/cwl005. In humans, B4GALNT4 is reported to have similar activity to B4GALNT3. Examples of human B4GALNT3 and B4GALNT4 and their murine homologs include human B4GALNT3 (GenBank accession no. AB089940; UniProt ID: Q6L9W6; SEQ ID NO: 919); murine b4galnt3 (GenBank accession no. AB114826: UniProt ID: Q6L8S8); human B4GALNT4 (GenBank accession no. AB089939.1; UniProt ID: Q76KP1.1); and murine b4galnt4 (GenBank accession no. AB114827; UniProt ID: Q766D5.1).

Fucosyltransferases (FUTs) are glycosyltransferases involved in the synthesis of cell-surface antigens through catalyzing the transfer of fucose from GDP-fucose to acceptor sugars on biomolecules. The FUT family includes enzymes catalyzing α1,2-, α1,3/4-, α1,6- and protein O-FUT linkages. The α1,3/4-FUT group includes at least eight members: FUT3, FUT4, FUT5, FUT6, FUT7, FUT9, FUT10, and FUT11. FUT4 is a known to catalyze the alpha (1->3) linkage of beta-L-fucose to the GlcNAc of type 2 lactosmaines Gal-beta (1->4) GlcNAc. See FIG. 16 for the structure of fucosylated LacdiNAc. See Lowe J B et al. “Molecular cloning of a human fucosyltransferase gene that determines expression of the Lewis x and VIM-2 epitopes but not ELAM-1-dependent cell adhesion”. J Biol Chem. 1991 Sep. 15; 266(26):17467-77. Examples of human FUTs include FUT3 (GenBank accession no. NM_000149; UniProt ID: P21217); FUT4 (SEQ ID NO: 920) (GenBank accession no. NM_002033; UniProt ID: P22083); FUT5 (GenBank accession no. NM_002034.2; UniProt ID: Q11128); FUT6 (GenBank accession no. NM_000150.2; NM_001040701.1; UniProt ID: P51993.1); FUT7 (GenBank accession no. NM_004479; UniProt ID: Q11130); FUT9 (GenBank accession no. NM_006581; UniProt ID: Q9Y231); FUT10 (GenBank accession no. AJ582015; UniProt ID: Q6P4F1); and FUT11 (GenBank accession no. BC036037 UniProt ID: Q495W5). Examples of murine FUTs, include FUT4 (GenBank accession no. NM_010242; UniProt ID: B2RPT3); FUT7 (GenBank accession no. NM_013524; UniProt ID: Q11131); FUT9 (GenBank accession no. NM_010243; UniProt ID: 088819); FUT10 (GenBank accession no. AJ880009; UniProt ID: Q5F2L2); and FUT11 (GenBank accession no. NM_028428.2; UniProt ID: Q8BHC9).

### Glycan Binders

AB-006410 is an example of a glycan binder. AB-006410 was discovered in antibody repertoires generated by Immune Repertoire Capture® (IRC®) technology from plasmablast B cells isolated from a melanoma patient who had undergone treatment with a pembrolizumab. The patient exhibited an active anti-tumor immune response evidenced by tumor-selective antibodies derived from their plasmablast B cells. The IRC® technology and its use in antibody discovery is well known and disclosed in. e.g., WO 2012148497A2, the entire content of which is herein incorporated by reference.

Glycan binder AB-006410, and other glycan binders provided herein, bind to tumors and the binding is dependent on the expression of one or more specific glycosyltransferases in the tumor, that is, in the absence of expression of the one or more glycosyltransferases, the glycan binder will not show detectable binding to the tumor. In one embodiment, one of the one or more glycosyltransferases has N-acetyl-galactosaminyltransferase activity, such as the activity of B4GALNT3 or B4GALNT4. In one embodiment, the one of the one or more glycosyltransferases has fucosyltransferase activity, such as the activity of FUT3. FUT4, FUT5, FUT6, FUT7, FUT9, FUT10, and FUT11. In one embodiment, the one of the one or more glycosyltransferases has N-acetyl-galactosaminyltransferase activity, such as the activity of B4GALNT3 or fucosyltransferase activity, such as the activity of FUT4. In one embodiment, the one of the one or more glycosyltransferases has N-acetyl-galactosaminyltransferase activity, such as the activity of B4GALNT3 and one of the one or more glycosyltransferases has fucosyltransferase activity, such as the activity of FUT4.

In one embodiment, the binding of the glycan binder to the tumor is dependent on the expression one or more specific glycosyltransferases in the tumor, that is, in the absence of the expression of the one or more glycosyltransferases, the glycan binder will not show detectable binding to the tumor.

In one embodiment, each of the one or more glycosyltransferases is selected from the group consisting of B4GALNT3, B4GALNT4, FUT3, FUT4, FUT5, FUT6, FUT7, FUT9, FUT10, and FUT11. In one embodiment, the binding is dependent on the expression of the glycosyltransferase B4GALNT3 or the glycosyltransferase FUT4. In one embodiment, the binding is dependent on the expression of B4GALNT3 and FUT4. In one embodiment, the glycan binder binds to a tumor cell that expresses a glycan comprising GalNAcbeta1,4GlcNAc (LacdiNAc). In one embodiment, the glycan binder binds to a tumor cell that expresses GalNAcbeta1,4GlcNAc (LacdiNAc). In one embodiment, the LacdiNAc is fucosylated.

In some embodiments, a glycan binder disclosed herein binds to an extracellular, tumor-associated glycan. The presence of the glycan (or display of the glycan on the tumor cell surface) is dependent on the expression of one or more specific glycosyltransferases in the tumor, that is, in the absence of expression of the one or more glycosyltransferases, the glycan will not be produced in the tumor cells or will not be displayed on the surface of tumor cells. In one embodiment, each of the one or more glycosyltransferases is selected from the group consisting of B4GALNT3, B4GALNT4, FUT3, FUT4, FUT5, FUT6, FUT7, FUT9, FUT10, and FUT11. In one embodiment, the glycan is dependent on the expression of the glycosyltransferase B4GALNT3 or the glycosyltransferase FUT4. In one embodiment, the glycan is dependent on the expression of B4GALNT3 and FUT4. In one embodiment, the glycan binder binds to a glycan comprising GalNAcbeta1,4GlcNAc (LacdiNAc). In one embodiment, the glycan binder binds to GalNAcbeta1,4GlcNAc (LacdiNAc). In one embodiment, the glycan binder binds to a tumor cell that expresses a glycan comprising GalNAcbeta1,4GlcNAc (LacdiNAc). In one embodiment, the glycan binder binds to a tumor cell that expresses GalNAcbeta1,4GlcNAc (LacdiNAc). In one embodiment, the LacdiNAc is fucosylated.

### Structures of the Glycan Binders

In some embodiments, a glycan binder comprises an HCDR1 of any one of SEQ ID NO:1-14 and 113-212 or a variant HCDR1 in which 1, 2, 3, 4, or 5 amino acids are substituted relative to the sequence; an HCDR2 of any one of SEQ ID NO:15-28 and 213-312), or a variant HCDR2 in which 1, 2, 3, 4, or 5 amino acid is substituted relative to the sequence; and an HCDR3 of any one of SEQ ID NO:29-42 and 313-412 or a variant HCDR3 in which 1, 2, 3, 4, or 5 amino acids are substituted relative to the sequence. In some embodiments, a glycan binder comprises a light chain variable region comprising: an LCDR1 of any one of SEQ ID NO:43-56 and 413-512, or a variant LCDR1 in which 1, 2, 3, 4, or 5 amino acid is substituted relative to the sequence; an LCDR2 of any one of SEQ ID NO:57-70 and 513-612, or variant LCDR2 in which 1, 2, or 3 amino acid is substituted relative to the sequence; and an LCDR3 of any one of SEQ ID NO:71-84 and 613-712, or a variant LCDR3 in which 1, 2, 3, 4, or 5 amino acid is substituted relative to the sequence.

In some embodiments, a glycan binder comprises a heavy chain variable region comprising an amino acid sequence at least 70%, at least 75%, at least 80%, at least 85%, at least 90%, or at least 95% identical to any one of SEQ ID NO: 85-98 and 713-812. In some embodiments, a glycan binder comprises a light chain variable region comprising an amino acid sequence at least 70%, at least 75%, at least 80%, at least 85%, at least 90%, or at least 95% identical to any one of SEQ ID NO: 99-112 and 813-912.

In some embodiments, a glycan binder comprises a heavy chain variable (V_{H}) region and a light chain variable (V_{L}) region. The V_{H} region has an amino acid sequence that is at least 70% identical to SEQ ID NO:85; and comprises a CDR1 of SEQ ID NO:1, or the CDR1 of SEQ ID NO:1 in which 1, 2, 3, 4, or 5 amino acids are substituted; a CDR2 of SEQ ID NO:15, or the CDR2 of SEQ ID NO:15 in which 1, 2, 3, 4, or 5 amino acids are substituted; a CDR3 of SEQ ID NO: 29 or the CDR3 of SEQ ID NO: 29 in which 1, 2, 3, 4, or 5 are substituted. The V_{L} region has an amino acid sequence that is at least 70% identical to SEQ ID NO: 99, and comprises a CDR1 of SEQ ID NO: 43 or the CDR1 of SEQ ID NO: 43 in which 1, 2, 3, 4, or 5 amino acids are substituted; a CDR2 of SEQ ID NO: 57, or the CDR2 of SEQ ID NO: 57 in which 1, 2, 3, 4, or 5 amino acids are substituted; a CDR3 of SEQ ID NO: 71 or the CDR3 of SEQ ID NO: 71 in which 1, 2, 3, 4, or 5 are substituted.

In some embodiments, a glycan binder comprises: a V_{H} region comprising amino acid sequence SEQ ID NO:85 and a V_{L} region comprising amino acid sequence SEQ ID NO: 99.

In some embodiments, a glycan binder of the present invention has one, two, or three CDRs of a VL sequence (LCDRs) in Table 2. In some embodiments, the glycan binder has at least one mutation and no more than 10, 20, 30, 40 or 50 mutations in the VL amino acid sequences compared to a VL sequence set forth in Table 3. In some embodiments, the VL amino acid sequence may comprise 1, 2, 3, 4, 5, 6, 7, 8, 9, or 10 amino acid insertions or deletions compared to a VL sequence set forth in Table 3. In some embodiments, the VL amino acid sequence may comprise a deletion or insertion, e.g., a 1, 2, 3, 4, 5, 6, or 7 amino acid deletion or insertion, relative to a CDR sequence shown in Table 2. In some embodiments, the VL region comprises a CDR1 having 1 or 2 substitutions in relative to a CDR1 sequence shown in Table 2. In some embodiments, a CDR1 has 3, 4, or 5 substitutions relative to a CDR1 sequence shown in Table 2. In some embodiments, the VL region comprises a CDR2 that has 1 or 2; or 1, 2, or 3; substitutions relative to the CDR2 sequence shown in Table 2. In some embodiments, the VL region comprises a CDR3 that has 1, 2, or 3; or 1, 2, 3, or 4; substitutions relative to a CDR3 sequence shown in Table 2. In some embodiments, a glycan binder of the present disclosure comprises a CDR1, CDR2, and CDR3, each having at least 70% identity to a CDR1, CDR2, and CDR3 as shown in Table 2. In some embodiments, a glycan binder of the present invention comprises a CDR1, CDR2, and CDR3, each having at least 80% identity to an LCDR1, LCDR2, and LCDR3 as shown in Table 2. In some embodiments, glycan binder of the present invention comprises an LCDR1, LCDR2, and LCDR3 as shown in Table 2. In some embodiments, a glycan binder of the present invention comprises a CDR1, CDR2, and CDR3 of an antibody designated as AB-006410, AB-011110, AB-011111, AB-011366, AB-011367, AB-011368, AB-011369, AB-011370, AB-011371, AB-011372, AB-011373, AB-011374, AB-011375, AB-011376, AB-011622, AB-011623. AB-011624, AB-011625, AB-011626, AB-011627, AB-011628, AB-011781, AB-011782, AB-011783, AB-011784, AB-011785, AB-011786, AB-011787, AB-011788, AB-011789, AB-011790. AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, or AB-011873.

In some embodiments, a glycan binder of the present invention has one, two, or three CDRs of a VH sequence (HCDRs) in Table 1. In some embodiments, the glycan binder has at least one mutation and no more than 10, 20, 30, 40 or 50 mutations in the VH amino acid sequences compared to a VH sequence set forth in Table 3. In some embodiments, the VH amino acid sequence may comprise 1, 2, 3, 4, 5, 6, 7, 8, 9, or 10 amino acid insertions or deletions compared to a VH sequence set forth in Table 3. In some embodiments, the VH amino acid sequence may comprise a deletion or insertion, e.g., a 1, 2, 3, 4, 5, 6, or 7 amino acid deletion or insertion, relative to a CDR sequence shown in Table 1. In some embodiments, the VH region comprises an HCDR1 having 1 or 2 substitutions in relative to an HCDR1 sequence shown in Table 1. In some embodiments, an HCDR1 has 3, 4, or 5 substitutions relative to a CDR1 sequence shown in Table 1. In some embodiments, the VH region comprises an HCDR2 that has 1 or 2; or 1, 2, or 3; substitutions relative to the HCDR2 sequence shown in Table 1. In some embodiments, the VH region comprises an HCDR3 that has 1, 2, or 3; or 1, 2, 3, or 4; substitutions relative to a CDR3 sequence shown in Table 1. In some embodiments, a glycan binder of the present disclosure comprises an HCDR1, HCDR2, and HCDR3, each having at least 70% identity to an HCDR1, HCDR2, and HCDR3 as shown in Table 1. In some embodiments, a glycan binder of the present invention comprises a CDR1, CDR2, and CDR3, each having at least 80% identity to an HCDR1, HCDR2, and HCDR3 as shown in Table 1. In some embodiments, a glycan binder of the present invention comprises an HCDR1, HCDR2, and HCDR3 as shown in Table 1. In some embodiments, a glycan binder of the present invention comprises an HCDR1, HCDR2, and HCDR3 of an antibody designated as AB-006410, AB-011110, AB-011111, AB-011366, AB-011367, AB-011368, AB-011369, AB-011370, AB-011371, AB-011372, AB-011373, AB-011374, AB-011375, AB-011376, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, or AB-011873.

In some embodiments, a glycan binder disclosed herein comprises a V_{H} region sequence and a V_{L} sequence shown in Table 3. In some embodiments, the glycan binders disclosed herein comprises a heavy chain variable region and a light chain variable region of an antibody designated as AB-006410 or other glycan binder disclosed in Table 3. The variant comprises a heavy chain variable region having a sequence that is at least 70%, at least 75%, at least 80%, at least 85%, at least 90%, at least 95% identical to that of the corresponding heavy chain variable region and a light chain variable region having a sequence that is at least 70%, at least 75%, at least 80%, at least 85%, at least 90%, at least 95% identical to the corresponding light chain variable region. For example, the variant may comprise a V_{H} that is at least 70%, at least 75%, at least 80%, at least 85%, at least 90%, at least 95% identical to that of AB-006410, AB-011110, AB-011111, AB-011366, AB-011367, AB-011368, AB-011369, AB-011370, AB-011371, AB-011372, AB-011373, AB-011374, AB-011375, AB-011376, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, or AB-011873; and/or a V_{L} that is at least 70%, at least 75%, at least 80%, at least 85%, at least 90%, at least 95% identical to any one of AB-006410, AB-011110, AB-011111, AB-011366, AB-011367. AB-011368, AB-011369, AB-011370, AB-011371, AB-011372, AB-011373, AB-011374, AB-011375, AB-011376, AB-011622, AB-011623, AB-011624, AB-011625, AB-011626, AB-011627. AB-011628, AB-011781, AB-011782, AB-011783, AB-011784, AB-011785, AB-011786, AB-011787, AB-011788, AB-011789, AB-011790, AB-011791, AB-011792, AB-011793, AB-011794. AB-011795, AB-011796, AB-011797, AB-011798, AB-011799, AB-011800, AB-011801, AB-011802, AB-011803, AB-011804, AB-011805, AB-011806, AB-011807, AB-011808, AB-011809. AB-011810, AB-011811, AB-011812, AB-011813, AB-011814, AB-011815, AB-011816, AB-011817, AB-011818, AB-011819, AB-011820, AB-011821, AB-011822, AB-011823, AB-011824. AB-011825, AB-011826, AB-011827, AB-011828, AB-011829, AB-011830, AB-011831, AB-011832, AB-011833, AB-011834, AB-011835, AB-011836, AB-011837, AB-011838, AB-011839. AB-011840, AB-011841, AB-011842, AB-011843, AB-011844, AB-011845, AB-011846, AB-011847, AB-011848, AB-011849, AB-011850, AB-011851, AB-011852, AB-011853, AB-011854. AB-011855, AB-011856, AB-011857, AB-011858, AB-011859, AB-011860, AB-011861, AB-011862, AB-011863, AB-011864, AB-011865, AB-011866, AB-011867, AB-011868, AB-011869. AB-011870, AB-011871, AB-011872, or AB-011873.

In some embodiments, a glycan binder disclosed herein comprises an HCDR1, HCDR2, HCDR3, LCDR1, LCDR2, and LCDR3 of AB-006410. Exemplary glycan binders include AB-006410, AB-011110, AB-011111, AB-011366, AB-011367, AB-011368, AB-011369, AB-011370, AB-011371. AB-011372, AB-011373, AB-011374, AB-011375, AB-011376, AB-011622, AB-011623, AB-011624, AB-011625, AB-011626, AB-011627, AB-011628, AB-011781, AB-011782, AB-011783. AB-011784, AB-011785, AB-011786, AB-011787, AB-011788, AB-011789, AB-011790, AB-011791, AB-011792, AB-011793, AB-011794, AB-011795, AB-011796, AB-011797, AB-011798. AB-011799, AB-011800, AB-011801, AB-011802, AB-011803, AB-011804, AB-011805, AB-011806, AB-011807, AB-011808, AB-011809, AB-011810, AB-011811, AB-011812, AB-011813. AB-011814, AB-011815, AB-011816, AB-011817, AB-011818, AB-011819, AB-011820, AB-011821, AB-011822, AB-011823, AB-011824, AB-011825, AB-011826, AB-011827, AB-011828. AB-011829, AB-011830, AB-011831, AB-011832, AB-011833, AB-011834, AB-011835, AB-011836, AB-011837, AB-011838, AB-011839, AB-011840, AB-011841, AB-011842, AB-011843. AB-011844, AB-011845, AB-011846, AB-011847, AB-011848, AB-011849, AB-011850, AB-011851, AB-011852, AB-011853, AB-011854, AB-011855, AB-011856, AB-011857, AB-011858. AB-011859, AB-011860, AB-011861, AB-011862, AB-011863, AB-011864, AB-011865, AB-011866, AB-011867, AB-011868, AB-011869, AB-011870, AB-011871, AB-011872, or AB-011873. The structural information of these glycan binders are shown in Tables 1-3.

TABLE 1
Heavy chain CDR sequences

| ID | HCDR1 | HCDR2 | HCDR3 |
|---|---|---|---|
| AB- | SFTFSNAWVN | RLRSDTEGWTKDYAPHLK | NTGYWPQSAFLTNRAYAM |
| 006410 | (SEQ ID NO: 1) | D | DV |
| | | (SEQ ID NO: 15) | (SEQ ID NO: 29) |
| AB- | SFTFSNAWVN | RLRSDTEGWTKDYAPHLK | NTGYWPQSAFLTNRAYAM |
| 011110 | (SEQ ID NO: 2) | D | DV |
| | | (SEQ ID NO: 16) | (SEQ ID NO: 30) |
| AB- | SFTFSNAWVN | RLRSDTEGWTKDYAPHLK | NTGYWPQSAFLTNRAYAM |
| 011111 | (SEQ ID NO: 3) | D | DV |
| | | (SEQ ID NO: 17) | (SEQ ID NO: 31) |
| AB- | TFTFNNAWV | RLRSDTEHWAKDYAAHM | NTGYWPQSVFLTNRAYAM |
| 011366 | N | KG | DV |
| | (SEQ ID NO: 4) | (SEQ ID NO: 18) | (SEQ ID NO: 32) |
| AB- | TFTFNNAWV | RLRSDTEHWAKDYAAHM | NTGYWPQSVFLTNRAYAM |
| 011367 | N | KG | DV |
| | (SEQ ID NO: 5) | (SEQ ID NO: 19) | (SEQ ID NO: 33) |
| AB- | SFTFSNAWVN | RLRSDTEGWTKDYAPHLK | NTGYWPQSAFLTNRAYAM |
| 011368 | (SEQ ID NO: 6) | D | DV |
| | | (SEQ ID NO: 20) | (SEQ ID NO: 34) |
| AB- | SFTFSNAWVN | RLRSDTEGWTKDYAPHLK | NTGYWPQSAFLTNRAYAM |
| 011369 | (SEQ ID NO: 7) | D | DV |
| | | (SEQ ID NO: 21) | (SEQ ID NO: 35) |
| AB- | SFTFSNAWVN | RLRSDTEGWTKDYAPHLK | NTGYWPQSAFLTNRAYAM |
| 011370 | (SEQ ID NO: 8) | D | DV |
| | | (SEQ ID NO: 22) | (SEQ ID NO: 36) |
| AB- | SFTFSNAWVN | RLRSDTEGWTKDYAPHLK | NTGYWPQSAFLTNRAYAM |
| 011371 | (SEQ ID NO: 9) | D | DV |
| | | (SEQ ID NO: 23) | (SEQ ID NO: 37) |
| AB- | SFTFSNAWVN | RLRSDTEGWTKDYAPHLK | NTGYWPQSAFLTNRAYAM |
| 011372 | (SEQ ID NO: | D | DV |
| | 10) | (SEQ ID NO: 24) | (SEQ ID NO: 38) |
| AB- | SFTFSNAWVN | RLRSDTEGWTKDYAPHLK | NTGYWPQSAFLTNRAYAM |
| 011373 | (SEQ ID NO: | D | DV |
| | 11) | (SEQ ID NO: 25) | (SEQ ID NO: 39) |
| AB- | SFTFSNAWVN | RLRSDTEGWTKDYAPHLK | NTGYWPQSAFLTNRAYAM |
| 011374 | (SEQ ID NO: | D | DV |
| | 12) | (SEQ ID NO: 26) | (SEQ ID NO: 40) |
| AB- | SFTFSNAWVN | RLRSDTEGWTKDYAPHLK | NTGYWPQSAFLTNRAYAM |
| 011375 | (SEQ ID NO: | D | DV |
| | 13) | (SEQ ID NO: 27) | (SEQ ID NO: 41) |
| AB- | SFTFSNAWVN | RLRSDTEGWTKDYAPHLK | NTGYWPQSAFLTNRAYAM |
| 011376 | (SEQ ID NO: | D | DV |
| | 14) | (SEQ ID NO: 28) | (SEQ ID NO: 42) |
| AB- | SFTFSNAWVN | RLRSDTEGWTKDYAPHLK | NTGYWPQSAFLTNRAYAM |
| 011622 | (SEQ ID NO: | D | DV |
| | 113) | (SEQ ID NO: 213) | (SEQ ID NO: 313) |
| AB- | SFTFSNAWVN | RLRSDTEGWTKDYAPHLK | NTGYWPQSAFLTNRAYAM |
| 011623 | (SEQ ID NO: | D | DV |
| | 114) | (SEQ ID NO: 214) | (SEQ ID NO: 314) |
| AB- | SFTFSNAWVN | RLRSDTEGWTKDYAPHLK | NTGYWPQSAFLTNRAYAM |
| 011624 | (SEQ ID NO: | D | DV |
| | 115) | (SEQ ID NO: 215) | (SEQ ID NO: 315) |
| AB- | SFTFSNAWVN | RLRSDTEGWTKDYAPHLK | NTGYWPQSAFLTNRAYAM |
| 011625 | (SEQ ID NO: | D | DV |
| | 116) | (SEQ ID NO: 216) | (SEQ ID NO: 316) |
| AB- | SFTFSNAWVN | RLRSDTEGWTKDYAPHLK | NTGYWPQSAFLTNRAYAM |
| 011626 | (SEQ ID NO: | D | DV |
| | 117) | (SEQ ID NO: 217) | (SEQ ID NO: 317) |
| AB- | SFTFSNAWVN | RLRSDTEGWTKDYAPHLK | NTGYWPQSAFLTNRAYAM |
| 011627 | (SEQ ID NO: | D | DV |
| | 118) | (SEQ ID NO: 218) | (SEQ ID NO: 318) |
| AB- | TFTFNNAWVN | RLRSDTEHWAKDYAAHM | NTGYWPQSVFLTNRAYAM |
| 011628 | (SEQ ID NO: | KG | DV |
| | 119) | (SEQ ID NO: 219) | (SEQ ID NO: 319) |
| AB- | SFTFDNAWVN | RLRSDTEGWTKDYAPHLK | NTGYWPQSAFLTNRAYAM |
| 011781 | (SEQ ID NO: | D | DV |
| | 120) | (SEQ ID NO: 220) | (SEQ ID NO: 320) |
| AB- | SFTFSAAWVN | RLRSDTEGWTKDYAPHLK | NTGYWPQSAFLTNRAYAM |
| 011782 | (SEQ ID NO: | D | DV |
| | 121) | (SEQ ID NO: 221) | (SEQ ID NO: 321) |
| AB- | SFTFSNAEVN | RLRSDTEGWTKDYAPHLK | NTGYWPQSAFLTNRAYAM |
| 011783 | (SEQ ID NO: | D | DV |
| | 122) | (SEQ ID NO: 222) | (SEQ ID NO: 322) |
| AB- | SFTFSNALVN | RLRSDTEGWTKDYAPHLK | NTGYWPQSAFLTNRAYAM |
| 011784 | (SEQ ID NO: | D | DV |
| | 123) | (SEQ ID NO: 223) | (SEQ ID NO: 323) |
| AB- | SFTFSNAWVN | ELRSDTEGWTKDYAPHLK | NTGYWPQSAFLTNRAYAM |
| 011785 | (SEQ ID NO: | D | DV |
| | 124) | (SEQ ID NO: 224) | (SEQ ID NO: 324) |
| AB- | SFTFSNAWVN | RLYSDTEGWTKDYAPHLK | NTGYWPQSAFLTNRAYAM |
| 011786 | (SEQ ID NO: | D | DV |
| | 125) | (SEQ ID NO: 225) | (SEQ ID NO: 325) |
| AB- | SFTFSNAWVN | RLASDTEGWTKDYAPHLK | NTGYWPQSAFLTNRAYAM |
| 011787 | (SEQ ID NO: | D | DV |
| | 126) | (SEQ ID NO: 226) | (SEQ ID NO: 326) |
| AB- | SFTFSNAWVN | RLRSKTEGWTKDYAPHLK | NTGYWPQSAFLTNRAYAM |
| 011788 | (SEQ ID NO: | D | DV |
| | 127) | (SEQ ID NO: 227) | (SEQ ID NO: 327) |
| AB- | SFTFSNAWVN | RLRSYTEGWTKDYAPHLK | NTGYWPQSAFLTNRAYAM |
| 011789 | (SEQ ID NO: | D | DV |
| | 128) | (SEQ ID NO: 228) | (SEQ ID NO: 328) |
| AB- | SFTFSNAWVN | RLRSDREGWTKDYAPHLK | NTGYWPQSAFLTNRAYAM |
| 011790 | (SEQ ID NO: | D | DV |
| | 129) | (SEQ ID NO: 229) | (SEQ ID NO: 329) |
| AB- | SFTFSNAWVN | RLRSDEEGWTKDYAPHLK | NTGYWPQSAFLTNRAYAM |
| 011791 | (SEQ ID NO: | D | DV |
| | 130) | (SEQ ID NO: 230) | (SEQ ID NO: 330) |
| AB- | SFTFSNAWVN | RLRSDYEGWTKDYAPHLK | NTGYWPQSAFLTNRAYAM |
| 011792 | (SEQ ID NO: | D | DV |
| | 131) | (SEQ ID NO: 231) | (SEQ ID NO: 331) |
| AB- | SFTFSNAWVN | RLRSDTKGWTKDYAPHLK | NTGYWPQSAFLTNRAYAM |
| 011793 | (SEQ ID NO: | D | DV |
| | 132) | (SEQ ID NO: 232) | (SEQ ID NO: 332) |
| AB- | SFTFSNAWVN | RLRSDTEYWTKDYAPHLK | NTGYWPQSAFLTNRAYAM |
| 011794 | (SEQ ID NO: | D | DV |
| | 133) | (SEQ ID NO: 233) | (SEQ ID NO: 333) |
| AB- | SFTFSNAWVN | RLRSDTEHWTKDYAPHLK | NTGYWPQSAFLTNRAYAM |
| 011795 | (SEQ ID NO: | D | DV |
| | 134) | (SEQ ID NO: 234) | (SEQ ID NO: 334) |
| AB- | SFTFSNAWVN | RLRSDTEGSTKDYAPHLK | NTGYWPQSAFLTNRAYAM |
| 011796 | (SEQ ID NO: | D | DV |
| | 135) | (SEQ ID NO: 235) | (SEQ ID NO: 335) |
| AB- | SFTFSNAWVN | RLRSDTEGWYKDYAPHLK | NTGYWPQSAFLTNRAYAM |
| 011797 | (SEQ ID NO: | D | DV |
| | 136) | (SEQ ID NO: 236) | (SEQ ID NO: 336) |
| AB- | SFTFSNAWVN | RLRSDTEGWEKDY APHLK | NTGYWPQSAFLTNRAYAM |
| 011798 | (SEQ ID NO: | D | DV |
| | 137) | (SEQ ID NO: 237) | (SEQ ID NO: 337) |
| AB- | SFTFSNAWVN | RLRSDTEGWTADYAPHLK | NTGYWPQSAFLTNRAYAM |
| 011799 | (SEQ ID NO: | D | DV |
| | 138) | (SEQ ID NO: 238) | (SEQ ID NO: 338) |
| AB- | SFTFSNAWVN | RLRSDTEGWTARYAPHLK | NTGYWPQSAFLTNRAYAM |
| 011800 | (SEQ ID NO: | D | DV |
| | 139) | (SEQ ID NO: 239) | (SEQ ID NO: 339) |
| AB- | SFTFSNAWVN | RLRSDTEGWTKDYAPHLA | NTGYWPQSAFLTNRAYAM |
| 011801 | (SEQ ID NO: | D | DV |
| | 140) | (SEQ ID NO: 240) | (SEQ ID NO: 340) |
| AB- | SFTFSNAWVN | RLRSDTEGWTKDYAPHLY | NTGYWPQSAFLTNRAYAM |
| 011802 | (SEQ ID NO: | D | DV |
| | 141) | (SEQ ID NO: 241) | (SEQ ID NO: 341) |
| AB- | SFTFSNAWVN | RLRSDTEGWTKDYAPHLK | NTGAWPQSAFLTNRAYAM |
| 011803 | (SEQ ID NO: | D | DV |
| | 142) | (SEQ ID NO: 242) | (SEQ ID NO: 342) |
| AB- | SFTFSNAWVN | RLRSDTEGWTKDYAPHLK | NTGRWPQSAFLTNRAYAM |
| 011804 | (SEQ ID NO: | D | DV |
| | 143) | (SEQ ID NO: 243) | (SEQ ID NO: 343) |
| AB- | SFTFSNAWVN | RLRSDTEGWTKDYAPHLK | NTGY APQSAFLTNRAYAM |
| 011805 | (SEQ ID NO: | D | DV |
| | 144) | (SEQ ID NO: 244) | (SEQ ID NO: 344) |
| AB- | SFTFSNAWVN | RLRSDTEGWTKDYAPHLK | NTGYEPQSAFLTNRAYAMD |
| 011806 | (SEQ ID NO: | D | V |
| | 145) | (SEQ ID NO: 245) | (SEQ ID NO: 345) |
| AB- | SFTFSNAWVN | RLRSDTEGWTKDYAPHLK | NTGYWPASAFLTNRAYAM |
| 011807 | (SEQ ID NO: | D | DV |
| | 146) | (SEQ ID NO: 246) | (SEQ ID NO: 346) |
| AB- | SFTFSNAWVN | RLRSDTEGWTKDYAPHLK | NTGYWPYSAFLTNRAYAM |
| 011808 | (SEQ ID NO: | D | DV |
| | 147) | (SEQ ID NO: 247) | (SEQ ID NO: 347) |
| AB- | SFTFSNAWVN | RLRSDTEGWTKDYAPHLK | NTGYWPQYAFLTNRAYAM |
| 011809 | (SEQ ID NO: | D | DV |
| | 148 | (SEQ ID NO: 248) | (SEQ ID NO: 348) |
| AB- | SFTFSNAWVN | RLRSDTEGWTKDYAPHLK | NTGYWPQEAFLTNRAYAM |
| 011810 | (SEQ ID NO: | D | DV |
| | 149 | (SEQ ID NO: 249) | (SEQ ID NO: 349) |
| AB- | SFTFSNAWVN | RLRSDTEGWTKDYAPHLK | NTGYWPQSYFLTNRAYAM |
| 011811 | (SEQ ID NO: | D | DV |
| | 150) | (SEQ ID NO: 250) | (SEQ ID NO: 350) |
| AB- | SFTFSNAWVN | RLRSDTEGWTKDYAPHLK | NTGYWPQSKFLTNRAYAM |
| 011812 | (SEQ ID NO: | D | DV |
| | 151) | (SEQ ID NO: 251) | (SEQ ID NO: 351) |
| AB- | SFTFSNAWVN | RLRSDTEGWTKDYAPHLK | NTGYWPQSAALTNRAYAM |
| 011813 | (SEQ ID NO: | D | DV |
| | 152) | (SEQ ID NO: 252) | (SEQ ID NO: 352) |
| AB- | SFTFSNAWVN | RLRSDTEGWTKDYAPHLK | NTGYWPQSAQLTNRAYAM |
| 011814 | (SEQ ID NO: | D | DV |
| | 153) | (SEQ ID NO: 253) | (SEQ ID NO: 353) |
| AB- | SFTFSNAWVN | RLRSDTEGWTKDYAPHLK | NTGYWPQSAFATNRAYAM |
| 011815 | (SEQ ID NO: | D | DV |
| | 154) | (SEQ ID NO: 254) | (SEQ ID NO: 354) |
| AB- | SFTFSNAWVN | RLRSDTEGWTKDYAPHLK | NTGYWPQSAFLENRAYAM |
| 011816 | (SEQ ID NO: | D | DV |
| | 155) | (SEQ ID NO: 255) | (SEQ ID NO: 355) |
| AB- | SFTFSNAWVN | RLRSDTEGWTKDYAPHLK | NTGYWPQSAFLYNRAYAM |
| 011817 | (SEQ ID NO: | D | DV |
| | 156) | (SEQ ID NO: 256) | (SEQ ID NO: 356) |
| AB- | SFTFSNAWVN | RLRSDTEGWTKDYAPHLK | NTGYWPQSAFLTQRAYAM |
| 011818 | (SEQ ID NO: | D | DV |
| | 157) | (SEQ ID NO: 257) | (SEQ ID NO: 357) |
| AB- | SFTFSNAWVN | RLRSDTEGWTKDYAPHLK | NTGYWPQSAFLTARAYAM |
| 011819 | (SEQ ID NO: | D | DV |
| | 158) | (SEQ ID NO: 258) | (SEQ ID NO: 358) |
| AB- | SFTFSNAWVN | RLRSDTEGWTKDYAPHLK | NTGYWPQSAFLTNEAYAM |
| 011820 | (SEQ ID NO: | D | DV |
| | 159) | (SEQ ID NO: 259) | (SEQ ID NO: 359) |
| AB- | SFTFSNAWVN | RLRSDTEGWTKDYAPHLK | NTGYWPQSAFLTNSAYAM |
| 011821 | (SEQ ID NO: | D | DV |
| | 160) | (SEQ ID NO: 260) | (SEQ ID NO: 360) |
| AB- | SFTFSNAWVN | RLRSDTEGWTKDYAPHLK | NTGYWPQSAFLTNRYYAM |
| 011822 | (SEQ ID NO: | D | DV |
| | 161) | (SEQ ID NO: 261) | (SEQ ID NO: 361) |
| AB- | SFTFSNAWVN | RLRSDTEGWTKDYAPHLK | NTGYWPQSAFLTNRNYAM |
| 011823 | (SEQ ID NO: | D | DV |
| | 162) | (SEQ ID NO: 262) | (SEQ ID NO: 362) |
| AB- | SFTFSNAWVN | RLRSDTEGWTKDYAPHLK | NTGYWPQSAFLTNRAAAM |
| 011824 | (SEQ ID NO: | D | DV |
| | 163) | (SEQ ID NO: 263) | (SEQ ID NO: 363) |
| AB- | SFTFSNAWVN | RLRSDTEGWTKDYAPHLK | NTGYWPQSAFLTNRAKAM |
| 011825 | (SEQ ID NO: | D | DV |
| | 164) | (SEQ ID NO: 264) | (SEQ ID NO: 364) |
| AB- | SFTFSNAWVN | RLRSDTEGWTKDYAPHLK | NTGYWPQSAFLTNRAYAM |
| 011826 | (SEQ ID NO: | D | DV |
| | 165) | (SEQ ID NO: 265) | (SEQ ID NO: 365) |
| AB- | SFTFSNAWVN | RLRSDTEGWTKDYAPHLK | NTGYWPQSAFLTNRAYAM |
| 011827 | (SEQ ID NO: | D | DV |
| | 166) | (SEQ ID NO: 266) | (SEQ ID NO: 366) |
| AB- | SFTFSNAWVN | RLRSDTEGWTKDYAPHLK | NTGYWPQSAFLTNRAYAM |
| 011828 | (SEQ ID NO: | D | DV |
| | 167) | (SEQ ID NO: 267) | (SEQ ID NO: 367) |
| AB- | SFTFSNAWVN | RLRSDTEGWTKDYAPHLK | NTGYWPQSAFLTNRAYAM |
| 011829 | (SEQ ID NO: | D | DV |
| | 168) | (SEQ ID NO: 268) | (SEQ ID NO: 368) |
| AB- | SFTFSNAWVN | RLRSDTEGWTKDYAPHLK | NTGYWPQSAFLTNRAYAM |
| 011830 | (SEQ ID NO: | D | DV |
| | 169) | (SEQ ID NO: 269) | (SEQ ID NO: 369) |
| AB- | SFTFSNAWVN | RLRSDTEGWTKDYAPHLK | NTGYWPQSAFLTNRAYAM |
| 011831 | (SEQ ID NO: | D | DV |
| | 170) | (SEQ ID NO: 270) | (SEQ ID NO: 370) |
| AB- | SFTFSNAWVN | RLRSDTEGWTKDYAPHLK | NTGYWPQSAFLTNRAYAM |
| 011832 | (SEQ ID NO: | D | DV |
| | 171) | (SEQ ID NO: 271) | (SEQ ID NO: 371) |
| AB- | SFTFSNAWVN | RLRSDTEGWTKDYAPHLK | NTGYWPQSAFLTNRAYAM |
| 011833 | (SEQ ID NO: | D | DV |
| | 172) | (SEQ ID NO: 272) | (SEQ ID NO: 372) |
| AB- | SFTFSNAWVN | RLRSDTEGWTKDYAPHLK | NTGYWPQSAFLTNRAYAM |
| 011834 | (SEQ ID NO: | D | DV |
| | 173) | (SEQ ID NO: 273) | (SEQ ID NO: 373) |
| AB- | SFTFSNAWVN | RLRSDTEGWTKDYAPHLK | NTGYWPQSAFLTNRAYAM |
| 011835 | (SEQ ID NO: | D | DV |
| | 174) | (SEQ ID NO: 274) | (SEQ ID NO: 374) |
| AB- | SFTFSNAWVN | RLRSDTEGWTKDYAPHLK | NTGYWPQSAFLTNRAYAM |
| 011836 | (SEQ ID NO: | D | DV |
| | 175) | (SEQ ID NO: 275) | (SEQ ID NO: 375) |
| AB- | SFTFSNAWVN | RLRSDTEGWTKDYAPHLK | NTGYWPQSAFLTNRAYAM |
| 011837 | (SEQ ID NO: | D | DV |
| | 176) | (SEQ ID NO: 276) | (SEQ ID NO: 376) |
| AB- | SFTFSNAWVN | RLRSDTEGWTKDYAPHLK | NTGYWPQSAFLTNRAYAM |
| 011838 | (SEQ ID NO: | D | DV |
| | 177) | (SEQ ID NO: 277) | (SEQ ID NO: 377) |
| AB- | SFTFSNAWVN | RLRSDTEGWTKDYAPHLK | NTGYWPQSAFLTNRAYAM |
| 011839 | (SEQ ID NO: | D | DV |
| | 178) | (SEQ ID NO: 278) | (SEQ ID NO: 378) |
| AB- | SFTFSNAWVN | RLRSDTEGWTKDYAPHLK | NTGYWPQSAFLTNRAYAM |
| 011840 | (SEQ ID NO: | D | DV |
| | 179) | (SEQ ID NO: 279) | (SEQ ID NO: 379) |
| AB- | SFTFSNAWVN | RLRSDTEGWTKDYAPHLK | NTGYWPQSAFLTNRAYAM |
| 011841 | (SEQ ID NO: | D | DV |
| | 180) | (SEQ ID NO: 280) | (SEQ ID NO: 380) |
| AB- | SFTFSNAWVN | RLRSDTEGWTKDYAPHLK | NTGYWPQSAFLTNRAYAM |
| 011842 | (SEQ ID NO: | D | DV |
| | 181) | (SEQ ID NO: 281) | (SEQ ID NO: 381) |
| AB- | SFTFSNAWVN | RLRSDTEGWTKDYAPHLK | NTGYWPQSAFLTNRAYAM |
| 011843 | (SEQ ID NO: | D | DV |
| | 182) | (SEQ ID NO: 282) | (SEQ ID NO: 382) |
| AB- | SFTFSNAWVN | RLRSDTEGWTKDYAPHLK | NTGYWPQSAFLTNRAYAM |
| 011844 | (SEQ ID NO: | D | DV |
| | 183) | (SEQ ID NO: 283) | (SEQ ID NO: 383) |
| AB- | SFTFSNAWVN | RLRSDTEGWTKDYAPHLK | NTGYWPQSAFLTNRAYAM |
| 011845 | (SEQ ID NO: | D | DV |
| | 184) | (SEQ ID NO: 284) | (SEQ ID NO: 384) |
| AB- | SFTFSNAWVN | RLRSDTEGWTKDYAPHLK | NTGYWPQSAFLTNRAYAM |
| 011846 | (SEQ ID NO: | D | DV |
| | 185) | (SEQ ID NO: 285) | (SEQ ID NO: 385) |
| AB- | SFTFSNAWVN | RLRSDTEGWTKDYAPHLK | NTGYWPQSAFLTNRAYAM |
| 011847 | (SEQ ID NO: | D | DV |
| | 186) | (SEQ ID NO: 286) | (SEQ ID NO: 386) |
| AB- | SFTFSNAWVN | RLRSDTEGWTKDYAPHLK | NTGYWPQSAFLTNRAYAM |
| 011848 | (SEQ ID NO: | D | DV |
| | 187) | (SEQ ID NO: 287) | (SEQ ID NO: 387) |
| AB- | SFTFSNAWVN | RLRSDTEGWTKDYAPHLK | NTGYWPQSAFLTNRAYAM |
| 011849 | (SEQ ID NO: | D | DV |
| | 188) | (SEQ ID NO: 288) | (SEQ ID NO: 388) |
| AB- | SFTFSNAWVN | RLRSDTEGWTKDYAPHLK | NTGYWPQSAFLTNRAYAM |
| 011850 | (SEQ ID NO: | D | DV |
| | 189) | (SEQ ID NO: 289) | (SEQ ID NO: 389) |
| AB- | SFTFSNAWVN | RLRSDTEGWTKDYAPHLK | NTGYWPQSAFLTNRAYAM |
| 011851 | (SEQ ID NO: | D | DV |
| | 190) | (SEQ ID NO: 290) | (SEQ ID NO: 390) |
| AB- | SFTFSNAWVN | RLRSDTEGWTKDYAPHLK | NTGYWPQSAFLTNRAYAM |
| 011852 | (SEQ ID NO: | D | DV |
| | 191) | (SEQ ID NO: 291) | (SEQ ID NO: 391) |
| AB- | SFTFSNAWVN | RLRSDTEGWTKDYAPHLK | NTGYWPQSAFLTNRAYAM |
| 011853 | (SEQ ID NO: | D | DV |
| | 192) | (SEQ ID NO: 292) | (SEQ ID NO: 392) |
| AB- | SFTFSNAWVN | RLRSDTEGWTKDYAPHLK | NTGYWPQSAFLTNRAYAM |
| 011854 | (SEQ ID NO: | D | DV |
| | 193) | (SEQ ID NO: 293) | (SEQ ID NO: 393) |
| AB- | SFTFSNAWVN | RLRSDTEGWTKDYAPHLK | NTGYWPQSAFLTNRAYAM |
| 011855 | (SEQ ID NO: | D | DV |
| | 194) | (SEQ ID NO: 294) | (SEQ ID NO: 394) |
| AB- | SFTFSNAWVN | RLRSDTEGWTKDYAPHLK | NTGYWPQSAFLTNRAYAM |
| 011856 | (SEQ ID NO: | D | DV |
| | 195) | (SEQ ID NO: 295) | (SEQ ID NO: 395) |
| AB- | SFTFSNAWVN | RLRSDTEGWTKDYAPHLK | NTGYWPQSAFLTNRAYAM |
| 011857 | (SEQ ID NO: | D | DV |
| | 196) | (SEQ ID NO: 296) | (SEQ ID NO: 396) |
| AB- | SFTFSNAWVN | RLRSDTEGWTKDYAPHLK | NTGYWPQSAFLTNRAYAM |
| 011858 | (SEQ ID NO: | D | DV |
| | 197) | (SEQ ID NO: 297) | (SEQ ID NO: 397) |
| AB- | SFTFSNAWVN | RLRSDTEGWTKDYAPHLK | NTGYWPQSAFLTNRAYAM |
| 011859 | (SEQ ID NO: | D | DV |
| | 198) | (SEQ ID NO: 298) | (SEQ ID NO: 398) |
| AB- | TFTFNNAWVN | RLQSDTEHWAKDYAAHM | NTGYWPQSVFLTNRAYAM |
| 011860 | (SEQ ID NO: | KG | DV |
| | 199) | (SEQ ID NO: 299) | (SEQ ID NO: 399) |
| AB- | TFTFNNAWVN | RLRSNTEHWAKDYAAHM | NTGYWPQSVFLTNRAYAM |
| 011861 | (SEQ ID NO: | KG | DV |
| | 200) | (SEQ ID NO: 300) | (SEQ ID NO: 400) |
| AB- | TFTFNNAWVN | RLRSDQEHWAKDYAAHM | NTGYWPQSVFLTNRAYAM |
| 011862 | (SEQ ID NO: | KG | DV |
| | 201) | (SEQ ID NO: 301) | (SEQ ID NO: 401) |
| AB- | TFTFNNAWVN | RLRSDYEHWAKDYAAHM | NTGYWPQSVFLTNRAYAM |
| 011863 | (SEQ ID NO: | KG | DV |
| | 202) | (SEQ ID NO: 302) | (SEQ ID NO: 402) |
| AB- | TFTFNNAWVN | RLRSDTQHWAKDYAAHM | NTGYWPQSVFLTNRAYAM |
| 011864 | (SEQ ID NO: | KG | DV |
| | 203) | (SEQ ID NO: 303) | (SEQ ID NO: 403) |
| AB- | TFTFNNAWVN | RLRSDTEHWAKDYAAHM | NTGYWPQSVFLTNRAYAM |
| 011865 | (SEQ ID NO: | KG | DV |
| | 204) | (SEQ ID NO: 304) | (SEQ ID NO: 404) |
| AB- | TFTFNNAWVN | RLRSDTEHWAKDYAAHM | NTGYWPQSVFLTNRAYAM |
| 011866 | (SEQ ID NO: | KG | DV |
| | 205) | (SEQ ID NO: 305) | (SEQ ID NO: 405) |
| AB- | TFTFNNAWVN | RLRSDTEHWAKDYAAHM | NTGYWPQSVFLTNRAYAM |
| 011867 | (SEQ ID NO: | KG | DV |
| | 206) | (SEQ ID NO: 306) | (SEQ ID NO: 406) |
| AB- | TFTFNNAWVN | RLRSDTEHWAKDYAAHM | NTGKWPQSVFLTNRAYAM |
| 011868 | (SEQ ID NO: | KG | DV |
| | 207) | (SEQ ID NO: 307) | (SEQ ID NO: 407) |
| AB- | TFTFNNAWVN | RLRSDTEHWAKDYAAHM | NTGYDPQSVFLTNRAYAM |
| 011869 | (SEQ ID NO: | KG | DV |
| | 208) | (SEQ ID NO: 308) | (SEQ ID NO: 408) |
| AB- | TFTFNNAWVN | RLRSDTEHWAKDYAAHM | NTGYWPASVFLTNRAYAM |
| 011870 | (SEQ ID NO: | KG | DV |
| | 209) | (SEQ ID NO: 309) | (SEQ ID NO: 409) |
| AB- | TFTFNNAWVN | RLRSDTEHWAKDYAAHM | NTGYWPQSVQLTNRAYAM |
| 011871 | (SEQ ID NO: | KG | DV |
| | 210) | (SEQ ID NO: 310) | (SEQ ID NO: 410) |
| AB- | TFTFNNAWVN | RLRSDTEHWAKDYAAHM | NTGYWPQSVFLTDRAYAM |
| 011872 | (SEQ ID NO: | KG | DV |
| | 211) | (SEQ ID NO: 311) | (SEQ ID NO: 411) |
| AB- | TFTFNNAWVN | RLRSDTEHWAKDYAAHM | NTGYWPQSVFLTNQAYAM |
| 011873 | (SEQ ID NO: | KG | DV |
| | 212) | (SEQ ID NO: 312) | (SEQ ID NO: 412) |

TABLE 2
Light chain CDR sequences

| ID | LCDR1 | LCDR2 | LCDR3 |
|---|---|---|---|
| AB-006410 | ASNESLRRGQYHFLD | LGFRRAS | MQALDPPLS |
| | (SEQ ID NO: 43) | (SEQ ID NO: 57) | (SEQ ID NO: 71) |
| AB-011110 | ASSESLRRGQYHFLD | LGFRRAS | MQALDPPLS |
| | (SEQ ID NO: 44) | (SEQ ID NO: 58) | (SEQ ID NO: 72) |
| AB-011111 | ASNEALRRGQYHFLD | LGFRRAS | MQALDPPLS |
| | (SEQ ID NO: 45) | (SEQ ID NO: 59) | (SEQ ID NO: 73) |
| AB-011366 | KSSESLHREGYNFLD | LGSRRAA | MQCLRPPLS |
| | (SEQ ID NO: 46) | (SEQ ID NO: 60) | (SEQ ID NO: 74) |
| AB-011367 | KSSESLHREGYNFLD | LGSRRAA | MQALRPPLS |
| | (SEQ ID NO: 47) | (SEQ ID NO: 61) | (SEQ ID NO: 75) |
| AB-011368 | ASSESLHRGQYHFLD | LGFRRAS | MQALDPPLS |
| | (SEQ ID NO: 48) | (SEQ ID NO: 62) | (SEQ ID NO: 76) |
| AB-011369 | ASSESLQRGQYHFLD | LGFRRAS | MQALDPPLS |
| | (SEQ ID NO: 49) | (SEQ ID NO: 63) | (SEQ ID NO: 77) |
| AB-011370 | ASSESLRSGQYHFLD | LGFRRAS | MQALDPPLS |
| | (SEQ ID NO: 50) | (SEQ ID NO: 64) | (SEQ ID NO: 78) |
| AB-011371 | ASSESLRRGQYHFLD | LGFNRAS | MQALDPPLS |
| | (SEQ ID NO: 51) | (SEQ ID NO: 65) | (SEQ ID NO: 79) |
| AB-011372 | ASSESLRRGQYHFLD | LGFSRAS | MQALDPPLS |
| | (SEQ ID NO: 52) | (SEQ ID NO: 66) | (SEQ ID NO: 80) |
| AB-011373 | ASSESLRRGQYHFLD | LGFRQAS | MQALDPPLS |
| | (SEQ ID NO: 53) | (SEQ ID NO: 67) | (SEQ ID NO: 81) |
| AB-011374 | ASSESLRRGQYHFLD | LGFRRAS | MQALNPPLS |
| | (SEQ ID NO: 54) | (SEQ ID NO: 68) | (SEQ ID NO: 82) |
| AB-011375 | ASSESLRRGQYHFLD | LGFRRAS | MQALRPPLS |
| | (SEQ ID NO: 55) | (SEQ ID NO: 69) | (SEQ ID NO: 83) |
| AB-011376 | ASSESLRRGQYHFLD | LGFRRAS | MQALDTPLS |
| | (SEQ ID NO: 56) | (SEQ ID NO: 70) | (SEQ ID NO: 84) |
| AB-011622 | ASSESLQRGQYHFLD | LGFRRAS | MQALNPPLS |
| | (SEQ ID NO: 413) | (SEQ ID NO: 513) | (SEQ ID NO: 613) |
| AB-011623 | ASSESLQRGQYHFLD | LGFNRAS | MQALNPPLS |
| | (SEQ ID NO: 414) | (SEQ ID NO: 514) | (SEQ ID NO: 614) |
| AB-011624 | ASSESLRSGQYHFLD | LGFRRAS | MQALNPPLS |
| | (SEQ ID NO: 415) | (SEQ ID NO: 515) | (SEQ ID NO: 615) |
| AB-011625 | ASSESLQSGQYHFLD | LGFRRAS | MQALNPPLS |
| | (SEQ ID NO: 416) | (SEQ ID NO: 516) | (SEQ ID NO: 616) |
| AB-011626 | ASSESLRSGQYHFLD | LGFNRAS | MQALNPPLS |
| | (SEQ ID NO: 417) | (SEQ ID NO: 517) | (SEQ ID NO: 617) |
| AB-011627 | ASSESLQSGQYHFLD | LGFNRAS | MQALNPPLS |
| | (SEQ ID NO: 418) | (SEQ ID NO: 518) | (SEQ ID NO: 618) |
| AB-011628 | KSSESLHREGYNFLD | LGSNRAA | MQALRPPLS |
| | (SEQ ID NO: 419) | (SEQ ID NO: 519) | (SEQ ID NO: 619) |
| AB-011781 | ASSESLQRGQYHFLD | LGFRRAS | MQALNPPLS |
| | (SEQ ID NO: 420) | (SEQ ID NO: 520) | (SEQ ID NO: 620) |
| AB-011782 | ASSESLQRGQYHFLD | LGFRRAS | MQALNPPLS |
| | (SEQ ID NO: 421) | (SEQ ID NO: 521) | (SEQ ID NO: 621) |
| AB-011783 | ASSESLQRGQYHFLD | LGFRRAS | MQALNPPLS |
| | (SEQ ID NO: 422) | (SEQ ID NO: 522) | (SEQ ID NO: 622) |
| AB-011784 | ASSESLQRGQYHFLD | LGFRRAS | MQALNPPLS |
| | (SEQ ID NO: 423) | (SEQ ID NO: 523) | (SEQ ID NO: 623) |
| AB-011785 | ASSESLQRGQYHFLD | LGFRRAS | MQALNPPLS |
| | (SEQ ID NO: 424) | (SEQ ID NO: 524) | (SEQ ID NO: 624) |
| AB-011786 | ASSESLQRGQYHFLD | LGFRRAS | MQALNPPLS |
| | (SEQ ID NO: 425) | (SEQ ID NO: 525) | (SEQ ID NO: 625) |
| AB-011787 | ASSESLQRGQYHFLD | LGFRRAS | MQALNPPLS |
| | (SEQ ID NO: 426) | (SEQ ID NO: 526) | (SEQ ID NO: 626) |
| AB-011788 | ASSESLQRGQYHFLD | LGFRRAS | MQALNPPLS |
| | (SEQ ID NO: 427) | (SEQ ID NO: 527) | (SEQ ID NO: 627) |
| AB-011789 | ASSESLQRGQYHFLD | LGFRRAS | MQALNPPLS |
| | (SEQ ID NO: 428) | (SEQ ID NO: 528) | (SEQ ID NO: 628) |
| AB-011790 | ASSESLQRGQYHFLD | LGFRRAS | MQALNPPLS |
| | (SEQ ID NO: 429) | (SEQ ID NO: 529) | (SEQ ID NO: 629) |
| AB-011791 | ASSESLQRGQYHFLD | LGFRRAS | MQALNPPLS |
| | (SEQ ID NO: 430) | (SEQ ID NO: 530) | (SEQ ID NO: 630) |
| AB-011792 | ASSESLQRGQYHFLD | LGFRRAS | MQALNPPLS |
| | (SEQ ID NO: 431) | (SEQ ID NO: 531) | (SEQ ID NO: 631) |
| AB-011793 | ASSESLQRGQYHFLD | LGFRRAS | MQALNPPLS |
| | (SEQ ID NO: 432) | (SEQ ID NO: 532) | (SEQ ID NO: 632) |
| AB-011794 | ASSESLQRGQYHFLD | LGFRRAS | MQALNPPLS |
| | (SEQ ID NO: 433) | (SEQ ID NO: 533) | (SEQ ID NO: 633) |
| AB-011795 | ASSESLQRGQYHFLD | LGFRRAS | MQALNPPLS |
| | (SEQ ID NO: 434) | (SEQ ID NO: 534) | (SEQ ID NO: 634) |
| AB-011796 | ASSESLQRGQYHFLD | LGFRRAS | MQALNPPLS |
| | (SEQ ID NO: 435) | (SEQ ID NO: 535) | (SEQ ID NO: 635) |
| AB-011797 | ASSESLQRGQYHFLD | LGFRRAS | MQALNPPLS |
| | (SEQ ID NO: 436) | (SEQ ID NO: 536) | (SEQ ID NO: 636) |
| AB-011798 | ASSESLQRGQYHFLD | LGFRRAS | MQALNPPLS |
| | (SEQ ID NO: 437) | (SEQ ID NO: 537) | (SEQ ID NO: 637) |
| AB-011799 | ASSESLQRGQYHFLD | LGFRRAS | MQALNPPLS |
| | (SEQ ID NO: 438) | (SEQ ID NO: 538) | (SEQ ID NO: 638) |
| AB-011800 | ASSESLORGQYHFLD | LGFRRAS | MQALNPPLS |
| | (SEQ ID NO: 439) | (SEQ ID NO: 539) | (SEQ ID NO: 639) |
| AB-011801 | ASSESLQRGQYHFLD | LGFRRAS | MQALNPPLS |
| | (SEQ ID NO: 440) | (SEQ ID NO: 540) | (SEQ ID NO: 640) |
| AB-011802 | ASSESLQRGQYHFLD | LGFRRAS | MQALNPPLS |
| | (SEQ ID NO: 441) | (SEQ ID NO: 541) | (SEQ ID NO: 641) |
| AB-011803 | ASSESLQRGQYHFLD | LGFRRAS | MQALNPPLS |
| | (SEQ ID NO: 442) | (SEQ ID NO: 542) | (SEQ ID NO: 642) |
| AB-011804 | ASSESLQRGQYHFLD | LGFRRAS | MQALNPPLS |
| | (SEQ ID NO: 443) | (SEQ ID NO: 543) | (SEQ ID NO: 643) |
| AB-011805 | ASSESLORGQYHFLD | LGFRRAS | MQALNPPLS |
| | (SEQ ID NO: 444) | (SEQ ID NO: 544) | (SEQ ID NO: 644) |
| AB-011806 | ASSESLQRGQYHFLD | LGFRRAS | MQALNPPLS |
| | (SEQ ID NO: 445) | (SEQ ID NO: 545) | (SEQ ID NO: 645) |
| AB-011807 | ASSESLQRGQYHFLD | LGFRRAS | MQALNPPLS |
| | (SEQ ID NO: 446) | (SEQ ID NO: 546) | (SEQ ID NO: 646) |
| AB-011808 | ASSESLQRGQYHFLD | LGFRRAS | MQALNPPLS |
| | (SEQ ID NO: 447) | (SEQ ID NO: 547) | (SEQ ID NO: 647) |
| AB-011809 | ASSESLQRGQYHFLD | LGFRRAS | MQALNPPLS |
| | (SEQ ID NO: 448) | (SEQ ID NO: 548) | (SEQ ID NO: 648) |
| AB-011810 | ASSESLQRGQYHFLD | LGFRRAS | MQALNPPLS |
| | (SEQ ID NO: 449) | (SEQ ID NO: 549) | (SEQ ID NO: 649) |
| AB-011811 | ASSESLQRGQYHFLD | LGFRRAS | MQALNPPLS |
| | (SEQ ID NO: 450) | (SEQ ID NO: 550) | (SEQ ID NO: 650) |
| AB-011812 | ASSESLQRGQYHFLD | LGFRRAS | MQALNPPLS |
| | (SEQ ID NO: 451) | (SEQ ID NO: 551) | (SEQ ID NO: 651) |
| AB-011813 | ASSESLQRGQYHFLD | LGFRRAS | MQALNPPLS |
| | (SEQ ID NO: 452) | (SEQ ID NO: 552) | (SEQ ID NO: 652) |
| AB-011814 | ASSESLORGQYHFLD | LGFRRAS | MQALNPPLS |
| | (SEQ ID NO: 453) | (SEQ ID NO: 553) | (SEQ ID NO: 653) |
| AB-011815 | ASSESLQRGQYHFLD | LGFRRAS | MQALNPPLS |
| | (SEQ ID NO: 454) | (SEQ ID NO: 554) | (SEQ ID NO: 654) |
| AB-011816 | ASSESLQRGQYHFLD | LGFRRAS | MQALNPPLS |
| | (SEQ ID NO: 455) | (SEQ ID NO: 555) | (SEQ ID NO: 655) |
| AB-011817 | ASSESLQRGQYHFLD | LGFRRAS | MQALNPPLS |
| | (SEQ ID NO: 456) | (SEQ ID NO: 556) | (SEQ ID NO: 656) |
| AB-011818 | ASSESLQRGQYHFLD | LGFRRAS | MQALNPPLS |
| | (SEQ ID NO: 457) | (SEQ ID NO: 557) | (SEQ ID NO: 657) |
| AB-011819 | ASSESLQRGQYHFLD | LGFRRAS | MQALNPPLS |
| | (SEQ ID NO: 458) | (SEQ ID NO: 558) | (SEQ ID NO: 658) |
| AB-011820 | ASSESLQRGQYHFLD | LGFRRAS | MQALNPPLS |
| | (SEQ ID NO: 459) | (SEQ ID NO: 559) | (SEQ ID NO: 659) |
| AB-011821 | ASSESLQRGQYHFLD | LGFRRAS | MQALNPPLS |
| | (SEQ ID NO: 460) | (SEQ ID NO: 560) | (SEQ ID NO: 660) |
| AB-011822 | ASSESLQRGQYHFLD | LGFRRAS | MQALNPPLS |
| | (SEQ ID NO: 461) | (SEQ ID NO: 561) | (SEQ ID NO: 661) |
| AB-011823 | ASSESLQRGQYHFLD | LGFRRAS | MQALNPPLS |
| | (SEQ ID NO: 462) | (SEQ ID NO: 562) | (SEQ ID NO: 662) |
| AB-011824 | ASSESLQRGQYHFLD | LGFRRAS | MQALNPPLS |
| | (SEQ ID NO: 463) | (SEQ ID NO: 563) | (SEQ ID NO: 663) |
| AB-011825 | ASSESLQRGQYHFLD | LGFRRAS | MQALNPPLS |
| | (SEQ ID NO: 464) | (SEQ ID NO: 564) | (SEQ ID NO: 664) |
| AB-011826 | ASSASLQRGQYHFLD | LGFRRAS | MQALNPPLS |
| | (SEQ ID NO: 465) | (SEQ ID NO: 565) | (SEQ ID NO: 665) |
| AB-011827 | ASSYSLQRGQYHFLD | LGFRRAS | MQALNPPLS |
| | (SEQ ID NO: 466) | (SEQ ID NO: 566) | (SEQ ID NO: 666) |
| AB-011828 | ASSEYLQRGQYHFLD | LGFRRAS | MQALNPPLS |
| | (SEQ ID NO: 467) | (SEQ ID NO: 567) | (SEQ ID NO: 667) |
| AB-011829 | ASSEELQRGQYHFLD | LGFRRAS | MQALNPPLS |
| | (SEQ ID NO: 468) | (SEQ ID NO: 568) | (SEQ ID NO: 668) |
| AB-011830 | ASSESLQREQYHFLD | LGFRRAS | MQALNPPLS |
| | (SEQ ID NO: 469) | (SEQ ID NO: 569) | (SEQ ID NO: 669) |
| AB-011831 | ASSESLORSQYHFLD | LGFRRAS | MQALNPPLS |
| | (SEQ ID NO: 470) | (SEQ ID NO: 570) | (SEQ ID NO: 670) |
| AB-011832 | ASSESLQRGYYHFLD | LGFRRAS | MQALNPPLS |
| | (SEQ ID NO: 471) | (SEQ ID NO: 571) | (SEQ ID NO: 671) |
| AB-011833 | ASSESLQRGSYHFLD | LGFRRAS | MQALNPPLS |
| | (SEQ ID NO: 472) | (SEQ ID NO: 572) | (SEQ ID NO: 672) |
| AB-011834 | ASSESLQRGQAHFLD | LGFRRAS | MQALNPPLS |
| | (SEQ ID NO: 473) | (SEQ ID NO: 573) | (SEQ ID NO: 673) |
| AB-011835 | ASSESLQRGQHHFLD | LGFRRAS | MQALNPPLS |
| | (SEQ ID NO: 474) | (SEQ ID NO: 574) | (SEQ ID NO: 674) |
| AB-011836 | ASSESLQRGQVHFLD | LGFRRAS | MQALNPPLS |
| | (SEQ ID NO: 475) | (SEQ ID NO: 575) | (SEQ ID NO: 675) |
| AB-011837 | ASSESLQRGQSHFLD | LGFRRAS | MQALNPPLS |
| | (SEQ ID NO: 476) | (SEQ ID NO: 576) | (SEQ ID NO: 676) |
| AB-011838 | ASSESLQRGQYAFLD | LGFRRAS | MQALNPPLS |
| | (SEQ ID NO: 477) | (SEQ ID NO: 577) | (SEQ ID NO: 677) |
| AB-011839 | ASSESLQRGQYNFLD | LGFRRAS | MQALNPPLS |
| | (SEQ ID NO: 478) | (SEQ ID NO: 578) | (SEQ ID NO: 678) |
| AB-011840 | ASSESLQRGQYHALD | LGFRRAS | MQALNPPLS |
| | (SEQ ID NO: 479) | (SEQ ID NO: 579) | (SEQ ID NO: 679) |
| AB-011841 | ASSESLQRGQYHKLD | LGFRRAS | MQALNPPLS |
| | (SEQ ID NO: 480) | (SEQ ID NO: 580) | (SEQ ID NO: 680) |
| AB-011842 | ASSESLQRGQYHFLN | LGFRRAS | MQALNPPLS |
| | (SEQ ID NO: 481) | (SEQ ID NO: 581) | (SEQ ID NO: 681) |
| AB-011843 | ASSESLQRGQYHFLS | LGFRRAS | MQALNPPLS |
| | (SEQ ID NO: 482) | (SEQ ID NO: 582) | (SEQ ID NO: 682) |
| AB-011844 | ASSESLQRGQYHFLD | AGFRRAS | MQALNPPLS |
| | (SEQ ID NO: 483) | (SEQ ID NO: 583) | (SEQ ID NO: 683) |
| AB-011845 | ASSESLQRGQYHFLD | KGFRRAS | MQALNPPLS |
| | (SEQ ID NO: 484) | (SEQ ID NO: 584) | (SEQ ID NO: 684) |
| AB-011846 | ASSESLQRGQYHFLD | LGLRRAS | MQALNPPLS |
| | (SEQ ID NO: 485) | (SEQ ID NO: 585) | (SEQ ID NO: 685) |
| AB-011847 | ASSESLQRGQYHFLD | LGERRAS | MQALNPPLS |
| | (SEQ ID NO: 486) | (SEQ ID NO: 586) | (SEQ ID NO: 686) |
| AB-011848 | ASSESLQRGQYHFLD | LGFHRAS | MQALNPPLS |
| | (SEQ ID NO: 487) | (SEQ ID NO: 587) | (SEQ ID NO: 687) |
| AB-011849 | ASSESLQRGQYHFLD | LGFRKAS | MQALNPPLS |
| | (SEQ ID NO: 488) | (SEQ ID NO: 588) | (SEQ ID NO: 688) |
| AB-011850 | ASSESLQRGQYHFLD | LGFRRHS | MQALNPPLS |
| | (SEQ ID NO: 489) | (SEQ ID NO: 589) | (SEQ ID NO: 689) |
| AB-011851 | ASSESLQRGQYHFLD | LGFRRAE | MQALNPPLS |
| | (SEQ ID NO: 490) | (SEQ ID NO: 590) | (SEQ ID NO: 690) |
| AB-011852 | ASSESLQRGQYHFLD | LGFRRAK | MQALNPPLS |
| | (SEQ ID NO: 491) | (SEQ ID NO: 591) | (SEQ ID NO: 691) |
| AB-011853 | ASSESLQRGQYHFLD | LGFRRAS | AQALNPPLS |
| | (SEQ ID NO: 492) | (SEQ ID NO: 592) | (SEQ ID NO: 692) |
| AB-011854 | ASSESLORGQYHFLD | LGFRRAS | MAALNPPLS |
| | (SEQ ID NO: 493) | (SEQ ID NO: 593) | (SEQ ID NO: 693) |
| AB-011855 | ASSESLORGQYHFLD | LGFRRAS | MQAFNPPLS |
| | (SEQ ID NO: 494) | (SEQ ID NO: 594) | (SEQ ID NO: 694) |
| AB-011856 | ASSESLORGQYHFLD | LGFRRAS | MQAENPPLS |
| | (SEQ ID NO: 495) | (SEQ ID NO: 595) | (SEQ ID NO: 695) |
| AB-011857 | ASSESLQRGQYHFLD | LGFRRAS | MQALNPDLS |
| | (SEQ ID NO: 496) | (SEQ ID NO: 596) | (SEQ ID NO: 696) |
| AB-011858 | ASSESLQRGQYHFLD | LGFRRAS | MQALNPPYS |
| | (SEQ ID NO: 497) | (SEQ ID NO: 597) | (SEQ ID NO: 697) |
| AB-011859 | ASSESLQRGQYHFLD | LGFRRAS | MQALNPPDS |
| | (SEQ ID NO: 498) | (SEQ ID NO: 598) | (SEQ ID NO: 698) |
| AB-011860 | KSSESLHREGYNFLD | LGSRRAA | MQALRPPLS |
| | (SEQ ID NO: 499) | (SEQ ID NO: 599) | (SEQ ID NO: 699) |
| AB-011861 | KSSESLHREGYNFLD | LGSRRAA | MQALRPPLS |
| | (SEQ ID NO: 500) | (SEQ ID NO: 600) | (SEQ ID NO: 700) |
| AB-011862 | KSSESLHREGYNFLD | LGSRRAA | MQALRPPLS |
| | (SEQ ID NO: 501) | (SEQ ID NO: 601) | (SEQ ID NO: 701) |
| AB-011863 | KSSESLHREGYNFLD | LGSRRAA | MQALRPPLS |
| | (SEQ ID NO: 502) | (SEQ ID NO: 602) | (SEQ ID NO: 702) |
| AB-011864 | KSSESLHREGYNFLD | LGSRRAA | MQALRPPLS |
| | (SEQ ID NO: 503) | (SEQ ID NO: 603) | (SEQ ID NO: 703) |
| AB-011865 | KSSESLHRQGYNFLD | LGSRRAA | MQALRPPLS |
| | (SEQ ID NO: 504) | (SEQ ID NO: 604) | (SEQ ID NO: 704) |
| AB-011866 | KSSESLHRGQYNFLD | LGSRRAA | MQALRPPLS |
| | (SEQ ID NO: 505) | (SEQ ID NO: 605) | (SEQ ID NO: 705) |
| AB-011867 | KSSESLHREGTNFLD | LGSRRAA | MQALRPPLS |
| | (SEQ ID NO: 506) | (SEQ ID NO: 606) | (SEQ ID NO: 706) |
| AB-011868 | KSSESLHREGYNFLD | LGSRRAA | MQALRPPLS |
| | (SEQ ID NO: 507) | (SEQ ID NO: 607) | (SEQ ID NO: 707) |
| AB-011869 | KSSESLHREGYNFLD | LGSRRAA | MQALRPPLS |
| | (SEQ ID NO: 508) | (SEQ ID NO: 608) | (SEQ ID NO: 708) |
| AB-011870 | KSSESLHREGYNFLD | LGSRRAA | MQALRPPLS |
| | (SEQ ID NO: 509) | (SEQ ID NO: 609) | (SEQ ID NO: 709) |
| AB-011871 | KSSESLHREGYNFLD | LGSRRAA | MQALRPPLS |
| | (SEQ ID NO: 510) | (SEQ ID NO: 610) | (SEQ ID NO: 710) |
| AB-011872 | KSSESLHREGYNFLD | LGSRRAA | MQALRPPLS |
| | (SEQ ID NO: 511) | (SEQ ID NO: 611) | (SEQ ID NO: 711) |
| AB-011873 | KSSESLHREGYNFLD | LGSRRAA | MQALRPPLS |
| | (SEQ ID NO: 512) | (SEQ ID NO: 612) | (SEQ ID NO: 712) |

TABLE 3
Heavy and Light variable region sequences

| ID | VH | VL |
|---|---|---|
| AB- | EVQVVESGGGEIKPGGSVRLSCTAN | DIVMTQSPLFLSVTPGESASMSCAS |
| 006410 | SFTFSNAWVNWVRQAPGRGLEWV | NESLRRGQYHFLDWYLQKPGQSPQ |
| | GRLRSDTEGWTKDYAPHLKDRVVI | LLIYLGFRRASGVPARFSARASDTH |
| | SREESGHTVFLEMASLQPQDTGVYY | YTLTISRVDTEDVGVYYCMQALDPP |
| | CNTGYWPQSAFLTNRAYAMDVWG | LSFGPGTTVDMK |
| | RGTTVVVSS | (SEQ ID NO: 99) |
| | (SEQ ID NO: 85) | |
| AB- | EVQVVESGGGEIKPGGSVRLSCTAN | DIVMTQSPLFLSVTPGESASMSCASS |
| 011110 | SFTFSNAWVNWVRQAPGRGLEWV | ESLRRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALDPPL |
| | CNTGYWPQSAFLTNRAYAMDVWG | SFGPGTTVDMK |
| | RGTTVVVSS | (SEQ ID NO: 100) |
| | (SEQ ID NO: 86) | |
| AB- | EVQVVESGGGEIKPGGSVRLSCTAN | DIVMTQSPLFLSVTPGESASMSCAS |
| 011111 | SFTFSNAWVNWVRQAPGRGLEWV | NEALRRGQYHFLDWYLQKPGQSPQ |
| | GRLRSDTEGWTKDYAPHLKDRVVI | LLIYLGFRRASGVPARFSARASDTH |
| | SREESGHTVFLEMASLQPQDTGVYY | YTLTISRVDTEDVGVYYCMQALDPP |
| | CNTGYWPQSAFLTNRAYAMDVWG | LSFGPGTTVDMK |
| | RGTTVVVSS | (SEQ ID NO: 101) |
| | (SEQ ID NO: 87) | |
| AB- | EVQVVESGGGVVKPGGSLRLSCTA | DIVVTQSPLSLPVTLGEPASMSCKSS |
| 011366 | NTFTFNNAWVNWVRQAPGRGLEW | ESLHREGYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQCLRPP |
| | YYCNTGYWPQSVFLTNRAYAMDV | LSFGPGTTVDIK |
| | WGRGTTVIVSS | (SEQ ID NO: 102) |
| | (SEQ ID NO: 88) | |
| AB- | EVQVVESGGGVVKPGGSLRLSCTA | DIVVTQSPLSLPVTLGEPASMSCKSS |
| 011367 | NTFTFNNAWVNWVRQAPGRGLEW | ESLHREGYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTNRAYAMDV | LSFGPGTTVDIK |
| | WGRGTTVIVSS | (SEQ ID NO: 103) |
| | (SEQ ID NO: 89) | |
| AB- | EVQVVESGGGEIKPGGSVRLSCTAN | DIVMTQSPLFLSVTPGESASMSCASS |
| 011368 | SFTFSNAWVNWVRQAPGRGLEWV | ESLHRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALDPPL |
| | CNTGYWPQSAFLTNRAYAMDVWG | SFGPGTTVDMK |
| | RGTTVVVSS | (SEQ ID NO: 104) |
| | (SEQ ID NO: 90) | |
| AB- | EVQVVESGGGEIKPGGSVRLSCTAN | DIVMTQSPLFLSVTPGESASMSCASS |
| 011369 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALDPPL |
| | CNTGYWPQSAFLTNRAYAMDVWG | SFGPGTTVDMK |
| | RGTTVVVSS | (SEQ ID NO: 105) |
| | (SEQ ID NO: 91) | |
| AB- | EVQVVESGGGEIKPGGSVRLSCTAN | DIVMTQSPLFLSVTPGESASMSCASS |
| 011370 | SFTFSNAWVNWVRQAPGRGLEWV | ESLRSGQYHFLDWYLQKPGQSPQLL |
| | GRLRSDTEGWTKDYAPHLKDRVVI | IYLGFRRASGVPARFSARASDTHYT |
| | SREESGHTVFLEMASLQPQDTGVYY | LTISRVDTEDVGVYYCMQALDPPLS |
| | CNTGYWPQSAFLTNRAYAMDVWG | FGPGTTVDMK |
| | RGTTVVVSS | (SEQ ID NO: 106) |
| | (SEQ ID NO: 92) | |
| AB- | EVQVVESGGGEIKPGGSVRLSCTAN | DIVMTQSPLFLSVTPGESASMSCASS |
| 011371 | SFTFSNAWVNWVRQAPGRGLEWV | ESLRRGQYHFLDWYLQKPGQSPQL |
| | GRLRSDTEGWTKDYAPHLKDRVVI | LIYLGFNRASGVPARFSARASDTHY |
| | SREESGHTVFLEMASLQPQDTGVYY | TLTISRVDTEDVGVYYCMQALDPPL |
| | CNTGYWPQSAFLTNRAYAMDVWG | SFGPGTTVDMK |
| | RGTTVVVSS | (SEQ ID NO: 107) |
| | (SEQ ID NO: 93) | |
| AB- | EVQVVESGGGEIKPGGSVRLSCTAN | DIVMTQSPLFLSVTPGESASMSCASS |
| 011372 | SFTFSNAWVNWVRQAPGRGLEWV | ESLRRGQYHFLDWYLQKPGQSPQL |
| | GRLRSDTEGWTKDYAPHLKDRVVI | LIYLGFSRASGVPARFSARASDTHY |
| | SREESGHTVFLEMASLQPQDTGVYY | TLTISRVDTEDVGVYYCMQALDPPL |
| | CNTGYWPQSAFLTNRAYAMDVWG | SFGPGTTVDMK |
| | RGTTVVVSS | (SEQ ID NO: 108) |
| | (SEQ ID NO: 94) | |
| AB- | EVQVVESGGGEIKPGGSVRLSCTAN | DIVMTQSPLFLSVTPGESASMSCASS |
| 011373 | SFTFSNAWVNWVRQAPGRGLEWV | ESLRRGQYHFLDWYLQKPGQSPQL |
| | GRLRSDTEGWTKDYAPHLKDRVVI | LIYLGFRQASGVPARFSARASDTHY |
| | SREESGHTVFLEMASLQPQDTGVYY | TLTISRVDTEDVGVYYCMQALDPPL |
| | CNTGYWPQSAFLTNRAYAMDVWG | SFGPGTTVDMK |
| | RGTTVVVSS | (SEQ ID NO: 109) |
| | (SEQ ID NO: 95) | |
| AB- | EVQVVESGGGEIKPGGSVRLSCTAN | DIVMTQSPLFLSVTPGESASMSCASS |
| 011374 | SFTFSNAWVNWVRQAPGRGLEWV | ESLRRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 110) |
| | (SEQ ID NO: 96) | |
| AB- | EVQVVESGGGEIKPGGSVRLSCTAN | DIVMTQSPLFLSVTPGESASMSCASS |
| 011375 | SFTFSNAWVNWVRQAPGRGLEWV | ESLRRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALRPPL |
| | CNTGYWPQSAFLTNRAYAMDVWG | SFGPGTTVDMK |
| | RGTTVVVSS | (SEQ ID NO: 111) |
| | (SEQ ID NO: 97) | |
| AB- | EVQVVESGGGEIKPGGSVRLSCTAN | DIVMTQSPLFLSVTPGESASMSCASS |
| 011376 | SFTFSNAWVNWVRQAPGRGLEWV | ESLRRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALDTPL |
| | CNTGYWPQSAFLTNRAYAMDVWG | SFGPGTTVDMK |
| | RGTTVVVSS | (SEQ ID NO: 112) |
| | (SEQ ID NO: 98) | |
| AB- | EVQVVESGGGEIKPGGSVRLSCTAN | DIVMTQSPLFLSVTPGESASMSCASS |
| 011622 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 813) |
| | (SEQ ID NO: 713) | |
| AB- | EVQVVESGGGEIKPGGSVRLSCTAN | DIVMTQSPLFLSVTPGESASMSCASS |
| 011623 | SFTFSNAWVNWVRQAPGRGLEWV | ESLQRGQYHFLDWYLQKPGQSPQL |
| | GRLRSDTEGWTKDYAPHLKDRVVI | LIYLGFNRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 814) |
| | (SEQ ID NO: 714) | |
| AB- | EVQVVESGGGEIKPGGSVRLSCTAN | DIVMTQSPLFLSVTPGESASMSCASS |
| 011624 | SFTFSNAWVNWVRQAPGRGLEWV | ESLRSGQYHFLDWYLQKPGQSPQLL |
| | GRLRSDTEGWTKDYAPHLKDRVVI | IYLGFRRASGVPARFSARASDTHYT |
| | SREESGHTVFLEMASLQPQDTGVYY | LTISRVDTEDVGVYYCMQALNPPLS |
| | CNTGYWPQSAFLTNRAYAMDVWG | FGPGTTVDMK |
| | RGTTVVVSS | (SEQ ID NO: 815) |
| | (SEQ ID NO: 715) | |
| AB- | EVQVVESGGGEIKPGGSVRLSCTAN | DIVMTQSPLFLSVTPGESASMSCASS |
| 011625 | SFTFSNAWVNWVRQAPGRGLEWV | ESLQSGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 816) |
| | (SEQ ID NO: 716) | |
| AB- | EVQVVESGGGEIKPGGSVRLSCTAN | DIVMTQSPLFLSVTPGESASMSCASS |
| 011626 | SFTFSNAWVNWVRQAPGRGLEWV | ESLRSGQYHFLDWYLQKPGQSPQLL |
| | GRLRSDTEGWTKDYAPHLKDRVVI | IYLGFNRASGVPARFSARASDTHYT |
| | SREESGHTVFLEMASLQPQDTGVYY | LTISRVDTEDVGVYYCMQALNPPLS |
| | CNTGYWPQSAFLTNRAYAMDVWG | FGPGTTVDMK |
| | RGTTVVVSS | (SEQ ID NO: 817) |
| | (SEQ ID NO: 717) | |
| AB- | EVQVVESGGGEIKPGGSVRLSCTAN | DIVMTQSPLFLSVTPGESASMSCASS |
| 011627 | SFTFSNAWVNWVRQAPGRGLEWV | ESLQSGQYHFLDWYLQKPGQSPQL |
| | GRLRSDTEGWTKDYAPHLKDRVVI | LIYLGFNRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 818) |
| | (SEQ ID NO: 718) | |
| AB- | EVQVVESGGGVVKPGGSLRLSCTA | DIVVTQSPLSLPVTLGEPASMSCKSS |
| 011628 | NTFTFNNAWVNWVRQAPGRGLEW | ESLHREGYNFLDWYLQKPGQSPQL |
| | VGRLRSDTEHWAKDYAAHMKGRV | LVYLGSNRAAGVPARFSGSGAGTD |
| | VISREESGRTVYLEMIGLKVEDTAV | YTLRISRVEAEDVGVYYCMQALRPP |
| | YYCNTGYWPQSVFLTNRAYAMDV | LSFGPGTTVDIK |
| | WGRGTTVIVSS | (SEQ ID NO: 819) |
| | (SEQ ID NO: 719) | |
| AB- | EVQVVESGGGEIKPGGSVRLSCTAN | DIVMTQSPLFLSVTPGESASMSCASS |
| 011781 | SFTFDNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 820) |
| | (SEQ ID NO: 720) | |
| AB- | EVQVVESGGGEIKPGGSVRLSCTAN | DIVMTQSPLFLSVTPGESASMSCASS |
| 011782 | SFTFSAAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 821) |
| | (SEQ ID NO: 721) | |
| AB- | EVQVVESGGGEIKPGGSVRLSCTAN | DIVMTQSPLFLSVTPGESASMSCASS |
| 011783 | SFTFSNAEVNWVRQAPGRGLEWVG | ESLQRGQYHFLDWYLQKPGQSPQL |
| | RLRSDTEGWTKDYAPHLKDRVVIS | LIYLGFRRASGVPARFSARASDTHY |
| | REESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 822) |
| | (SEQ ID NO: 722) | |
| AB- | EVQVVESGGGEIKPGGSVRLSCTAN | DIVMTQSPLFLSVTPGESASMSCASS |
| 011784 | SFTFSNALVNWVRQAPGRGLEWVG | ESLQRGQYHFLDWYLQKPGQSPQL |
| | RLRSDTEGWTKDYAPHLKDRVVIS | LIYLGFRRASGVPARFSARASDTHY |
| | REESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 823) |
| | (SEQ ID NO: 723) | |
| AB- | EVQVVESGGGEIKPGGSVRLSCTAN | DIVMTQSPLFLSVTPGESASMSCASS |
| 011785 | SFTFSNAWVNWVRQAPGRGLEWV | ESLQRGQYHFLDWYLQKPGQSPQL |
| | GELRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 824) |
| | (SEQ ID NO: 724) | |
| AB- | EVQVVESGGGEIKPGGSVRLSCTAN | DIVMTQSPLFLSVTPGESASMSCASS |
| 011786 | SFTFSNAWVNWVRQAPGRGLEWV | ESLQRGQYHFLDWYLQKPGQSPQL |
| | GRLYSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 825) |
| | (SEQ ID NO: 725) | |
| AB- | EVQVVESGGGEIKPGGSVRLSCTAN | DIVMTQSPLFLSVTPGESASMSCASS |
| 011787 | SFTFSNAWVNWVRQAPGRGLEWV | ESLQRGQYHFLDWYLQKPGQSPQL |
| | GRLASDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 826) |
| | (SEQ ID NO: 726) | |
| AB- | EVQVVESGGGEIKPGGSVRLSCTAN | DIVMTQSPLFLSVTPGESASMSCASS |
| 011788 | SFTFSNAWVNWVRQAPGRGLEWV | ESLQRGQYHFLDWYLQKPGQSPQL |
| | GRLRSKTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 827) |
| | (SEQ ID NO: 727) | |
| AB- | EVQVVESGGGEIKPGGSVRLSCTAN | DIVMTQSPLFLSVTPGESASMSCASS |
| 011789 | SFTFSNAWVNWVRQAPGRGLEWV | ESLQRGQYHFLDWYLQKPGQSPQL |
| | GRLRSYTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 828) |
| | (SEQ ID NO: 728) | |
| AB- | EVQVVESGGGEIKPGGSVRLSCTAN | DIVMTQSPLFLSVTPGESASMSCASS |
| 011790 | SFTFSNAWVNWVRQAPGRGLEWV | ESLQRGQYHFLDWYLQKPGQSPQL |
| | GRLRSDREGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 829) |
| | (SEQ ID NO: 729) | |
| AB- | EVQVVESGGGEIKPGGSVRLSCTAN | DIVMTQSPLFLSVTPGESASMSCASS |
| 011791 | SFTFSNAWVNWVRQAPGRGLEWV | ESLQRGQYHFLDWYLQKPGQSPQL |
| | GRLRSDEEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 830) |
| | (SEQ ID NO: 730) | |
| AB- | EVQVVESGGGEIKPGGSVRLSCTAN | DIVMTQSPLFLSVTPGESASMSCASS |
| 011792 | SFTFSNAWVNWVRQAPGRGLEWV | ESLQRGQYHFLDWYLQKPGQSPQL |
| | GRLRSDYEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 831) |
| | (SEQ ID NO: 731) | |
| AB- | EVQVVESGGGEIKPGGSVRLSCTAN | DIVMTQSPLFLSVTPGESASMSCASS |
| 011793 | SFTFSNAWVNWVRQAPGRGLEWV | ESLQRGQYHFLDWYLQKPGQSPQL |
| | GRLRSDTKGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 832) |
| | (SEQ ID NO: 732) | |
| AB- | EVQVVESGGGEIKPGGSVRLSCTAN | DIVMTQSPLFLSVTPGESASMSCASS |
| 011794 | SFTFSNAWVNWVRQAPGRGLEWV | ESLQRGQYHFLDWYLQKPGQSPQL |
| | GRLRSDTEYWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 833) |
| | (SEQ ID NO: 733) | |
| AB- | EVQVVESGGGEIKPGGSVRLSCTAN | DIVMTQSPLFLSVTPGESASMSCASS |
| 011795 | SFTFSNAWVNWVRQAPGRGLEWV | ESLQRGQYHFLDWYLQKPGQSPQL |
| | GRLRSDTEHWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 834) |
| | (SEQ ID NO: 734) | |
| AB- | EVQVVESGGGEIKPGGSVRLSCTAN | DIVMTQSPLFLSVTPGESASMSCASS |
| 011796 | SFTFSNAWVNWVRQAPGRGLEWV | ESLQRGQYHFLDWYLQKPGQSPQL |
| | GRLRSDTEGSTKDYAPHLKDRVVIS | LIYLGFRRASGVPARFSARASDTHY |
| | REESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 835) |
| | (SEQ ID NO: 735) | |
| AB- | EVQVVESGGGEIKPGGSVRLSCTAN | DIVMTQSPLFLSVTPGESASMSCASS |
| 011797 | SFTFSNAWVNWVRQAPGRGLEWV | ESLQRGQYHFLDWYLQKPGQSPQL |
| | GRLRSDTEGWYKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 836) |
| | (SEQ ID NO: 736) | |
| AB- | EVQVVESGGGEIKPGGSVRLSCTAN | DIVMTQSPLFLSVTPGESASMSCASS |
| 011798 | SFTFSNAWVNWVRQAPGRGLEWV | ESLQRGQYHFLDWYLQKPGQSPQL |
| | GRLRSDTEGWEKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 837) |
| | (SEQ ID NO: 737) | |
| AB- | EVQVVESGGGEIKPGGSVRLSCTAN | DIVMTQSPLFLSVTPGESASMSCASS |
| 011799 | SFTFSNAWVNWVRQAPGRGLEWV | ESLQRGQYHFLDWYLQKPGQSPQL |
| | GRLRSDTEGWTADYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 838) |
| | (SEQ ID NO: 738) | |
| AB- | EVQVVESGGGEIKPGGSVRLSCTAN | DIVMTQSPLFLSVTPGESASMSCASS |
| 011800 | SFTFSNAWVNWVRQAPGRGLEWV | ESLQRGQYHFLDWYLQKPGQSPQL |
| | GRLRSDTEGWTARYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 839) |
| | (SEQ ID NO: 739) | |
| AB- | EVQVVESGGGEIKPGGSVRLSCTAN | DIVMTQSPLFLSVTPGESASMSCASS |
| 011801 | SFTFSNAWVNWVRQAPGRGLEWV | ESLQRGQYHFLDWYLQKPGQSPQL |
| | GRLRSDTEGWTKDYAPHLADRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 840) |
| | (SEQ ID NO: 740) | |
| AB- | EVQVVESGGGEIKPGGSVRLSCTAN | DIVMTQSPLFLSVTPGESASMSCASS |
| 011802 | SFTFSNAWVNWVRQAPGRGLEWV | ESLQRGQYHFLDWYLQKPGQSPQL |
| | GRLRSDTEGWTKDYAPHLYDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 841) |
| | (SEQ ID NO: 741) | |
| AB- | EVQVVESGGGEIKPGGSVRLSCTAN | DIVMTQSPLFLSVTPGESASMSCASS |
| 011803 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGAWPQSAFLTNRAYAMDVWG | SFGPGTTVDMK |
| | RGTTVVVSS | (SEQ ID NO: 842) |
| | (SEQ ID NO: 742) | |
| AB- | EVQVVESGGGEIKPGGSVRLSCTAN | DIVMTQSPLFLSVTPGESASMSCASS |
| 011804 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGRWPQSAFLTNRAYAMDVWG | SFGPGTTVDMK |
| | RGTTVVVSS | (SEQ ID NO: 843) |
| | (SEQ ID NO: 743) | |
| AB- | EVQVVESGGGEIKPGGSVRLSCTAN | DIVMTQSPLFLSVTPGESASMSCASS |
| 011805 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYAPQSAFLTNRAYAMDVWG | SFGPGTTVDMK |
| | RGTTVVVSS | (SEQ ID NO: 844) |
| | (SEQ ID NO: 744) | |
| AB- | EVQVVESGGGEIKPGGSVRLSCTAN | DIVMTQSPLFLSVTPGESASMSCASS |
| 011806 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYEPQSAFLTNRAYAMDVWG | SFGPGTTVDMK |
| | RGTTVVVSS | (SEQ ID NO: 845) |
| | (SEQ ID NO: 745) | |
| AB- | EVQVVESGGGEIKPGGSVRLSCTAN | DIVMTQSPLFLSVTPGESASMSCASS |
| 011807 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPASAFLTNRAYAMDVWG | SFGPGTTVDMK |
| | RGTTVVVSS | (SEQ ID NO: 846) |
| | (SEQ ID NO: 746) | |
| AB- | EVQVVESGGGEIKPGGSVRLSCTAN | DIVMTQSPLFLSVTPGESASMSCASS |
| 011808 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPYSAFLTNRAYAMDVWG | SFGPGTTVDMK |
| | RGTTVVVSS | (SEQ ID NO: 847) |
| | (SEQ ID NO: 747) | |
| AB- | EVQVVESGGGEIKPGGSVRLSCTAN | DIVMTQSPLFLSVTPGESASMSCASS |
| 011809 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQYAFLTNRAYAMDVWG | SFGPGTTVDMK |
| | RGTTVVVSS | (SEQ ID NO: 848) |
| | (SEQ ID NO: 748) | |
| AB- | EVQVVESGGGEIKPGGSVRLSCTAN | DIVMTQSPLFLSVTPGESASMSCASS |
| 011810 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQEAFLTNRAYAMDVWG | SFGPGTTVDMK |
| | RGTTVVVSS | (SEQ ID NO: 849) |
| | (SEQ ID NO: 749) | |
| AB- | EVQVVESGGGEIKPGGSVRLSCTAN | DIVMTQSPLFLSVTPGESASMSCASS |
| 011811 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSYFLTNRAYAMDVWG | SFGPGTTVDMK |
| | RGTTVVVSS | (SEQ ID NO: 850) |
| | (SEQ ID NO: 750) | |
| AB- | EVQVVESGGGEIKPGGSVRLSCTAN | DIVMTQSPLFLSVTPGESASMSCASS |
| 011812 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSKFLTNRAYAMDVWG | SFGPGTTVDMK |
| | RGTTVVVSS | (SEQ ID NO: 851) |
| | (SEQ ID NO: 751) | |
| AB- | EVQVVESGGGEIKPGGSVRLSCTAN | DIVMTQSPLFLSVTPGESASMSCASS |
| 011813 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAALTNRAYAMDVWG | SFGPGTTVDMK |
| | RGTTVVVSS | (SEQ ID NO: 852) |
| | (SEQ ID NO: 752) | |
| AB- | EVQVVESGGGEIKPGGSVRLSCTAN | DIVMTQSPLFLSVTPGESASMSCASS |
| 011814 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAQLTNRAYAMDVWG | SFGPGTTVDMK |
| | RGTTVVVSS | (SEQ ID NO: 853) |
| | (SEQ ID NO: 753) | |
| AB- | EVQVVESGGGEIKPGGSVRLSCTAN | DIVMTQSPLFLSVTPGESASMSCASS |
| 011815 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFATNRAYAMDVWG | SFGPGTTVDMK |
| | RGTTVVVSS | (SEQ ID NO: 854) |
| | (SEQ ID NO: 754) | |
| AB- | EVQVVESGGGEIKPGGSVRLSCTAN | DIVMTQSPLFLSVTPGESASMSCASS |
| 011816 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLENRAYAMDVWG | SFGPGTTVDMK |
| | RGTTVVVSS | (SEQ ID NO: 855) |
| | (SEQ ID NO: 755) | |
| AB- | EVQVVESGGGEIKPGGSVRLSCTAN | DIVMTQSPLFLSVTPGESASMSCASS |
| 011817 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLYNRAYAMDVWG | SFGPGTTVDMK |
| | RGTTVVVSS | (SEQ ID NO: 856) |
| | (SEQ ID NO: 756) | |
| AB- | EVQVVESGGGEIKPGGSVRLSCTAN | DIVMTQSPLFLSVTPGESASMSCASS |
| 011818 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTQRAYAMDVWG | SFGPGTTVDMK |
| | RGTTVVVSS | (SEQ ID NO: 857) |
| | (SEQ ID NO: 757) | |
| AB- | EVQVVESGGGEIKPGGSVRLSCTAN | DIVMTQSPLFLSVTPGESASMSCASS |
| 011819 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTARAYAMDVWG | SFGPGTTVDMK |
| | RGTTVVVSS | (SEQ ID NO: 858) |
| | (SEQ ID NO: 758) | |
| AB- | EVQVVESGGGEIKPGGSVRLSCTAN | DIVMTQSPLFLSVTPGESASMSCASS |
| 011820 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNEAYAMDVWG | SFGPGTTVDMK |
| | RGTTVVVSS | (SEQ ID NO: 859) |
| | (SEQ ID NO: 759) | |
| AB- | EVQVVESGGGEIKPGGSVRLSCTAN | DIVMTQSPLFLSVTPGESASMSCASS |
| 011821 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNSAYAMDVWG | SFGPGTTVDMK |
| | RGTTVVVSS | (SEQ ID NO: 860) |
| | (SEQ ID NO: 760) | |
| AB- | EVQVVESGGGEIKPGGSVRLSCTAN | DIVMTQSPLFLSVTPGESASMSCASS |
| 011822 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRYYAMDVWG | SFGPGTTVDMK |
| | RGTTVVVSS | (SEQ ID NO: 861) |
| | (SEQ ID NO: 761) | |
| AB- | EVQVVESGGGEIKPGGSVRLSCTAN | DIVMTQSPLFLSVTPGESASMSCASS |
| 011823 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRNYAMDVWG | SFGPGTTVDMK |
| | RGTTVVVSS | (SEQ ID NO: 862) |
| | (SEQ ID NO: 762) | |
| AB- | EVQVVESGGGEIKPGGSVRLSCTAN | DIVMTQSPLFLSVTPGESASMSCASS |
| 011824 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAAAMDVWG | SFGPGTTVDMK |
| | RGTTVVVSS | (SEQ ID NO: 863) |
| | (SEQ ID NO: 763) | |
| AB- | EVQVVESGGGEIKPGGSVRLSCTAN | DIVMTQSPLFLSVTPGESASMSCASS |
| 011825 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAKAMDVWG | SFGPGTTVDMK |
| | RGTTVVVSS | (SEQ ID NO: 864) |
| | (SEQ ID NO: 764) | |
| AB- | EVQVVESGGGEIKPGGSVRLSCTAN | DIVMTQSPLFLSVTPGESASMSCASS |
| 011826 | SFTFSNAWVNWVRQAPGRGLEWV | ASLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 865) |
| | (SEQ ID NO: 765) | |
| AB- | EVQVVESGGGEIKPGGSVRLSCTAN | DIVMTQSPLFLSVTPGESASMSCASS |
| 011827 | SFTFSNAWVNWVRQAPGRGLEWV | YSLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 866) |
| | (SEQ ID NO: 766) | |
| AB- | EVQVVESGGGEIKPGGSVRLSCTAN | DIVMTQSPLFLSVTPGESASMSCASS |
| 011828 | SFTFSNAWVNWVRQAPGRGLEWV | EYLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 867) |
| | (SEQ ID NO: 767) | |
| AB- | EVQVVESGGGEIKPGGSVRLSCTAN | DIVMTQSPLFLSVTPGESASMSCASS |
| 011829 | SFTFSNAWVNWVRQAPGRGLEWV | EELQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 868) |
| | (SEQ ID NO: 768) | |
| AB- | EVQVVESGGGEIKPGGSVRLSCTAN | DIVMTQSPLFLSVTPGESASMSCASS |
| 011830 | SFTFSNAWVNWVRQAPGRGLEWV | ESLQREQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 869) |
| | (SEQ ID NO: 769) | |
| AB- | EVQVVESGGGEIKPGGSVRLSCTAN | DIVMTQSPLFLSVTPGESASMSCASS |
| 011831 | SFTFSNAWVNWVRQAPGRGLEWV | ESLQRSQYHFLDWYLQKPGQSPQLL |
| | GRLRSDTEGWTKDYAPHLKDRVVI | IYLGFRRASGVPARFSARASDTHYT |
| | SREESGHTVFLEMASLQPQDTGVYY | LTISRVDTEDVGVYYCMQALNPPLS |
| | CNTGYWPQSAFLTNRAYAMDVWG | FGPGTTVDMK |
| | RGTTVVVSS | (SEQ ID NO: 870) |
| | (SEQ ID NO: 770) | |
| AB- | EVQVVESGGGEIKPGGSVRLSCTAN | DIVMTQSPLFLSVTPGESASMSCASS |
| 011832 | SFTFSNAWVNWVRQAPGRGLEWV | ESLQRGYYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 871) |
| | (SEQ ID NO: 771) | |
| AB- | EVQVVESGGGEIKPGGSVRLSCTAN | DIVMTQSPLFLSVTPGESASMSCASS |
| 011833 | SFTFSNAWVNWVRQAPGRGLEWV | ESLQRGSYHFLDWYLQKPGQSPQLL |
| | GRLRSDTEGWTKDYAPHLKDRVVI | IYLGFRRASGVPARFSARASDTHYT |
| | SREESGHTVFLEMASLQPQDTGVYY | LTISRVDTEDVGVYYCMQALNPPLS |
| | CNTGYWPQSAFLTNRAYAMDVWG | FGPGTTVDMK |
| | RGTTVVVSS | (SEQ ID NO: 872) |
| | (SEQ ID NO: 772) | |
| AB- | EVQVVESGGGEIKPGGSVRLSCTAN | DIVMTQSPLFLSVTPGESASMSCASS |
| 011834 | SFTFSNAWVNWVRQAPGRGLEWV | ESLQRGQAHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 873) |
| | (SEQ ID NO: 773) | |
| AB- | EVQVVESGGGEIKPGGSVRLSCTAN | DIVMTQSPLFLSVTPGESASMSCASS |
| 011835 | SFTFSNAWVNWVRQAPGRGLEWV | ESLQRGQHHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 874) |
| | (SEQ ID NO: 774) | |
| AB- | EVQVVESGGGEIKPGGSVRLSCTAN | DIVMTQSPLFLSVTPGESASMSCASS |
| 011836 | SFTFSNAWVNWVRQAPGRGLEWV | ESLQRGQVHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 875) |
| | (SEQ ID NO: 775) | |
| AB- | EVQVVESGGGEIKPGGSVRLSCTAN | DIVMTQSPLFLSVTPGESASMSCASS |
| 011837 | SFTFSNAWVNWVRQAPGRGLEWV | ESLQRGQSHFLDWYLQKPGQSPQLL |
| | GRLRSDTEGWTKDYAPHLKDRVVI | IYLGFRRASGVPARFSARASDTHYT |
| | SREESGHTVFLEMASLQPQDTGVYY | LTISRVDTEDVGVYYCMQALNPPLS |
| | CNTGYWPQSAFLTNRAYAMDVWG | FGPGTTVDMK |
| | RGTTVVVSS | (SEQ ID NO: 876) |
| | (SEQ ID NO: 776) | |
| AB- | EVQVVESGGGEIKPGGSVRLSCTAN | DIVMTQSPLFLSVTPGESASMSCASS |
| 011838 | SFTFSNAWVNWVRQAPGRGLEWV | ESLQRGQYAFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 877) |
| | (SEQ ID NO: 777) | |
| AB- | EVQVVESGGGEIKPGGSVRLSCTAN | DIVMTQSPLFLSVTPGESASMSCASS |
| 011839 | SFTFSNAWVNWVRQAPGRGLEWV | ESLQRGQYNFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 878) |
| | (SEQ ID NO: 778) | |
| AB- | EVQVVESGGGEIKPGGSVRLSCTAN | DIVMTQSPLFLSVTPGESASMSCASS |
| 011840 | SFTFSNAWVNWVRQAPGRGLEWV | ESLQRGQYHALDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 879) |
| | (SEQ ID NO: 779) | |
| AB- | EVQVVESGGGEIKPGGSVRLSCTAN | DIVMTQSPLFLSVTPGESASMSCASS |
| 011841 | SFTFSNAWVNWVRQAPGRGLEWV | ESLQRGQYHKLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 880) |
| | (SEQ ID NO: 780) | |
| AB- | EVQVVESGGGEIKPGGSVRLSCTAN | DIVMTQSPLFLSVTPGESASMSCASS |
| 011842 | SFTFSNAWVNWVRQAPGRGLEWV | ESLQRGQYHFLNWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 881) |
| | (SEQ ID NO: 781) | |
| AB- | EVQVVESGGGEIKPGGSVRLSCTAN | DIVMTQSPLFLSVTPGESASMSCASS |
| 011843 | SFTFSNAWVNWVRQAPGRGLEWV | ESLQRGQYHFLSWYLQKPGQSPQLL |
| | GRLRSDTEGWTKDYAPHLKDRVVI | IYLGFRRASGVPARFSARASDTHYT |
| | SREESGHTVFLEMASLQPQDTGVYY | LTISRVDTEDVGVYYCMQALNPPLS |
| | CNTGYWPQSAFLTNRAYAMDVWG | FGPGTTVDMK |
| | RGTTVVVSS | (SEQ ID NO: 882) |
| | (SEQ ID NO: 782) | |
| AB- | EVQVVESGGGEIKPGGSVRLSCTAN | DIVMTQSPLFLSVTPGESASMSCASS |
| 011844 | SFTFSNAWVNWVRQAPGRGLEWV | ESLQRGQYHFLDWYLQKPGQSPQL |
| | GRLRSDTEGWTKDYAPHLKDRVVI | LIYAGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 883) |
| | (SEQ ID NO: 783) | |
| AB- | EVQVVESGGGEIKPGGSVRLSCTAN | DIVMTQSPLFLSVTPGESASMSCASS |
| 011845 | SFTFSNAWVNWVRQAPGRGLEWV | ESLQRGQYHFLDWYLQKPGQSPQL |
| | GRLRSDTEGWTKDYAPHLKDRVVI | LIYKGFRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 884) |
| | (SEQ ID NO: 784) | |
| AB- | EVQVVESGGGEIKPGGSVRLSCTAN | DIVMTQSPLFLSVTPGESASMSCASS |
| 011846 | SFTFSNAWVNWVRQAPGRGLEWV | ESLQRGQYHFLDWYLQKPGQSPQL |
| | GRLRSDTEGWTKDYAPHLKDRVVI | LIYLGLRRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 885) |
| | (SEQ ID NO: 785) | |
| AB- | EVQVVESGGGEIKPGGSVRLSCTAN | DIVMTQSPLFLSVTPGESASMSCASS |
| 011847 | SFTFSNAWVNWVRQAPGRGLEWV | ESLQRGQYHFLDWYLQKPGQSPQL |
| | GRLRSDTEGWTKDYAPHLKDRVVI | LIYLGERRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 886) |
| | (SEQ ID NO: 786) | |
| AB- | EVQVVESGGGEIKPGGSVRLSCTAN | DIVMTQSPLFLSVTPGESASMSCASS |
| 011848 | SFTFSNAWVNWVRQAPGRGLEWV | ESLQRGQYHFLDWYLQKPGQSPQL |
| | GRLRSDTEGWTKDYAPHLKDRVVI | LIYLGFHRASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 887) |
| | (SEQ ID NO: 787) | |
| AB- | EVQVVESGGGEIKPGGSVRLSCTAN | DIVMTQSPLFLSVTPGESASMSCASS |
| 011849 | SFTFSNAWVNWVRQAPGRGLEWV | ESLQRGQYHFLDWYLQKPGQSPQL |
| | GRLRSDTEGWTKDYAPHLKDRVVI | LIYLGFRKASGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 888) |
| | (SEQ ID NO: 788) | |
| AB- | EVQVVESGGGEIKPGGSVRLSCTAN | DIVMTQSPLFLSVTPGESASMSCASS |
| 011850 | SFTFSNAWVNWVRQAPGRGLEWV | ESLQRGQYHFLDWYLQKPGQSPQL |
| | GRLRSDTEGWTKDYAPHLKDRVVI | LIYLGFRRHSGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 889) |
| | (SEQ ID NO: 789) | |
| AB- | EVQVVESGGGEIKPGGSVRLSCTAN | DIVMTQSPLFLSVTPGESASMSCASS |
| 011851 | SFTFSNAWVNWVRQAPGRGLEWV | ESLQRGQYHFLDWYLQKPGQSPQL |
| | GRLRSDTEGWTKDYAPHLKDRVVI | LIYLGFRRAEGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 890) |
| | (SEQ ID NO: 790) | |
| AB- | EVQVVESGGGEIKPGGSVRLSCTAN | DIVMTQSPLFLSVTPGESASMSCASS |
| 011852 | SFTFSNAWVNWVRQAPGRGLEWV | ESLQRGQYHFLDWYLQKPGQSPQL |
| | GRLRSDTEGWTKDYAPHLKDRVVI | LIYLGFRRAKGVPARFSARASDTHY |
| | SREESGHTVFLEMASLQPQDTGVYY | TLTISRVDTEDVGVYYCMQALNPPL |
| | CNTGYWPQSAFLTNRAYAMDVWG | SFGPGTTVDMK |
| | RGTTVVVSS | (SEQ ID NO: 891) |
| | (SEQ ID NO: 791) | |
| AB- | EVQVVESGGGEIKPGGSVRLSCTAN | DIVMTQSPLFLSVTPGESASMSCASS |
| 011853 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCAQALNPPL |
| | CNTGYWPQSAFLTNRAYAMDVWG | SFGPGTTVDMK |
| | RGTTVVVSS | (SEQ ID NO: 892) |
| | (SEQ ID NO: 792) | |
| AB- | EVQVVESGGGEIKPGGSVRLSCTAN | DIVMTQSPLFLSVTPGESASMSCASS |
| 011854 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMAALNPPL |
| | CNTGYWPQSAFLTNRAYAMDVWG | SFGPGTTVDMK |
| | RGTTVVVSS | (SEQ ID NO: 893) |
| | (SEQ ID NO: 793) | |
| AB- | EVQVVESGGGEIKPGGSVRLSCTAN | DIVMTQSPLFLSVTPGESASMSCASS |
| 011855 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQAFNPPL |
| | CNTGYWPQSAFLTNRAYAMDVWG | SFGPGTTVDMK |
| | RGTTVVVSS | (SEQ ID NO: 894) |
| | (SEQ ID NO: 794) | |
| AB- | EVQVVESGGGEIKPGGSVRLSCTAN | DIVMTQSPLFLSVTPGESASMSCASS |
| 011856 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQAENPPL |
| | CNTGYWPQSAFLTNRAYAMDVWG | SFGPGTTVDMK |
| | RGTTVVVSS | (SEQ ID NO: 895) |
| | (SEQ ID NO: 795) | |
| AB- | EVQVVESGGGEIKPGGSVRLSCTAN | DIVMTQSPLFLSVTPGESASMSCASS |
| 011857 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPD |
| | CNTGYWPQSAFLTNRAYAMDVWG | LSFGPGTTVDMK |
| | RGTTVVVSS | (SEQ ID NO: 896) |
| | (SEQ ID NO: 796) | |
| AB- | EVQVVESGGGEIKPGGSVRLSCTAN | DIVMTQSPLFLSVTPGESASMSCASS |
| 011858 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPY |
| | CNTGYWPQSAFLTNRAYAMDVWG | SFGPGTTVDMK |
| | RGTTVVVSS | (SEQ ID NO: 897) |
| | (SEQ ID NO: 797) | |
| AB- | EVQVVESGGGEIKPGGSVRLSCTAN | DIVMTQSPLFLSVTPGESASMSCASS |
| 011859 | SFTFSNAWVNWVRQAPGRGLEWV | ESLQRGQYHFLDWYLQKPGQSPQL |
| | GRLRSDTEGWTKDYAPHLKDRVVI | LIYLGFRRASGVPARFSARASDTHY |
| | SREESGHTVFLEMASLQPQDTGVYY | TLTISRVDTEDVGVYYCMQALNPPD |
| | CNTGYWPQSAFLTNRAYAMDVWG | SFGPGTTVDMK |
| | RGTTVVVSS | (SEQ ID NO: 898) |
| | (SEQ ID NO: 798) | |
| AB- | EVQVVESGGGVVKPGGSLRLSCTA | DIVVTQSPLSLPVTLGEPASMSCKSS |
| 011860 | NTFTFNNAWVNWVRQAPGRGLEW | ESLHREGYNFLDWYLQKPGQSPQL |
| | VGRLQSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTNRAYAMDV | LSFGPGTTVDIK |
| | WGRGTTVIVSS | (SEQ ID NO: 899) |
| | (SEQ ID NO: 799) | |
| AB- | EVQVVESGGGVVKPGGSLRLSCTA | DIVVTQSPLSLPVTLGEPASMSCKSS |
| 011861 | NTFTFNNAWVNWVRQAPGRGLEW | ESLHREGYNFLDWYLQKPGQSPQL |
| | VGRLRSNTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTNRAYAMDV | LSFGPGTTVDIK |
| | WGRGTTVIVSS | (SEQ ID NO: 900) |
| | (SEQ ID NO: 800) | |
| AB- | EVQVVESGGGVVKPGGSLRLSCTA | DIVVTQSPLSLPVTLGEPASMSCKSS |
| 011862 | NTFTFNNAWVNWVRQAPGRGLEW | ESLHREGYNFLDWYLQKPGQSPQL |
| | VGRLRSDQEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTNRAYAMDV | LSFGPGTTVDIK |
| | WGRGTTVIVSS | (SEQ ID NO: 901) |
| | (SEQ ID NO: 801) | |
| AB- | EVQVVESGGGVVKPGGSLRLSCTA | DIVVTQSPLSLPVTLGEPASMSCKSS |
| 011863 | NTFTFNNAWVNWVRQAPGRGLEW | ESLHREGYNFLDWYLQKPGQSPQL |
| | VGRLRSDYEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTNRAYAMDV | LSFGPGTTVDIK |
| | WGRGTTVIVSS | (SEQ ID NO: 902) |
| | (SEQ ID NO: 802) | |
| AB- | EVQVVESGGGVVKPGGSLRLSCTA | DIVVTQSPLSLPVTLGEPASMSCKSS |
| 011864 | NTFTFNNAWVNWVRQAPGRGLEW | ESLHREGYNFLDWYLQKPGQSPQL |
| | VGRLRSDTQHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTNRAYAMDV | LSFGPGTTVDIK |
| | WGRGTTVIVSS | (SEQ ID NO: 903) |
| | (SEQ ID NO: 803) | |
| AB- | EVQVVESGGGVVKPGGSLRLSCTA | DIVVTQSPLSLPVTLGEPASMSCKSS |
| 011865 | NTFTFNNAWVNWVRQAPGRGLEW | ESLHRQGYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTNRAYAMDV | LSFGPGTTVDIK |
| | WGRGTTVIVSS | (SEQ ID NO: 904) |
| | (SEQ ID NO: 804) | |
| AB- | EVQVVESGGGVVKPGGSLRLSCTA | DIVVTQSPLSLPVTLGEPASMSCKSS |
| 011866 | NTFTFNNAWVNWVRQAPGRGLEW | ESLHRGQYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTNRAYAMDV | LSFGPGTTVDIK |
| | WGRGTTVIVSS | (SEQ ID NO: 905) |
| | (SEQ ID NO: 805) | |
| AB- | EVQVVESGGGVVKPGGSLRLSCTA | DIVVTQSPLSLPVTLGEPASMSCKSS |
| 011867 | NTFTFNNAWVNWVRQAPGRGLEW | ESLHREGTNFLDWYLQKPGQSPQLL |
| | VGRLRSDTEHWAKDYAAHMKGRV | VYLGSRRAAGVPARFSGSGAGTDY |
| | VISREESGRTVYLEMIGLKVEDTAV | TLRISRVEAEDVGVYYCMQALRPPL |
| | YYCNTGYWPQSVFLTNRAYAMDV | SFGPGTTVDIK |
| | WGRGTTVIVSS | (SEQ ID NO: 906) |
| | (SEQ ID NO: 806) | |
| AB- | EVQVVESGGGVVKPGGSLRLSCTA | DIVVTQSPLSLPVTLGEPASMSCKSS |
| 011868 | NTFTFNNAWVNWVRQAPGRGLEW | ESLHREGYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGKWPQSVFLTNRAYAMDV | LSFGPGTTVDIK |
| | WGRGTTVIVSS | (SEQ ID NO: 907) |
| | (SEQ ID NO: 807) | |
| AB- | EVQVVESGGGVVKPGGSLRLSCTA | DIVVTQSPLSLPVTLGEPASMSCKSS |
| 011869 | NTFTFNNAWVNWVRQAPGRGLEW | ESLHREGYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYDPQSVFLTNRAYAMDV | LSFGPGTTVDIK |
| | WGRGTTVIVSS | (SEQ ID NO: 908) |
| | (SEQ ID NO: 808) | |
| AB- | EVQVVESGGGVVKPGGSLRLSCTA | DIVVTQSPLSLPVTLGEPASMSCKSS |
| 011870 | NTFTFNNAWVNWVRQAPGRGLEW | ESLHREGYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPASVFLTNRAYAMDV | LSFGPGTTVDIK |
| | WGRGTTVIVSS | (SEQ ID NO: 909) |
| | (SEQ ID NO: 809) | |
| AB- | EVQVVESGGGVVKPGGSLRLSCTA | DIVVTQSPLSLPVTLGEPASMSCKSS |
| 011871 | NTFTFNNAWVNWVRQAPGRGLEW | ESLHREGYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVQLTNRAYAMDV | LSFGPGTTVDIK |
| | WGRGTTVIVSS | (SEQ ID NO: 910) |
| | (SEQ ID NO: 810) | |
| AB- | EVQVVESGGGVVKPGGSLRLSCTA | DIVVTQSPLSLPVTLGEPASMSCKSS |
| 011872 | NTFTFNNAWVNWVRQAPGRGLEW | ESLHREGYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTDRAYAMDV | LSFGPGTTVDIK |
| | WGRGTTVIVSS | (SEQ ID NO: 911) |
| | (SEQ ID NO: 811) | |
| AB- | EVQVVESGGGVVKPGGSLRLSCTA | DIVVTQSPLSLPVTLGEPASMSCKSS |
| 011873 | NTFTFNNAWVNWVRQAPGRGLEW | ESLHREGYNFLDWYLQKPGQSPQL |
| | VGRLRSDTEHWAKDYAAHMKGRV | LVYLGSRRAAGVPARFSGSGAGTD |
| | VISREESGRTVYLEMIGLKVEDTAV | YTLRISRVEAEDVGVYYCMQALRPP |
| | YYCNTGYWPQSVFLTNQAYAMDV | LSFGPGTTVDIK |
| | WGRGTTVIVSS | (SEQ ID NO: 912) |
| | (SEQ ID NO: 812) | |

### Variants

In some embodiments, variants of any of the glycan binders disclosed herein can be generated by introducing mutations to the heavy chain and/or light chain sequences. In some embodiments, the mutation(s) are introduced into one or more of the CDRs of a glycan binder disclosed herein, e.g., AB-006410 or other glycan binder disclosed in Table 1 or 2. In some embodiments, the mutation(s) are introduced in the framework regions. In some embodiments, a variant is engineered to be as much like self as possible to minimize immunogenicity. One approach to do so is to identify a close germline sequence and mutate one of the glycan binders at as many mismatched positions (also known as “germline deviations”) to the germline residue type as possible.

For any the mutation disclosed in this application, the name indicates whether the mutation is a heavy chain or light chain, the position in the heavy chain or light chain of the mutation (with the numbering based on the sequences as provided in Table 3 and not on Kabat or other amino acid sequence numbering conventions), the amino acid residue at the position before introduction of the mutation, and the amino acid at the position after introduction of the mutation. For example, L26NS, refers to that the asparagine (N) in the light chain position 26 is mutated to a serine (S). In some embodiments, the one or more of the CDRs of AB-006410 or other glycan binder disclosed in Table 1 or 2 are mutated to generate variants with improved properties. In some embodiments, the LCDR1 of AB-006410 is mutated to remove an N-linked glycosylation site. In some embodiments, the LCDR1 contains the mutation L26NS. Exemplary variants of AB-006410 include AB-011110, AB-011111, AB-011366, AB-011367, AB-011368, AB-011369, AB-011370, AB-011371, AB-011372, AB-011373, AB-011374, AB-011375, AB-011376, AB-011622, AB-011623, AB-011624, AB-011625, AB-011626. AB-011627, AB-011628, AB-011781, AB-011782, AB-011783, AB-011784, AB-011785, AB-011786, AB-011787, AB-011788, AB-011789, AB-011790, AB-011791, AB-011792, AB-011793. AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, or AB-011873 and various sequences of the antibodies are shown in Tables 1-3.

Methods of generating variants are further described in the section entitled “ENGINEERING VARIANTS” and Example 2 below.

### Additional Fc Variants

In addition to the variants discussed above, there are several useful Fc amino acid modifications that can be made for a variety of reasons, including, but not limited to, altering binding to one or more FcγR receptors, altered binding to FcRn receptors and the like as discussed below. Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any heterodimeric protein.

### FcγR Variants

Accordingly, there are several useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the antibodies described herein include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D/332E/330L, 243A, 243L, 264 A, 264V and 299T.

In addition, there are additional Fc substitutions that find use in increased binding to the FcRn receptor and increased serum half-life, as specifically disclosed in U.S. Ser. No. 12/341,769, hereby incorporated by reference in its entirety, including, but not limited to, 434S, 434 A, 428L, 308F, 259I, 428L/434S, 259I/308F, 436I/428L, 436I or V/434S, 436V/428L and 259I/308F/428L. Such modification may be included in one or both Fe domains of the subject antibody.

In some embodiments, a glycan binder disclosed herein, including antibody fragments, of the present disclosure comprises an Fc region that has effector function, e.g., exhibits antibody-dependent cellular cytotoxicity (ADCC), antibody-dependent cellular phagocytosis (ADCP), and/or complement-dependent cytotoxicity (CDC). In some embodiments, the Fc region may be an Fc region engineered to alter one or more functional properties of the antibody, such as serum half-life, complement fixation, Fc receptor binding, and/or ADCC. Accordingly, an Fc region can comprise additional mutations to increase or decrease effector functions, i.e., the ability to induce certain biological functions upon binding to an Fc receptor expressed on an immune cell. Immune cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells.

In some embodiments, an Fc region described herein can include additional modifications that modulate effector function. Examples of Fc region amino acid mutations that modulate an effector function include, but are not limited to, one or more substitutions at positions 228, 233, 234, 235, 236, 237, 238, 239, 243, 265, 269, 270, 297, 298, 318, 326, 327, 329, 330, 331, 332, 333, and 334 (EU numbering scheme) of an Fc region.

Illustrative substitutions that decrease effector functions include the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions that decrease effector functions include S228P, E233P, L235E, N297A, N297D, and P331S. Multiple substitutions may also be present, e.g., L234A and L235A of a human IgG1 Fc region; L234A, L235A, and P329G of a human TgG1 Fc region; S228P and L235E of a human TgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fe region, to decrease effectors functions. Examples of substitutions that increase effector functions include, e.g., E333A, K326W/E333S, S239D/I332E/G236A, S239D/A330L/I332E, G236A/S239D/A330L/I332E, F243L, G236A, and S298A/E333A/K334A. In some embodiments, the Fe mutations include P329G, L234A, L235A, or a combination thereof. Descriptions of amino acid mutations in an Fc region that can increase or decrease effector functions can be found in, e.g., Wang et al., Protein Cell. 9(1): 63-73, 2018; Saunders, Front Immunol. June 7, eCollection, 2019; Kellner et al., Transfus Med Hemother. 44(5): 327-336, 2017; and Lo et al., J Biol Chem. 292(9):3900-3908, 2017.

In some embodiments, an Fc region may have one or more amino acid substitutions that modulate ADCC, e.g., substitutions at positions 298, 333, and/or 334 of the Fc region, according to the EU numbering scheme. Specifically, S298A, E333A, and K334A can be introduced to an Fc region to increase the affinity of the Fc region to FcγRIIIa and decrease the affinity of the Fc region to FcγRIIa and FcγRIIb.

An Fc region can also comprise additional mutations to increase serum half-life. Through enhanced binding to the neonatal Fc receptor (FcRn), such mutations in an Fc region can improve the pharmacokinetics of the antibody. Examples of substitutions in an Fc region that increase the serum half-life of an antibody include, e.g., M252Y/S254T/T256E, T250Q/M428L, N434A, N434H, T307A/E380A/N434A, M428L/N434S, M252Y/M428L, D259I/V308F, N434S, V308W, V308Y, and V308F. Descriptions of amino acid mutations in an Fc region that can increase the serum half-life of an antibody can be found in, e.g., Dumet et al., MAbs. 26:1-10, 2019; Booth et al., MAbs. 10(7):1098-1110, 2018; and Dall'Acqua et al., J Biol Chem. 281(33):23514-24, 2006.

In some embodiments, a glycan binder described herein comprise an Fc region having altered glycosylation that increases the ability of the antibody to recruit NK cells and/or increase ADCC. In some embodiments, the Fc region comprises glycan containing no fucose (i.e., the Fc region is afucosylated). Afucosylated antibodies can be produced using cell lines that express a heterologous enzyme that depletes the fucose pool inside the cell (e.g., GlymaxX® by ProBioGen AG, Berlin, Germany). Non-fucosylated antibodies can also be produced using a host cell line in which the endogenous α-1,6-fucosyltransferase (FUT8) gene is deleted. See Satoh, M. et al., “Non-fucosylated therapeutic antibodies as next-generation therapeutic antibodies,” Expert Opinion on Biological Therapy, 6:11, 1161-1173, DOT: 10.1517/14712598.6.11.1161.

Furthermore, in some embodiments, an antibody of the disclosure may be chemically modified (e.g., one or more chemical moieties can be attached to the antibody) or be modified, e.g., produced in cell lines and/or in cell culture conditions to alter its glycosylation (e.g., hypofucosylation, afucosylation, or increased sialylation), to alter one or more functional properties of the antibody. For example, the antibody can be linked to one of a variety of polymers, for example, polyethylene glycol. In some embodiments, an antibody may comprise mutations to facilitate linkage to a chemical moiety and/or to alter residues that are subject to post-translational modifications, e.g., glycosylation.

### Activity

### Tumor-Binding Activity

The activity of the glycan binders as described herein can be assessed for binding in binding assays. Nonlimiting examples of suitable assays include surface plasmon resonance analysis using a biosensor system such as a Biacore® system or a flow cytometry assay, which are further described in the EXAMPLES section.

In some embodiments, binding to glycans protein is assessed in a competitive assay format with a reference antibody AB-006410 or a reference antibody having the variable regions of AB-006410. In some embodiments, a variant glycan binder in accordance with the present disclosure may block binding of the reference antibody in a competition assay by about 50% or more.

In some embodiments, binding assays to assess variant activity are performed on tumor tissues or tumor cells ex vivo, e.g., on tumor cells that were grown as a tumor graft in a syngeneic (immune-matched) mouse in vivo then harvested and processed within 24-48 hrs. Binding can be assessed by any number of means including flow cytometry and immunohistochemistry or immunofluorescence-based assays.

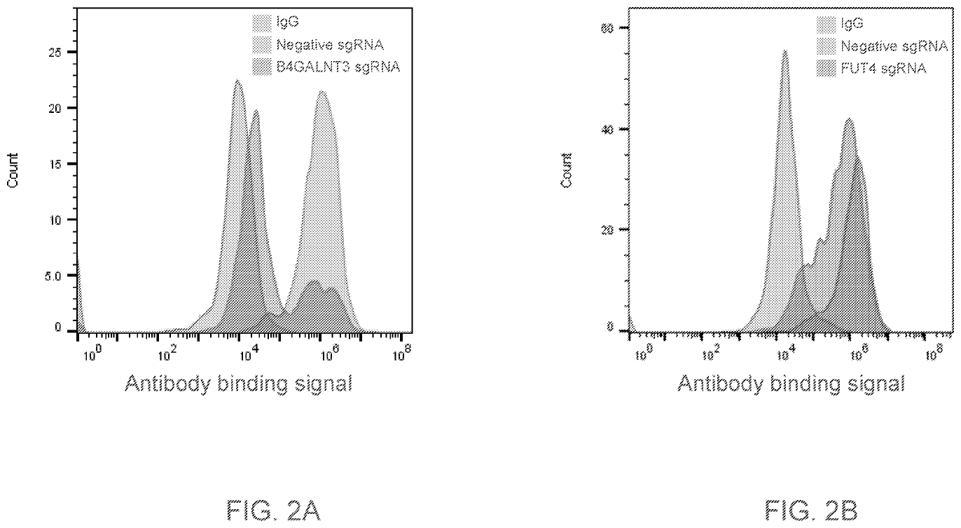

In some embodiments the antibody is added to a cancer cell line and the binding is analyzed by flow cytometry. In one illustrative example, AB-006410 was shown to bind A549 cells and the binding of AB-006410 diminished in A549 cells in which one or more selected glycosyltransferase have been knocked out. In one embodiment, the glycosyltransferase is B4GALNT3 or FUT4. In one embodiment, the glycosyltransferase is B4GALNT3 and FUT4. FIG. 2.

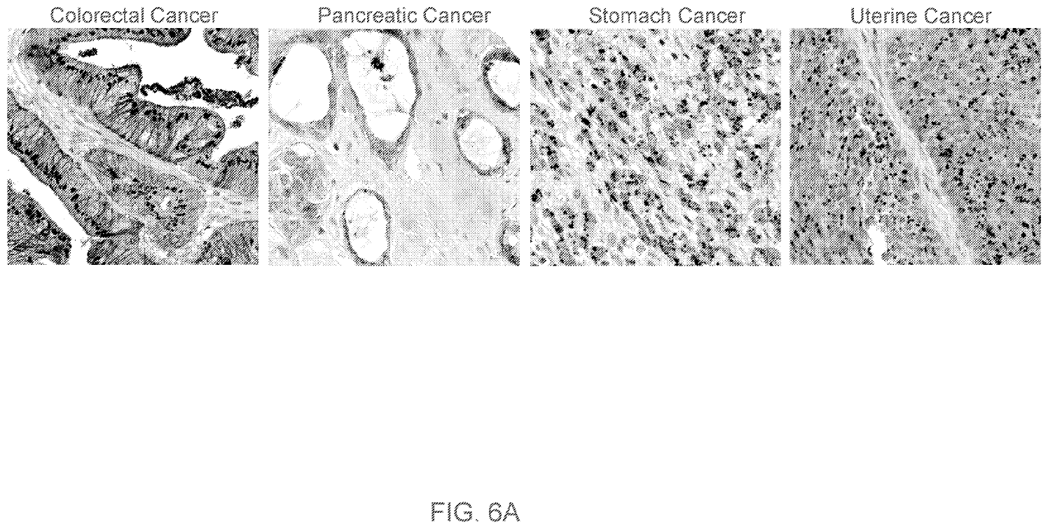

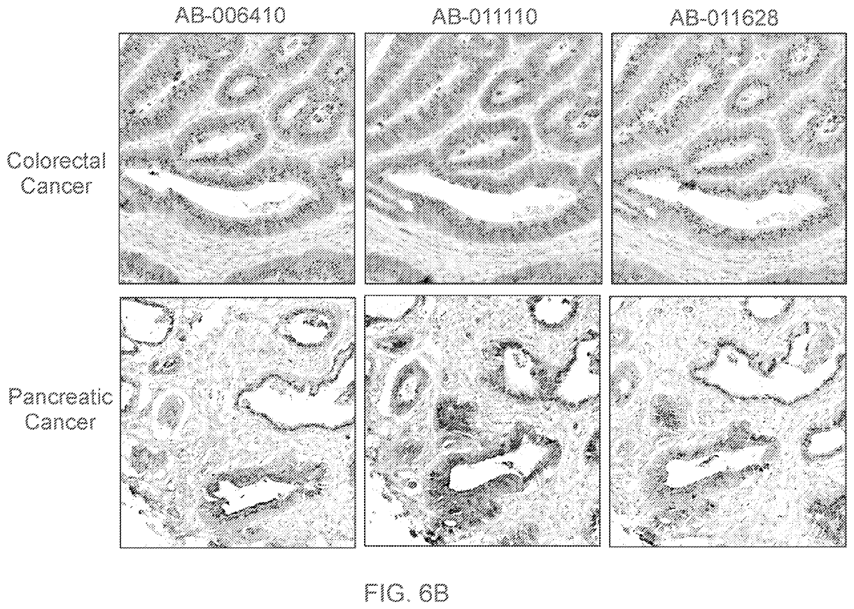

In some embodiments, the binding of the antibodies to bind to tumor cells are assessed by immunofluorescence methods, as described in the EXAMPLES. The glycan binders preferentially bind to various tumors but not to normal human tissues (FIG. 6A). In one illustrative example, the AB-006410 showed preferential binding to ovarian, lung, pancreatic, and esophageal cancer tissues relative to the respective tumor adjacent tissues (TATs).

In some embodiments, the antibody's binding activity is assessed by determining EC₅₀ values, and in some embodiments additionally determining delta activity, i.e., the difference in specific activity between lower and upper plateaus of the activation curve expressed as percent of activity of a selected antibody having known in vitro activity. In typical embodiments, EC₅₀ values are compared to a reference antibody. For purposes of this disclosure, an antibody comprising the VH and VL regions of a glycan binder disclosed herein and a mouse IgG2a Fc region when testing ex vivo binding using a mouse tumor model, is employed as a reference antibody and included in an assay to assess variant activity relative to the reference antibody. The fold over EC₅₀ is calculated by dividing the EC₅₀ of the reference antibody by the EC₅₀ of the test antibody. Based on the resulting values, the antibodies were assigned to groups and given a ranking from 0-4 as follows: 0=(>500 nM); 1=<0.5; 2=0.5 to 2; 3=2 to 4; 4=>4.

### Fc Effector Function

In some embodiments, a glycan binder of the present disclosure comprises an Fc region that has effector function. Examples of effector functions include, but are not limited to, C1q binding and complement-dependent cytotoxicity (CDC), Fc receptor binding (e.g., FcγR binding), ADCC, antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, the Fc region of the glycan binders disclosed herein may be an Fc region engineered to alter one or more functional properties of the antibody, such as serum half-life, complement fixation, Fc receptor binding, and/or ADCC. Accordingly, an Fc region can comprise additional mutations to increase or decrease effector functions, i.e., the ability to induce certain biological functions upon binding to an Fc receptor expressed on an immune cell. Immune cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells. In some embodiments, an antibody of the present disclosure has enhanced ADCC and/or serum stability compared to antibody AB-006410 when the antibody is assayed in a human IgG1 isotype format.

The glycan binders of the present disclosure may be evaluated in various assays for their ability to mediate FcR-dependent activity. In one assay, the binding activity of a glycan binder is evaluated in an Fc receptor engagement assay. For purposes of testing variants, “engagement” of an Fc receptor occurs when a variant antibody binds to both a target tumor cell via its Fv region and an FcγR present on an immune cell via the antibody Fc region in such as manner to transduce a signal. If the Fc region is kept constant among variants that differ in their Fv regions, then the assay allows an evaluation of tumor binding activity across such variants in the context of potential signal transduction through a particular Fc region binding a particular Fc receptor. In some embodiments, binding of the antibody Fc region can result in clustering and/or internalization of the FcR, resulting in a luminescence signal in cells harboring a NFAT-RE-Luciferase reporter construct.

### ADCC Activity

In some embodiments, a glycan binder of the present disclosure has ADCC when the antibodies are assayed in a mouse IgG2a isotype format.

### ADCP Activity

In some embodiments, ADCP activity of a glycan binder is assessed using fluorescently labeled, in vitro cultured tumor cells and Raw264.7 murine macrophages. In certain embodiments, opsonization of the tumor cell by the antibody leads to phagocytosis detected by flow cytometry. Variations of this assay have been described and can include co-labeling of tumor and effector cells or assessment of phagocytosis through FcγRIIa engagement (e.g., FcγRIIa-H ADCP Reporter Bioassay from Promega).

### ADC Activity

A glycan binder antibody is deemed to have ADC activity if, when the antibody is conjugated to a drug molecule (toxin) to form an antibody drug conjugate (ADC), said ADC can kill target cells. In some embodiments, the antibody is deemed to have ADC activity if the EC₅₀ of the assay measuring the cell killing activity of the ADC is less than 1E-08. In one exemplary assay, the ADC activity of glycan binder is evaluated using a drug-conjugated secondary antibody. The antibody-drug conjugate assay involves tumor target cells, primary antibodies of interest (the glycan binder to be tested), and a secondary antibody that is conjugated to a drug molecule, where the secondary antibody recognizes the primary antibody. Briefly, primary antibody dilutions are incubated with target cells at room temperature for a first period (for example, 10-30 minutes). The drug-conjugated secondary antibody is then added to the incubation mixture containing the target cells and the primary antibody. The mixture is then incubated for second period before measuring the extent of target cell lysis. In some embodiments, the assay generates a 100% cell lysis value by adding cell lysis buffer directly to target cell sample, which are not treated by the primary or drug-conjugated second antibody, and cell killing data from samples treated the antibody mixtures as disclosed above can be normalized to the value of 100% cell lysis. The results of the assay can be used to predict whether an ADC produced by conjugating a particular antibody and the drug molecule can kill target cells.

### In Vivo Activity

In some embodiments, activity of a glycan binder is evaluated in vivo in a suitable animal tumor model. A reduction in tumor load of a subject treated with a test article relative to the tumor load of a subject treated with a control article reflects the anti-tumor function of an antibody. A glycan binder, or glycan antibody immunoconjugate, disclosed herein can reduce tumor load of a subject by at least 20%, at least 30%, at least 40%, at least 50%, at least 50%, or at least 60%, or 70%, or greater relative to the tumor load of a control subject.

In some embodiments, a variant of an antibody as described herein has at least 20%, at least 30%, at least 40%, at least 50%, or at least 60%, or 70%, or greater, of the anti-tumor activity of a reference antibody as shown in Tables 1-3 when evaluated under the same assay conditions to measure the anti-tumor activity in vivo. In some embodiments, an anti-tumor antibody exhibits improved activity, i.e., greater than 100% activity, compared to the reference antibody.

### Antibody Formats

In some embodiments a glycan binder in accordance with the present disclosure is in a monovalent format. In some embodiments, the tumor-targeting antibody is in a fragment format, e.g., a Fv, Fab, Fab′, scFv, diabody, or F(ab′)₂ fragment. In a further aspect of the disclosure, a glycan binder in accordance with the disclosure may be an antibody fragment, e.g., a Fv, Fab, Fab′, scFv, diabody, or F(ab′)₂ fragment. In another embodiment, the antibody is a substantially full-length antibody, e.g., an IgG antibody or other antibody class or isotype as defined herein. For a review of certain antibody fragments, see Hudson et al. Nat. Med. 9: 129-134 (2003). Antibody fragments can be made by various techniques, including but not limited to proteolytic digestion of an intact antibody as well as production by recombinant host cells.

In some embodiments, a glycan binder according to the present disclosure that is administered to a patient is an IgG of the IgG1 subclass. In some embodiments, such an antibody is an IgG of the IgG2, IgG3, or IgG4 subclass. In some embodiments, such an antibody is an IgM. In some embodiments, such an antibody has a lambda light chain constant region. In some embodiments, such an antibody has a kappa light chain constant region.

In some embodiments, a glycan binder of the present disclosure is employed in a bispecific or multi-specific format, e.g., a tri-specific format. For example, in some embodiments, the antibody may be incorporated into a bispecific or multi-specific antibody that comprises a further binding domain that binds to the same or a different antigen.

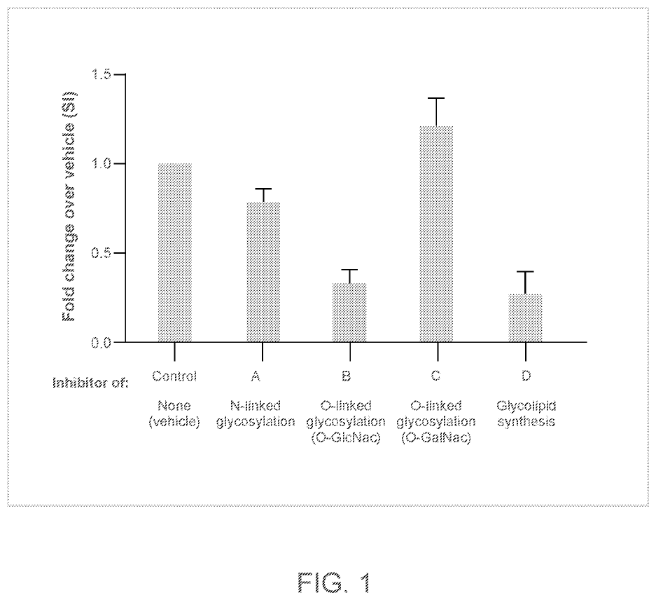

There are a variety of possible formats that can be used in bispecific or multi-specific antibodies. The formats can vary elements such as the number of binding arms, the format of each binding arm (e.g., Fab, scFv, scFab, or VH-only), the number of antigen binding domains present on the binding arms, the connectivity and geometry of each arm with respect to each other, the presence or absence of an Fc domain, the Ig class (e.g., IgG or IgM), the Fc subclass (e.g., hIgG1, hIgG2, or hIgG4), and any mutations to the Fc (e.g., mutations to reduce or increase effector function or extend serum half-life). Also see Speiss, et al., Alternative Molecular Formats and Therapeutic Applications for Bispecific Antibodies, Mol Immunol, 67, 95-106 (2015), particularly FIG. 1, for examples of bispecific and multispecific formats.

In one embodiment of any of the above bispecific or multispecific antibody constructs, the tumor-targeting binding domain comprises all six CDRs (HCDR1, HCDR2, HCDR3, LCDR1, LCDR2, and LCDR3) sequences from the individual antibodies disclosed in Tables 1 and 2.

In one embodiment of any of the above bispecific or multispecific antibody constructs, the tumor-targeting binding domain comprises the VH and VL sequences from the individual antibodies disclosed in Tables 3.

In one embodiment of any of the above bispecific or multispecific antibody constructs, the tumor-targeting binding domain comprises all six CDRs (HCDR1, HCDR2, HCDR3, LCDR1, LCDR2, and LCDR3) sequences from any one of antibodies AB-006410, AB-011110, AB-011111, AB-011366, AB-011367, AB-011368, AB-011369, AB-011370, AB-011371, AB-011372, AB-011373, AB-011374, AB-011375, AB-011376, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, and AB-011873.

In one embodiment of any of the above bispecific or multispecific antibody constructs, the tumor-targeting binding domain comprises the VH and VL sequences of AB-006410, AB-011110, AB-011111, AB-011366, AB-011367, AB-011368, AB-011369, AB-011370, AB-011371, AB-011372, AB-011373, AB-011374, AB-011375, AB-011376, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792. AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, and AB-011873.

In some embodiments, a glycan binder disclosed herein is constructed as a multivalent antibody. In some embodiments a glycan binder is constructed as a tetravalent molecule, comprising four glycan-binding arms per molecule. Such constructs exhibit increased ADCC activity, as well as increased binding to tumor cells a measured by flow cytometry.

In some embodiments, a glycan binder of the present disclosure is employed in a bispecific or multi-specific format, e.g., a tri-specific format. For example, in some embodiments, the antibody may be incorporated into a bispecific or multi-specific antibody that comprises a further binding domain that binds to the same or a different antigen.

There are a variety of possible formats that can be used in bispecific or multi-specific antibodies. The formats can vary elements such as the number of binding arms, the format of each binding arm (e.g., Fab, scFv, scFab, or VH-only), the number of antigen binding domains present on the binding arms, the connectivity and geometry of each arm with respect to each other, the presence or absence of an Fc domain, the Ig class (e.g., IgG or IgM), the Fc subclass (e.g., hIgG1, hIgG2, or hIgG4), and any mutations to the Fc (e.g., mutations to reduce or increase effector function or extend serum half-life). Also see Speiss, et al., Alternative Molecular Formats and Therapeutic Applications for Bispecific Antibodies, Mol Immunol, 67, 95-106 (2015), FIG. 1, for examples of bispecific and multispecific formats.

Illustrative antigens that can be targeted by a further binding domain in a bispecific or multi-specific antibody that comprises an antigen binding domain of a glycan binder described herein, include, but are not limited to, antigens on T cells to enhance T cell engagement and/or activate T cells. Illustrative examples of such an antigen include, but are not limited to, CD3, CD2, CD4, CD5, CD6, CD8, CD28, CD40L, CD44, IL-15Rα, CD122, CD132, or CD25. In some embodiments, the antigen is CD3. In some embodiments, the antigen is in a T cell activating pathway, such as a 4-1BB/CD137, 4-1BBL/CD137L, OX40, OX40L, GITRL, GITR, CD27, CD70, CD28, ICOS, HVEM, or LIGHT antigen.

Multi-Specific Formats that Bind to CD3

In some embodiments, a glycan binder is incorporated into a bispecific or multi-specific antibody that comprises a binding domain that binds to a T-cell antigen. These bispecific antibodies or multi-specific antibodies can direct T cells to attach and lyse targeted tumor cells, i.e., the glycan expressing tumor cells. In some embodiments, the bispecific or multispecific antibody comprises a binding domain that binds to CD3. In some embodiments, the bispecific or multispecific antibody comprises a binding domain that binds to human CD3 comprising the anti-tumor antibodies described herein.

As will be appreciated by those in the art, any collection of anti-CD3 CDRs, anti-CD3 variable light and variable heavy domains, Fabs and scFvs as depicted in any of the Figures can be used. Similarly, any of the anti-glycans antigen binding domains can be used, whether CDRs, variable light and variable heavy domains, Fabs and scFvs, can be used, optionally and independently combined in any combination.

Multi-Specific Formats that Bind to 4-1BB

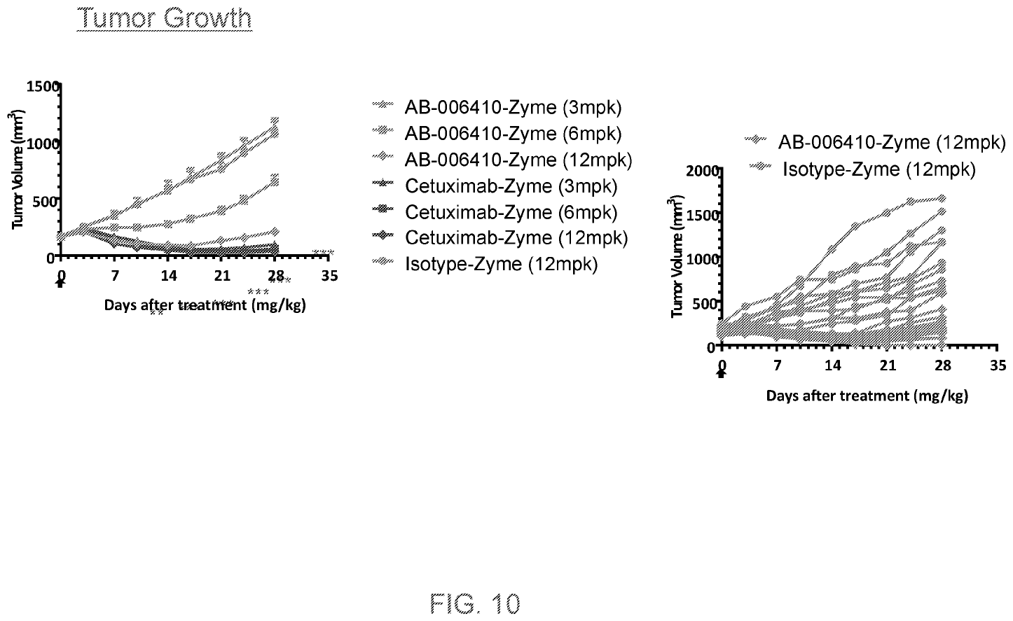

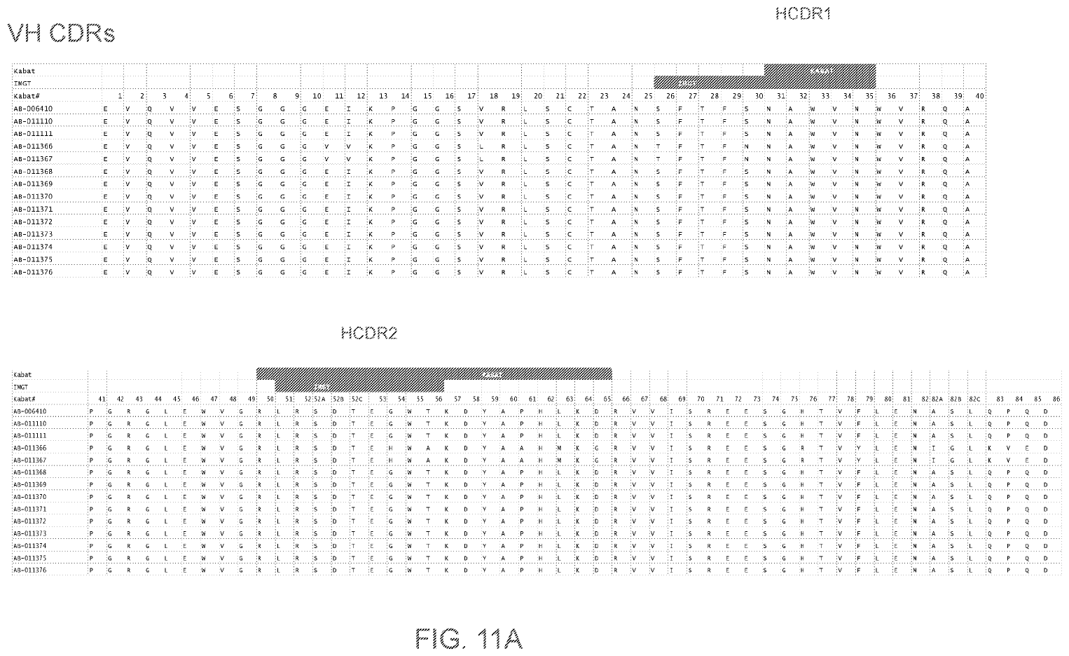

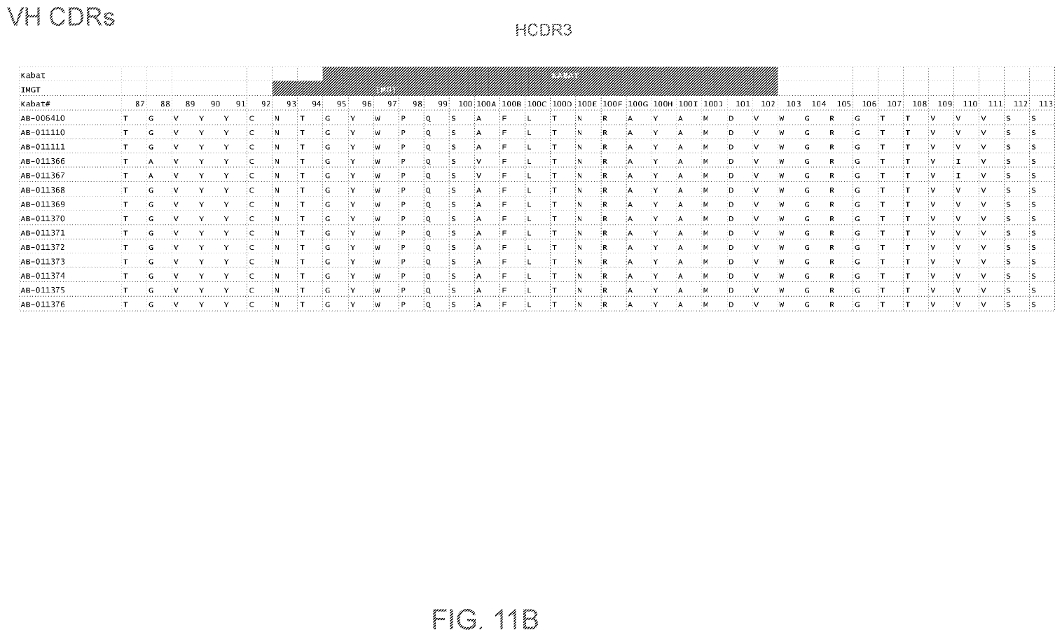

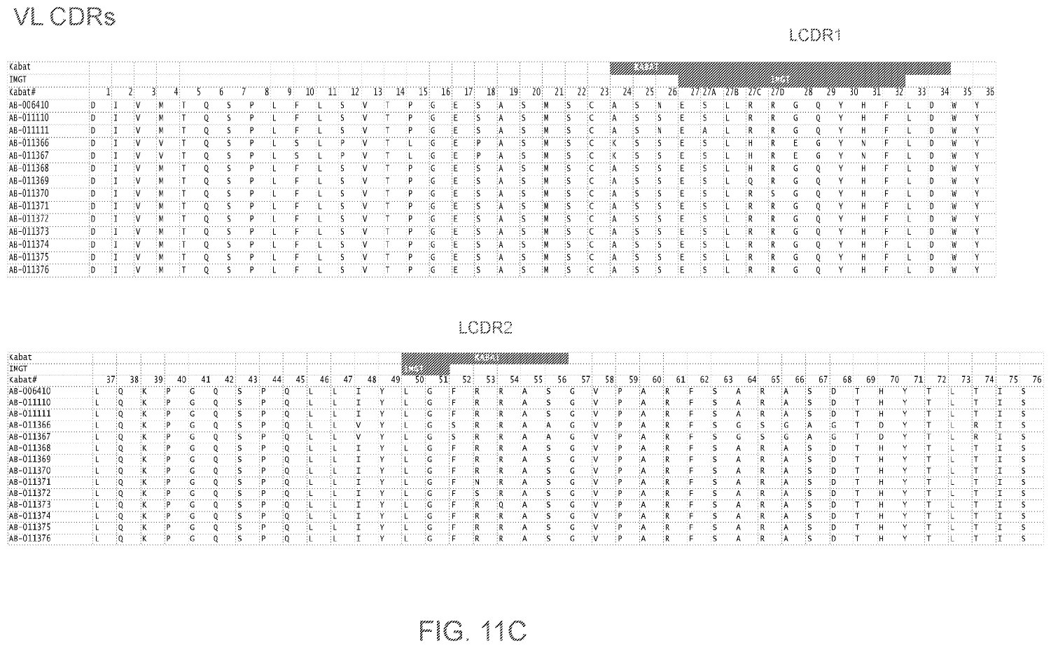

In some embodiments, a glycan binder is incorporated into a multi-specific antibody that comprises a binding domain from an agonist antibody that binds to 4-1BB. In one embodiment, the 4-1BB agonist antibody is a bispecific antibody that is capable of binding to both glycans and 4-1BB. For purposes of this application, the term “4-1BB engager,” refers to the portion of a molecule (e.g., a bispecific antibody capable of binding to both 4-1BB and glycans) that binds to 4-1BB. In some embodiments, the 4-1BB engager is an antibody or an antibody fragment (e.g., scFv) that binds to 4-1BB. In some embodiments, the 4-1BB engager is a multimeric 4-1BB ligand (“4-1BBL”), for example, a 4-1BBL trimer. In some embodiments, as further described below, the bispecific antibody comprises one or more scFv fragments of an anti-4-1BB antibody and a glycan binder disclosed herein. In one embodiment, the 4-1BB agonist antibody is a trispecific antibody. Examples of bispecific and trispecific antibody constructs are described in US 20190010248, FIG. 1; WO2020025659, FIG. 1; Berezhnoy A et al. Converting PD-L1-induced T-lymphocyte Inhibition into CD137-mediated Costimulation via PD-L1×CD137 Bispecific DART© Molecules. Poster presented at 30^{th} EORTC/AACR/NCI Symposium, Nov. 13-16, 2018, Dublin, Ireland, Compte, M. et al. A tumor-targeted trimeric 4-1BB-agonistic antibody induces potent tumor-targeting immunity without systemic toxicity. Nat Com 9, 4809 (2018). FIG. 1; U.S. Ser. No. 10/239,949, FIG. 10; and WO2019/092452, Example 2.

In one embodiment, the fusion molecule comprises a silenced human IgG1 with three human 4-1BB ligand ectodomains attached via flexible linkers. See WO2019086499, FIGS. 1-3. Other 4-1BBL fusion molecules can be utilized with the tumor-targeting antibodies described herein, see Zhang er al., Targeted and Untargeted CD137L Fusion Proteins for the Immunotherapy of Experimental Solid Tumors, Clin Cancer Res., 2758-2767 (2007), FIG. 1; (Kermer et al., Combining Antibody-Directed Presentation of IL-15 and 4-1BBL in a Trifunctional Fusion Protein for Cancer Immunotherapy, Mol Cancer Ther, 112-121 (2014), FIG. 1.

In some embodiments, a glycan binder is incorporated into a fusion molecule comprising one or more 4-1BB ligands (4-1BBL). In one embodiment, a trimer of 4-1BBL is fused to the C-terminal of either the light chain or heavy chain of a glycan binder. In one embodiment, one or more individual 4-1BBL domains are connected via linkers, with one of the domains additionally fused to the gly can binder via a linker. The 4-1BBL domains comprise the entire ECD portion of the molecule or truncated forms that can still bind and activate 4-1BB. See WO2019086499, FIGS. 1-3.

### Generation of Antibodies

The glycan binders can be produced using vectors and recombinant methodology well known in the art (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors. Accordingly, in a further aspect of the disclosure, provided herein are isolated nucleic acids encoding a V_{H} and/or V_{L} region, or fragment thereof, of any of the tumor-targeting antibodies as described herein; vectors comprising such nucleic acids and host cells into which the nucleic acids are introduced that are used to replicate the antibody-encoding nucleic acids and/or to express the antibodies. Such nucleic acids may encode an amino acid sequence containing the V_{L} and/or an amino acid sequence containing the V_{H} of the tumor-targeting antibody (e.g., the light and/or heavy chains of the antibody). In some embodiments, the host cell contains (1) a vector containing a polynucleotide that encodes the V_{L} amino acid sequence and a polynucleotide that encodes the V_{H} amino acid sequence, or (2) a first vector containing a polynucleotide that encodes the V_{L} amino acid sequence and a second vector containing a polynucleotide that encodes the V_{H} amino acid sequence.

In a further aspect, the invention provides a method of making a glycan binder as described herein. In some embodiments, the method includes culturing a host cell as described in the preceding paragraph under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally can self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1 plasmids, pCR1, RP4, phage DNAs, and shuttle vectors. These and many other cloning vectors are available from commercial vendors.

Expression vectors generally are replicable polynucleotide constructs that contain a nucleic acid of the present disclosure. The expression vector can be replicable in the host cells either as episomes or as an integral part of the chromosomal DNA. Suitable expression vectors include but are not limited to plasmids and viral vectors, including adenoviruses, adeno-associated viruses, retroviruses, and any other vector.

Suitable host cells for expressing a glycan binder as described herein include both prokaryotic and eukaryotic cells. For example, a glycan binder may be produced in bacteria when glycosylation and Fc effector function are not needed. After expression, the antibody may be isolated from the bacterial cell paste in a soluble fraction and can be further purified. Alternatively, the host cell may be a eukaryotic host cell, including eukaryotic microorganisms, such as filamentous fungi or yeast, including fungi and yeast strains whose glycosylation pathways have been “humanized,” resulting in the production of an antibody with a partially or fully human glycosylation pattern, vertebrate, invertebrate, and plant cells. Examples of invertebrate cells include insect cells. Numerous baculoviral strains have been identified which may be used in conjunction with insect cells. Plant cell cultures can also be utilized as host cells.

In some embodiments, vertebrate host cells are used for producing a glycan binder of the present disclosure. For example, mammalian cell lines such as a monkey kidney CV1 line transformed by SV40 (COS-7); human embryonic kidney line (293 or 293 cells as described, e.g., in Graham et al., J. Gen Virol. 36:59,1977; baby hamster kidney cells (BHK); mouse sertoli cells (TM4 cells as described, e.g., in Mather, Biol. Reprod. 23:243-251, 1980 monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68, 1982; MRC 5 cells; and FS4 cells may be used to express an tumor-targeting antibodies. Other useful mammalian host cell lines include Chinese hamster ovary (CHO) cells, including DHFR-CHO cells (Urlaub et al., Proc. Natl. Acad Sci. USA 77:4216, 1980); and myeloma cell lines such as Y0, NS0 and Sp2/0. Host cells of the present disclosure also include, without limitation, isolated cells, in vitro cultured cells, and ex vivo cultured cells. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268, 2003.

In some embodiments, a glycan binder of the present invention is produced by a CHO cell line, e.g., the CHO-K1 cell line. One or more expression plasmids can be introduced that encode heavy and light chain sequences. For example, in one embodiment, an expression plasmid encoding a heavy chain disclosed herein, and an expression plasmid encoding a light chain disclosed herein are transfected into host cells. The expression plasmids can be introduced as linearized plasmids at a ratio of 1:1 in the CHO-K1 host cell line using reagents such as Freestyle Max reagent. Fluorescence-activated cell sorting (FACS) coupled with single cell imaging can be used as a cloning method to obtain a production cell line.

A host cell transfected with an expression vector encoding a glycan binder of the present disclosure, or fragment thereof, can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

In some embodiments, a glycan binder of the present disclosure can be produced by in vitro synthesis (see, e.g., Sutro Biopharma biochemical protein synthesis platform).

In some embodiments, provided herein is a method of generating variants of a glycan binder as disclosed herein. Thus, for example, a construct encoding a variant of a V_{H} CDR3 as described herein can be modified and the V_{H} region encoded by the modified construct can be tested for binding activity to LoVo cells and/or in vivo tumor-targeting activity in the context of a V_{H} region as described herein, that is paired with a V_{L} region or variant region as described herein. Similarly, a construct encoding a variant of a V_{L} CDR3 as described herein can be modified and the V_{L} region encoded by the modified construct can be tested for binding to CT26 cells, or other tumor cells, and/or in vivo tumor-targeting activity efficacy. Such an analysis can also be performed with other CDRs or framework regions and an antibody having the desired activity can then be selected.

### Tumor-Targeting Antibody Conjugates/Co-Stimulatory Agents

In a further aspect, a glycan binder disclosed herein may be conjugated or linked to therapeutic, imaging/detectable moieties, or enzymes. For example, the glycan binder may be conjugated to a detectable marker, a cytotoxic agent, an immunomodulating agent, an imaging agent, a therapeutic agent, an oligonucleotide, or an enzyme. Methods for conjugating or linking antibodies to a desired molecule are well known in the art. The moiety may be linked to the antibody covalently or by non-covalent linkages.

In some embodiments, the antibody is conjugated, either directly or via a cleavable or non-cleavable linker, to a cytotoxic moiety or other moiety that exerts their effects on critical cellular processes required for survival (“payload”) to form an antibody-drug conjugate (“ADC”).

In some embodiments, the glycan binder is conjugated to an auristatin to form an ADC. In some embodiments, the ADC comprises a glycan binder conjugated to a ZymeLink™ Auristatin (ZLA) payload. Such ADC constructs are referred to as ZymeLink™ ADC constructs, as disclosed in U.S. Pat. No. 9,879,086 (incorporated by reference herein) and further disclosed below.

In some embodiments, ZymeLink™ ADCs of the present disclosure comprise a glycan binder conjugated to an auristatin analogue ZLA having structure 1 (also referred to herein as “Compound 1”) via a linker L.

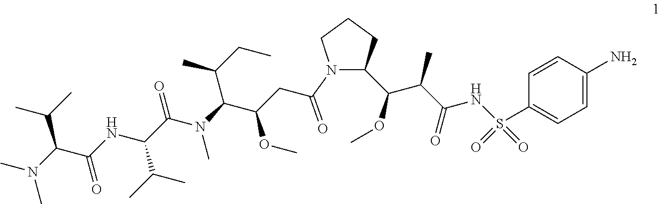 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

In certain embodiments, the ZymeLink™ ADCs have Formula (I):

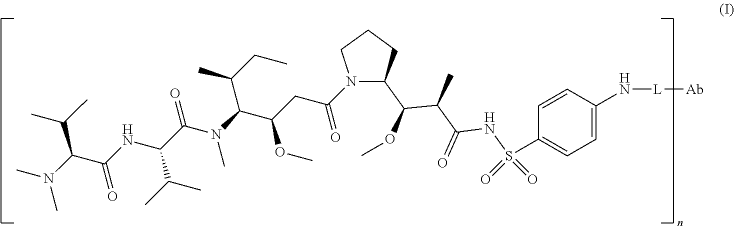

wherein:
L is a cleavable linker;
n is the drug-to-antibody ratio (DAR) and is an integer from 1 to 12, and
Ab is the antibody.

In some embodiments, the each of the n drugs in formula (I) is independently conjugated to the antibody Ab via one of n linkers L.

In some embodiments, in the ZymeLink™ ADCs of general Formula (I), linker L is a protease-cleavable linker.

In some embodiments, in the ZymeLink™ ADCs of Formula (I), linker L is a peptide-containing linker. In some embodiments, in the ADCs of Formula (I), linker L comprises a dipeptide selected from Val-Lys, Ala-Lys, Phe-Lys, Val-Cit, Phe-Cit, Leu-Cit, Ile-Cit and Trp-Cit.

In some embodiments, in the ZymeLink™ ADCs of Formula (I), linker L comprises a dipeptide and a stretcher.

In certain embodiments, linker L in ZymeLink™ ADCs of Formula (I) is conjugated to the antibody via a cysteine residue or a lysine residue on the antibody. In some embodiments, linker L in ADCs of Formula (I) is conjugated to the antibody via a lysine residue on the antibody.

In some embodiments, in the ZymeLink™ ADCs of Formula (I), n is an integer from 1 to 8. In some embodiments, in the ADCs of Formula (I), n has a value from 2 to 8, e.g., from 2 to 6, from 3 to 7, or from 4 to 8. In terms of upper limits, n can be less than 12, e.g., less than 11, less than 10, less than 9, less than 8, less than 7, less than 6, less than 5, less than 4, or less than 3. In terms of lower limits, n can be greater than 2, e.g., greater than 3, greater than 4, greater than 5, greater than 6, greater than 7, greater than 8, greater than 9, greater than 10, or greater than 11. Larger values of n, e.g., greater than 12, are also contemplated.

Combinations of any of the foregoing embodiments for ZymeLink™ ADCs of Formula (I) are also contemplated and each combination forms a separate embodiment for the purposes of the present disclosure.

In certain embodiments, the ZymeLink™ ADCs of the present disclosure have Formula (II):

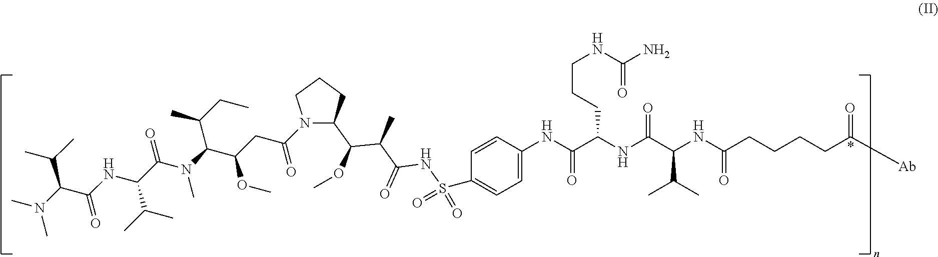

wherein:
n is the drug-to-antibody ratio (DAR) and is an integer from 1 to 12, and
Ab is the antibody.

In some embodiments, the each of the n drugs in formula (II) is independently conjugated to the antibody Ab via one of n linkers L.

In certain embodiments, the carbonyl group of Formula (II) marked with an asterisk forms a peptide bond with the side chain amine group of a lysine residue on the antibody (Ab).

In some embodiments, in the ZymeLink™ ADCs of general Formula (II), n is an integer from 1 to 8. In some embodiments, in the ADCs of general Formula (II), n has a value from 2 to 8, e.g., from 2 to 6, from 3 to 7, or from 4 to 8. In terms of upper limits, n can be less than 12, e.g., less than 11, less than 10, less than 9, less than 8, less than 7, less than 6, less than 5, less than 4, or less than 3. In terms of lower limits, n can be greater than 2, e.g., greater than 3, greater than 4, greater than 5, greater than 6, greater than 7, greater than 8, greater than 9, greater than 10, or greater than 11. Larger values of n, e.g., greater than 12, are also contemplated.

### Linker L

In the ZymeLink™ ADCs of the present disclosure, the antibody is linked to an auristatin analogue ZLA disclosed herein by a linker L. Linkers are bifunctional or multifunctional moieties capable of linking one or more drug molecules to an antibody. A linker may be bifunctional (or monovalent) such that it links a single drug to a single site on the antibody, or it may be multifunctional (or polyvalent) such that it links more than one drug molecule to a single site on the antibody. Linkers capable of linking one drug molecule to more than one site on the antibody may also be multifunctional.

Certain linkers useful in the present invention can be up to 30 carbon atoms in length. For example, the linkers can each independently be from 5 to 20 carbon atoms in length. The types of bonds used to link the linker to the cytotoxic agent and antibody of the present disclosure include, but are not limited to, amides, amines, esters, carbamates, ureas, thioethers, thiocarbamates, thiocarbonate and thioureas. One of skill in the art will appreciate that other types of bonds are useful in the provided ZymeLink™ ADCs.

Attachment of a linker to an antibody can be accomplished in a variety of ways, such as through surface lysines on the antibody, reductive coupling to oxidized carbohydrates on the antibody, or through cysteine residues on the antibody liberated by reducing interchain disulfide linkages. Alternatively, attachment of a linker to an antibody may be achieved by modification of the antibody to include additional cysteine residues (see, for example, U.S. Pat. Nos. 7,521,541; 8,455,622 and 9,000,130) or non-natural amino acids that provide reactive handles, such as selenomethionine, p-acetylphenylalanine, formylglycine or p-azidomethvl-L-phenylalanine (see, for example, Hofer et al., Biochemistry, 48:12047-12057 (2009); Axup et al., PNAS, 109:16101-16106 (2012); Wu et al., PNAS, 106:3000-3005 (2009); Zimmerman et al., Bioconj. Chem., 25:351-361 (2014)), to allow for site-specific conjugation.

Linkers include at least one functional group capable of reacting with the target group or groups on the antibody, and one or more functional groups capable of reacting with a target group on the drug. Suitable functional groups are known in the art and include those described, for example, in Bioconjugate Techniques (G. T. Hermanson, 2013, Academic Press).

Examples of target groups on the antibody to which a linker may be conjugated include the thiol groups of cysteine residues and the amine groups of lysine residues. Non-limiting examples of functional groups for reacting with free cysteines or thiols include maleimide, haloacetamide, haloacetyl, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Also useful in this context are “self-stabilizing” maleimides as described in Lyon et al., Nat. Biotechnol., 32:1059-1062 (2014). Non-limiting examples of functional groups for reacting with free amines include activated esters (such as N-hydroxysuccinamide (NHS) esters, sulfo-NHS esters, imido esters such as Traut's reagent, tetrafluorophenyl (TFP) esters and sulfodichlorophenyl esters), isothiocyanates, aldehydes and acid anhydrides (such as diethylenetriaminepentaacetic anhydride (DTPA)). Other examples include succinimido-1,1,3,3-tetra-methyluronium tetrafluoroborate (TSTU) and benzotriazol-1-yl-oxytripyrrolidinophosphonium hexafluorophosphate (PyBOP).

Other linkers include those having a functional group that allows for bridging of two interchain cysteines on the antibody, such as a ThioBridge® linker (Badescu et al., Bioconjug. Chem., 25:1124-1136 (2014)), a dithiomaleimide (DTM) linker (Behrens et al., Mol. Pharm., 12:3986-3998 (2015)), a dithioarylpyridazinedione-based linker (Lee et al., Chem. Sci., 7:799-802 (2016)), a dibromopyridazinedione-based linker (Maruani et al., Nat. Commun., 6:6645 (2015)) and others known in the art.

A linker may comprise one or more linker components. Typically, a linker will comprise two or more linker components. Exemplary linker components include functional groups for reaction with the antibody, functional groups for reaction with the drug, stretchers, peptide components, self-immolative groups, self-elimination groups, hydrophilic moieties, and the like. Various linker components are known in the art, some of which are described below.

Certain useful linker components can be obtained from various commercial sources, such as Pierce Biotechnology, Inc. (now Thermo Fisher Scientific Corporation, Waltham, MA) and Molecular Biosciences Inc. (Boulder, Colo.), or may be synthesized in accordance with procedures described in the art (see, for example. Toki et al., J. Org. Chem., 67:1866-1872 (2002); Dubowchik et al., Tetrahedron Letters, 38:5257-60 (1997); Walker, M. A., J. Org. Chem., 60:5352-5355 (1995); Frisch et al., Bioconjugate Chem. 7:180-186 (1996); U.S. Pat. Nos. 6,214,345 and 7,553,816, and International Patent Application Publication No. WO 02/088172).

Examples of linker components include, but are not limited to, N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (STAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(β-maleimidopropionamido)hexanoate](SMPH), iminothiolane (IT), sulfo-EMCS, sulfo-GMBS, sulfo-KMUS, sulfo-MBS, sulfo-SIAB, sulfo-SMCC, sulfo-SMPB and succinimidyl-(4-vinylsulfone)benzoate (SVSB).

Additional examples include bis-maleimide reagents such as dithiobismaleimidoethane (DTME), bis-maleimido-trioxyethylene glycol (BMPEO), 1,4-bismaleimidobutane (BMB), 1,4 bismaleimidyl-2,3-dihydroxybutane (BMDB), bismaleimidohexane (BMH), bismaleimidoethane (BMOE), BM(PEG)2 and BM(PEG)3; bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniunbenzoyl)-ethylenediamine), diisocyanates (such as toluene 2,6-diisocyanate), and bis-active fluorine compounds (such as 1,5-difluoro-2,4-dinitrobenzene).

Suitable linkers typically are more chemically stable to conditions outside the cell than to conditions inside the cell, although less stable linkers may be contemplated in certain situations, such as when the drug is selective or targeted and has a low toxicity to normal cells. Linkers may be “cleavable linkers” or “non-cleavable linkers.” A cleavable linker is typically susceptible to cleavage under intracellular conditions, for example, through lysosomal processes. Examples include linkers that are protease-sensitive, acid-sensitive, reduction-sensitive or photolabile. Non-cleavable linkers by contrast, rely on the degradation of the antibody in the cell, which typically results in the release of an amino acid-linker-toxin moiety.

In accordance with the present disclosure, linker L comprised by the ADCs of Formula (I) is a cleavable linker. Suitable cleavable linkers include, for example, linkers comprising a peptide component that includes two or more amino acids and is cleavable by an intracellular protease, such as lysosomal protease or an endosomal protease. A peptide component may comprise amino acid residues that occur naturally and/or minor amino acids and/or non-naturally occurring amino acid analogues, such as citrulline. Peptide components may be designed and optimized for enzymatic cleavage by an enzyme, for example, a tumor-associated protease, cathepsin B, C or D, or a plasmin protease.

In certain embodiments, linker L comprised by the ADCs of Formula (I) may be a peptide-containing linker. In some embodiments, linker L comprised by the ADCs may be a dipeptide-containing linker, such as a linker containing valine-citrulline (Val-Cit) or phenylalanine-lysine (Phe-Lys). Other examples of suitable dipeptides for inclusion in linker L include Val-Lys, Ala-Lys, Me-Val-Cit, Phe-homoLys, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me3Lys-Pro, PhenylGly-(D)Lys, Met-(D)Lys, Asn-(D)Lys, Pro-(D)Lys and Met-(D)Lys. Cleavable linkers may also include longer peptide components such as tripeptides, tetrapeptides or pentapeptides. Examples include, but are not limited to, the tripeptides Met-Cit-Val, Gly-Cit-Val, (D)Phe-Phe-Lys and (D)Ala-Phe-Lys, and the tetrapeptides Gly-Phe-Leu-Gly, Gly-Gly-Phe-Gly and Ala-Leu-Ala-Leu. In some embodiments, linker L comprised by the ADCs may be a peptide-containing linker, where the peptide is between two and five amino acids in length, for example, between two and four amino acids in length.

Additional examples of cleavable linkers include disulfide-containing linkers, such as, N-succinimydyl-4-(2-pyridyldithio) butanoate (SPBD) and N-succinimydyl-4-(2-pyridyldithio)-2-sulfo butanoate (sulfo-SPBD). Disulfide-containing linkers may optionally include additional groups to provide steric hindrance adjacent to the disulfide bond to improve the extracellular stability of the linker, for example, inclusion of a geminal dimethyl group. Other suitable linkers include linkers hydrolyzable at a specific pH or within a pH range, such as hydrazone linkers. Linkers comprising combinations of these functionalities may also be useful, for example, linkers comprising both a hydrazone and a disulfide are known in the art.

A further example of a cleavable linker is a linker comprising a β-glucuronide, which is cleavable by β-glucuronidase, an enzyme present in lysosomes and tumor interstitium (see, for example, De Graaf et al., Curr. Pharm. Des., 8:1391-1403 (2002)).

Cleavable linkers may optionally further comprise one or more additional components such as self-immolative and self-elimination groups, stretchers or hydrophilic moieties.

Self-immolative and self-elimination groups that find use in linkers include, for example, p-aminobenzyloxycarbonyl (PABC) and p-aminobenzyl ether (PABE) groups, and methylated ethylene diamine (MED). Other examples of self-immolative groups include, but are not limited to, aromatic compounds that are electronically similar to the PABC or PABE group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., Chemistry Biology, 2:223-227 (1995)) and 2-aminophenylpropionic acid amides (Amsberry et al., J. Org. Chem., 55:5867-5877 (1990)).

Stretchers that find use in linkers for ADCs include, for example, alkylene groups and stretchers based on aliphatic acids, diacids, amines or diamines, such as diglycolate, malonate, caproate and caproamide. Other stretchers include, for example, glycine-based stretchers, polyethylene glycol (PEG) stretchers and monomethoxy polyethylene glycol (mPEG) stretchers. PEG and mPEG stretchers also function as hydrophilic moieties.

Examples of components commonly found in cleavable linkers include, but are not limited to, SPBD, sulfo-SPBD, hydrazone, Val-Cit, maleidocaproyl (MC), MC-Val-Cit, MC-Val-Cit-PABC, Phe-Lys, MC-Phe-Lys, MC-Phe-Lys-PABC, maleimido triethylene glycolate (MT), MT-Val-Cit, MT-Phe-Lys, TFP and adipate (AD).

Selection of an appropriate linker for a given ZymeLink™ ADC may be readily made by the skilled person having knowledge of the art and taking into account relevant factors, such as the site of attachment to the antibody, any structural constraints of the payload drug and the hydrophobicity of the payload drug (see, for example, review in Nolting, Chapter 5, Antibody-Drug Conjugates: Methods in Molecular Biology, 2013, Ducry (Ed.), Springer).

In certain embodiments, linker L included in the ZymeLink™ ADCs of the present disclosure is a peptide-based linker having Formula (V):

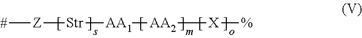

wherein:
Z is a linking group that joins the linker to a target group on the antibody;
Str is a stretcher;
AA₁ and AA₂ are each independently an amino acid, wherein AA₁-[AA₂]ₘ forms a protease cleavage site;
X is a self-immolative group;
s is 0 or 1;
m is 1, 2, 3 or 4;
o is 0, 1 or 2;
# is the point of attachment to the antibody, and
% is the point of attachment to the auristatin analogue.

In some embodiments, in Formula (V), m is 1, 2 or 3.

In some embodiments, in Formula (V), s is 1.

In some embodiments, in Formula (V), o is 0 (i.e., X is absent).

In some embodiments, in Formula (V):

### Z is

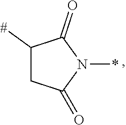

where # is the point of attachment to the antibody, and * is the point of attachment to the remainder of the linker.

In some embodiments, in Formula (V), Z is a carbonyl group (—C(O)—).

In some embodiments, in Formula (V), Str is selected from:

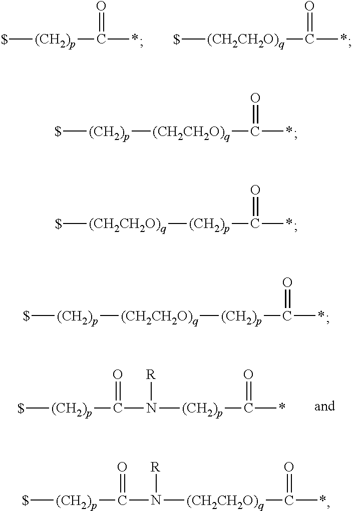

wherein:
each R is independently H or C₁-C₆ alkyl;
each p is independently an integer from 2 to 10;
each q is independently an integer from 1 to 10,
$ is the point of attachment to Z, and
* is the point of attachment to the remainder of the linker.

In some embodiments, in Formula (V), Str is:

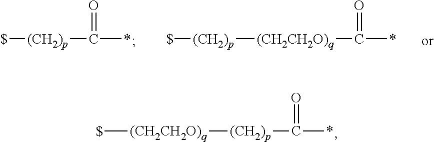

where p, q, $ and * are as defined above.

In some embodiments, in Formula (V), Str is:

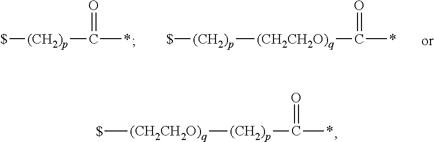

where $ and * are as defined above, p is an integer from 2 to 6, and q is an integer from 2 to 8.

In some embodiments, in Formula (V), AA1-[AA2]m is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys. Asn-(D)Lys, Val-(D)Asp, NorVal-(D)Asp, Ala-(D)Asp, Me3Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly and Ala-Leu-Ala-Leu.

In some embodiments, in Formula (V), m is 1 (i.e., AA1-[AA2]m is a dipeptide).

In some embodiments, in Formula (V), AA1-[AA2]m is a dipeptide selected from Val-Lys, Ala-Lys, Phe-Lys, Val-Cit, Phe-Cit, Leu-Cit, Ile-Cit and Trp-Cit.

In some embodiments, in Formula (V):
Z is a carbonyl group (—C(O)—);
Str is

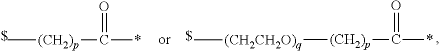

where $ is the point of attachment to Z, * is the point of attachment to the remainder of the linker, p is an integer from 2 to 6, and q is an integer from 2 to 8;
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit;
s is 1; and
o is 0.

In some embodiments, in Formula (V):
Z is

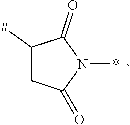

where # is the point of attachment to the antibody, and * is the point of attachment to the remainder of the linker;
Str is

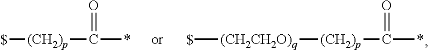

where $ is the point of attachment to Z, * is the point of attachment to the remainder of the linker, p is an integer from 2 to 6, and q is an integer from 2 to 8;
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit;
s is 1; and
o is 0.

In certain embodiments, the linker included in the ZymeLink™ ADCs of the present disclosure has Formula (VI):

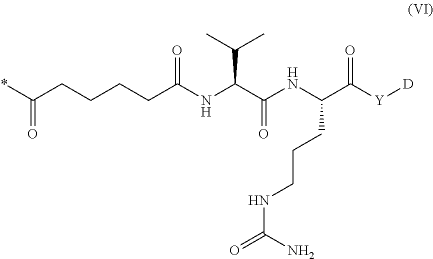

where:
* is the point of attachment to the antibody;
Y is one or more additional linker components, or is absent; and
D is the point of attachment to the auristatin analogue.

In some embodiments, in the linker of Formula (VI), Y is absent.

In certain embodiments, the linker included in the ADCs of the present disclosure has Formula (VII):

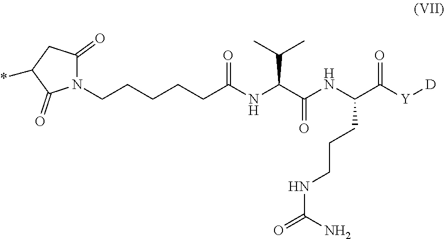

where:
* is the point of attachment to the antibody;
Y is one or more additional linker components, or is absent; and
D is the point of attachment to the auristatin analogue.

In some embodiments, in the linker of Formula (VII), Y is absent.

In certain embodiments, the linker included in the ZymeLink™ ADCs of the present disclosure has Formula (VIII):

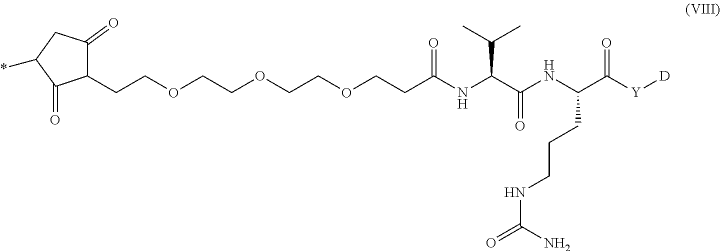

where:
* is the point of attachment to the antibody;
Y is one or more additional linker components, or is absent, and
D is the point of attachment to the auristatin analogue.

In some embodiments, in the linker of Formula (VIII), Y is absent.

In some embodiments, an ADC of the present disclosure is conjugated to a microtubule inhibitor that induces apoptosis in cells undergoing mitosis by, for example, causing cell cycle arrest at G2/M. Nonlimiting examples of microtubule inhibitors that can be used include maytansine derivatives (DM1/DM4), or auristatins (MMAE/MMAF) and variants thereof, such as monomethyl auristatin D, PF-06380101, duostatin5, AS269, Tap18Hr1, AGD-0182, HPA-Auristatin F. In some embodiments, the payload is a tubulin-targeting agent, for example, hemiasterlin, tubulysin, or eribulin. In some embodiments, the payloads are DNA-damaging payloads, which include enediynes (calicheamicin), duocarmycin derivatives, pyrrolobenzodiazepine dimers (PBD dimers), and indolinobenzodiazepine pseudo-dimers.

In some embodiments, the antibody is conjugated to a cytotoxic agent including, but not limited to, e.g., ricin A chain, doxorubicin, daunorubicin, a maytansinoid, taxol, ethidium bromide, mitomycin, etoposide, tenoposide, vincristine, vinblastine, colchicine, dihydroxy anthracin dione, methotrexact, actinomycin, a diphtheria toxin, extotoxin A from Pseudomonas, Pseudomonas exotoxin40, abrin, abrin A chain, modeccin A chain, alpha sarcin, gelonin, mitogellin, restrictocin, cobran venom factor, a ribonuclease, engineered Shiga toxin, phenomycin, enomycin, curicin, crotin, calicheamicin. Saponaria officinalis inhibitor, glucocorticoid, auristatin, auromycin, yttrium, bismuth, combrestatin, duocarmycins, dolastatin, cc1065, or a cisplatin. In some embodiments, the antibody may be linked to an agent such as an enzyme inhibitor, a proliferation inhibitor, a lytic agent, a DNA or RNA synthesis inhibitors, a membrane permeability modifier, a DNA metabolite, a dichloroethylsulfide derivative, a protein production inhibitor, a ribosome inhibitor, or an inducer of apoptosis. In some embodiments, the antibody is conjugated to a drug such as a topoisomeriase Inhibitor, e.g., a topoisomeraise I inhibitor. Topoisomeraise I inhibitors include but are not limited to quinoline alkaloids (e.g., SN-38, DXd).

In some embodiments, the antibody is conjugated to one or more of the cytotoxic and/or anti-mitotic compounds as disclosed in WO2014144871A1 and WO2016041082A1, the entire content of both applications are herein incorporated by reference.

In some embodiments, a glycan binder as described herein is joined to a molecule that facilitates transport of the antibody across a biological membrane, e.g., by enhancing penetration of the membrane, facilitating protein translocation across membranes. Thus, for example, the antibody may be linked to a cell penetration agent, such as a cell-penetrating peptide. Examples of cell penetrating peptides include TAT, penetrating, polyarginine molecules, Kunitz domain-derived peptides, e.g., angiopep-2, SynB, buforin, transportan, amphiphathic peptides and others. In some embodiments, the antibody may be conjugated with a cationic molecule such as a polyamine. In some embodiments, the antibody may be conjugated to an agent that facilitates transport across the blood brain barrier, e.g., transcytosis. Thus, for example, the antibody may be conjugated to an agent that binds to endothelial cell receptors that are internalized, e.g., transferrin receptor, insulin receptor, insulin-like growth factor receptor, or a low-density lipoprotein receptor, and the like. In some embodiments, the antibody may be conjugated to a toxin facilitating entry of the antibody into the cytoplasm, e.g., Shiga toxin. In some embodiments, a glycan binder as described herein can be conjugated to an engineered toxin body (ETBs) to facilitate internalization of the antibody into a cell.

In some embodiments, a glycan binder described herein is conjugated or administered with a polypeptide immunomodulating agent, e.g., an adjuvant. Examples of immunomodulating agents include, but are not limited to, cytokines (e.g., transforming growth factor-β (TGFβ)), growth factors, lymphotoxins, tumor necrosis factor (TNF), hematopoietic factors, interleukins (e.g., interleukin-1 (IL-1), IL-2, IL-3, IL-6, IL-10, IL-12, IL-15, an IL-15/IL-15Ra, e.g., sushi domain, complex, IL-18, and IL-21), colony stimulating factors (e.g., granulocyte-colony stimulating factor (G-CSF) and granulocyte macrophage-colony stimulating factor (GM-CSF), interferons (e.g., interferon-α, -β or -γ, erythropoietin and thrombopoietin, or a combination thereof. In some embodiments, the antibody is linked or administered with a compound that stimulates the innate immune system, such as an adjuvant, a Toll-like receptor (TLR) agonist, a C-type lectin receptor (CLR) agonist, a retinoic acid-inducible gene I-like receptor (RLR) agonist, a saponin, a polysaccharide such as chitin, chitosan, β-glucan, an ISCOM, QS-21, a stimulator of interferon genes (STTNG) agonist, or another immunopotentiating agent.

In some embodiments, a glycan binder described herein is conjugated to or administered with an IL-15 receptor agonist, such as an IL-15 fusion construct, an IL-15:IL-15Rα fusion construct or a single-chain IL-15:IL-15Rα (sushi) fusion construct. In one embodiment, the tumor-targeting antibody conjugated to an IL-15 receptor agonist is a bispecific or multispecific antibody. In some embodiments, the antibody is a bispecific or multispecific antibody comprising an antigen binding domain described herein that further comprises an IL-15 receptor agonist.

In one embodiment, a glycan binder described herein is administered with a single-chain IL-15:IL-15Rα (sushi) fusion construct. In some embodiments, a glycan binder is administered with a polymer-conjugated IL-15 construct, such as NKTR-255.

The IL-15:IL-15Rα single chain constructs can be administered to a subject in a therapeutically effective dose, for example, a dose in a range from less than 0.01 mg/kg body weight to about 25 mg/kg body weight, for example, 0.1-10 mg/kg body weight. In some embodiments, the constructs can be administered in a dose of 1 mg-2 g per patient, or approximately 50 mg-1000 mg/patient.

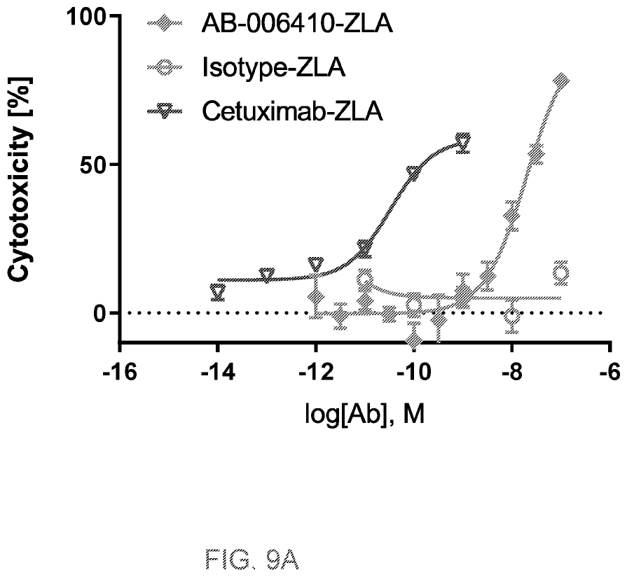

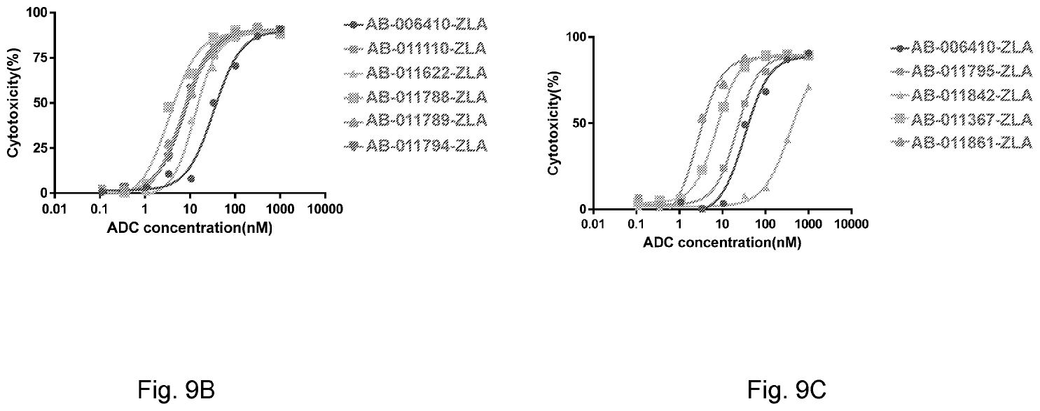

In one embodiment, the single-chain IL-15 fusion construct comprises IL-15 joined to IL-15Rα (sushi) with a polypeptide linker. In one embodiment, the single-chain IL-15 fusion construct is joined via a polypeptide linker to another protein, such as an Fe for long half-life. See, for example, FIG. 9B in WO2018071919A1 (corresponding to U.S. patent Ser. No. 10/550,185). In one embodiment, the IL-15 is joined or fused to the N-terminus of the heavy chain of an Fc, and IL-15Rα(sushi) is joined or fused to the other Fc heavy chain N-terminus, using a heavy chain heterodimerization technology to form the desired hybrid Fc. See, for example, FIG. 9A in WO2018071919A1.

In one embodiment, the IL-15:IL-15Rα (sushi) single chain constructs are fused to the C-terminus of an antibody light chain, or the C-terminus of an antibody heavy chain, in both cases producing a molecule with two tumor-targeting binding sites (the Fab arms), and two IL15:IL15Rα units. In another embodiment, one copy of an IL15:IL15Rα fusion construct is fused to a glycan binder, thereby producing an antibody molecule comprising two tumor-targeting binding sites (the Fab arms) and only one IL15:IL15Rα unit, for example using a knob-in-holes approach to heavy chain heterodimerization, or other heterodimerization technology.

In some embodiments, the IL-15:IL-15Rα (sushi) fusion constructs or the antibodies comprising the fusion constructs comprise a low affinity IL-15 variant having improved pharmacokinetics (PK). In some embodiments, the IL-15:IL-15Rα (sushi) fusion constructs comprise a high affinity IL-15 variant having increased agonist activity. In some embodiments, the high affinity IL-15 variant has an N72D mutation. In some embodiments, the high affinity variant is fused to a dimeric IL-15Rα sushi domain-IgG1 Fc fusion protein. In some embodiments, the IL-15:IL-15Rα (sushi) fusion construct is ALT-803. See Liu B et al. (November 2016). “A Novel Fusion of ALT-803 (Interleukin (IL)-15 Superagonist) with an Antibody Demonstrates Antigen-specific Antitumor Responses”. The Journal of Biological Chemistry. 291 (46): 23869-23881. Doi:10.1074/jbc.M116.733600. Sequences of the IL-15:IL-15Rα fusion constructs and linkers are provided in the Examples.

In some embodiments, antibodies comprising the IL15:IL15Rα fusion construct comprise one or more mutations in the Fe region described herein, for example E333A, K326W/E333S, S239D/1332E/G236A, S239D/A330L/I332E, G236A/S239D/A330L/I332E. F243L, G236A, and S298A/E333A/K334A. In some embodiments, antibodies comprising the IL15:IL15Rα fusion comprise one or more mutations in the Fc region that increase binding of the antibody to tumor cells, for example the mutations P329G, L234A, L235A, or a combination thereof.

In some embodiments, a glycan binder described herein is conjugated to or administered with an IL-2 receptor agonist. In one embodiment, the tumor-targeting antibody conjugated to an IL-2 receptor agonist is a bispecific or multispecific antibody. In some embodiments, the antibody is a bispecific or multispecific antibody comprising an antigen binding domain of an antibody described herein (e.g., AB-006410) that further comprises an IL-2 receptor agonist. In some embodiments, the IL-2 receptor agonist is pegylated IL-2.

In some embodiments, a glycan binder described herein is conjugated to or administered with a construct that can act as a trap for transforming growth factor-β (TGFβ). In one embodiment, the TGFβ trap comprises the extracellular domain (ECD) of TGFβ. In one embodiment, the TGFβ trap comprises the extracellular domain (ECD) of TGFβRII. In one embodiment, the TGFβ trap is in the form of a bispecific antibody (see US2018/0118832A1, FIG. 1). The TGFβRTT ECD can preferably trap TGFβ1, and its low affinity to TGFβ2 may mitigate potential cardiac toxicity.

Thus, in another aspect, a glycan binder described herein comprises an extracellular domain (ECD) of the TGFβ Receptor fused to the C-terminus of the heavy chain or to the C-terminus of the light chain. In some embodiments, the TGFβ trap is a single trap construct. In some embodiments, the single TGFβ trap is a bispecific tumor-targeting TGFβ trap comprising a TGFβ RII ECD fused to any one of the antibodies disclosed herein via a flexible linker to the C-terminus of the heavy chain or to the C-terminus of the light chain.

In some embodiments, the TGFβ trap is a tandem trap construct. In some embodiments, the tandem TGFβ trap comprises an IgG fused to two TGFβRII ECDs. In some embodiments, the tandem TGFβ trap comprises two TGFβ2RII ECDs. In some embodiments, the two TGFβ2RII ECDs are fused in series and are linked by a short linker (for example L10 or L25). In some embodiments, the two TGFβ2RII ECDs are fused directly in series without a llinker (L0). In some embodiments, the tandem TGFβRII ECDs are fused to the C-terminus of the heavy chain (HC-Cter), and the heavy chains were designed as an asymmetric pair such that the tandem-Trap is on only one heavy chain. In some embodiments, the asymmetric pair of heavy chains comprise knob-in-hole mutation that promote pairing of the heavy chains. For example, in some embodiments, one heavy chain comprises the amino acid substitutions T366S+L368A+Y407V (and optionally Y349C), and the other heavy chain comprises the amino acid substitution T336W (and optionally S354C). In some embodiments, the asymmetric single heavy chain C-ter fusion improves steric access of the Fc region to Fc gamma receptors and thereby improve function.

In some embodiments, the tandem TGFβ trap is fused to the C-terminus of the light chain (LC-Cter), such that both light chains comprise two TGFβRII ECDs. In these embodiments, the net molecule exhibits twice the TGFβ trapping capacity per molecule, and therefore may exhibit improved function.

In some embodiments, the bispecific TGFβ trap construct comprises human variable regions. In some embodiments, the bispecific TGFβ trap construct comprises a IgG1 or IgG2 constant region. In some embodiments, the bispecific TGFβ trap construct comprises a human IgG1 constant region. In some embodiments, the bispecific TGFβ trap construct comprises a mouse IgG2a constant region. In some embodiments, the variable regions of the TGFβ trap construct are fused in frame to the IgG constant regions.

Binding of the TGFβ trap construct can be determined using an ELISA assay, as described in the Examples. The ability of TGFβ trap constructs to bind to target tumor cells can be determined, for example, using flow-cytometry, as described in the Examples. The ability of TGFβ trap constructs to engage and stimulate Fc-gamma Receptor in the presence of target tumor cells can be determined using a reporter bioassay, as described in the Examples. The ability of TGFβ trap constructs to inhibit tumor growth can be determined, for example, in a syngeneic mouse model, as described in the Examples.

In some embodiments, the antibody may be linked to a radionuclide, an iron-related compound, a dye, a fluorescent agent, or an imaging agent. In some embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

### Methods of Inducing an Immune Response

In a further aspect, provided herein are methods of inducing an immune response by administering a glycan binder as described herein to a subject that has a tumor. In some embodiments, the glycan binder is an antibody set forth in Tables 1-4, or a variant thereof. In some embodiments, the antibody or variant thereof comprises a modified Fc region comprising mutations described herein. For example, in some embodiments, the antibody comprises an Fc mutation that increases effect function selected from E333A, K326W/E333S, S239D/I332E/G236A, S239D/A330L/I332E, G236A/S239D/A330L/I332E, F243L, G236A, S298A/E333A/K334A, and P329G/L234A/L235A, or a combination thereof. In some embodiments, the antibody comprises a modified Fc region that is afucosylated. In some embodiments, the antibody is conjugated to or administered with an IL-15 receptor agonist, a TGFβ trap, a TLR agonist, or an agonist anti-4-1BB antibody. In some embodiments, the antibody is a bispecific or multispecific antibody described herein.

An immune response induced by administration of an antibody as described herein can be either an innate or adaptive immune response. In some embodiments, the antibody activates an immune response directly, e.g., via binding of the antibody to a target tumor cell and engagement with an Fc receptor on an effector cell such that the effector cell is activated. In some embodiments, the antibody indirectly activates an immune response by inducing immune responses that are initiated by antibody binding to the target cell and an effector cell with subsequent induction of downstream immune responses. In some embodiments, the antibody activates monocytes, myeloid cells, and/or NK cells, e.g., macrophages, neutrophils, dendritic cells, mast cells, basophils, eosinophile, and/or NK cells. In some embodiments, the antibody activates T lymphocytes and/or B cells.

### Treatment of Cancer

In a further aspect, a glycan binder as provided herein, or a variant thereof, can be used as a therapeutic agent to treat cancer. In some embodiments, the glycan binder comprises a modified Fc region comprising mutations described herein. For example, in some embodiments, the antibody comprises an Fc mutation that increases effector function selected from E333A, K326W/E333S, S239D/I332E/G236A, S239D/A330L/I332E, G236A/S239D/A330L/I332E, F243L, G236A, S298A/E333A/K334A, and P329G/L234A/L235A, or a combination thereof. In some embodiments, the antibody comprises a modified Fc region that is afucosylated. In some embodiments, the antibody is conjugated to or administered with an IL-15 receptor agonist, a TGFβ trap, a TLR agonist, or an agonist anti-4-1BB antibody. In some embodiments, the antibody is a bispecific or multispecific antibody described herein.

In some aspects, the disclosure additionally provides methods of identifying subjects who are candidates for treatment with a glycan binder having tumor-targeting effects. Thus, in one embodiment, the disclosure provides a method of identifying a patient who can benefit from treatment with a glycan binder disclosed herein. In one embodiment, the patient has a tumor that expresses a tumor-associated glycan. In some embodiments, the tumor sample is from a primary tumor. In alternative embodiments, the tumor sample is a metastatic lesion. Binding of antibody to tumor cells through a binding interaction with the glycans can be measured using any assay, such as immunohistochemistry or flow cytometry. In some embodiments, binding of antibody to at least 0.2%, 0.5%, or 1%, or at least 5% or 10%, or at least 20%, 30%, or 50%, of the tumor cells in a sample may be used as a selection criterion for determining a patient to be treated with a glycan binder as described herein.

A glycan binder or a glycan antibody immunoconjugate (e.g., a glycan antibody-drug conjugate) disclosed herein can be used to treat several different cancers. In some embodiments, a cancer patient who can benefit from the treatment of the glycan binder or a glycan antibody immunoconjugate (e.g., a glycan antibody-drug conjugate) has a cancer expressing a tumnor-associated glycan. In some embodiments, the cancer is a carcinoma, a melanoma, or a sarcoma. In some embodiments, the cancer is colorectal, pancreatic, gastric, or uterine cancer. In some embodiments, the cancer is a hematological cancer. In some embodiments, the cancer is breast cancer, prostate cancer, testicular cancer, renal cell cancer, bladder cancer, ovarian cancer, cervical cancer, endometrial cancer, lung cancer, colorectal cancer, anal cancer, pancreatic cancer, gastric cancer, esophageal cancer, hepatocellular cancer, head and neck cancer, a brain cancer, e.g., glioblastoma, melanoma, or a bone or soft tissue sarcoma. In one embodiment, the cancer is acral melanoma. In some embodiments, the cancer is acute lymphoblastic leukemia, acute myeloid leukemia, adrenocortical carcinoma, astrocytoma, basal-cell carcinoma, bile duct cancer, bone tumor, brainstem glioma, cerebellar astrocytoma, cerebral astrocytoma, ependymoma, medulloblastoma, supratentorial primitive neuroectodermal tumors, visual pathway and hypothalamic glioma, bronchial adenomas, Burkitt's lymphoma, central nervous system lymphoma, cerebellar astrocytoma, chondrosarcoma, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, epithelioid hemangioendothelioma (EHE), esophageal cancer, Ewing's sarcoma, extracranial germ cell tumor, extragonadal germ cell tumor, extrahepatic bile duct cancer, a cancer of the eye, intraocular melanoma, retinoblastoma, gallbladder cancer, gastrointestinal carcinoid tumor, gastrointestinal stromal tumor (GIST), germ cell tumor, gestational trophoblastic tumor, gastric carcinoma, hairy cell leukemia, hepatocellular carcinoma, Hodgkin lymphoma, hypopharyngeal cancer, hypothalamic and visual pathway glioma, childhood, intraocular melanoma, islet cell carcinoma, Kaposi sarcoma, kidney cancer, laryngeal cancer, leukemias, lip and oral cavity cancer, liposarcoma, liver cancer, non-small cell lung cancer, small-cell lung cancer, lymphomas, macroglobulinemia, male breast cancer, malignant fibrous histiocytoma of bone, medulloblastoma, Merkel cell cancer, mesothelioma, metastatic squamous neck cancer, mouth cancer, multiple endocrine neoplasia syndrome, multiple myeloma, mycosis fungoides, myelodysplastic syndromes, myelogenous leukemia, myeloid leukemia, adult acute, myeloproliferative disorders, chronic, myxoma, nasal cavity and paranasal sinus cancer, nasopharyngeal carcinoma, neuroblastoma, non-Hodgkin lymphoma, oligodendroglioma, oral cancer, oropharyngeal cancer, osteosarcoma, ovarian epithelial cancer, ovarian germ cell tumor, ovarian low malignant potential tumor, paranasal sinus and nasal cavity cancer, parathyroid cancer, penile cancer, pharyngeal cancer, pheochromocytoma, pineal astrocytoma, pineal germinoma, pineoblastoma, supratentorial primitive neuroectodermal tumors, pituitary adenoma. Plasma cell neoplasia, pleuropulmonary blastoma, primary central nervous system lymphoma, rectal cancer, renal cell carcinoma, retinoblastoma, rhabdomyosarcoma, salivary gland cancer, Ewing sarcoma, Kaposi sarcoma, soft tissue sarcoma, uterine sarcoma, Sézary syndrome, non-melanoma skin cancer, melanoma, small intestine cancer, squamous cell carcinoma, squamous neck cancer, stomach cancer, cutaneous T-Cell lymphoma, throat cancer, thymoma, thyroid cancer, transitional cell cancer of the renal pelvis and ureter, trophoblastic tumor, gestational, urethral cancer, uterine cancer, vaginal cancer, vulvar cancer, Waldenström macroglobulinemia, or Wilms tumor.

In some embodiments, the cancer is lung cancer, e.g., non-small cell lung adenocarcinoma or squamous cell carcinoma; breast cancer, e.g., Triple⁻, ER/PR⁺Her2⁻, ER/PR⁻Her2⁺, or Triple⁻; colorectal cancer, e.g., adenocarcinoma, mucinous adenocarcinoma, or papillary adenocarcinoma; esophageal cancer; stomach cancer; kidney cancer, e.g., kidney clear cell cancer; ovarian cancer, e.g., ovarian endometrioid carcinoma, ovarian mucinous cystadenocarcinoma, or ovarian serous cystadenomcarcinoma; melanoma, e.g., acral melanoma, cutaneous melanoma, or mucosal melanoma; uterine or cervical cancer; liver cancer, e.g., hepatocellular carcinoma or bile duct carcinoma; bladder cancer, e.g., transitional or urothelial bladder cancer; or testicular cancer.

In some embodiments, the cancer is pancreatic adenocarcinoma, esophageal adenocarcinoma, NSCLC adenocarcinoma, or ovarian mucinous adenocarcinoma.

In one aspect, methods of the disclosure comprise administering a glycan binder disclosed herein, or a variant thereof, as a pharmaceutical composition to a cancer patient in a therapeutically effective amount using a dosing regimen suitable for treatment of the cancer. The composition can be formulated for use in a variety of drug delivery systems. One or more physiologically acceptable excipients or carriers can also be included in the compositions for proper formulation. Suitable formulations for use in the present invention are found, e.g., in Remington: The Science and Practice of Pharmacy, 21^{st} Edition, Philadelphia, PA. Lippincott Williams & Wilkins, 2005.

The glycan antibody is provided in a solution suitable for administration to the patient, such as a sterile isotonic aqueous solution for injection. The antibody is dissolved or suspended at a suitable concentration in an acceptable carrier. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

### Administration

The pharmaceutical compositions are administered to a patient in an amount sufficient to cure or at least partially arrest the disease or symptoms of the disease and its complications. An amount adequate to accomplish this is defined as a “therapeutically effective dose.” A therapeutically effective dose is determined by monitoring a patient's response to therapy. Typical benchmarks indicative of a therapeutically effective dose includes the amelioration of symptoms of the disease in the patient. Amounts effective for this use will depend upon the severity of the disease and the general state of the patient's health, including other factors such as age, weight, gender, administration route, and the like Single or multiple administrations of the antibody may be administered depending on the dosage and frequency as required and tolerated by the patient. In any event, the methods provide a sufficient quantity of tumor-targeting antibody to effectively treat the patient.

A glycan binder can be administered by any suitable means, including, for example, parenteral, intrapulmonary, and intranasal, administration, as well as local administration, such as intratumor administration. Parenteral infusions include intramuscular, intravenous, intraarterial, intraperitoneal, or subcutaneous administration. In some embodiments, the antibody may be administered by insufflation. In an illustrative embodiment, the antibody may be stored at 10 mg/ml in sterile isotonic aqueous saline solution for injection at 4° C. and is diluted in either 100 ml or 200 ml 0.9% sodium chloride for injection prior to administration to the patient. In some embodiments, the antibody is administered by intravenous infusion over the course of 1 hour at a dose of between 0.01 and 25 mg/kg. In other embodiments, the antibody is administered by intravenous infusion over a period of between 15 minutes and 2 hours. In still other embodiments, the administration procedure is via sub-cutaneous bolus injection.

The dose of antibody is chosen to provide effective therapy for the patient and is in the range of less than 0.01 mg/kg body weight to about 25 mg/kg body weight or in the range 1 mg-2 g per patient. Preferably the dose is in the range 0.1-10 mg/kg or approximately 50 mg-1000 mg/patient. The dose may be repeated at an appropriate frequency which may be in the range once per day to once every three months, or every six months, depending on the pharmacokinetics of the antibody (e.g., half-life of the antibody in the circulation) and the pharmacodynamic response (e.g., the duration of the therapeutic effect of the antibody). In some embodiments, the in vivo half-life of between about 7 and about 25 days and antibody dosing is repeated between once per week and once every 3 months or once every 6 months. In other embodiments, the antibody is administered approximately once per month.

In an illustrative embodiment, the antibody may be stored at 10 mg/ml or 20 mg/ml in a sterile isotonic aqueous solution. The solution can comprise agents such as buffering agents and stabilizing agents. For example, in some embodiments, a buffering agent such as histidine is included to maintain a formulation pH of about 5.5. Additional reagents such as sucrose or alternatives can be added to prevent aggregation and fragmentation in solution and during freezing and thawing. Agents such as polysorbate 80 or an alternative can be included to lower surface tension and stabilizes the antibody against agitation-induced denaturation and air-liquid and ice-liquid surface denaturation. In some embodiments, the solution for injection is stored at 4° C. and is diluted in either 100 ml or 200 ml 0.9% sodium chloride for injection prior to administration to the patient.

### Combination Therapy

A glycan binder may be administered with one or more additional therapeutic agents, e.g., radiation therapy, chemotherapeutic agents and/or immunotherapeutic agents.

In some embodiments, a glycan binder can be administered in conjunction with an agent that targets an immune checkpoint antigen. In one aspect, the agent is a biologic therapeutic or a small molecule. In another aspect, the agent is a monoclonal antibody, a humanized antibody, a human antibody, a fusion protein, or a combination thereof. In certain embodiments, the agents inhibit, e.g., by blocking ligand binding to receptor, a checkpoint antigen that may be PD1, PDL1, CTLA-4, ICOS, PDL2, IDO1, IDO2, B7-H3, B7-H4, BTLA, HVEM, TIM3, GAL9, GITR, HAVCR2, LAG3, KIR, LAIR1, LIGHT, MARCO, OX-40, SLAM, 2B4, CD2, CD27, CD28, CD30, CD40, CD70, CD80, CD86, CD137 (4-1BB), CD160, CD39, VISTA, TIGIT, a SIGLEC, CGEN-15049, 2B4, CHK1, CHK2, A2aR, B-7 family ligands or their receptors, or a combination thereof. In some embodiments, the agent targets PD-1, e.g., an antibody that blocks PD-L1 binding to PD-1 or otherwise inhibits PD-1. In some embodiments, the agent targets CTLA-4. In some embodiments, the agent targets LAG3. In some embodiments, the agent targets TIM3. In some embodiments, the agents target ICOS.

In some embodiments, a glycan binder can be administered in conjunction with a therapeutic antibody, such as an antibody that targets a tumor cell antigen. Examples of therapeutic antibodies include as rituximab, trastuzumab, tositumomab, ibritumomab, alemtuzumab, atezolizunab, avelumab, durvalumab, pidilizumnab, AMP-224, AMP-514, PDR001, cemiplimab, BMS-936559, CK-301, epratuzumab, bevacizumab, elotuzumab, necitumumab, blinatumomab, brentuximab, cetuximab, daratumumab, denosumab, dinutuximab, gemtuzumab ibritumomab ipilimumab, nivolumab, obinutuzumab, ofatumumab, ado-trastuzumab, panitumumab, pembrolizumab, pertuzumab, ramucirumab, and ranibizumab. In some embodiments, a glycan binder can be administered in conjunction with a therapeutic antibody that binds an extracellular RNA-protein complex comprising polyadenylated RNA, such as the antibody designated ATRC-101, see WO2020168231 incorporated herein in its entirety.

In some embodiments, a glycan binder is administered with a chemotherapeutic agent. Examples of cancer chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside; cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid derivatives such as bexarotene, alitretinoin; denileukin diftitox; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, mifepristone, aromatase inhibiting 4(5)-imidazoles, 4-hydroxytamoxifen, trioxifene, keoxifene. LY 1 17018, onapristone, and toremifene (Fareston); and anti-androgens such as flutamide, nilutamide, bicalutamide, leuprolide, and goserelin; and pharmaceutically acceptable salts, acids or derivatives of any of the above. Further cancer therapeutic agents include sorafenib and other protein kinase inhibitors such as afatinib, axitinib, crizotinib, dasatinib, erlotinib, fostamatinib, gefitinib, imatinib, lapatinib, lenvatinib, mubritinib, nilotinib, pazopanib, pegaptanib, ruxolitinib, vandetanib, vemurafenib, and sunitinib; sirolimus (rapamycin), everolimus and other mTOR inhibitors. Examples of additional chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexatc, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea). Illustrative chemotherapeutic agents additionally include paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinasel and other inhibitors of proteins or enzymes known to be upregulated, over-expressed or activated in cancers, the inhibition of which down regulates cell replication. Additional agents include asparaginase and a Bacillus Calmete-Guérin preparation.

In some embodiments, a glycan binder as described herein is administered after, or at the same time, as a therapeutic agent, e.g., a chemotherapeutic agent, such as doxorubicin, that induces stress granules (“SG-inducing agent”). Increasing the amount of stress granules in cancer cells can promote targeting the tumor cells by the tumor-targeting antibody. Other exemplary therapeutic agents that can induce stress granules include pyrimidine analogs (e.g., 5-FU, under trade names of Adrucil®, Carac®, Efudex®, Efudix®); protease inhibitors (e.g., Bortezomib, under the trade name of Velcade®); kinase inhibitors (e.g, Sorafenib and Imatinib, under the trade names of Nexavar® and Gleevec®, respectively); Arsenic compounds (e.g., Arsenic trioxide, under the trade name of Trisenox®); Platinum-based compounds that induce DNA damage (e.g., Cisplatin and Oxaliplatin®, under the trade names of Platinol® and Eloxatin®, respectively); agents that disrupt microtubules (e.g., Vinblastin, under the trade name of Velban® or alkabban-AQ®; vincristin, under the trade name of Vincasar®, Marqibo®, or Oncovin®; Vinorelbin, under the trade name of Navelbin®); topoisomerase II inhibitor (e.g., Etoposide, under the trade name of Etopophos, Toposar®, VePesid®); and agents that induce DNA damage, e.g., irradiation. Several exemplary therapeutic agents that can induce stress granules formation are disclosed in Mahboubi et al., Biochimica et Biophysica Acta 1863 (2017) 884-895.

Various combinations with the glycan binder and the SG-inducing agent (or a combination of such agents) described herein may be employed to treat a cancer patient. By “combination therapy” or “in combination with”, it is not intended to imply that the therapeutic agents must be administered at the same time and/or formulated for delivery together, although these methods of delivery are within the scope described herein. The tumor-targeting antibody and the SG-inducing agent can be administered following the same or different dosing regimen. In some embodiments, the tumor-targeting antibody and the SG-inducing agent is administered sequentially in any order during the entire or portions of the treatment period. In some embodiments, the tumor-targeting antibody and the SG-inducing agent is administered simultaneously or approximately simultaneously (e.g., within about 1, 5, 10, 15, 20, or 30 minutes of each other). In still other embodiments, the SG-inducing agent may be administered 1, 2, 3, 4, 5, 6, 7, 8, 9, 10, or more days before administration of the tumor-targeting antibody. In some embodiments, the SG-inducing agent is administered from 1 to 4 weeks, or longer, before the tumor-targeting antibody is administered.

A glycan binder may also be administered to a cancer patient in conjunction with a cell-based therapy, such as natural killer (NK) cell therapy or a cancer vaccine. In some instances, a cancer vaccine is a peptide-based vaccine, a nucleic acid-based vaccine, a cell-based vaccine, a virus-based or viral fragment-based vaccine or an antigen presenting cell (APC) based vaccine (e.g., dendritic cell-based vaccine). Cancer vaccines include Gardasil®, Cervarix®, sipuleucel-T (Provenge®), NeuVax™, HER-2 ICD peptide-based vaccine, HER-2/neu peptide vaccine, AdHER2/neu dendritic cell vaccine, HER-2 pulsed DC1 vaccine, Ad-sig-hMUC-1/ecdCD40L fusion protein vaccine, MVX-ONCO-1, hTERT/survivin/CMV multipeptide vaccine, E39, J65, P10s-PADRE, rV-CEA-Tricom, GVAX®, Lucanix®, HER2 VRP, AVX901, ONT-10, ISAlO1, ADXS1 1-001, VGX-3100, INO-9012, GSK1437173A, BPX-501, AGS-003, IDC-G305, HyperAcute®-Renal (HAR) immunotherapy, Prevenarl3, MAGER-3.A1, NA17.A2, DCVax-Direct, latent membrane protein-2 (LMP2)-loaded dendritic cell vaccine (NCT02115126), HS410-101 (NCT02010203, Heat Biologies), EAU RF 2010-01 (NCT01435356, GSK), 140036 (NCT02015104, Rutgers Cancer Institute of New Jersey), 130016 (NCTO 1730118, National Cancer Institute), MVX-201101 (NCT02193503, Maxivax SA), ITL-007-ATCR-MBC (NCT01741038, Immunovative Therapies, Limited), CDR0000644921 (NCT00923143, Abramson cancer center of the University of Pennsylvania), SuMo-Sec-01 (NCT00108875, Julius Maximilians Universitaet Hospital), or MCC-15651 (NCT01176474, Medarex, Inc, BMS).

In some embodiments, the glycan binder can be administered with an agent, e.g., a corticosteroid, that mitigates side-effects resulting from stimulation of the immune system.

In the context of the present invention a therapeutic agent that is administered in conjunction with a glycan binder of the present invention can be administered prior to administrations of the tumor-targeting antibody or after administration of the tumor-targeting antibody. In some embodiments, a glycan binder may be administered at the same time as the additional therapeutic agent. In some embodiments, a glycan binder and an additional therapeutic agent described above can be administered following the same or different dosing regimens. In some embodiments, the tumor-targeting antibody and the therapeutic agent are administered sequentially in any order during the entire treatment period or portions thereof. In some embodiments, the tumor-targeting antibody and the therapeutic agent are administered simultaneously or approximately simultaneously (e.g., within about 1, 5, 10, 15, 20, or 30 minutes of each other). In still other embodiments, the therapeutic agent may be administered 1, 2, 3, 4, 5, 6, 7, 8, 9, 10, or more days before the administration of the tumor-targeting antibody. In still other embodiments, the therapeutic agent may be administered 1, 2, 3, 4, 5, 6, 7, 8, 9, 10, or more days after the administration of the tumor-targeting antibody.

### Functional Assays

Also described herein are functional assays that can be used to determine the ability of the antibodies described herein to mediate FcR-dependent activity. In some embodiments, the assay measures antibody dependent cellular cytotoxicity (ADCC), antibody dependent cellular phagocytosis (ADCP), or complement-dependent cytoxicity (CDC).

In some embodiments, binding and activation of FeγRIII by an antibody described herein is determined. In some embodiments, binding and activation of FcγRIIa by an antibody described herein is determined. In some embodiments, binding and activation of the FcR is determined using an ex vivo bioluminescent cell-based assay. In the bioluminescent cell-based assay, primary effector cells are replaced with a Jurkat cell line that stably expresses human a FcγR and a NFAT-induced luciferase. Following engagement with the Fc region by an antibody described herein bound to a target tumor cell, the effector cells expressing FcγRIIa or FcγRIII transduce intracellular signals resulting in NFAT-mediated luciferase activity that can be easily quantified. See Promega website at promega.com/Products/Reporter-Bioassays/fc-effector-activity-bioassays/ADCP-Bioassays/catNum=G9991.

### In Vivo Assays

In some embodiments, the activity of the antibodies is evaluated in vivo in an animal model that is known for specific human tumors. One exemplary model is the CT26 mouse model. Tumor-targeting activity of these antibodies in vivo may be assessed by using several assays, including but not limited to using flow cytometry to analyze the immune profiling of the blood and tumor, monitoring tumor growth, and performing immunofluorescence to semi-quantitative estimate tumor infiltration. In some embodiments, the effect of the antibody can be assessed using Survival, a normalized area above the curve metric (NAAC), and a normalized growth rate metric (NGRM), where NAAC and NGRM were both developed at Atreca. An “in vivo active” determination can be based on the in vivo activity was assessed by a p-value ≤0.05 in at least one of the analyses of survival, NAAC, and NGRM. i.e., if an antibody exhibited a p-value of less than or equal to 0.05 for survival, NAAC, and/or NGRM (any one alone being sufficient), the antibody is considered “in vivo active”.

In one aspect, provided herein are antibodies that exhibit inhibitory effects on tumors, including decreasing rate of tumor growth, size, tumor invasion and/or metastasis. Such antibodies exhibit tumor-targeting effects in vivo, e.g., when administered to subjects that has a tumor expressing a tumor-associated glycan.

### Engineering Variants

In some embodiments, an antibody or variant thereof described herein is modified to have improved developability (i.e., reduced development liabilities), including but not limited to, decreased heterogeneity, increased yield, increased stability, improved net charges to improve pharmacokinetics, and or/reduced immunogenicity. In some embodiments, antibodies having improved developability can be obtained by introducing mutations to reduce or eliminate potential development liabilities. In some embodiments, antibodies having improved developability possess modifications as compared to a reference or control antibody in their amino acid sequence.

In some embodiments, the antibodies or variants thereof disclosed herein have improved developability while maintaining comparable or improved binding affinity to the target antigen as compared to a reference or control (unmodified) antibody. In some embodiments, the antibodies or variants thereof disclosed herein have improved developability while maintaining activities similar to a reference or control (unmodified) antibody.

In some embodiments, the antibodies or variants thereof have improved developability, e.g., as identified through various in vitro assays, such as aggregation assessment by HPLC or UPLC, hydrophobic interaction chromatography (HIC), polyspecificity assays (e.g., baculovirus particle binding), self-interaction nanoparticle spectroscopy (SINS), or mass spec analysis after incubation in an accelerated degradation condition such as high temperature, low pH, high pH, or oxidative H₂O₂. Mutations are successful if activity is maintained (or enhanced) while removing or reducing the severity of the liability.

Improved properties of antibodies or variants thereof as described herein include: (1) fits a standard platform (expression, purification, formulation); (2) high yield; (3) low heterogeneity (glycosylation, chemical modification, and the like); (4) consistent manufacturability (batch-to-batch, and small-to-large scale); (5) high stability (years in liquid formulation), e.g., minimal chemical degradation, fragmentation, and aggregation; and (6) long PK (in vivo half-life), e.g., no off-target binding, no impairment of FcRn recycling, and stable. Antibody liabilities are further described in Table 8.

TABLE 8
Description of potential development liabilities

| | | Sequence | |
|---|---|---|---|
| | | comprises an odd | |
| | Yield, heterogeneity, stability, | number of | |
| Free cysteine⁶ | activity | cysteines | High |
| N-linked glycosylation | Yield, heterogeneity, activity | N(~P)(S, T)¹ | High |
| Abnormal net charge | Platform fit, PK | Sharma 2014² | High |
| Patches of hydrophobicity | Stability, PK | Sharma 2014 | High |
| Patches of same charge | Stability, PK | N/A (based on | Medium |
| | | structure) | |
| Proteolysis | Stability, PK | (K, R)(K, R)³ | Medium |
| Proteolysis | Stability, PK | DP | Medium |
| Asparagine deamidation | Heterogeneity, stability, activity | NG; N(A, N, S, T)⁴ | Medium; |
| | | | Low |
| Aspartate isomerization | Heterogeneity, stability, activity | DG; D(A, D, S, T)⁵ | Medium; |
| | | | Low |
| Lysine glycation | Heterogeneity, stability, activity | K | Low |
| Methionine oxidation | Heterogeneity, stability, activity | M | Low |
| Tryptophan oxidation | Heterogeneity, stability, activity | W | Low |

Note:
¹The N-linked glycosylation site is N-X-S/T, where X is any residue other than proline.
²Sharma et al., Proc. Natl. Acad. Sci. USA 111: 18601-18606, 2014
³This motif consists of a K or R, followed by a K or R. Stated differently, the motif can be KK, KR, RK, or RR.
⁴The dipeptide NG poses a medium risk of development liability. The dipeptides NA, NN, NS, and NT pose a low risk of development liability. N may also exhibit low risk of liability for other successor residues, e.g., D, H, or P. Stated differently, dipeptide ND, NH, or NP poses a low risk of development liability.
⁵Similarly to the above, the dipeptide DG poses a medium risk of development liability. The dipeptides DA, DD, DS, and DT pose a low risk of development liability. D may also exhibit low risk of development liability for other successor residues, e.g., N, H, or P.
⁶“Free cysteine” refers to a cysteine that does not form a disulfide bond with another cysteine and thus is left “free” as thiols. The presence of free cysteines in the antibody can be a potential development liability. Typically, an odd net number of cysteines in the protein shows a likelihood there is a free cysteine.

Another goal for engineering variants is to reduce the risk of clinical immunogenicity: the generation of anti-drug antibodies against the therapeutic antibody. To reduce risk, the antibody sequences are evaluated to identify residues that can be engineered to increase similarity to the intended population's native immunoglobulin variable region sequences.

The factors that drive clinical immunogenicity can be classified into two groups. First are factors that are intrinsic to the drug, such as: sequence; post-translational modifications; aggregates; degradation products; and contaminants. Second are factors related to how the drug is used, such as: dose level; dose frequency; route of administration; patient immune status; and patient HLA type.

One approach to engineering a variant to be as much like self as possible is to identify a close germline sequence and mutate as many mismatched positions (also known as “germline deviations”) to the germline residue type as possible. This approach applies for germline genes IGHV, IGHJ, IGKV, IGKJ, IGLV, and IGLJ, and accounts for all variable heavy (VH) and variable light (VL) regions except for part of H-CDR3. Germline gene IGHD codes for part of the H-CDR3 region but typically exhibits too much variation in how it is recombined with IGHV and IGHJ (e.g., forward, or reverse orientation, any of three translation frames, and 5′ and 3′ modifications and non-templated additions) to present a “self” sequence template from a population perspective.

Each germline gene can present as different alleles in the population. The least immunogenic drug candidate, in terms of minimizing the percent of patients with an immunogenic response, would likely be one which matches an allele commonly found in the patient population. Single nucleotide polymorphism (SNP) data from the human genome can be used to approximate the frequency of alleles in the population.

Another approach to engineering a lead for reduced immunogenicity risk is to use in silico predictions of immunogenicity, such as the prediction of T cell epitopes, or use in vitro assays of immunogenicity, such as ex vivo human T cell activation. For example, services such as those offered by Lonza, United Kingdom, are available that employ platforms for prediction of HLA binding and in vitro assessment to further identify potential epitopes.

Antibody variants can be designed to enhance the efficacy of the antibody. In some embodiments, design parameters can focus on CDRs, e.g., CDR3. Positions to be mutated can be identified based on structural analysis of antibody-antigen co-crystals (Oyen et al., Proc. Natl. Acad Sci. USA 114: E10438-E10445, 2017; Epub Nov. 14, 2017) and based on sequence information of other antibodies from the same lineage.

### Approaches to Mutation Design

Development liabilities can be removed or reduced by one or more mutations. Mutations are designed to preserve antibody structure and function while removing or reducing development liabilities and to improve function. In some embodiments, mutations to chemically similar residues can be identified that maintain size, shape, charge, and/or polarity. Illustrative mutations are described in Table 9.

TABLE 9
Preferred mutations to remove development liabilities

| | | | |
|---|---|---|---|
| Free cysteine | Odd # C | High | C→(A, S) |
| N-linked glycosylation | N(~P)(S, T) | High | N→(Q, D, S, A); (S, T)→(A, N) |
| Proteolytic cleavage | (K, R)(K, R) | Medium | K, R→(Q, S, A) |
| Proteolytic cleavage | DP | Medium | D→(E, S, A) |
| Asparagine deamidation | NG; N(A, N, S, T)* | Medium; Low | N→(Q, S, A); G→(A, S) |
| Aspartate isomcrization | DG; D(A, D, S, T)* | Medium; Low | D→(E, S, A); G→(A, S) |
| Lysine glycation | K | Low | K→(R, Q, S, A) |
| Methionine oxidation | M | Low | M→(Q, L, S, A) |
| Tryptophan oxidation | W | Low | W→(Y, F) |

Note:
the last column in Table 9 shows preferred mutations. For example, C→(A, S) refers to C can be mutated to either A or S to remove development liabilities.

### Methods for Altering the Glycosylation of an Antibody

In another aspect, the antibodies described herein comprise an Fe region having altered glycosylation that increase the ability of the antibody to recruit NK cells and/or increase ADCC. In some embodiments, the Fc region comprises glycan containing no fucose (i.e., the Fc region is afucosylated). Fucosylated antibodies can be produced using cell lines that express a heterologous enzyme that depletes the fucose pool inside the cell (e.g., GlymaxX® by ProBioGen AG, Berlin, Germany). Non-fucosylated antibodies can also be produced using a host cell line in which the endogenous α-1,6-fucosyltransferase (FUT8) gene is deleted. See Satoh, M. et al., “Non-fucosylated therapeutic antibodies as next-generation therapeutic antibodies,” Expert Opinion on Biological Therapy, 6:11, 1161-1173, DOI: 10.1517/14712598.6.11.1161.

### EMBODIMENTS

Embodiment 1 is an antibody that binds a tumor, wherein the binding of the antibody to the tumor is dependent on the expression of one or more glycosyltransferases in the tumor, wherein one of the one or more glycosyltransferases has N-acetyl-galactosaminyltransferase activity.

Embodiment 2 is the antibody of Embodiment 1, wherein one of the one or more glycosyltransferases has fucosyltransferase activity.

Embodiment 3 is the antibody of Embodiment 1 or 2, wherein the glycosyltransferase that has N-acetyl-galactosaminyltransferase activity is selected from the group consisting of B4GALNT3 and B4GALNT4.

Embodiment 4 is the antibody of Embodiment 2 or 3, wherein one of the one or more glycosyltransferases that has fucosyltransferase activity is selected from the group consisting of FUT3, FUT4, FUT5, FUT6, FUT7, FUT9, FUT10, and FUT1l.

Embodiment 5 is the antibody of any one of Embodiments 1-4, wherein the tumor expresses a tumor-associated glycan.

Embodiment 6 is the antibody of Embodiment(s) 5, wherein the tumor-associated glycan is an extracellular glycan.

Embodiment 7 is the antibody of Embodiment 1, wherein the one or more glycosyltransferases is selected from the group consisting of B4GALNT3 and B4GALNT4.

Embodiment 8 is the antibody of any of Embodiments 1-3, wherein the one or more glycosyltransferases is selected from the group consisting of FUT3, FUT4, FUT5, FUT6, FUT7, FUT9, FUT10, and FUT11.

Embodiment 9 is the antibody of Embodiment 8, wherein the glycosyltransferase is FUT4.

Embodiment 10 is the antibody of any of Embodiment 1-9, wherein the glycosyltransferase is B4GALNT3.

Embodiment 11 is the antibody of Embodiment 5, wherein the presence of the tumor-associated glycan is dependent on the expression of B4GALNT3 and FUT4 in the tumor.

Embodiment 12 is the antibody of any of Embodiments 1-11, wherein the antibody preferentially binds to a tumor tissue relative to a normal tissue.

Embodiment 13 is the antibody of any of Embodiments 1-12, wherein the antibody is internalized by the tumor cells upon contacting the tumor.

Embodiment 14 is an antibody that binds to a tumor, wherein the antibody comprises: a heavy chain variable region comprising: an HCDR1, HCDR2, and/or HCDR3 amino acid sequence listed in Table 1, or variants of the HCDR1, HCDR2, and/or HCDR3 amino acid sequence in which 1, 1, 2, 3, 4, 5, or more amino acids are substituted; and/or a light chain variable region comprising: an LCDR1, LCDR2, and/or LCDR3 amino acid sequence listed in Table 2, or variants of the LCDR1, LCDR2, and/or LCDR3 amino acid sequence in which 1, 2, 3, 4, 5, or more amino acid are substituted.

The antibody of any one of Embodiments 1-13 that binds to a tumor, wherein the antibody comprises: a heavy chain variable region comprising:an HCDR1, HCDR2, and/or HCDR3 amino acid sequence listed in Table 1, or variants of the HCDR1, HCDR2, and/or HCDR3 amino acid sequence in which 1, 1, 2, 3, 4, 5, or more amino acids are substituted; and/or a light chain variable region comprising:an LCDR1, LCDR2, and/or LCDR3 amino acid sequence listed in Table 2, or variants of the LCDR1, LCDR2, and/or LCDR3 amino acid sequence in which 1, 2, 3, 4, 5, or more amino acid are substituted.

Embodiment 15 is the antibody of Embodiment 14, wherein the tumor expresses a tumor-associated glycan.

Embodiment 16 is the antibody of Embodiment 14 or 15, wherein the antibody comprises: wherein the antibody comprises all six CDRs of an antibody selected from the group consisting of AB-006410, AB-011110, AB-011111, AB-011366, AB-011367, AB-011368, AB-011369, AB-011370, AB-011371, AB-011372, AB-011373, AB-011374, AB-011375, AB-011376, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, and AB-011873.

Embodiment 17 is the antibody of any one of Embodiments 14-16, wherein the antibody comprises a VH region comprising a VH amino acid sequence in Table 3 or an amino sequence having at least 70%, at least 75%, at least 80%, at least 85%, at least 90%, or at least 95% identity to the VH amino acid sequence in Table 3, and/or wherein the antibody comprises a VL region comprising a VL amino acid sequence in Table 3; and an amino sequence having at least 70%, at least 75%, at least 80%, at least 85%, at least 90%, or at least 95% identity to the VL amino acid sequence in Table 3.

Embodiment 18 is the antibody of any one of Embodiments 14-17, wherein the antibody comprises the VH of an antibody selected from the group consisting of AB-006410, AB-011110, AB-011111, AB-011366, AB-011367, AB-011368, AB-011369, AB-011370, AB-011371, AB-011372, AB-011373, AB-011374, AB-011375, AB-01137, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, AB-011873, and a variant thereof.

Embodiment 19 is the antibody of any one of Embodiment 14-18 wherein the antibody comprises the VL of an antibody selected from the group consisting of AB-006410, AB-011110, AB-011111, AB-011366, AB-011367, AB-011368, AB-011369, AB-011370, AB-011371, AB-011372, AB-011373, AB-011374, AB-011375, AB-01137, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, AB-011873, and a variant thereof.

Embodiment 20 is the antibody of any one of Embodiments 14-19, wherein the antibody comprises both the VH and VL of an antibody selected from the group consisting of AB-006410, AB-011110, AB-011111, AB-011366, AB-011367, AB-011368, AB-011369, AB-011370, AB-011371, AB-011372, AB-011373, AB-011374, AB-011375, AB-01137, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, AB-011873 and a variant thereof.

Embodiment 21 is the antibody of any one of Embodiments 14-19, wherein the antibody comprises both the VH and VL of an antibody selected from the group consisting of AB-011110, AB-011788, AB-011789, AB-011794, AB-011367, and AB-011861.

Embodiment 22 is the antibody of any one of Embodiments 14-20, wherein the binding of antibody to the tumor is dependent on the expression of one or more glycosyltransferases in the tumor, wherein one of the one or more glycosyltransferases has N-acetyl-galactosaminyltransferase activity.

Embodiment 23 is the antibody of Embodiment 22, wherein the one or more glycosyltransferases has fucosyltransferase activity.

Embodiment 24 is the antibody of Embodiment 22 or 23, wherein the glycosyltransferase that has N-acetyl-galactosaminyltransferase activity is selected from the group consisting of B4GALNT3 and B4GALNT4.

Embodiment 25 is the antibody of any one of Embodiments 23-25, wherein one of the one or more glycosyltransferases that has fucosyltransferase activity is selected from the group consisting of FUT3, FUT4, FUT5, FUT6, FUT7, FUT9, FUT10, and FUT11.

Embodiment 26 is the antibody of any one of Embodiments 22-25, wherein the tumor expresses a tumor-associated glycan.

Embodiment 27 is the antibody of Embodiment 26, wherein the tumor-associated glycan is an extracellular glycan.

Embodiment 28 is the antibody of Embodiment 22, wherein the one or more glycosyltransferases is selected from the group consisting of B4GALNT3 and B4GALNT4.

Embodiment 29 is the antibody of any one of Embodiments 22-28, wherein the one or more glycosyltransferases is selected from the group consisting of FUT3, FUT4, FUT5, FUT6, FUT7, FUT9, FUT10, and FUT11.

Embodiment 30 is the antibody of Embodiment 29, wherein the glycosyltransferase is FUT4.

Embodiment 31 is the antibody of any one of Embodiments 22-30, wherein the glycosyltransferase is B4GALNT3.

Embodiment 32 is the antibody of Embodiment 26, wherein the presence of the tumor-associated glycan is dependent on the expression of B4GALNT3 and FUT4 in the tumor.

Embodiment 33 is the antibody of any one of Embodiments 14-32, wherein the antibody preferentially binds to a tumor tissue relative to a normal tissue.

Embodiment 34 is the antibody of any one of Embodiments 14-33, wherein the antibody is internalized by the cells in the tumor upon contacting the tumor.

Embodiment 35 is the antibody of Embodiments 14-34, wherein at least 1 or 2 of the substitutions are conservative substitutions; at least 50% of the substitutions are conservative substitutions; or all of the substitutions are conservative substitutions.

Embodiment 36 is the antibody of any one of Embodiments 14-35, wherein the antibody is a non-natural antibody.

Embodiment 37 is an antibody that competes for binding with the antibody of any one of Embodiments 14-36.

Embodiment 38 is the antibody of any one of Embodiments 1-37, wherein the antibody is a chimeric antibody, a multispecific antibody, a bispecific antibody, an scFv, or a Fab.

Embodiment 39 is the antibody of any one of Embodiments 1-38, wherein the antibody is a bispecific antibody comprising a first and second antigen binding domain, wherein the first binding domain binds to an antigen on the tumor and the second binding domain binds to second antigen.

Embodiment 40 is the antibody of Embodiment 39, wherein the second antigen is 4-1BB or CD3.

Embodiment 41 is an immunoconjugate comprising the antibody of any one of Embodiments 1-39 and a cytotoxic agent.

Embodiment 42 is the immunoconjugate of Embodiment 41, wherein the cytotoxic agent is Auristatin.

Embodiment 43 is the immunoconjugate of Embodiment 41, wherein the cytotoxic agent is ZymeLink™ Auristatin (ZLA).

Embodiment 44 is the immunoconjugate of Embodiments 41-43, wherein the wherein the immunoconjugate comprises Formula (I) or (II):

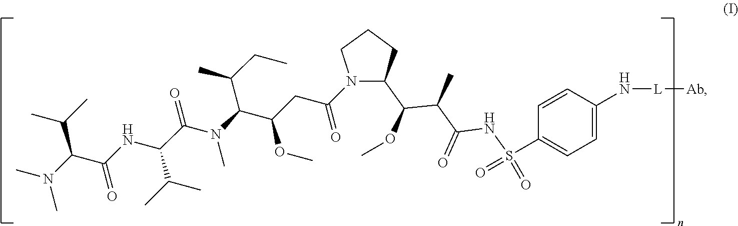

wherein: L is a cleavable linker; n is the drug-to-antibody ratio (DAR) and is an integer from 1 to 12, and Ab is the antibody of any one of Embodiments 1-40; or

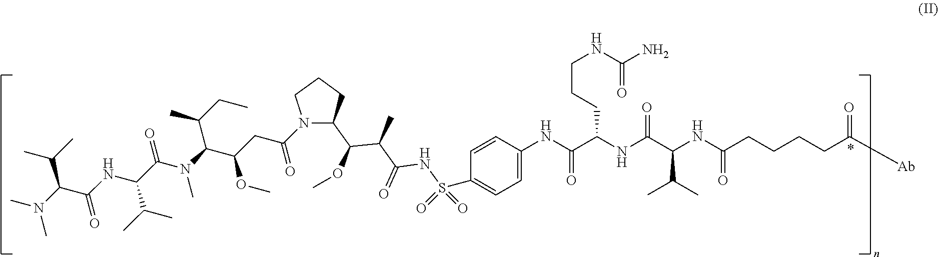

wherein: n is the drug-to-antibody ratio (DAR) and is an integer from 1 to 12, and Ab is the antibody of any one of the claims 1-40.

Embodiment 45 is an immunoconjugate comprising the antibody of any one of Embodiments 1-40 and an IL-15 receptor agonist, a TGFβ trap, a TLR agonist, or a 4-1BB ligand (4-1BBL).

Embodiment 46 is a polypeptide comprising (1) a VH sequence having at least 70% amino acid sequence identity to a VH amino acid sequence in Table 3 and/or a VL sequence having at least 70% amino acid sequence identity to a VL amino acid sequence in Table 3.

Embodiment 47 is the polypeptide of Embodiment 46, wherein the VH amino acid sequence is selected from the group consisting of SEQ ID NOs 85-98 and 713-812, and wherein the VL amino acid sequence is selected from the group consisting of SEQ ID NOs: 99-113 and 813-912.

Embodiment 48 is the polypeptide of Embodiment 46, wherein the VH amino acid sequence is selected from the group consisting of SEQ ID NOs: 86, 727, 728, 733, 89, and 800, and wherein the VL amino acid sequence is selected from the group consisting of SEQ ID NOs: 100, 827, 828, 833, 103, and 900.

Embodiment 49 is a polynucleotide encoding the polypeptide of any one of Embodiments 46-48.

Embodiment 50 is an expression vector comprising a polynucleotide encoding the VH region and/or the VL region of the antibody of any one of Embodiments 1-40.

Embodiment 51 is a host cell that comprises an expression vector of Embodiment 50.

Embodiment 52 is a host cell comprising a polynucleotide that encodes the VH region and/or the VL region of the antibody of any one of Embodiments 1-40.

Embodiment 53 is a pharmaceutical composition comprising an antibody of any one of Embodiments 1-40 and a pharmaceutically acceptable carrier.

Embodiment 54 is a method of treating a cancer patient, the method comprising administering the antibody of any one of Embodiments 1-39 to the patient or the immunoconjugate of any of Embodiments 41-45 to the patient.

Embodiment 55 is the method of Embodiment 54, wherein the cancer is colorectal or gastric cancer.

Embodiment 56 is the method of Embodiment 54 or 55, wherein the method further comprises administering chemotherapy and/or radiation therapy.

Embodiment 57 is a method of identifying a patient having a tumor suitable for treatment with an antibody, wherein the binding of the antibody to the tumor is dependent on the expression of one or more glycotransferases in the tumor, wherein one of the one or more glycosyltransferases has N-acetyl-galactosaminyltransferase activity, wherein the method comprises: contacting a tumor sample from the patient with an antibody of any one of Embodiments 1-40, and detecting binding of the antibody to the tumor sample, wherein detection of the binding identifies the patient having a tumor suitable for treatment with the antibody.

Embodiment 58 is the method of Embodiment 57, wherein one of the one or more glycosyltransferases has fucosyltransferase activity.

Embodiment 59 is the method of Embodiment 57 or 58, wherein the glycosyltransferase that has N-acetyl-galactosaminyltransferase activity is selected from the group consisting of B4GALNT3 and B4GALNT4.

Embodiment 60 is the method of Embodiment 58 or 59, wherein one of the one or more glycosyltransferases that has fucosyltransferase activity is selected from the group consisting of FUT3, FUT4, FUT5, FUT6, FUT7, FUT9, FUT10, and FUT11.

Embodiment 61 is the method of any one of Embodiment 57-60, wherein the tumor expresses a tumor-associated glycan.

Embodiment 62 is the method of Embodiment 61, wherein the tumor-associated glycan is an extracellular glycan.

Embodiment 63 is the method of Embodiment 57, wherein the glycosyltransferaseis selected from the group consisting of B4GALNT3 and B4GALNT4.

Embodiment 64 is the method of any one of Embodiments 57-63, wherein the one or more glycosyltransferases is selected from the group consisting of FUT3, FUT4, FUT5, FUT6, FUT7, FUT9, FUT10, and FUT11.

Embodiment 65 is the method of Embodiment 64, wherein the glycosyltransferase is FUT4.

Embodiment 66 is the method of any of Embodiment 57-65, wherein the glycosyltransferase is B4GALNT3.

Embodiment 67 the method of Embodiment 62, wherein the presence of the tumor-associated glycan is dependent on the expression of B4GALNT3 and FUT4 in the tumor.

Embodiment 68 is a method of producing an antibody, the method comprising culturing a host cell of Embodiment 49 under conditions in which the polynucleotide encoding the VH amino acid sequence and the polynucleotide encoding VL amino acid sequence are expressed.

Embodiment 69 is a method of selecting an anti-tumor antibody, the method comprising (1) contacting a candidate antibody with a tumor cell (or a lysate thereof) or a control cell (or a lysate thereof), wherein the tumor cell (or a lysate thereof) comprises (i) one or more glycosyltransferases in the tumor, wherein one of the one or more glycosyltransferases has N-acetyl-galactosaminyltransferase activity, (2) detecting binding of the candidate antibody with the tumor cell (or the lysate thereof) or with the control cell (or a lysate thereof), and (3) selecting the candidate antibody as the anti-tumor antibody if the binding of the candidate antibody to the tumor cell (or the lysate thereof) is greater than the binding of the candidate antibody to the control cell (or the lysate thereof).

Embodiment 70 is the method of Embodiment 69, wherein the control cell lacks the one or more of glycosyltransferases.

Embodiment 71 is the method of Embodiment 69, wherein the control cell is a tumor cell lacking one or more of glycosyltransferases.

Embodiment 72 is the method of Embodiment 69, wherein one of the one or more glycosyltransferases has fucosyltransferase activity.

Embodiment 73 is the method of Embodiment 69 of 70, wherein the glycosyltransferase that has N-acetyl-galactosaminyltransferase activity is selected from the group consisting of B4GALNT3 andB4GALNT4.

Embodiment 74 is the method of Embodiment 70 or 71, wherein one of the one or more glycosyltransferases that has fucosyltransferase activity is selected from the group consisting of FUT3, FUT4, FUT5, FUT6, FUT7, FUT9, FUT10, and FUT11.

Embodiment 75 is the method of any one of Embodiments 69-74, wherein the tumor expresses a tumor-associated glycan.

Embodiment 76 is the method of Embodiment 75, wherein the tumor-associated glycan is an extracellular glycan.

Embodiment 77 is the method of Embodiment 1, wherein the one or more glycosyltransferases is selected from the group consisting of B4GALNT3 and B4GALNT4.

Embodiment 78 is the method of any one of Embodiments 69-77, wherein the one or more glycosyltransferases is selected from the group consisting of FUT3, FUT4, FUT5, FUT6, FUT7, FUT9, FUT10, and FUT11.

Embodiment 79 is the method of Embodiment 78, wherein the glycosyltransferase is FUT4.

Embodiment 80 is the method of any of Embodiments 69-79, wherein the glycosyltransferase is B4GALNT3.

Embodiment 81 is the method of Embodiment 75, wherein the presence of the tumor-associated glycan is dependent on the expression of B4GALNT3 and FUT4 in the tumor.

Embodiment 82 is the method of any one of Embodiment 69-81 wherein the candidate antibody is an antibody isolated from a cancer patient.

Embodiment 83 is the method of any one of Embodiments 69-82, wherein the candidate antibody is a variant of an antibody isolated from a cancer patient.

Embodiment 84 is the method of Embodiment 83, wherein the variant is produced by mutagenizing a polynucleotide encoding a VH or a VL CDR3 of an antibody of any one of Embodiments 1-39 and expressing the variant comprising the mutagenized VH or VL CDR3.

Embodiment 85 is the method of any one of Embodiments 69-84, wherein the candidate antibody is a variant of an antibody of any one of Embodiments 1-40.

Embodiment 86 is a method of selecting an antibody having tumor-targeting activity, the method comprising: mutagenizing a polynucleotide encoding a VH or a VL CDR3 of an antibody of any one of Embodiments 1-40, expressing an antibody comprising the mutagenized VH or VL CDR3; and selecting the antibody as the antibody having tumor-targeting activity if it inhibits tumor growth or decreases tumor size, tumor invasion, and/or metastasis in vivo.

Embodiment 87. Use of the antibody of any one of Embodiments 1-40 or the immunoconjugate of any of Embodiments 41-45 for a method of treating cancer.

Embodiment 88 is the use of the antibody of Embodiment 87, wherein the cancer express a tumor-associated glycan, wherein the presence of the tumor-associated glycan is dependent on the expression of one or more glycosyltransferases in the tumor, wherein one of the one or more glycosyltransferases has N-acetyl-galactosaminyltransferase activity.

Embodiment 89 is the use of Embodiment 88, wherein one of the one or more glycosyltransferases has fucosyltransferase activity.

Embodiment 90 is the use of Embodiment 88 of 89, wherein the glycosyltransferase that has N-acetyl-galactosaminyltransferase activity is selected from the group consisting of B4GALNT3 and B4GALNT4.

Embodiment 91 is the use of Embodiment 89 or 90, wherein one of the one or more glycosyltransferases that has fucosyltransferase activity is selected from the group consisting of FUT3, FUT4, FUT5, FUT6, FUT7, FUT9, FUT10, and FUT11.

Embodiment 92 is the use of any one of Embodiments 88-91, wherein the tumor expresses a tumor-associated glycan.

Embodiment 93 is the use of Embodiment 92, wherein the tumor-associated glycan is an extracellular glycan.

Embodiment 94 is the use of Embodiment 88, wherein the one or more glycosyltransferases is selected from the group consisting of B4GALNT3 and B4GALNT4.

Embodiment 95 is the use of any one of Embodiments 88-94, wherein the one or more glycosyltransferases is selected from the group consisting of FUT3, FUT4, FUT5, FUT6, FUT7, FUT9, FUT10, and FUT11.

Embodiment 96 is the use of Embodiment 95, wherein the glycosyltransferase is FUT4.

Embodiment 97 is the use of any of Embodiments 88-96, wherein the glycosyltransferase is B4GALNT3.

Embodiment 98 is the use of Embodiment 92, wherein the presence of the tumor-associated glycan is dependent on the expression of B4GALNT3 and FUT4 in the tumor.

Embodiment 99 is the use of Embodiment 87, wherein the cancer is colorectal or gastric cancer.

Embodiment 100. Means for binding a tumor expressing a tumor-associated glycan, wherein the presence of the tumor-associated glycan is dependent on the expression of one or more glycosyltransferases in the tumor, wherein each of the one or more glycosyltransferases is selected from the group consisting of B4GALNT3, B4GALNT4, FUT3, FUT4, FUT5, FUT6, FUT7, FUT9, FUT10, and FUT11.

Embodiment 101 is the antibody of Embodiments above, wherein the tumor express a tumor-associated glycan, and wherein the tumor-associated glycan comprises GalNAcbeta1,4GlcNAc (LacdiNAc).

Embodiment 102 is the antibody of Embodiment(s) 101, wherein the LacdiNAc is fucoslyated.

Embodiment 103 is an antibody that binds to a tumor cell that expresses GalNAcbeta1,4GlcNAc (LacdiNAc).

### EXAMPLES

### Example 1. Target Identification

A functional genomic screen was performed in an AB-006410 flow-positive CRC cell line LoVo expressing the Cas9 nuclease. Cells were transfected with a genome-wide CRISPR sgRNA library and assessed for disruption of AB-006410 binding by flow cytometry. Two glycosyltransferases, B4GALNT3 and FUT4, and a fucose transporter, SLC35C1 were among the top hits from the screen.

AB-006410 binding to tumor cells in the presence of one of the glycosyltransferase inhibitors A-D and DMSO (control) was analyzed by flow cytometry, A, tunicamycin, inhibits N-linked glycosylation; B, BADGP, inhibits O-linked glycosylation (O-GlcNac); C, luteolin, inhibits O-linked glycosylation (O-GalNac); D, PPMP, inhibits glycolipid synthesis. The results show that as compared to DMSO, glycosyltransferase inhibitors weakened the binding between AB-006410 and the tumor cells, indicating the binding of AB-006410 to tumor cells depends on glycosyltransferases. FIG. 1.

The binding of AB-006410 to LoVo cells in which a glycosyltransferase is knocked out by CRISPR/Cas9 was examined. Briefly, Cas9 RNPs was delivered into LoVo cell lines using electroporation with the Neon system (Invitrogen, Carlsbad, USA) according to the manufacturer's instructions. For RNP delivery experiments, purified Cas9 protein and synthetic single guide RNAs (sgRNAs) were assembled at a 1:9 molar ratio (Cas9 protein:synthetic sgRNAs) in 10 mins at room temperature with buffer R (provided with Neon™ Transfection System). After complexing, the RNPs were electroporated into cell lines. 72 hours after RNP electroporation, the impact of knock out for each gene on the antibody staining phenotype was analyzed by flow cytometry. The results show that as compared to the control sgRNA treated cells, LoVo cells treated with sgRNA targeting B4GALNT3 and with sgRNA targeting FUT4 showed diminished binding to AB-006410. Knock-out of B4GLANT3 reduced the AB-006410 binding almost to isotype control levels (FIG. 2A) while deletion of FUT4 resulted in a lower but significant reduction of AB-006410 binding (FIG. 2B).

To ascertain which glycosyltransferases were important in presenting the glycans responsible for AB-006410 binding to cells, A549 cells which do not exhibit AB-006410 binding had their glycan profiles altered by glycosyltransferase overexpression. A549 cells were modified to express the CRISPRa dCas9-VRP protein and electroporated with sgRNAs targeting promoters of specific glycosyltransferases. B4GALNT3 or FUT4, or non-human sequences as control (negative sgRNAs). Duplicate assays were run using the following sets of sgRNAs: Fut4_guide RNA1: GGCGCCGCAGGAGGCTCCCG (SEQ ID NO: 913); Fut4_guide RNA2: GAGGCTCCCGGGGCCTGGTC (SEQ ID NO: 914); B4GALNT3_guide RNA1: GGCCTGCGACGGGAGAGGCG (SEQ ID NO: 915); B4GALNT3_guide RNA2: GAGGCCATTCGGCTTCCCTA (SEQ ID NO: 916); Non targeting (negative control 1): GTGTCGTGATGCGTAGACGG (SEQ ID NO: 917); Non targeting (negative control 2) GTCATCAAGGAGCATTCCGT (SEQ ID NO: 918).

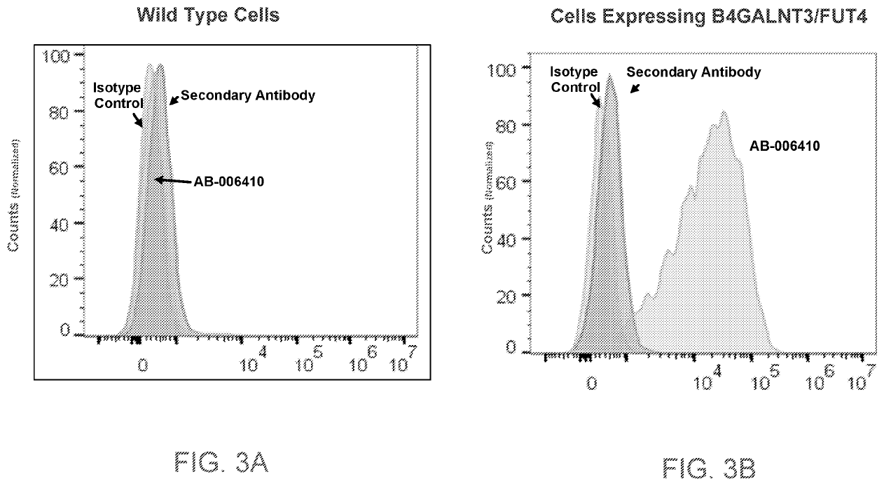

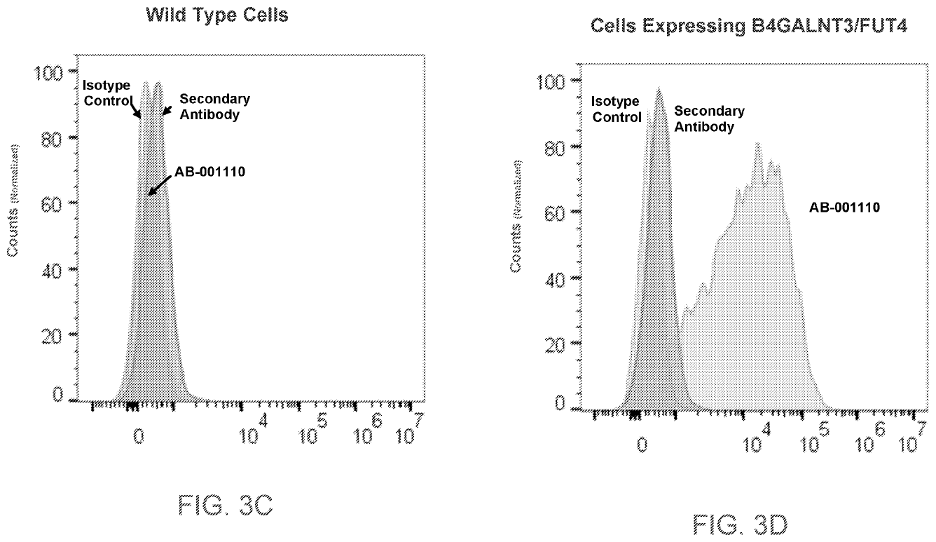

Results of the assays indicated that the overexpression of either B4GALNT3 or FUT4 induces low to medium binding of AB-006410 and AB-011110. Overexpression of both B4GALNT3 and FUT4 results in a greater increase in binding of both AB-006410 and AB-011110 as compared to overexpression of either glycosyltransferase alone. FIG. 3A-D shows the results of the overexpression of both B4GALNT3 and FUT4 using B4GALNT3_guide RNA2 (SEQ ID NO: 916), Fut4_guide RNA2: GAGGCTCCCGGGGCCTGGTC (SEQ ID NO: 914), and non-targeting (negative control 2) GTCATCAAGGAGCATTCCGT (SEQ ID NO: 918).

Expression of B4GALNT3 or FUT4 and AB-006410 binding were examined using immunofluorescence staining. The staining was performed on formalin-fixed paraffin-embedded tissue sections of human cancers of AB-006410 high, low, and medium prevalence (colorectal, ovarian and head and neck, respectively). Dewaxing and antigen retrieval techniques were performed followed by standard immunostaining protocols. Briefly, fixed tissues were washed with 3% donkey serum to block non-specific binding. Serial tissue sections were incubated with a primary antibody directed to AB-006410. B4GALNT3 or FUT4 in 3% donkey serum diluent. Sections were incubated with Alexa-647 conjugated secondary antibody and counterstained with Hoechst, mounted in anti-fade mounting reagent. Serial slides were stained using H&E. The primary antibodies were utilized with species-specific secondary antibodies and detected using standard methodology.

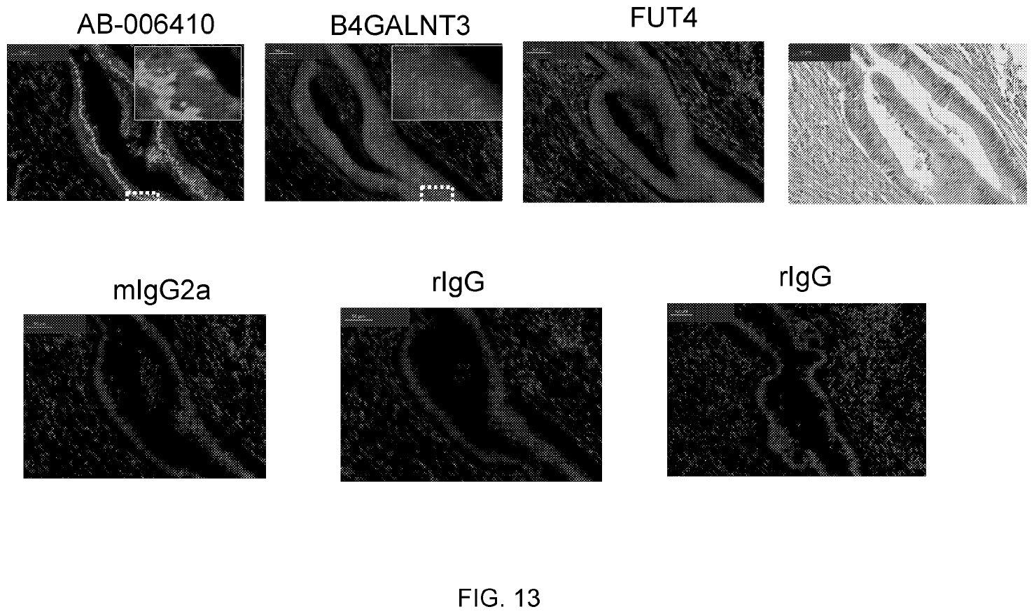

Percent concordance was based on the agreement of observed signal (i.e., all antibodies show positive or negative signal) between AB-006410 and B4GALNT3 and/or FUT4. A greater than 50 percent concordance between AB-006410 and B4GALNT3 and/or FUT4 was observed across all three human cancer indications suggesting that B4GALNT3 and/or FUT4 are likely required for the expression of the target of AB-006410 and the binding of AB-006410 to the target. Less than 10 percent of tumors showed AB-006410 binding in cases where B4GALNT3 and FUT4 signal was not observed, further suggesting that B4GALNT3 and FUT4 were involved in the expression of the target of AB-006410. FIG. 13 shows representative images of binding of AB-006410, B4GALNT3 and FUT4 antibodies to colorectal cancer tissues, and the results indicate the bindings profiles were similar.

### Example 2. AB-006410 Variants

The sequence of AB-006410 was analyzed for potential liabilities. AB-006410 was mutated to remove an N-linked glycosylation site in the light chain CDR1 to generate AB-011110 and AB-011111. AB-011110 was then used as the basis for generation of additional variants designed to address other potential liabilities. One of these variants, AB-011622, contained the mutations R30Q in the light chain CDR1 and D97N in the light chain CDR3 designed to remove two liabilities. Additional variants were generated based on AB-011622, including AB-011788 which contains the mutation D54K in the heavy chain CDR2. AB-011367 was generated using a consensus of three sibling antibodies with a light chain CDR1 N-glycosylation site and a light chain CDR3 free cysteine removed. Additional variants were generated based on AB-011367, including AB-011628, which contains the mutation R57N in the light chain CDR2 and AB-011861 which contains the mutation D54N in the heavy chain HCDR2. The variants tested retained the thermostability of the parental antibodies.

### Example 3. Glycan Binder In Vitro Studies

### Surface Binding in Tumor Cells

Surface binding to in vitro grown cell lines was assessed by flow cytometry. Tumor cells were detached from their culture plate using Versene and counted. Cells were staining in BSA-containing buffer with primary antibody for 30 minutes at 4° C. with shaking. Cells were then washed and stained with secondary PE-labeled antibody for 30 minutes at 4° C. with shaking. Before analysis on an Intellicyt iQue3 scanner, cells were counterstained with DAPI. MedFI values from live, single cells was expressed as fold over isotype control. Results of the assay showed that AB-006410 has a very distinct binding profile, predominantly binding to colorectal cancer lines. Binding also observed on gastric cancer line NUGC4. Table 10.

TABLE 10
Surface binding of AB-006410 to tumor cell lines

| Colorectal | | | | | Gastric | | | | |
|---|---|---|---|---|---|---|---|---|---|
| LoVo | HT29 | HT55 | LS174T | | LS411N | | SW1116 | COLO205 | T84 |
| 3.33 | 3 | 4 | 3 | | 2 | | 2 | 1.33 | 1 |
| | Pancreatic | | | | | | | | |
| | RKO | NUGC4 | BxPC3 | | PANC1 | | | | |
| | 2 | 2 | 0 | | 0 | | | | |
| Renal | | | | | | | | | |
| Clear | | | | | | | | | |
| Cell | Melanoma | | | Lung | | | | | Bladder |
| 786O | A375 | SKMEL28 | SKMEL30 | A549 | H522 | HCC2935 | HCC4006 | SW900 | SW780 |
| 0 | 0 | 0 | 0 | 0 | 0 | 1 | 0 | 1 | 0 |
| | Breast | | | | | | | | |
| | ER+, | | ER−, | | | | | | |
| | PR+/−, | | PR−, | | | | | | |
| | Her2+ | Triple− | Her2+ | Ovarian | Prostate | | | | |
| | BT474 | MDAMB231 | SKBR3 | SKOV3 | PC3 | | | | |
| | 0 | 0 | 0 | 0 | 0 | 0 | | | |

AB-006410 showed no binding on the normal cell lines tested, including colon (CCD841CoN), skin (Detroit551), and intestine (Hs738.St/Int and HIEC-6). AB-011110 also showed no binding on normal cell lines tested, including colon (CCD841CoN) and intestine (Hs738.St/Int and HIEC-6).

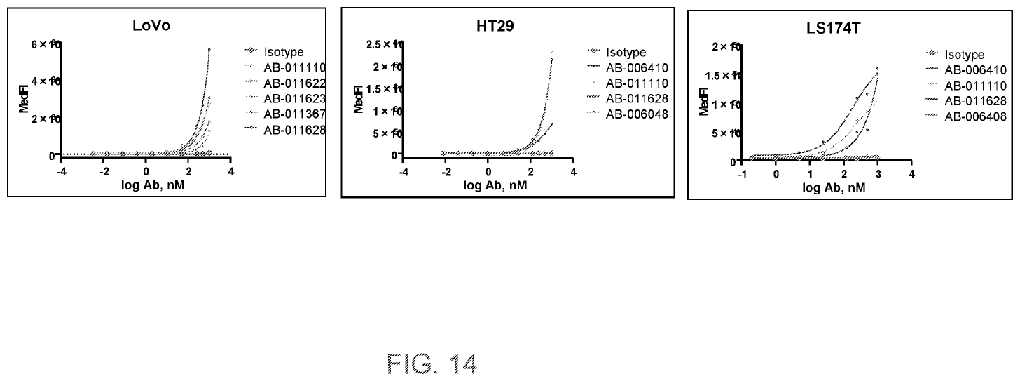

The flow cytometry assay was repeated to test binding of variant glycan binders to colorectal cancer cell lines LoVo, HT29, and LS174T. Results of the assay indicated that all the variants tested, AB-011110, AB-011622, AB-011623, AB-011367, and AB-011628, retained binding to the cell lines. FIG. 14.

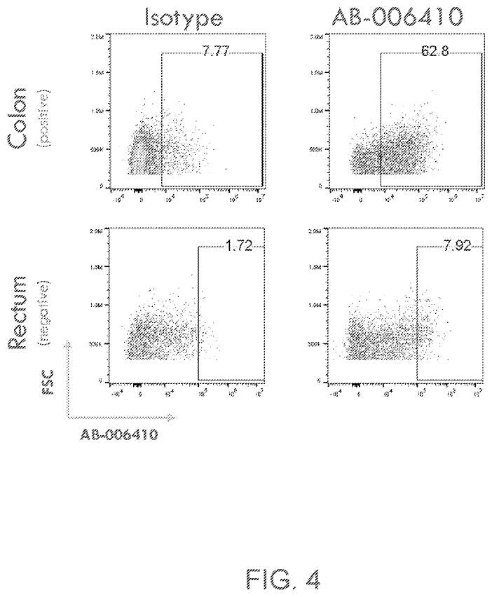

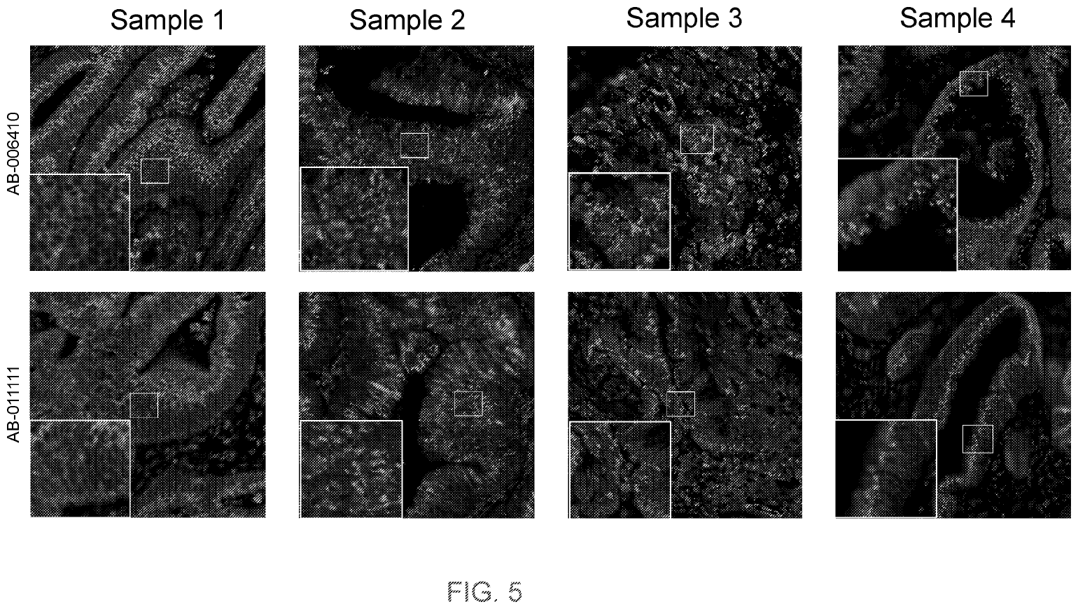

The surface binding of AB-006410 to human dissociated colorectal cancer samples was then assessed. AB-006410 was conjugated using Thermo's SiteClick™ R-PE Antibody Labeling Kit for testing on dissociated colorectal carcinoma cells from commercial sources. These cells include cells from colon, rectum, and splenic flexure tumors. Dissociated cells were thawed and cells were blocked with Human TruStain FcX™ (Fc Receptor Blocking Solution) Antibody and stained with PE-conjugated antibodies, EpCAM-BV421 and CD45-BV605 for 30 min at 4° C. Cells were washed 3 times with 200 μl 1% FBS/1 mM EDTA/PBS and resuspended in assay buffer containing DAPI. Cells were analyzed using the Cytoflex and FlowJo_v10.7.1. The results showed that AB-006410 bound to the surface of six out of nine dissociated cancer samples with no binding to CD45+ tumor-infiltrating leukocytes See FIG. 4. AB-011111 demonstrated a concordant immunofluorescence profile to AB-006410. FIG. 5. No binding was detected in eight dissociated ovarian cancer samples.

### Binding in Tumor Tissues

Immunohistochemistry staining was performed on formalin-fixed paraffin-embedded tissue sections of several human tumors. Standard dewaxing and antigen retrieval techniques were performed followed by standard immunostaining protocols. Briefly, fixed tissues were incubated with Dako serum free protein to block non-specific binding. Sections were incubated with a primary antibody in Dako background reducing diluent. Sections were incubated with a polymer HRP conjugated secondary antibody and stained using Hematoxylin. The primary antibodies were utilized with species-specific secondary antibodies and detected using standard methodology. AB-006410 showed robust & tumor-selective signal in ≥50% of tumor cells. The top five tumor types that are most reactive with AB-006410 include colorectal adenocarcinoma, pancreatic adenocarcinoma, stomach adenocarcinoma, uterine endometrioid adenocarcinoma, and NSCLC (squamous cell carcinoma and adenocarcinoma), indicating the presence of the glycan target of this antibody in these tumor types. Table 4.

TABLE 4
Reactivity of AB-006410 to a panel of human cancers (Subtypes in italics)

| Indication | Total Cores | Reactive | Non-Reactive | Percent Reactive |
|---|---|---|---|---|
| Colorectal | 19 | 18 | 1 | 95% |
| Adenocarcinoma | 19 | 18 | 1 | 95% |
| Pancreatic | 17 | 10 | 7 | 59% |
| Adenocarcinoma | 17 | 10 | 7 | 59% |
| Stomach | 16 | 9 | 7 | 56% |
| Adenocarcinoma | 16 | 9 | 7 | 56% |
| Uterine | 17 | 9 | 8 | 53% |
| Endometrioid Adenocarcinoma | 17 | 9 | 8 | 53% |
| Lung | 19 | 6 | 13 | 32% |
| Squamous Cell Carcinoma | 9 | 2 | 7 | 26% |
| Adenocarcinoma | 10 | 4 | 6 | 67% |
| Prostate | 16 | 5 | 11 | 31% |
| Adenocarcinoma | 16 | 5 | 11 | 31% |
| Esophageal | 20 | 6 | 14 | 30% |
| Squamous Cell Carcinoma | 10 | 1 | 9 | 11% |
| Adenocarcinoma | 10 | 5 | 5 | 50% |
| Thyroid | 18 | 5 | 13 | 28% |
| Papillary Carcinoma | 10 | 4 | 6 | 40% |
| Follicular Carcinoma | 8 | 1 | 7 | 13% |
| Ovarian | 13 | 3 | 10 | 23% |
| Low / High grade Serous Ca | 8 | 0 | 8 | 0% |
| Adenocarcinoma | 2 | 2 | 0 | 100% |
| Mucinous Adenocarcinoma | 2 | 1 | 1 | 50% |
| Telium tumor | 1 | 0 | 1 | 0% |
| Head and Neck | 19 | 3 | 16 | 18% |
| Squamous Cell Carcinoma | 19 | 3 | 16 | 18% |
| Brain | 19 | 2 | 17 | 11% |
| Astrocytoma | 8 | 0 | 8 | 0% |
| Glioblastoma | 5 | 0 | 5 | 0% |
| Anaplastic Glioma | 6 | 2 | 4 | 33% |
| Bladder | 17 | 1 | 16 | 6% |
| Urothelial carcinoma | 17 | 1 | 16 | 6% |
| Liver | 18 | 1 | 17 | 6% |
| Hepatocellular carcinoma | 18 | 1 | 17 | 6% |
| Cervical | 19 | 1 | 18 | 5% |
| Squamous Cell Carcinoma | 19 | 1 | 18 | 5% |
| Kidney | 20 | 1 | 19 | 5% |
| Clear Cell Carcinoma | 20 | 1 | 19 | 5% |
| Melanoma | 20 | 1 | 19 | 5% |
| Breast | 19 | 1 | 18 | 5% |
| Invasive Ductal Carcinoma | 19 | 1 | 18 | 5% |
| Testes | 19 | 1 | 18 | 5% |
| Seminoma | 14 | 1 | 13 | 7% |
| Embryonal Carcinoma | 5 | 0 | 5 | 0% |
| Lymph Node | 20 | 0 | 20 | 0% |
| Hodgkin's Lymphoma | 10 | 0 | 10 | 0% |
| Diffuse B-cell carcinoma | 9 | 0 | 9 | 0% |
| Anaplastic Large cell | 1 | 0 | 1 | 0% |
| Lymphoma | | | | |
| Skeletal Muscle | 14 | 0 | 14 | 0% |
| Fibrosarcoma | 10 | 0 | 10 | 0% |
| Liposarcoma | 4 | 0 | 4 | 0% |

AB-006410 showed robust signal in binding to colorectal, pancreatic, stomach and uterine cancer. FIG. 6A. No detectable binding of AB-006410 to frozen tissues derived from 26 types of normal human tissues, and only faint to moderate cytoplasmic immunoreactivity was observed in normal human stomach.

Immunohistochemistry staining was performed on formalin-fixed paraffin-embedded tissue sections from a variety of human tumors. Dewaxing and antigen retrieval techniques were performed followed by standard immunostaining protocols. Briefly, fixed tissues were incubated with Dako serum free protein block to block non-specific binding. Sections were incubated with a primary antibody in Dako background reducing diluent. Sections were incubated with a polymer HRP conjugated secondary antibody and stained using Hematoxylin. The primary antibodies were utilized with species-specific secondary antibodies and detected using standard methodology. AB-006410, AB-011110 and AB-011628 showed robust & tumor-selective signal in ≥50% of tumor cells. Representative images of colorectal adenocarcinoma and pancreatic adenocarcinoma are shown in FIG. 6B, indicating the presence of the glycan target of these antibodies in these tumor types. Staining intensity was the highest for AB-011110, followed by AB-006410 and AB-011628. All three antibodies were shown to bind to identical tumor cores, suggesting conservation of paratope/epitope recognition among these variants.

### Internalization

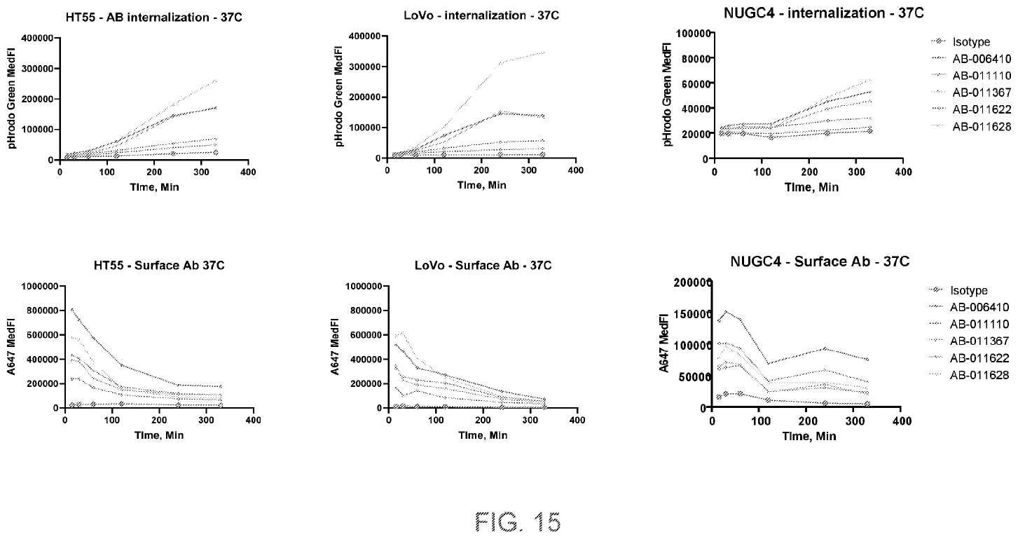

Internalization on cell lines grown in vitro was assessed by flow cytometry. Antibodies were conjugated to a pH-sensitive dye using Thermo's pHrodo™ iFL Green Microscale Protein Labeling Kits. Cells were detached with Versene, seeded into a 96-well plate, and incubated with 250 nM conjugated antibodies at 37° C. After incubation, cells were washed and stained with A647 conjugated anti-mouse IgG secondary antibody for 20 min at 4° C. and washed to evaluate the remaining antibodies on the cell surface. Cells were stained with DAPI and both internalization and surface signals were measured using Intellicyt's iQue3 scanner. Results showed that AB-006410 and variants of AB-006410 were internalized at 37° C. over time as indicated by the pH-sensitive pHrodo Green dye that fluoresces when the conjugated antibody reaches the early endosome. At the same time, a decrease was observed in surface bound antibodies in three different colorectal cancer cell lines, corroborating the above finding of the internalization of antibody-bound targets in these three different colorectal cancer cell lines. FIG. 15.

### ADC

The antibody-drug conjugate (ADC) activity of the glycan binders were assessed on LoVo cells. Briefly, the antibodies were tested for ADC activity using a secondary, toxin-conjugated antibody. Target cells were detached from the culture plate and cell concentration was adjusted to 31,250 cells/mL in assay media. 2,500 cells were added to each well of a 96 well plate and incubated with different concentrations of primary antibody for 15 min at room temperature. Following, secondary Fab anti-mouse IgG Fc conjugated to Duocarmycin with a cleavable linker (Moradec, #AM-202-DD) was added at a final concentration of 250 ng/mL. Cells were incubated for 72 h at 37° C. and 5% CO₂. At the end of the incubation period, 100 μl CellTiter-Glo® was added to each well and allowed to incubate for 5-10 min at room temperature before reading luminescence in a BMG ClarioSTAR plate reader. Data was then normalized to a maximum lysis control and plotted using graph pad prism. Cetuximab was included as a positive control.

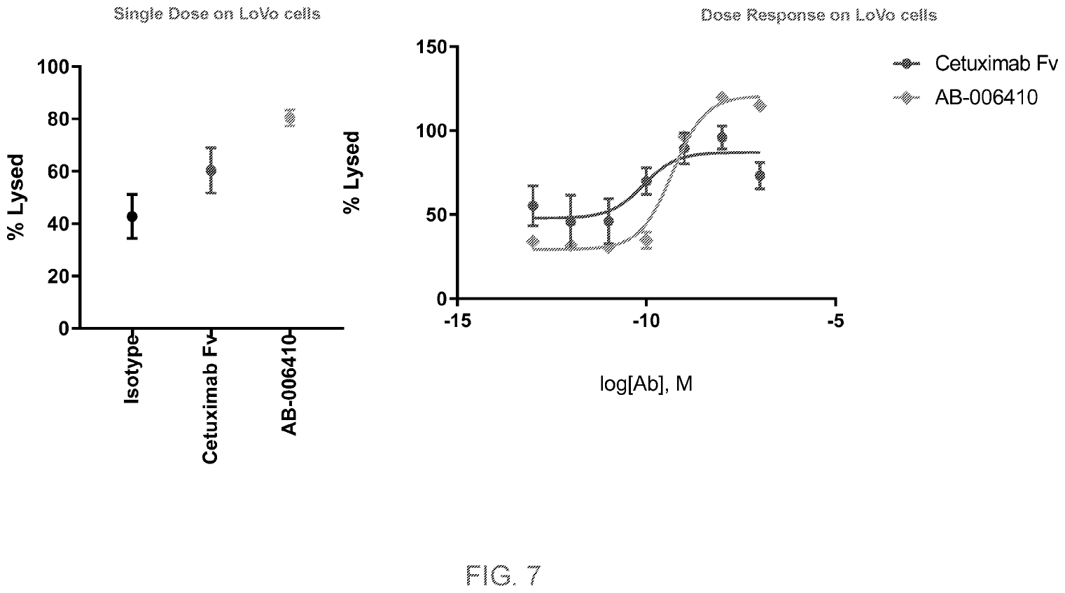

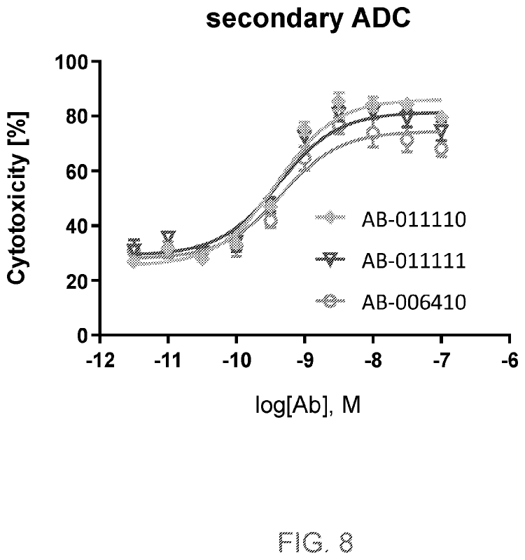

Results of this assay showed that the AB-006410 exhibits dose responsive ADC activity in this model. FIG. 7. ADC activity of variants mutated to remove the N-linked glycosylation motif (NES) in the CDR-L1 of AB-006410 was assessed relative to the ADC activity of AB-006410. Results of the assay showed that the variants exhibited the same level of cytotoxicity as AB-006410. FIG. 8.

ADC activity for additional glycan binder variants was assessed with results shown in Table 5. All the glycan binders showed dose responsive ADC activity and several variants showed similar cytotoxicity to AB-011110.

TABLE 5
ADC data of the AB-006410 variant shown relative to AB-011110.

| | EC50 (nM) | | | | Delta Cytotoxicity | | | |
|---|---|---|---|---|---|---|---|---|
| | Run | | Run | Aver- | Run | Run | Run | Aver- |
| Antibody | 1 | Run | 3 | age | 1 | 2 | 3 | age |
| AB-011366 | 4.37 | 4.56 | 20.22 | 9.72 | 62.8 | 57.0 | 31.4 | 50.4 |
| AB-011367 | 1.07 | 1.27 | 1.77 | 1.37 | 85.1 | 78.2 | 54.5 | 72.6 |
| AB-011368 | 0.67 | 1.12 | 2.13 | 1.31 | 64.7 | 42.7 | 34.6 | 47.3 |
| AB-011110 | 0.32 | 0.70 | 0.95 | 0.66 | 99.1 | 58.5 | 63.3 | 73.6 |
| AB-011369 | 0.61 | 0.51 | 3.40 | 1.51 | 96.6 | 64.6 | 33.2 | 64.8 |
| AB-011370 | 0.56 | 0.08 | 1.25 | 0.63 | 96.3 | 50.2 | 48.2 | 64.9 |
| AB-011371 | 1.03 | 0.70 | 3.10 | 1.61 | 66.0 | 36.7 | 14.5 | 39.1 |
| AB-011110 | 0.44 | 0.69 | 1.00 | 0.71 | 95.5 | 62.8 | 58.2 | 72.2 |
| AB-011372 | 0.65 | 1.00 | 18.73 | 6.79 | 62.9 | 48.3 | 19.4 | 43.5 |
| AB-011373 | 1.50 | 16.67 | 44.17 | 20.78 | 76.5 | 64.2 | 23.4 | 54.7 |
| AB-011374 | 0.48 | 0.66 | 0.56 | 0.57 | 96.1 | 105.9 | 42.7 | 81.6 |
| AB-011110 | 0.33 | 1.08 | 1.11 | 0.84 | 89.8 | 68.1 | 49.0 | 69.0 |
| AB-011375 | 0.57 | 1.19 | 0.71 | 0.82 | 88.4 | 84.9 | 41.9 | 71.7 |
| AB-011376 | 0.68 | 1.30 | 3.77 | 1.92 | 84.2 | 47.0 | 48.7 | 60.0 |
| AB-000129 | 0.01 | 0.01 | 0.02 | 0.02 | 53.7 | 106.4 | 67.4 | 75.8 |
| AB-011110 | 0.46 | 0.74 | 0.83 | 0.68 | 91.4 | 76.8 | 51.1 | 73.1 |

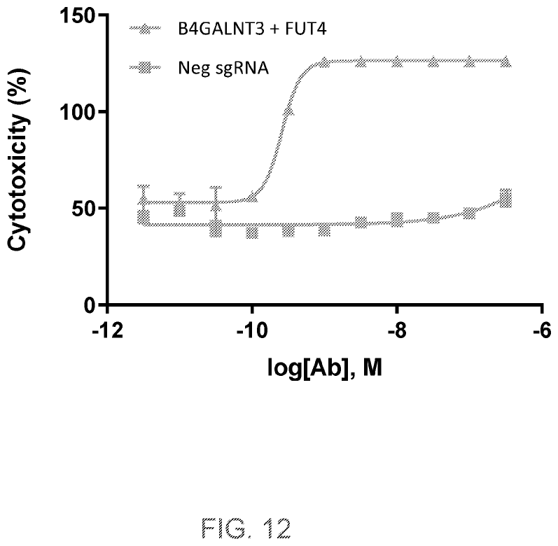

To assess the effect of the glycosyltransferases on the activity of AB-006410, A549 cells which do not exhibit AB-006410 binding had their glycan profiles altered by overexpressing both B4GALNT3 and FUT4 as described in Example 1. The results showed that overexpression of these glycosyltransferases restored AB-006410 reactivity and sensitized these cells to ADC killing. Introducing a control sgRNA to these cells did not affect their reactivity to the ADC. FIG. 12. Overexpression of either B4GALNT3 or FUT4 also sensitized cells to ADC killing but to a lesser extent that dual overexpression as determined in this assay.

### ADC Direct Conjugated

Antibody-drug conjugate activity was tested on antibodies directly conjugated with a ZymeLink™ Auristatin payload. LoVo target cells were detached from the culture plate and cell concentration was adjusted to 31,250 cells/mL in assay media. 2,500 cells were added to each well of a 96 well plate and incubated with different concentrations of directly conjugated primary antibody for 15 min at room temperature. Cells were than incubated for 72 h at 37° C. and 5% CO2. At the end of the incubation period, 100 μl CellTiter-Glo® was added to each well and allowed to incubate for 5-10 min at room temperature before reading luminescence in a BMG ClarioSTAR plate reader. Data was then normalized to a maximum lysis control and plotted using graph pad prism.

Results of this assay showed that AB-006410 in the Zymelink™ Auristatin format exhibited cytotoxicity on the LoVo cell line. Cetuximab-auristatin ZymeLink™ ADC (Cetuximab-ZLA) was included as a positive control and Isotype-auristatin ZymeLink™ ADC (Isotype-ZLA) was included as the negative control. FIG. 9A.

Additional variants of AB-006410 were expressed and conjugated with the ZymeLink™ Auristatin payload. ADC cytotoxicity was assessed on LoVo target cells using the same protocol with results shown in Table 6. Many of the variants maintained the ability to lyse LoVo cells at 100 nM. A subset of conjugated variants were assessed for cytotoxicity on LoVo cells over a wider concentration range, starting with 1000 nM (FIGS. 9B and 9C; Table 7). Some variant conjugates had more potent cytotoxicity than the AB-006410 conjugate.

TABLE 6
Cytotoxicity of AB-006410 variants conjugated with ZymeLink ™ Auristatin payload

| | Max. | | Max. | | Max | | Max. |
|---|---|---|---|---|---|---|---|
| Antibody | Cytotoxicity | | Cytotoxicity | | Cytotoxicity | | Cytotoxicity |
| ID | (%) | Antibody ID | (%) | Antibody ID | (%) | Antibody ID | (%) |
| AB-006410 | 35.75 | AB-011802 | 23 | AB-011827 | 17 | AB-011852 | 23 |
| AB-011110 | 21 | AB-011803 | 0.86 | AB-011828 | 25 | AB-011853 | 10 |
| AB-011367 | 38 | AB-011804 | 4.6 | AB-011829 | 12 | AB-011854 | 22 |
| AB-011622 | 35 | AB-011805 | −2.3 | AB-011830 | 22 | AB-011855 | 19 |
| AB-011781 | 8.3 | AB-011806 | 11 | AB-011831 | 21 | AB-011856 | 21 |
| AB-011782 | 18 | AB-011807 | 2.6 | AB-011832 | 19 | AB-011857 | 3.5 |
| AB-011783 | −0.95 | AB-011808 | 9.3 | AB-011833 | 11 | AB-011858 | −0.25 |
| AB-011784 | −4.1 | AB-011809 | 0.83 | AB-011834 | −3.1 | AB-011859 | 2 |
| AB-011785 | 12 | AB-011810 | 6.3 | AB-011835 | 5.8 | AB-011860 | 16 |
| AB-011786 | 18 | AB-011811 | 16 | AB-011836 | −1 | AB-011861 | 54 |
| AB-011787 | 8.7 | AB-011812 | 6.5 | AB-011837 | 0.14 | AB-011862 | 13 |
| AB-011788 | 30 | AB-011813 | −0.09 | AB-011838 | 16 | AB-011863 | 18 |
| AB-011789 | 17 | AB-011814 | 2.6 | AB-011839 | 15 | AB-011864 | 28 |
| AB-011790 | −0.2 | AB-011815 | 1.8 | AB-011840 | 17 | AB-011865 | 26 |
| AB-011791 | 12 | AB-011816 | 12 | AB-011841 | 2.3 | AB-011866 | 27 |
| AB-011792 | 5.2 | AB-011817 | −4.4 | AB-011842 | 14 | AB-011867 | 6.8 |
| AB-011793 | 5 | AB-011818 | 7.1 | AB-011843 | 5.6 | AB-011868 | 16 |
| AB-011794 | 33 | AB-011819 | 4.9 | AB-011844 | 5.9 | AB-011869 | 5.7 |
| AB-011795 | 24 | AB-011820 | 6.9 | AB-011845 | 14 | AB-011870 | 0.9 |
| AB-011796 | 9.3 | AB-011821 | 3.5 | AB-011846 | 4.5 | AB-011871 | 1 |
| AB-011797 | 9.1 | AB-011822 | 7 | AB-011847 | 14 | AB-011872 | 24 |
| AB-011798 | 17 | AB-011823 | −7 | AB-011848 | 13 | AB-011873 | 2.9 |
| AB-011799 | −0.06 | AB-011824 | 4.8 | AB-011849 | 11 | | |
| AB-011800 | 7.4 | AB-011825 | 2 | AB-011850 | −1.9 | | |
| AB-011801 | 21 | AB-011826 | 19 | AB-011851 | 23 | | |

TABLE 7
Dose-dependent cytotoxicity of AB-006410 variants conjugated
with ZymeLink ™ Auristatin payload

| | EC50 (nM) | | | Max. Cytotoxicity (%) | | |
|---|---|---|---|---|---|---|
| Antibody ID | Run 1 | Run 2 | Avg | Run 1 | Run 2 | Avg |
| AB-006410 | 24.06 | 33.96 | 29.01 | 92.07 | 90.78 | 91.43 |
| AB-011110 | 6.03 | 7.02 | 6.525 | 91.92 | 92.37 | 92.15 |
| AB-011622 | 13.1 | 12.98 | 13.04 | 90.66 | 89.77 | 90.22 |
| AB-011788 | 3.89 | 3.5 | 3.695 | 91.24 | 90.48 | 90.86 |
| AB-011789 | 6.18 | 6.16 | 6.17 | 91.29 | 89.86 | 90.58 |
| AB-011794 | 19.24 | 7.64 | 8.44 | 90.31 | 90.77 | 90.54 |
| AB-006410 | 33.28 | 33.45 | 33.365 | 90.65 | 90.72 | 90.69 |
| AB-011367 | 7.93 | 7.33 | 7.63 | 91.72 | 89.93 | 90.82 |
| AB-011795 | 21.74 | 21.58 | 21.66 | 89.45 | 90.26 | 89.86 |
| AB-011842 | >300 | >300 | >300 | 73.17 | 71.09 | 72.13 |
| AB-011861 | 3.46 | 2.68 | 3.07 | 90.39 | 89.69 | 90.04 |

Many of the variant antibodies, including AB-011110, AB-011788, AB-011789, AB-011794, AB-011367, and AB-011861 showed improvements in ADC activity as conjugates, with AB-011861 having a 10-fold improved ADC activity as compared to AB-006410.

### Example 4. AB-006410 In Vivo Studies

AB-006410 in the ZymeLink™ ADC construct format was also tested in mice carrying tumors from LoVo tumor cells. In brief, 1E7 LoVo tumor cells were injection subcutaneously into female BALB/c nude mice. Tumors were allowed to establish and randomized at around 150 mm³. Dosing was performed at day of randomization. As compared to the group that were treated with isotype-Zymelink™ conjugate, the group of mice that were treated with AB-006410-Zymelink™ conjugate (Zyme) (6 mpk) or AB-006410-Zyme (12 mpk) mice showed reduced tumor growth during the treatment period. FIG. 10. The results showed a single dose injection of the antibodies was able to induce a strong tumor reduction response in a dose-dependent manner.

### INCORPORATION BY REFERENCE

Each and every publication and patent document referred to in this disclosure is incorporated herein by reference in its entirety for all purposes to the same extent as if each such publication or document was specifically and individually indicated to be incorporated herein by reference.

While the invention has been described with reference to the specific examples and illustrations, changes can be made and equivalents can be substituted to adapt to a particular context or intended use as a matter of routine development and optimization and within the purview of one of ordinary skill in the art, thereby achieving benefits of the invention without departing from the scope of what is claimed and their equivalents.

What is claimed is:

1. An antibody that binds a tumor, wherein the binding of the antibody to the tumor is dependent on the expression of one or more glycosyltransferases in the tumor, wherein one of the one or more glycosyltransferases has N-acetyl-galactosaminyltransferase activity.

2. The antibody of claim 1, wherein one of the one or more glycosyltransferases has fucosyltransferase activity.

3. The antibody of claim 1 of 2, wherein the glycosyltransferase that has N-acetyl-galactosaminyltransferase activity is selected from the group consisting of B4GALNT3 and B4GALNT4.

4. The antibody of claim 2 or 3, wherein one of the one or more glycosyltransferases that has fucosyltransferase activity is selected from the group consisting of FUT3, FUT4, FUT5, FUT6, FUT7, FUT9, FUT10, and FUT11.

5. The antibody of any one of claims 1-4, wherein the tumor expresses a tumor-associated glycan.

6. The antibody of claim 5, wherein the tumor-associated glycan is an extracellular glycan.

7. The antibody of claim 1, wherein the one or more glycosyltransferases is selected from the group consisting of B4GALNT3 and B4GALNT4.

8. The antibody of any one of claims 1-7, wherein the one or more glycosyltransferases is selected from the group consisting of FUT3, FUT4, FUT5, FUT6, FUT7, FUT9, FUT10, and FUT11.

9. The antibody of claim 8, wherein the glycosyltransferase is FUT4.

10. The antibody of any of claims 1-9, wherein the glycosyltransferase is B4GALNT3.

11. The antibody of claim 5, wherein the presence of the tumor-associated glycan is dependent on the expression of B4GALNT3 and FUT4 in the tumor.

12. The antibody of any of claims 1-11, wherein the antibody preferentially binds to a tumor tissue relative to a normal tissue.

13. The antibody of any of claims 1-12, wherein the antibody is internalized by the tumor cells upon contacting the tumor.

14. The antibody of any one of claims 1-13 that binds to a tumor, wherein the antibody comprises:
a heavy chain variable region comprising:
an HCDR1, HCDR2, and/or HCDR3 amino acid sequence listed in Table 1, or variants of the HCDR1, HCDR2, and/or HCDR3 amino acid sequence in which 1, 1, 2, 3, 4, 5, or more amino acids are substituted; and/or
a light chain variable region comprising:
an LCDR1, LCDR2, and/or LCDR3 amino acid sequence listed in Table 2, or variants of the LCDR1, LCDR2, and/or LCDR3 amino acid sequence in which 1, 2, 3, 4, 5, or more amino acid are substituted.

15. The antibody of claim 14, wherein the antibody comprises:
wherein the antibody comprises all six CDRs of an antibody selected from the group consisting of AB-006410, AB-011110, AB-011111, AB-011366, AB-011367, AB-011368, AB-011369, AB-011370, AB-011371, AB-011372, AB-011373, AB-011374, AB-011375, AB-011376, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, and AB-011873.

16. The antibody of any one of claims 14-15, wherein the antibody comprises a V_{H} region comprising a V_{H} amino acid sequence in Table 3 or an amino sequence having at least 70%, at least 75%, at least 80%, at least 85%, at least 90%, or at least 95% identity to the V_{H} amino acid sequence in Table 3, and/or
wherein the antibody comprises a V_{L} region comprising a V_{L} amino acid sequence in Table 3; and an amino sequence having at least 70%, at least 75%, at least 80%, at least 85%, at least 90%, or at least 95% identity to the V_{L} amino acid sequence in Table 3.

17. The antibody of any one of claims 14-16, wherein the antibody comprises both the V_{H} and V_{L} of an antibody selected from the group consisting of AB-006410, AB-011110, AB-011111, AB-011366, AB-011367, AB-011368, AB-011369, AB-011370, AB-011371, AB-011372, AB-011373, AB-011374, AB-011375, AB-01137, AB-011622, AB-011623, AB-011624, AB-011625, AB-011626, AB-011627, AB-011628, AB-011781, AB-011782, AB-011783, AB-011784, AB-011785, AB-011786, AB-011787, AB-011788, AB-011789, AB-011790, AB-011791, AB-011792, AB-011793, AB-011794, AB-011795, AB-011796, AB-011797, AB-011798, AB-011799, AB-011800, AB-011801, AB-011802, AB-011803, AB-011804, AB-011805, AB-011806, AB-011807, AB-011808, AB-011809, AB-011810, AB-011811, AB-011812, AB-011813, AB-011814, AB-011815, AB-011816, AB-011817, AB-011818, AB-011819, AB-011820, AB-011821, AB-011822, AB-011823, AB-011824, AB-011825, AB-011826, AB-011827, AB-011828, AB-011829, AB-011830, AB-011831, AB-011832, AB-011833, AB-011834, AB-011835, AB-011836, AB-011837, AB-011838, AB-011839, AB-011840, AB-011841, AB-011842, AB-011843, AB-011844, AB-011845, AB-011846, AB-011847, AB-011848, AB-011849, AB-011850, AB-011851, AB-011852, AB-011853, AB-011854, AB-011855, AB-011856, AB-011857, AB-011858, AB-011859, AB-011860, AB-011861, AB-011862, AB-011863, AB-011864, AB-011865, AB-011866, AB-011867, AB-011868, AB-011869, AB-011870, AB-011871, AB-011872, AB-011873 and a variant thereof.

18. The antibody of any one of claims 14-17, wherein the the antibody comprises both the V_{H} and V_{L} of an antibody selected from the group consisting of AB-011110, AB-011788, AB-011789, AB-011794, AB-011367, and AB-011861.

19. An antibody that competes for binding with the antibody of any one of claims 14-18.

20. An immunoconjugate comprising the antibody of any one of claims 1-19 and a cytotoxic agent.

21. The immunoconjugate of claim 20, wherein the cytotoxic agent is Auristatin.

22. The immunoconjugate of claim 20, wherein the cytotoxic agent is ZymeLink™ Auristatin (ZLA).

23. The immunoconjugate of claims 20-22, wherein the wherein the immunoconjugate comprises Formula (I) or (II): 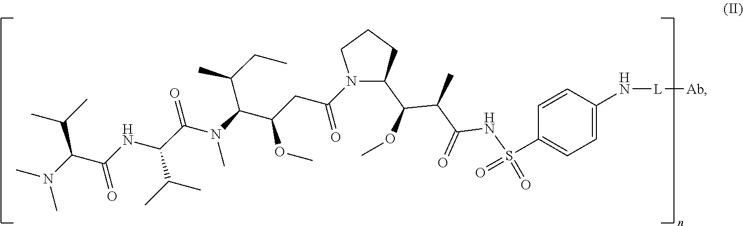
wherein:
L is a cleavable linker;
n is the drug-to-antibody ratio (DAR) and is an integer from 1 to 12, and
Ab is the antibody of any one of claims 1-19; or 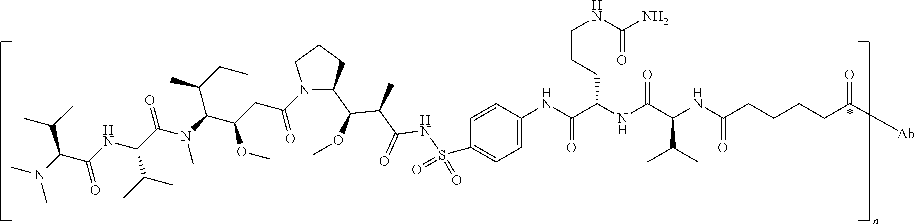
wherein:
n is the drug-to-antibody ratio (DAR) and is an integer from 1 to 12, and
Ab is the antibody of any one of the claims 1-23.

24. A polynucleotide encoding a polypeptide comprising a V_{H} sequence of an antibody of any one of claims 1-19, and/or a V_{L} sequence of an antibody of any one of claims 1-19.

25. An expression vector comprising a polynucleotide encoding the V_{H} region and/or the V_{L} region of the antibody of any one of claims 1-19.

26. A host cell that comprises an expression vector of claim 25.

27. A pharmaceutical composition comprising an antibody of any one of claims 1-19 or the immunoconjugate of any of claims 20-23 and a pharmaceutically acceptable carrier.

28. A method of treating a cancer patient, the method comprising administering the antibody of any of claims 1-19 to the patient.

29. A method of identifying a patient having a tumor suitable for treatment with an antibody, wherein the binding of the antibody to the tumor is dependent on the expression of one or more glycotransferases in the tumor, wherein one of the one or more glycosyltransferases has N-acetyl-galactosaminyltransferase activity, wherein the method comprises:
contacting a tumor sample from the patient with an antibody of any one of claims 1-23, and
detecting binding of the antibody to the tumor sample, wherein detection of the binding identifies the patient having a tumor suitable for treatment with the antibody.

30. A method of selecting an anti-tumor antibody, the method comprising
(1) contacting a candidate antibody with a tumor cell (or a lysate thereof) or a control cell (or a lysate thereof), wherein the tumor cell (or a lysate thereof) comprises
(i) one or more glycosyltransferases in the tumor, wherein one of the one or more glycosyltransferases has N-acetyl-galactosaminyltransferase activity,
(2) detecting binding of the candidate antibody with the tumor cell (or the lysate thereof) or with the control cell (or a lysate thereof), and
(3) selecting the candidate antibody as the anti-tumor antibody if the binding of the candidate antibody to the tumor cell (or the lysate thereof) is greater than the binding of the candidate antibody to the control cell (or the lysate thereof).

31. Use of the antibody of any one of claims 1-19 or the immunoconjugate of any of claims 20-23 for a method of treating cancer.


SEQUENCE LISTING (920 sequences)

SEQ ID NO: 1 - protein, 10 aa, synthetic construct
SFTFSNAWVN

SEQ ID NO: 2 - protein, 10 aa, synthetic construct
SFTFSNAWVN

SEQ ID NO: 3 - protein, 10 aa, synthetic construct
SFTFSNAWVN

SEQ ID NO: 4 - protein, 10 aa, synthetic construct
TFTFNNAWVN

SEQ ID NO: 5 - protein, 10 aa, synthetic construct
TFTFNNAWVN

SEQ ID NO: 6 - protein, 10 aa, synthetic construct
SFTFSNAWVN

SEQ ID NO: 7 - protein, 10 aa, synthetic construct
SFTFSNAWVN

SEQ ID NO: 8 - protein, 10 aa, synthetic construct
SFTFSNAWVN

SEQ ID NO: 9 - protein, 10 aa, synthetic construct
SFTFSNAWVN

SEQ ID NO: 10 - protein, 10 aa, synthetic construct
SFTFSNAWVN

SEQ ID NO: 11 - protein, 10 aa, synthetic construct
SFTFSNAWVN

SEQ ID NO: 12 - protein, 10 aa, synthetic construct
SFTFSNAWVN

SEQ ID NO: 13 - protein, 10 aa, synthetic construct
SFTFSNAWVN

SEQ ID NO: 14 - protein, 10 aa, synthetic construct
SFTFSNAWVN

SEQ ID NO: 15 - protein, 19 aa, synthetic construct
RLRSDTEGWTKDYAPHLKD

SEQ ID NO: 16 - protein, 19 aa, synthetic construct
RLRSDTEGWTKDYAPHLKD

SEQ ID NO: 17 - protein, 19 aa, synthetic construct
RLRSDTEGWTKDYAPHLKD

SEQ ID NO: 18 - protein, 19 aa, synthetic construct
RLRSDTEHWAKDYAAHMKG

SEQ ID NO: 19 - protein, 19 aa, synthetic construct
RLRSDTEHWAKDYAAHMKG

SEQ ID NO: 20 - protein, 19 aa, synthetic construct
RLRSDTEGWTKDYAPHLKD

SEQ ID NO: 21 - protein, 19 aa, synthetic construct
RLRSDTEGWTKDYAPHLKD

SEQ ID NO: 22 - protein, 19 aa, synthetic construct
RLRSDTEGWTKDYAPHLKD

SEQ ID NO: 23 - protein, 19 aa, synthetic construct
RLRSDTEGWTKDYAPHLKD

SEQ ID NO: 24 - protein, 19 aa, synthetic construct
RLRSDTEGWTKDYAPHLKD

SEQ ID NO: 25 - protein, 19 aa, synthetic construct
RLRSDTEGWTKDYAPHLKD

SEQ ID NO: 26 - protein, 19 aa, synthetic construct
RLRSDTEGWTKDYAPHLKD

SEQ ID NO: 27 - protein, 19 aa, synthetic construct
RLRSDTEGWTKDYAPHLKD

SEQ ID NO: 28 - protein, 19 aa, synthetic construct
RLRSDTEGWTKDYAPHLKD

SEQ ID NO: 29 - protein, 20 aa, synthetic construct
NTGYWPQSAFLTNRAYAMDV

SEQ ID NO: 30 - protein, 20 aa, synthetic construct
NTGYWPQSAFLTNRAYAMDV

SEQ ID NO: 31 - protein, 20 aa, synthetic construct
NTGYWPQSAFLTNRAYAMDV

SEQ ID NO: 32 - protein, 20 aa, synthetic construct
NTGYWPQSVFLTNRAYAMDV

SEQ ID NO: 33 - protein, 20 aa, synthetic construct
NTGYWPQSVFLTNRAYAMDV

SEQ ID NO: 34 - protein, 20 aa, synthetic construct
NTGYWPQSAFLTNRAYAMDV

SEQ ID NO: 35 - protein, 20 aa, synthetic construct
NTGYWPQSAFLTNRAYAMDV

SEQ ID NO: 36 - protein, 20 aa, synthetic construct
NTGYWPQSAFLTNRAYAMDV

SEQ ID NO: 37 - protein, 20 aa, synthetic construct
NTGYWPQSAFLTNRAYAMDV

SEQ ID NO: 38 - protein, 20 aa, synthetic construct
NTGYWPQSAFLTNRAYAMDV

SEQ ID NO: 39 - protein, 20 aa, synthetic construct
NTGYWPQSAFLTNRAYAMDV

SEQ ID NO: 40 - protein, 20 aa, synthetic construct
NTGYWPQSAFLTNRAYAMDV

SEQ ID NO: 41 - protein, 20 aa, synthetic construct
NTGYWPQSAFLTNRAYAMDV

SEQ ID NO: 42 - protein, 20 aa, synthetic construct
NTGYWPQSAFLTNRAYAMDV

SEQ ID NO: 43 - protein, 15 aa, synthetic construct
ASNESLRRGQYHFLD

SEQ ID NO: 44 - protein, 15 aa, synthetic construct
ASSESLRRGQYHFLD

SEQ ID NO: 45 - protein, 15 aa, synthetic construct
ASNEALRRGQYHFLD

SEQ ID NO: 46 - protein, 15 aa, synthetic construct
KSSESLHREGYNFLD

SEQ ID NO: 47 - protein, 15 aa, synthetic construct
KSSESLHREGYNFLD

SEQ ID NO: 48 - protein, 15 aa, synthetic construct
ASSESLHRGQYHFLD

SEQ ID NO: 49 - protein, 15 aa, synthetic construct
ASSESLQRGQYHFLD

SEQ ID NO: 50 - protein, 15 aa, synthetic construct
ASSESLRSGQYHFLD

SEQ ID NO: 51 - protein, 15 aa, synthetic construct
ASSESLRRGQYHFLD

SEQ ID NO: 52 - protein, 15 aa, synthetic construct
ASSESLRRGQYHFLD

SEQ ID NO: 53 - protein, 15 aa, synthetic construct
ASSESLRRGQYHFLD

SEQ ID NO: 54 - protein, 15 aa, synthetic construct
ASSESLRRGQYHFLD

SEQ ID NO: 55 - protein, 15 aa, synthetic construct
ASSESLRRGQYHFLD

SEQ ID NO: 56 - protein, 15 aa, synthetic construct
ASSESLRRGQYHFLD

SEQ ID NO: 57 - protein, 7 aa, synthetic construct
LGFRRAS

SEQ ID NO: 58 - protein, 7 aa, synthetic construct
LGFRRAS

SEQ ID NO: 59 - protein, 7 aa, synthetic construct
LGFRRAS

SEQ ID NO: 60 - protein, 7 aa, synthetic construct
LGSRRAA

SEQ ID NO: 61 - protein, 7 aa, synthetic construct
LGSRRAA

SEQ ID NO: 62 - protein, 7 aa, synthetic construct
LGFRRAS

SEQ ID NO: 63 - protein, 7 aa, synthetic construct
LGFRRAS

SEQ ID NO: 64 - protein, 7 aa, synthetic construct
LGFRRAS

SEQ ID NO: 65 - protein, 7 aa, synthetic construct
LGFNRAS

SEQ ID NO: 66 - protein, 7 aa, synthetic construct
LGFSRAS

SEQ ID NO: 67 - protein, 7 aa, synthetic construct
LGFRQAS

SEQ ID NO: 68 - protein, 7 aa, synthetic construct
LGFRRAS

SEQ ID NO: 69 - protein, 7 aa, synthetic construct
LGFRRAS

SEQ ID NO: 70 - protein, 7 aa, synthetic construct
LGFRRAS

SEQ ID NO: 71 - protein, 9 aa, synthetic construct
MQALDPPLS

SEQ ID NO: 72 - protein, 9 aa, synthetic construct
MQALDPPLS

SEQ ID NO: 73 - protein, 9 aa, synthetic construct
MQALDPPLS

SEQ ID NO: 74 - protein, 9 aa, synthetic construct
MQCLRPPLS

SEQ ID NO: 75 - protein, 9 aa, synthetic construct
MQALRPPLS

SEQ ID NO: 76 - protein, 9 aa, synthetic construct
MQALDPPLS

SEQ ID NO: 77 - protein, 9 aa, synthetic construct
MQALDPPLS

SEQ ID NO: 78 - protein, 9 aa, synthetic construct
MQALDPPLS

SEQ ID NO: 79 - protein, 9 aa, synthetic construct
MQALDPPLS

SEQ ID NO: 80 - protein, 9 aa, synthetic construct
MQALDPPLS

SEQ ID NO: 81 - protein, 9 aa, synthetic construct
MQALDPPLS

SEQ ID NO: 82 - protein, 9 aa, synthetic construct
MQALNPPLS

SEQ ID NO: 83 - protein, 9 aa, synthetic construct
MQALRPPLS

SEQ ID NO: 84 - protein, 9 aa, synthetic construct
MQALDTPLS

SEQ ID NO: 85 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLTNRAYAMDVWGRGTTVVVSS

SEQ ID NO: 86 - protein, 129 aa, synthetic construct; identical to SEQ ID NO: 85

SEQ ID NO: 87 - protein, 129 aa, synthetic construct; identical to SEQ ID NO: 85

SEQ ID NO: 88 - protein, 129 aa, synthetic construct
EVQVVESGGGVVKPGGSLRLSCTANTFTFNNAWVNWVRQAPGRGLEWVGRLRSDTEHWAKDYAAHMKGRVVISREESGRTVYLEMIGLKVEDTAVYYCNTGYWPQSVFLTNRAYAMDVWGRGTTVIVSS

SEQ ID NO: 89 - protein, 129 aa, synthetic construct; identical to SEQ ID NO: 88

SEQ ID NO: 90 - protein, 129 aa, synthetic construct; identical to SEQ ID NO: 85

SEQ ID NO: 91 - protein, 129 aa, synthetic construct; identical to SEQ ID NO: 85

SEQ ID NO: 92 - protein, 129 aa, synthetic construct; identical to SEQ ID NO: 85

SEQ ID NO: 93 - protein, 129 aa, synthetic construct; identical to SEQ ID NO: 85

SEQ ID NO: 94 - protein, 129 aa, synthetic construct; identical to SEQ ID NO: 85

SEQ ID NO: 95 - protein, 129 aa, synthetic construct; identical to SEQ ID NO: 85

SEQ ID NO: 96 - protein, 129 aa, synthetic construct; identical to SEQ ID NO: 85

SEQ ID NO: 97 - protein, 129 aa, synthetic construct; identical to SEQ ID NO: 85

SEQ ID NO: 98 - protein, 129 aa, synthetic construct; identical to SEQ ID NO: 85

SEQ ID NO: 99 - protein, 111 aa, synthetic construct
DIVMTQSPLFLSVTPGESASMSCASNESLRRGQYHFLDWYLQKPGQSPQLLIYLGFRRASGVPARFSARASDTHYTLTISRVDTEDVGVYYCMQALDPPLSFGPGTTVDMK

SEQ ID NO: 100 - protein, 111 aa, synthetic construct
DIVMTQSPLFLSVTPGESASMSCASSESLRRGQYHFLDWYLQKPGQSPQLLIYLGFRRASGVPARFSARASDTHYTLTISRVDTEDVGVYYCMQALDPPLSFGPGTTVDMK

SEQ ID NO: 101 - protein, 111 aa, synthetic construct
DIVMTQSPLFLSVTPGESASMSCASNEALRRGQYHFLDWYLQKPGQSPQLLIYLGFRRASGVPARFSARASDTHYTLTISRVDTEDVGVYYCMQALDPPLSFGPGTTVDMK

SEQ ID NO: 102 - protein, 111 aa, synthetic construct
DIVVTQSPLSLPVTLGEPASMSCKSSESLHREGYNFLDWYLQKPGQSPQLLVYLGSRRAAGVPARFSGSGAGTDYTLRISRVEAEDVGVYYCMQCLRPPLSFGPGTTVDIK

SEQ ID NO: 103 - protein, 111 aa, synthetic construct
DIVVTQSPLSLPVTLGEPASMSCKSSESLHREGYNFLDWYLQKPGQSPQLLVYLGSRRAAGVPARFSGSGAGTDYTLRISRVEAEDVGVYYCMQALRPPLSFGPGTTVDIK

SEQ ID NO: 104 - protein, 111 aa, synthetic construct
DIVMTQSPLFLSVTPGESASMSCASSESLHRGQYHFLDWYLQKPGQSPQLLIYLGFRRASGVPARFSARASDTHYTLTISRVDTEDVGVYYCMQALDPPLSFGPGTTVDMK

SEQ ID NO: 105 - protein, 111 aa, synthetic construct
DIVMTQSPLFLSVTPGESASMSCASSESLQRGQYHFLDWYLQKPGQSPQLLIYLGFRRASGVPARFSARASDTHYTLTISRVDTEDVGVYYCMQALDPPLSFGPGTTVDMK

SEQ ID NO: 106 - protein, 111 aa, synthetic construct
DIVMTQSPLFLSVTPGESASMSCASSESLRSGQYHFLDWYLQKPGQSPQLLIYLGFRRASGVPARFSARASDTHYTLTISRVDTEDVGVYYCMQALDPPLSFGPGTTVDMK

SEQ ID NO: 107 - protein, 111 aa, synthetic construct
DIVMTQSPLFLSVTPGESASMSCASSESLRRGQYHFLDWYLQKPGQSPQLLIYLGFNRASGVPARFSARASDTHYTLTISRVDTEDVGVYYCMQALDPPLSFGPGTTVDMK

SEQ ID NO: 108 - protein, 111 aa, synthetic construct
DIVMTQSPLFLSVTPGESASMSCASSESLRRGQYHFLDWYLQKPGQSPQLLIYLGFSRASGVPARFSARASDTHYTLTISRVDTEDVGVYYCMQALDPPLSFGPGTTVDMK

SEQ ID NO: 109 - protein, 111 aa, synthetic construct
DIVMTQSPLFLSVTPGESASMSCASSESLRRGQYHFLDWYLQKPGQSPQLLIYLGFRQASGVPARFSARASDTHYTLTISRVDTEDVGVYYCMQALDPPLSFGPGTTVDMK

SEQ ID NO: 110 - protein, 111 aa, synthetic construct
DIVMTQSPLFLSVTPGESASMSCASSESLRRGQYHFLDWYLQKPGQSPQLLIYLGFRRASGVPARFSARASDTHYTLTISRVDTEDVGVYYCMQALNPPLSFGPGTTVDMK

SEQ ID NO: 111 - protein, 111 aa, synthetic construct
DIVMTQSPLFLSVTPGESASMSCASSESLRRGQYHFLDWYLQKPGQSPQLLIYLGFRRASGVPARFSARASDTHYTLTISRVDTEDVGVYYCMQALRPPLSFGPGTTVDMK

SEQ ID NO: 112 - protein, 111 aa, synthetic construct
DIVMTQSPLFLSVTPGESASMSCASSESLRRGQYHFLDWYLQKPGQSPQLLIYLGFRRASGVPARFSARASDTHYTLTISRVDTEDVGVYYCMQALDTPLSFGPGTTVDMK

SEQ ID NO: 113 - protein, 10 aa, synthetic construct
SFTFSNAWVN

SEQ ID NO: 114 - protein, 10 aa, synthetic construct
SFTFSNAWVN

SEQ ID NO: 115 - protein, 10 aa, synthetic construct
SFTFSNAWVN

SEQ ID NO: 116 - protein, 10 aa, synthetic construct
SFTFSNAWVN

SEQ ID NO: 117 - protein, 10 aa, synthetic construct
SFTFSNAWVN

SEQ ID NO: 118 - protein, 10 aa, synthetic construct
SFTFSNAWVN

SEQ ID NO: 119 - protein, 10 aa, synthetic construct
TFTFNNAWVN

SEQ ID NO: 120 - protein, 10 aa, synthetic construct
SFTFDNAWVN

SEQ ID NO: 121 - protein, 10 aa, synthetic construct
SFTFSAAWVN

SEQ ID NO: 122 - protein, 10 aa, synthetic construct
SFTFSNAEVN

SEQ ID NO: 123 - protein, 10 aa, synthetic construct
SFTFSNALVN

SEQ ID NO: 124 - protein, 10 aa, synthetic construct
SFTFSNAWVN

SEQ ID NO: 125 - protein, 10 aa, synthetic construct
SFTFSNAWVN

SEQ ID NO: 126 - protein, 10 aa, synthetic construct
SFTFSNAWVN

SEQ ID NO: 127 - protein, 10 aa, synthetic construct
SFTFSNAWVN

SEQ ID NO: 128 - protein, 10 aa, synthetic construct
SFTFSNAWVN

SEQ ID NO: 129 - protein, 10 aa, synthetic construct
SFTFSNAWVN

SEQ ID NO: 130 - protein, 10 aa, synthetic construct
SFTFSNAWVN

SEQ ID NO: 131 - protein, 10 aa, synthetic construct
SFTFSNAWVN

SEQ ID NO: 132 - protein, 10 aa, synthetic construct
SFTFSNAWVN

SEQ ID NO: 133 - protein, 10 aa, synthetic construct
SFTFSNAWVN

SEQ ID NO: 134 - protein, 10 aa, synthetic construct
SFTFSNAWVN

SEQ ID NO: 135 - protein, 10 aa, synthetic construct
SFTFSNAWVN

SEQ ID NO: 136 - protein, 10 aa, synthetic construct
SFTFSNAWVN

SEQ ID NO: 137 - protein, 10 aa, synthetic construct
SFTFSNAWVN

SEQ ID NO: 138 - protein, 10 aa, synthetic construct
SFTFSNAWVN

SEQ ID NO: 139 - protein, 10 aa, synthetic construct
SFTFSNAWVN

SEQ ID NO: 140 - protein, 10 aa, synthetic construct
SFTFSNAWVN

SEQ ID NO: 141 - protein, 10 aa, synthetic construct
SFTFSNAWVN

SEQ ID NO: 142 - protein, 10 aa, synthetic construct
SFTFSNAWVN

SEQ ID NO: 143 - protein, 10 aa, synthetic construct
SFTFSNAWVN

SEQ ID NO: 144 - protein, 10 aa, synthetic construct
SFTFSNAWVN

SEQ ID NO: 145 - protein, 10 aa, synthetic construct
SFTFSNAWVN

SEQ ID NO: 146 - protein, 10 aa, synthetic construct
SFTFSNAWVN

SEQ ID NO: 147 - protein, 10 aa, synthetic construct
SFTFSNAWVN

SEQ ID NO: 148 - protein, 10 aa, synthetic construct
SFTFSNAWVN

SEQ ID NO: 149 - protein, 10 aa, synthetic construct
SFTFSNAWVN

SEQ ID NO: 150 - protein, 10 aa, synthetic construct
SFTFSNAWVN

SEQ ID NO: 151 - protein, 10 aa, synthetic construct
SFTFSNAWVN

SEQ ID NO: 152 - protein, 10 aa, synthetic construct
SFTFSNAWVN

SEQ ID NO: 153 - protein, 10 aa, synthetic construct
SFTFSNAWVN

SEQ ID NO: 154 - protein, 10 aa, synthetic construct
SFTFSNAWVN

SEQ ID NO: 155 - protein, 10 aa, synthetic construct
SFTFSNAWVN

SEQ ID NO: 156 - protein, 10 aa, synthetic construct
SFTFSNAWVN

SEQ ID NO: 157 - protein, 10 aa, synthetic construct
SFTFSNAWVN

SEQ ID NO: 158 - protein, 10 aa, synthetic construct
SFTFSNAWVN

SEQ ID NO: 159 - protein, 10 aa, synthetic construct
SFTFSNAWVN

SEQ ID NO: 160 - protein, 10 aa, synthetic construct
SFTFSNAWVN

SEQ ID NO: 161 - protein, 10 aa, synthetic construct
SFTFSNAWVN

SEQ ID NO: 162 - protein, 10 aa, synthetic construct
SFTFSNAWVN

SEQ ID NO: 163 - protein, 10 aa, synthetic construct
SFTFSNAWVN

SEQ ID NO: 164 - protein, 10 aa, synthetic construct
SFTFSNAWVN

SEQ ID NO: 165 - protein, 10 aa, synthetic construct
SFTFSNAWVN

SEQ ID NO: 166 - protein, 10 aa, synthetic construct
SFTFSNAWVN

SEQ ID NO: 167 - protein, 10 aa, synthetic construct
SFTFSNAWVN

SEQ ID NO: 168 - protein, 10 aa, synthetic construct
SFTFSNAWVN

SEQ ID NO: 169 - protein, 10 aa, synthetic construct
SFTFSNAWVN

SEQ ID NO: 170 - protein, 10 aa, synthetic construct
SFTFSNAWVN

SEQ ID NO: 171 - protein, 10 aa, synthetic construct
SFTFSNAWVN

SEQ ID NO: 172 - protein, 10 aa, synthetic construct
SFTFSNAWVN

SEQ ID NO: 173 - protein, 10 aa, synthetic construct
SFTFSNAWVN

SEQ ID NO: 174 - protein, 10 aa, synthetic construct
SFTFSNAWVN

SEQ ID NO: 175 - protein, 10 aa, synthetic construct
SFTFSNAWVN

SEQ ID NO: 176 - protein, 10 aa, synthetic construct
SFTFSNAWVN

SEQ ID NO: 177 - protein, 10 aa, synthetic construct
SFTFSNAWVN

SEQ ID NO: 178 - protein, 10 aa, synthetic construct
SFTFSNAWVN

SEQ ID NO: 179 - protein, 10 aa, synthetic construct
SFTFSNAWVN

SEQ ID NO: 180 - protein, 10 aa, synthetic construct
SFTFSNAWVN

SEQ ID NO: 181 - protein, 10 aa, synthetic construct
SFTFSNAWVN

SEQ ID NO: 182 - protein, 10 aa, synthetic construct
SFTFSNAWVN

SEQ ID NO: 183 - protein, 10 aa, synthetic construct
SFTFSNAWVN

SEQ ID NO: 184 - protein, 10 aa, synthetic construct
SFTFSNAWVN

SEQ ID NO: 185 - protein, 10 aa, synthetic construct
SFTFSNAWVN

SEQ ID NO: 186 - protein, 10 aa, synthetic construct
SFTFSNAWVN

SEQ ID NO: 187 - protein, 10 aa, synthetic construct
SFTFSNAWVN

SEQ ID NO: 188 - protein, 10 aa, synthetic construct
SFTFSNAWVN

SEQ ID NO: 189 - protein, 10 aa, synthetic construct
SFTFSNAWVN

SEQ ID NO: 190 - protein, 10 aa, synthetic construct
SFTFSNAWVN

SEQ ID NO: 191 - protein, 10 aa, synthetic construct
SFTFSNAWVN

SEQ ID NO: 192 - protein, 10 aa, synthetic construct
SFTFSNAWVN

SEQ ID NO: 193 - protein, 10 aa, synthetic construct
SFTFSNAWVN

SEQ ID NO: 194 - protein, 10 aa, synthetic construct
SFTFSNAWVN

SEQ ID NO: 195 - protein, 10 aa, synthetic construct
SFTFSNAWVN

SEQ ID NO: 196 - protein, 10 aa, synthetic construct
SFTFSNAWVN

SEQ ID NO: 197 - protein, 10 aa, synthetic construct
SFTFSNAWVN

SEQ ID NO: 198 - protein, 10 aa, synthetic construct
SFTFSNAWVN

SEQ ID NO: 199 - protein, 10 aa, synthetic construct
TFTFNNAWVN

SEQ ID NO: 200 - protein, 10 aa, synthetic construct
TFTFNNAWVN

SEQ ID NO: 201 - protein, 10 aa, synthetic construct
TFTFNNAWVN

SEQ ID NO: 202 - protein, 10 aa, synthetic construct
TFTFNNAWVN

SEQ ID NO: 203 - protein, 10 aa, synthetic construct
TFTFNNAWVN

SEQ ID NO: 204 - protein, 10 aa, synthetic construct
TFTFNNAWVN

SEQ ID NO: 205 - protein, 10 aa, synthetic construct
TFTFNNAWVN

SEQ ID NO: 206 - protein, 10 aa, synthetic construct
TFTFNNAWVN

SEQ ID NO: 207 - protein, 10 aa, synthetic construct
TFTFNNAWVN

SEQ ID NO: 208 - protein, 10 aa, synthetic construct
TFTFNNAWVN

SEQ ID NO: 209 - protein, 10 aa, synthetic construct
TFTFNNAWVN

SEQ ID NO: 210 - protein, 10 aa, synthetic construct
TFTFNNAWVN

SEQ ID NO: 211 - protein, 10 aa, synthetic construct
TFTFNNAWVN

SEQ ID NO: 212 - protein, 10 aa, synthetic construct
TFTFNNAWVN

SEQ ID NO: 213 - protein, 19 aa, synthetic construct
RLRSDTEGWTKDYAPHLKD

SEQ ID NO: 214 - protein, 19 aa, synthetic construct
RLRSDTEGWTKDYAPHLKD

SEQ ID NO: 215 - protein, 19 aa, synthetic construct
RLRSDTEGWTKDYAPHLKD

SEQ ID NO: 216 - protein, 19 aa, synthetic construct
RLRSDTEGWTKDYAPHLKD

SEQ ID NO: 217 - protein, 19 aa, synthetic construct
RLRSDTEGWTKDYAPHLKD

SEQ ID NO: 218 - protein, 19 aa, synthetic construct
RLRSDTEGWTKDYAPHLKD

SEQ ID NO: 219 - protein, 19 aa, synthetic construct
RLRSDTEHWAKDYAAHMKG

SEQ ID NO: 220 - protein, 19 aa, synthetic construct
RLRSDTEGWTKDYAPHLKD

SEQ ID NO: 221 - protein, 19 aa, synthetic construct
RLRSDTEGWTKDYAPHLKD

SEQ ID NO: 222 - protein, 19 aa, synthetic construct
RLRSDTEGWTKDYAPHLKD

SEQ ID NO: 223 - protein, 19 aa, synthetic construct
RLRSDTEGWTKDYAPHLKD

SEQ ID NO: 224 - protein, 19 aa, synthetic construct
ELRSDTEGWTKDYAPHLKD

SEQ ID NO: 225 - protein, 19 aa, synthetic construct
RLYSDTEGWTKDYAPHLKD

SEQ ID NO: 226 - protein, 19 aa, synthetic construct
RLASDTEGWTKDYAPHLKD

SEQ ID NO: 227 - protein, 19 aa, synthetic construct
RLRSKTEGWTKDYAPHLKD

SEQ ID NO: 228 - protein, 19 aa, synthetic construct
RLRSYTEGWTKDYAPHLKD

SEQ ID NO: 229 - protein, 19 aa, synthetic construct
RLRSDREGWTKDYAPHLKD

SEQ ID NO: 230 - protein, 19 aa, synthetic construct
RLRSDEEGWTKDYAPHLKD

SEQ ID NO: 231 - protein, 19 aa, synthetic construct
RLRSDYEGWTKDYAPHLKD

SEQ ID NO: 232 - protein, 19 aa, synthetic construct
RLRSDTKGWTKDYAPHLKD

SEQ ID NO: 233 - protein, 19 aa, synthetic construct
RLRSDTEYWTKDYAPHLKD

SEQ ID NO: 234 - protein, 19 aa, synthetic construct
RLRSDTEHWTKDYAPHLKD

SEQ ID NO: 235 - protein, 19 aa, synthetic construct
RLRSDTEGSTKDYAPHLKD

SEQ ID NO: 236 - protein, 19 aa, synthetic construct
RLRSDTEGWYKDYAPHLKD

SEQ ID NO: 237 - protein, 19 aa, synthetic construct
RLRSDTEGWEKDYAPHLKD

SEQ ID NO: 238 - protein, 19 aa, synthetic construct
RLRSDTEGWTADYAPHLKD

SEQ ID NO: 239 - protein, 19 aa, synthetic construct
RLRSDTEGWTARYAPHLKD

SEQ ID NO: 240 - protein, 19 aa, synthetic construct
RLRSDTEGWTKDYAPHLAD

SEQ ID NO: 241 - protein, 19 aa, synthetic construct
RLRSDTEGWTKDYAPHLYD

SEQ ID NO: 242 - protein, 19 aa, synthetic construct
RLRSDTEGWTKDYAPHLKD

SEQ ID NO: 243 - protein, 19 aa, synthetic construct
RLRSDTEGWTKDYAPHLKD

SEQ ID NO: 244 - protein, 19 aa, synthetic construct
RLRSDTEGWTKDYAPHLKD

SEQ ID NO: 245 - protein, 19 aa, synthetic construct
RLRSDTEGWTKDYAPHLKD

SEQ ID NO: 246 - protein, 19 aa, synthetic construct
RLRSDTEGWTKDYAPHLKD

SEQ ID NO: 247 - protein, 19 aa, synthetic construct
RLRSDTEGWTKDYAPHLKD

SEQ ID NO: 248 - protein, 19 aa, synthetic construct
RLRSDTEGWTKDYAPHLKD

SEQ ID NO: 249 - protein, 19 aa, synthetic construct
RLRSDTEGWTKDYAPHLKD

SEQ ID NO: 250 - protein, 19 aa, synthetic construct
RLRSDTEGWTKDYAPHLKD

SEQ ID NO: 251 - protein, 19 aa, synthetic construct
RLRSDTEGWTKDYAPHLKD

SEQ ID NO: 252 - protein, 19 aa, synthetic construct
RLRSDTEGWTKDYAPHLKD

SEQ ID NO: 253 - protein, 19 aa, synthetic construct
RLRSDTEGWTKDYAPHLKD

SEQ ID NO: 254 - protein, 19 aa, synthetic construct
RLRSDTEGWTKDYAPHLKD

SEQ ID NO: 255 - protein, 19 aa, synthetic construct
RLRSDTEGWTKDYAPHLKD

SEQ ID NO: 256 - protein, 19 aa, synthetic construct
RLRSDTEGWTKDYAPHLKD

SEQ ID NO: 257 - protein, 19 aa, synthetic construct
RLRSDTEGWTKDYAPHLKD

SEQ ID NO: 258 - protein, 19 aa, synthetic construct
RLRSDTEGWTKDYAPHLKD

SEQ ID NO: 259 - protein, 19 aa, synthetic construct
RLRSDTEGWTKDYAPHLKD

SEQ ID NO: 260 - protein, 19 aa, synthetic construct
RLRSDTEGWTKDYAPHLKD

SEQ ID NO: 261 - protein, 19 aa, synthetic construct
RLRSDTEGWTKDYAPHLKD

SEQ ID NO: 262 - protein, 19 aa, synthetic construct
RLRSDTEGWTKDYAPHLKD

SEQ ID NO: 263 - protein, 19 aa, synthetic construct
RLRSDTEGWTKDYAPHLKD

SEQ ID NO: 264 - protein, 19 aa, synthetic construct
RLRSDTEGWTKDYAPHLKD

SEQ ID NO: 265 - protein, 19 aa, synthetic construct
RLRSDTEGWTKDYAPHLKD

SEQ ID NO: 266 - protein, 19 aa, synthetic construct
RLRSDTEGWTKDYAPHLKD

SEQ ID NO: 267 - protein, 19 aa, synthetic construct
RLRSDTEGWTKDYAPHLKD

SEQ ID NO: 268 - protein, 19 aa, synthetic construct
RLRSDTEGWTKDYAPHLKD

SEQ ID NO: 269 - protein, 19 aa, synthetic construct
RLRSDTEGWTKDYAPHLKD

SEQ ID NO: 270 - protein, 19 aa, synthetic construct
RLRSDTEGWTKDYAPHLKD

SEQ ID NO: 271 - protein, 19 aa, synthetic construct
RLRSDTEGWTKDYAPHLKD

SEQ ID NO: 272 - protein, 19 aa, synthetic construct
RLRSDTEGWTKDYAPHLKD

SEQ ID NO: 273 - protein, 19 aa, synthetic construct
RLRSDTEGWTKDYAPHLKD

SEQ ID NO: 274 - protein, 19 aa, synthetic construct
RLRSDTEGWTKDYAPHLKD

SEQ ID NO: 275 - protein, 19 aa, synthetic construct
RLRSDTEGWTKDYAPHLKD

SEQ ID NO: 276 - protein, 19 aa, synthetic construct
RLRSDTEGWTKDYAPHLKD

SEQ ID NO: 277 - protein, 19 aa, synthetic construct
RLRSDTEGWTKDYAPHLKD

SEQ ID NO: 278 - protein, 19 aa, synthetic construct
RLRSDTEGWTKDYAPHLKD

SEQ ID NO: 279 - protein, 19 aa, synthetic construct
RLRSDTEGWTKDYAPHLKD

SEQ ID NO: 280 - protein, 19 aa, synthetic construct
RLRSDTEGWTKDYAPHLKD

SEQ ID NO: 281 - protein, 19 aa, synthetic construct
RLRSDTEGWTKDYAPHLKD

SEQ ID NO: 282 - protein, 19 aa, synthetic construct
RLRSDTEGWTKDYAPHLKD

SEQ ID NO: 283 - protein, 19 aa, synthetic construct
RLRSDTEGWTKDYAPHLKD

SEQ ID NO: 284 - protein, 19 aa, synthetic construct
RLRSDTEGWTKDYAPHLKD

SEQ ID NO: 285 - protein, 19 aa, synthetic construct
RLRSDTEGWTKDYAPHLKD

SEQ ID NO: 286 - protein, 19 aa, synthetic construct
RLRSDTEGWTKDYAPHLKD

SEQ ID NO: 287 - protein, 19 aa, synthetic construct
RLRSDTEGWTKDYAPHLKD

SEQ ID NO: 288 - protein, 19 aa, synthetic construct
RLRSDTEGWTKDYAPHLKD

SEQ ID NO: 289 - protein, 19 aa, synthetic construct
RLRSDTEGWTKDYAPHLKD

SEQ ID NO: 290 - protein, 19 aa, synthetic construct
RLRSDTEGWTKDYAPHLKD

SEQ ID NO: 291 - protein, 19 aa, synthetic construct
RLRSDTEGWTKDYAPHLKD

SEQ ID NO: 292 - protein, 19 aa, synthetic construct
RLRSDTEGWTKDYAPHLKD

SEQ ID NO: 293 - protein, 19 aa, synthetic construct
RLRSDTEGWTKDYAPHLKD

SEQ ID NO: 294 - protein, 19 aa, synthetic construct
RLRSDTEGWTKDYAPHLKD

SEQ ID NO: 295 - protein, 19 aa, synthetic construct
RLRSDTEGWTKDYAPHLKD

SEQ ID NO: 296 - protein, 19 aa, synthetic construct
RLRSDTEGWTKDYAPHLKD

SEQ ID NO: 297 - protein, 19 aa, synthetic construct
RLRSDTEGWTKDYAPHLKD

SEQ ID NO: 298 - protein, 19 aa, synthetic construct
RLRSDTEGWTKDYAPHLKD

SEQ ID NO: 299 - protein, 19 aa, synthetic construct
RLQSDTEHWAKDYAAHMKG

SEQ ID NO: 300 - protein, 19 aa, synthetic construct
RLRSNTEHWAKDYAAHMKG

SEQ ID NO: 301 - protein, 19 aa, synthetic construct
RLRSDQEHWAKDYAAHMKG

SEQ ID NO: 302 - protein, 19 aa, synthetic construct
RLRSDYEHWAKDYAAHMKG

SEQ ID NO: 303 - protein, 19 aa, synthetic construct
RLRSDTQHWAKDYAAHMKG

SEQ ID NO: 304 - protein, 19 aa, synthetic construct
RLRSDTEHWAKDYAAHMKG

SEQ ID NO: 305 - protein, 19 aa, synthetic construct
RLRSDTEHWAKDYAAHMKG

SEQ ID NO: 306 - protein, 19 aa, synthetic construct
RLRSDTEHWAKDYAAHMKG

SEQ ID NO: 307 - protein, 19 aa, synthetic construct
RLRSDTEHWAKDYAAHMKG

SEQ ID NO: 308 - protein, 19 aa, synthetic construct
RLRSDTEHWAKDYAAHMKG

SEQ ID NO: 309 - protein, 19 aa, synthetic construct
RLRSDTEHWAKDYAAHMKG

SEQ ID NO: 310 - protein, 19 aa, synthetic construct
RLRSDTEHWAKDYAAHMKG

SEQ ID NO: 311 - protein, 19 aa, synthetic construct
RLRSDTEHWAKDYAAHMKG

SEQ ID NO: 312 - protein, 19 aa, synthetic construct
RLRSDTEHWAKDYAAHMKG

SEQ ID NO: 313 - protein, 20 aa, synthetic construct
NTGYWPQSAFLTNRAYAMDV

SEQ ID NO: 314 - protein, 20 aa, synthetic construct
NTGYWPQSAFLTNRAYAMDV

SEQ ID NO: 315 - protein, 20 aa, synthetic construct
NTGYWPQSAFLTNRAYAMDV

SEQ ID NO: 316 - protein, 20 aa, synthetic construct
NTGYWPQSAFLTNRAYAMDV

SEQ ID NO: 317 - protein, 20 aa, synthetic construct
NTGYWPQSAFLTNRAYAMDV

SEQ ID NO: 318 - protein, 20 aa, synthetic construct
NTGYWPQSAFLTNRAYAMDV

SEQ ID NO: 319 - protein, 20 aa, synthetic construct
NTGYWPQSVFLTNRAYAMDV

SEQ ID NO: 320 - protein, 20 aa, synthetic construct
NTGYWPQSAFLTNRAYAMDV

SEQ ID NO: 321 - protein, 20 aa, synthetic construct
NTGYWPQSAFLTNRAYAMDV

SEQ ID NO: 322 - protein, 20 aa, synthetic construct
NTGYWPQSAFLTNRAYAMDV

SEQ ID NO: 323 - protein, 20 aa, synthetic construct
NTGYWPQSAFLTNRAYAMDV

SEQ ID NO: 324 - protein, 20 aa, synthetic construct
NTGYWPQSAFLTNRAYAMDV

SEQ ID NO: 325 - protein, 20 aa, synthetic construct
NTGYWPQSAFLTNRAYAMDV

SEQ ID NO: 326 - protein, 20 aa, synthetic construct
NTGYWPQSAFLTNRAYAMDV

SEQ ID NO: 327 - protein, 20 aa, synthetic construct
NTGYWPQSAFLTNRAYAMDV

SEQ ID NO: 328 - protein, 20 aa, synthetic construct
NTGYWPQSAFLTNRAYAMDV

SEQ ID NO: 329 - protein, 20 aa, synthetic construct
NTGYWPQSAFLTNRAYAMDV

SEQ ID NO: 330 - protein, 20 aa, synthetic construct
NTGYWPQSAFLTNRAYAMDV

SEQ ID NO: 331 - protein, 20 aa, synthetic construct
NTGYWPQSAFLTNRAYAMDV

SEQ ID NO: 332 - protein, 20 aa, synthetic construct
NTGYWPQSAFLTNRAYAMDV

SEQ ID NO: 333 - protein, 20 aa, synthetic construct
NTGYWPQSAFLTNRAYAMDV

SEQ ID NO: 334 - protein, 20 aa, synthetic construct
NTGYWPQSAFLTNRAYAMDV

SEQ ID NO: 335 - protein, 20 aa, synthetic construct
NTGYWPQSAFLTNRAYAMDV

SEQ ID NO: 336 - protein, 20 aa, synthetic construct
NTGYWPQSAFLTNRAYAMDV

SEQ ID NO: 337 - protein, 20 aa, synthetic construct
NTGYWPQSAFLTNRAYAMDV

SEQ ID NO: 338 - protein, 20 aa, synthetic construct
NTGYWPQSAFLTNRAYAMDV

SEQ ID NO: 339 - protein, 20 aa, synthetic construct
NTGYWPQSAFLTNRAYAMDV

SEQ ID NO: 340 - protein, 20 aa, synthetic construct
NTGYWPQSAFLTNRAYAMDV

SEQ ID NO: 341 - protein, 20 aa, synthetic construct
NTGYWPQSAFLTNRAYAMDV

SEQ ID NO: 342 - protein, 20 aa, synthetic construct
NTGAWPQSAFLTNRAYAMDV

SEQ ID NO: 343 - protein, 20 aa, synthetic construct
NTGRWPQSAFLTNRAYAMDV

SEQ ID NO: 344 - protein, 20 aa, synthetic construct
NTGYAPQSAFLTNRAYAMDV

SEQ ID NO: 345 - protein, 20 aa, synthetic construct
NTGYEPQSAFLTNRAYAMDV

SEQ ID NO: 346 - protein, 20 aa, synthetic construct
NTGYWPASAFLTNRAYAMDV

SEQ ID NO: 347 - protein, 20 aa, synthetic construct
NTGYWPYSAFLTNRAYAMDV

SEQ ID NO: 348 - protein, 20 aa, synthetic construct
NTGYWPQYAFLTNRAYAMDV

SEQ ID NO: 349 - protein, 20 aa, synthetic construct
NTGYWPQEAFLTNRAYAMDV

SEQ ID NO: 350 - protein, 20 aa, synthetic construct
NTGYWPQSYFLTNRAYAMDV

SEQ ID NO: 351 - protein, 20 aa, synthetic construct
NTGYWPQSKFLTNRAYAMDV

SEQ ID NO: 352 - protein, 20 aa, synthetic construct
NTGYWPQSAALTNRAYAMDV

SEQ ID NO: 353 - protein, 20 aa, synthetic construct
NTGYWPQSAQLTNRAYAMDV

SEQ ID NO: 354 - protein, 20 aa, synthetic construct
NTGYWPQSAFATNRAYAMDV

SEQ ID NO: 355 - protein, 20 aa, synthetic construct
NTGYWPQSAFLENRAYAMDV

SEQ ID NO: 356 - protein, 20 aa, synthetic construct
NTGYWPQSAFLYNRAYAMDV

SEQ ID NO: 357 - protein, 20 aa, synthetic construct
NTGYWPQSAFLTQRAYAMDV

SEQ ID NO: 358 - protein, 20 aa, synthetic construct
NTGYWPQSAFLTARAYAMDV

SEQ ID NO: 359 - protein, 20 aa, synthetic construct
NTGYWPQSAFLTNEAYAMDV

SEQ ID NO: 360 - protein, 20 aa, synthetic construct
NTGYWPQSAFLTNSAYAMDV

SEQ ID NO: 361 - protein, 20 aa, synthetic construct
NTGYWPQSAFLTNRYYAMDV

SEQ ID NO: 362 - protein, 20 aa, synthetic construct
NTGYWPQSAFLTNRNYAMDV

SEQ ID NO: 363 - protein, 20 aa, synthetic construct
NTGYWPQSAFLTNRAAAMDV

SEQ ID NO: 364 - protein, 20 aa, synthetic construct
NTGYWPQSAFLTNRAKAMDV

SEQ ID NO: 365 - protein, 20 aa, synthetic construct
NTGYWPQSAFLTNRAYAMDV

SEQ ID NO: 366 - protein, 20 aa, synthetic construct
NTGYWPQSAFLTNRAYAMDV

SEQ ID NO: 367 - protein, 20 aa, synthetic construct
NTGYWPQSAFLTNRAYAMDV

SEQ ID NO: 368 - protein, 20 aa, synthetic construct
NTGYWPQSAFLTNRAYAMDV

SEQ ID NO: 369 - protein, 20 aa, synthetic construct
NTGYWPQSAFLTNRAYAMDV

SEQ ID NO: 370 - protein, 20 aa, synthetic construct
NTGYWPQSAFLTNRAYAMDV

SEQ ID NO: 371 - protein, 20 aa, synthetic construct
NTGYWPQSAFLTNRAYAMDV

SEQ ID NO: 372 - protein, 20 aa, synthetic construct
NTGYWPQSAFLTNRAYAMDV

SEQ ID NO: 373 - protein, 20 aa, synthetic construct
NTGYWPQSAFLTNRAYAMDV

SEQ ID NO: 374 - protein, 20 aa, synthetic construct
NTGYWPQSAFLTNRAYAMDV

SEQ ID NO: 375 - protein, 20 aa, synthetic construct
NTGYWPQSAFLTNRAYAMDV

SEQ ID NO: 376 - protein, 20 aa, synthetic construct
NTGYWPQSAFLTNRAYAMDV

SEQ ID NO: 377 - protein, 20 aa, synthetic construct
NTGYWPQSAFLTNRAYAMDV

SEQ ID NO: 378 - protein, 20 aa, synthetic construct
NTGYWPQSAFLTNRAYAMDV

SEQ ID NO: 379 - protein, 20 aa, synthetic construct
NTGYWPQSAFLTNRAYAMDV

SEQ ID NO: 380 - protein, 20 aa, synthetic construct
NTGYWPQSAFLTNRAYAMDV

SEQ ID NO: 381 - protein, 20 aa, synthetic construct
NTGYWPQSAFLTNRAYAMDV

SEQ ID NO: 382 - protein, 20 aa, synthetic construct
NTGYWPQSAFLTNRAYAMDV

SEQ ID NO: 383 - protein, 20 aa, synthetic construct
NTGYWPQSAFLTNRAYAMDV

SEQ ID NO: 384 - protein, 20 aa, synthetic construct
NTGYWPQSAFLTNRAYAMDV

SEQ ID NO: 385 - protein, 20 aa, synthetic construct
NTGYWPQSAFLTNRAYAMDV

SEQ ID NO: 386 - protein, 20 aa, synthetic construct
NTGYWPQSAFLTNRAYAMDV

SEQ ID NO: 387 - protein, 20 aa, synthetic construct
NTGYWPQSAFLTNRAYAMDV

SEQ ID NO: 388 - protein, 20 aa, synthetic construct
NTGYWPQSAFLTNRAYAMDV

SEQ ID NO: 389 - protein, 20 aa, synthetic construct
NTGYWPQSAFLTNRAYAMDV

SEQ ID NO: 390 - protein, 20 aa, synthetic construct
NTGYWPQSAFLTNRAYAMDV

SEQ ID NO: 391 - protein, 20 aa, synthetic construct
NTGYWPQSAFLTNRAYAMDV

SEQ ID NO: 392 - protein, 20 aa, synthetic construct
NTGYWPQSAFLTNRAYAMDV

SEQ ID NO: 393 - protein, 20 aa, synthetic construct
NTGYWPQSAFLTNRAYAMDV

SEQ ID NO: 394 - protein, 20 aa, synthetic construct
NTGYWPQSAFLTNRAYAMDV

SEQ ID NO: 395 - protein, 20 aa, synthetic construct
NTGYWPQSAFLTNRAYAMDV

SEQ ID NO: 396 - protein, 20 aa, synthetic construct
NTGYWPQSAFLTNRAYAMDV

SEQ ID NO: 397 - protein, 20 aa, synthetic construct
NTGYWPQSAFLTNRAYAMDV

SEQ ID NO: 398 - protein, 20 aa, synthetic construct
NTGYWPQSAFLTNRAYAMDV

SEQ ID NO: 399 - protein, 20 aa, synthetic construct
NTGYWPQSVFLTNRAYAMDV

SEQ ID NO: 400 - protein, 20 aa, synthetic construct
NTGYWPQSVFLTNRAYAMDV

SEQ ID NO: 401 - protein, 20 aa, synthetic construct
NTGYWPQSVFLTNRAYAMDV

SEQ ID NO: 402 - protein, 20 aa, synthetic construct
NTGYWPQSVFLTNRAYAMDV

SEQ ID NO: 403 - protein, 20 aa, synthetic construct
NTGYWPQSVFLTNRAYAMDV

SEQ ID NO: 404 - protein, 20 aa, synthetic construct
NTGYWPQSVFLTNRAYAMDV

SEQ ID NO: 405 - protein, 20 aa, synthetic construct
NTGYWPQSVFLTNRAYAMDV

SEQ ID NO: 406 - protein, 20 aa, synthetic construct
NTGYWPQSVFLTNRAYAMDV

SEQ ID NO: 407 - protein, 20 aa, synthetic construct
NTGKWPQSVFLTNRAYAMDV

SEQ ID NO: 408 - protein, 20 aa, synthetic construct
NTGYDPQSVFLTNRAYAMDV

SEQ ID NO: 409 - protein, 20 aa, synthetic construct
NTGYWPASVFLTNRAYAMDV

SEQ ID NO: 410 - protein, 20 aa, synthetic construct
NTGYWPQSVQLTNRAYAMDV

SEQ ID NO: 411 - protein, 20 aa, synthetic construct
NTGYWPQSVFLTDRAYAMDV

SEQ ID NO: 412 - protein, 20 aa, synthetic construct
NTGYWPQSVFLTNQAYAMDV

SEQ ID NO: 413 - protein, 15 aa, synthetic construct
ASSESLQRGQYHFLD

SEQ ID NO: 414 - protein, 15 aa, synthetic construct
ASSESLQRGQYHFLD

SEQ ID NO: 415 - protein, 15 aa, synthetic construct
ASSESLRSGQYHFLD

SEQ ID NO: 416 - protein, 15 aa, synthetic construct
ASSESLQSGQYHFLD

SEQ ID NO: 417 - protein, 15 aa, synthetic construct
ASSESLRSGQYHFLD

SEQ ID NO: 418 - protein, 15 aa, synthetic construct
ASSESLQSGQYHFLD

SEQ ID NO: 419 - protein, 15 aa, synthetic construct
KSSESLHREGYNFLD

SEQ ID NO: 420 - protein, 15 aa, synthetic construct
ASSESLQRGQYHFLD

SEQ ID NO: 421 - protein, 15 aa, synthetic construct
ASSESLQRGQYHFLD

SEQ ID NO: 422 - protein, 15 aa, synthetic construct
ASSESLQRGQYHFLD

SEQ ID NO: 423 - protein, 15 aa, synthetic construct
ASSESLQRGQYHFLD

SEQ ID NO: 424 - protein, 15 aa, synthetic construct
ASSESLQRGQYHFLD

SEQ ID NO: 425 - protein, 15 aa, synthetic construct
ASSESLQRGQYHFLD

SEQ ID NO: 426 - protein, 15 aa, synthetic construct
ASSESLQRGQYHFLD

SEQ ID NO: 427 - protein, 15 aa, synthetic construct
ASSESLQRGQYHFLD

SEQ ID NO: 428 - protein, 15 aa, synthetic construct
ASSESLQRGQYHFLD

SEQ ID NO: 429 - protein, 15 aa, synthetic construct
ASSESLQRGQYHFLD

SEQ ID NO: 430 - protein, 15 aa, synthetic construct
ASSESLQRGQYHFLD

SEQ ID NO: 431 - protein, 15 aa, synthetic construct
ASSESLQRGQYHFLD

SEQ ID NO: 432 - protein, 15 aa, synthetic construct
ASSESLQRGQYHFLD

SEQ ID NO: 433 - protein, 15 aa, synthetic construct
ASSESLQRGQYHFLD

SEQ ID NO: 434 - protein, 15 aa, synthetic construct
ASSESLQRGQYHFLD

SEQ ID NO: 435 - protein, 15 aa, synthetic construct
ASSESLQRGQYHFLD

SEQ ID NO: 436 - protein, 15 aa, synthetic construct
ASSESLQRGQYHFLD

SEQ ID NO: 437 - protein, 15 aa, synthetic construct
ASSESLQRGQYHFLD

SEQ ID NO: 438 - protein, 15 aa, synthetic construct
ASSESLQRGQYHFLD

SEQ ID NO: 439 - protein, 15 aa, synthetic construct
ASSESLQRGQYHFLD

SEQ ID NO: 440 - protein, 15 aa, synthetic construct
ASSESLQRGQYHFLD

SEQ ID NO: 441 - protein, 15 aa, synthetic construct
ASSESLQRGQYHFLD

SEQ ID NO: 442 - protein, 15 aa, synthetic construct
ASSESLQRGQYHFLD

SEQ ID NO: 443 - protein, 15 aa, synthetic construct
ASSESLQRGQYHFLD

SEQ ID NO: 444 - protein, 15 aa, synthetic construct
ASSESLQRGQYHFLD

SEQ ID NO: 445 - protein, 15 aa, synthetic construct
ASSESLQRGQYHFLD

SEQ ID NO: 446 - protein, 15 aa, synthetic construct
ASSESLQRGQYHFLD

SEQ ID NO: 447 - protein, 15 aa, synthetic construct
ASSESLQRGQYHFLD

SEQ ID NO: 448 - protein, 15 aa, synthetic construct
ASSESLQRGQYHFLD

SEQ ID NO: 449 - protein, 15 aa, synthetic construct
ASSESLQRGQYHFLD

SEQ ID NO: 450 - protein, 15 aa, synthetic construct
ASSESLQRGQYHFLD

SEQ ID NO: 451 - protein, 15 aa, synthetic construct
ASSESLQRGQYHFLD

SEQ ID NO: 452 - protein, 15 aa, synthetic construct
ASSESLQRGQYHFLD

SEQ ID NO: 453 - protein, 15 aa, synthetic construct
ASSESLQRGQYHFLD

SEQ ID NO: 454 - protein, 15 aa, synthetic construct
ASSESLQRGQYHFLD

SEQ ID NO: 455 - protein, 15 aa, synthetic construct
ASSESLQRGQYHFLD

SEQ ID NO: 456 - protein, 15 aa, synthetic construct
ASSESLQRGQYHFLD

SEQ ID NO: 457 - protein, 15 aa, synthetic construct
ASSESLQRGQYHFLD

SEQ ID NO: 458 - protein, 15 aa, synthetic construct
ASSESLQRGQYHFLD

SEQ ID NO: 459 - protein, 15 aa, synthetic construct
ASSESLQRGQYHFLD

SEQ ID NO: 460 - protein, 15 aa, synthetic construct
ASSESLQRGQYHFLD

SEQ ID NO: 461 - protein, 15 aa, synthetic construct
ASSESLQRGQYHFLD

SEQ ID NO: 462 - protein, 15 aa, synthetic construct
ASSESLQRGQYHFLD

SEQ ID NO: 463 - protein, 15 aa, synthetic construct
ASSESLQRGQYHFLD

SEQ ID NO: 464 - protein, 15 aa, synthetic construct
ASSESLQRGQYHFLD

SEQ ID NO: 465 - protein, 15 aa, synthetic construct
ASSASLQRGQYHFLD

SEQ ID NO: 466 - protein, 15 aa, synthetic construct
ASSYSLQRGQYHFLD

SEQ ID NO: 467 - protein, 15 aa, synthetic construct
ASSEYLQRGQYHFLD

SEQ ID NO: 468 - protein, 15 aa, synthetic construct
ASSEELQRGQYHFLD

SEQ ID NO: 469 - protein, 15 aa, synthetic construct
ASSESLQREQYHFLD

SEQ ID NO: 470 - protein, 15 aa, synthetic construct
ASSESLQRSQYHFLD

SEQ ID NO: 471 - protein, 15 aa, synthetic construct
ASSESLQRGYYHFLD

SEQ ID NO: 472 - protein, 15 aa, synthetic construct
ASSESLQRGSYHFLD

SEQ ID NO: 473 - protein, 15 aa, synthetic construct
ASSESLQRGQAHFLD

SEQ ID NO: 474 - protein, 15 aa, synthetic construct
ASSESLQRGQHHFLD

SEQ ID NO: 475 - protein, 15 aa, synthetic construct
ASSESLQRGQVHFLD

SEQ ID NO: 476 - protein, 15 aa, synthetic construct
ASSESLQRGQSHFLD

SEQ ID NO: 477 - protein, 15 aa, synthetic construct
ASSESLQRGQYAFLD

SEQ ID NO: 478 - protein, 15 aa, synthetic construct
ASSESLQRGQYNFLD

SEQ ID NO: 479 - protein, 15 aa, synthetic construct
ASSESLQRGQYHALD

SEQ ID NO: 480 - protein, 15 aa, synthetic construct
ASSESLQRGQYHKLD

SEQ ID NO: 481 - protein, 15 aa, synthetic construct
ASSESLQRGQYHFLN

SEQ ID NO: 482 - protein, 15 aa, synthetic construct
ASSESLQRGQYHFLS

SEQ ID NO: 483 - protein, 15 aa, synthetic construct
ASSESLQRGQYHFLD

SEQ ID NO: 484 - protein, 15 aa, synthetic construct
ASSESLQRGQYHFLD

SEQ ID NO: 485 - protein, 15 aa, synthetic construct
ASSESLQRGQYHFLD

SEQ ID NO: 486 - protein, 15 aa, synthetic construct
ASSESLQRGQYHFLD

SEQ ID NO: 487 - protein, 15 aa, synthetic construct
ASSESLQRGQYHFLD

SEQ ID NO: 488 - protein, 15 aa, synthetic construct
ASSESLQRGQYHFLD

SEQ ID NO: 489 - protein, 15 aa, synthetic construct
ASSESLQRGQYHFLD

SEQ ID NO: 490 - protein, 15 aa, synthetic construct
ASSESLQRGQYHFLD

SEQ ID NO: 491 - protein, 15 aa, synthetic construct
ASSESLQRGQYHFLD

SEQ ID NO: 492 - protein, 15 aa, synthetic construct
ASSESLQRGQYHFLD

SEQ ID NO: 493 - protein, 15 aa, synthetic construct
ASSESLQRGQYHFLD

SEQ ID NO: 494 - protein, 15 aa, synthetic construct
ASSESLQRGQYHFLD

SEQ ID NO: 495 - protein, 15 aa, synthetic construct
ASSESLQRGQYHFLD

SEQ ID NO: 496 - protein, 15 aa, synthetic construct
ASSESLQRGQYHFLD

SEQ ID NO: 497 - protein, 15 aa, synthetic construct
ASSESLQRGQYHFLD

SEQ ID NO: 498 - protein, 15 aa, synthetic construct
ASSESLQRGQYHFLD

SEQ ID NO: 499 - protein, 15 aa, synthetic construct
KSSESLHREGYNFLD

SEQ ID NO: 500 - protein, 15 aa, synthetic construct
KSSESLHREGYNFLD

SEQ ID NO: 501 - protein, 15 aa, synthetic construct
KSSESLHREGYNFLD

SEQ ID NO: 502 - protein, 15 aa, synthetic construct
KSSESLHREGYNFLD

SEQ ID NO: 503 - protein, 15 aa, synthetic construct
KSSESLHREGYNFLD

SEQ ID NO: 504 - protein, 15 aa, synthetic construct
KSSESLHRQGYNFLD

SEQ ID NO: 505 - protein, 15 aa, synthetic construct
KSSESLHRGQYNFLD

SEQ ID NO: 506 - protein, 15 aa, synthetic construct
KSSESLHREGTNFLD

SEQ ID NO: 507 - protein, 15 aa, synthetic construct
KSSESLHREGYNFLD

SEQ ID NO: 508 - protein, 15 aa, synthetic construct
KSSESLHREGYNFLD

SEQ ID NO: 509 - protein, 15 aa, synthetic construct
KSSESLHREGYNFLD

SEQ ID NO: 510 - protein, 15 aa, synthetic construct
KSSESLHREGYNFLD

SEQ ID NO: 511 - protein, 15 aa, synthetic construct
KSSESLHREGYNFLD

SEQ ID NO: 512 - protein, 15 aa, synthetic construct
KSSESLHREGYNFLD

SEQ ID NO: 513 - protein, 7 aa, synthetic construct
LGFRRAS

SEQ ID NO: 514 - protein, 7 aa, synthetic construct
LGFNRAS

SEQ ID NO: 515 - protein, 7 aa, synthetic construct
LGFRRAS

SEQ ID NO: 516 - protein, 7 aa, synthetic construct
LGFRRAS

SEQ ID NO: 517 - protein, 7 aa, synthetic construct
LGFNRAS

SEQ ID NO: 518 - protein, 7 aa, synthetic construct
LGFNRAS

SEQ ID NO: 519 - protein, 7 aa, synthetic construct
LGSNRAA

SEQ ID NO: 520 - protein, 7 aa, synthetic construct
LGFRRAS

SEQ ID NO: 521 - protein, 7 aa, synthetic construct
LGFRRAS

SEQ ID NO: 522 - protein, 7 aa, synthetic construct
LGFRRAS

SEQ ID NO: 523 - protein, 7 aa, synthetic construct
LGFRRAS

SEQ ID NO: 524 - protein, 7 aa, synthetic construct
LGFRRAS

SEQ ID NO: 525 - protein, 7 aa, synthetic construct
LGFRRAS

SEQ ID NO: 526 - protein, 7 aa, synthetic construct
LGFRRAS

SEQ ID NO: 527 - protein, 7 aa, synthetic construct
LGFRRAS

SEQ ID NO: 528 - protein, 7 aa, synthetic construct
LGFRRAS

SEQ ID NO: 529 - protein, 7 aa, synthetic construct
LGFRRAS

SEQ ID NO: 530 - protein, 7 aa, synthetic construct
LGFRRAS

SEQ ID NO: 531 - protein, 7 aa, synthetic construct
LGFRRAS

SEQ ID NO: 532 - protein, 7 aa, synthetic construct
LGFRRAS

SEQ ID NO: 533 - protein, 7 aa, synthetic construct
LGFRRAS

SEQ ID NO: 534 - protein, 7 aa, synthetic construct
LGFRRAS

SEQ ID NO: 535 - protein, 7 aa, synthetic construct
LGFRRAS

SEQ ID NO: 536 - protein, 7 aa, synthetic construct
LGFRRAS

SEQ ID NO: 537 - protein, 7 aa, synthetic construct
LGFRRAS

SEQ ID NO: 538 - protein, 7 aa, synthetic construct
LGFRRAS

SEQ ID NO: 539 - protein, 7 aa, synthetic construct
LGFRRAS

SEQ ID NO: 540 - protein, 7 aa, synthetic construct
LGFRRAS

SEQ ID NO: 541 - protein, 7 aa, synthetic construct
LGFRRAS

SEQ ID NO: 542 - protein, 7 aa, synthetic construct
LGFRRAS

SEQ ID NO: 543 - protein, 7 aa, synthetic construct
LGFRRAS

SEQ ID NO: 544 - protein, 7 aa, synthetic construct
LGFRRAS

SEQ ID NO: 545 - protein, 7 aa, synthetic construct
LGFRRAS

SEQ ID NO: 546 - protein, 7 aa, synthetic construct
LGFRRAS

SEQ ID NO: 547 - protein, 7 aa, synthetic construct
LGFRRAS

SEQ ID NO: 548 - protein, 7 aa, synthetic construct
LGFRRAS

SEQ ID NO: 549 - protein, 7 aa, synthetic construct
LGFRRAS

SEQ ID NO: 550 - protein, 7 aa, synthetic construct
LGFRRAS

SEQ ID NO: 551 - protein, 7 aa, synthetic construct
LGFRRAS

SEQ ID NO: 552 - protein, 7 aa, synthetic construct
LGFRRAS

SEQ ID NO: 553 - protein, 7 aa, synthetic construct
LGFRRAS

SEQ ID NO: 554 - protein, 7 aa, synthetic construct
LGFRRAS

SEQ ID NO: 555 - protein, 7 aa, synthetic construct
LGFRRAS

SEQ ID NO: 556 - protein, 7 aa, synthetic construct
LGFRRAS

SEQ ID NO: 557 - protein, 7 aa, synthetic construct
LGFRRAS

SEQ ID NO: 558 - protein, 7 aa, synthetic construct
LGFRRAS

SEQ ID NO: 559 - protein, 7 aa, synthetic construct
LGFRRAS

SEQ ID NO: 560 - protein, 7 aa, synthetic construct
LGFRRAS

SEQ ID NO: 561 - protein, 7 aa, synthetic construct
LGFRRAS

SEQ ID NO: 562 - protein, 7 aa, synthetic construct
LGFRRAS

SEQ ID NO: 563 - protein, 7 aa, synthetic construct
LGFRRAS

SEQ ID NO: 564 - protein, 7 aa, synthetic construct
LGFRRAS

SEQ ID NO: 565 - protein, 7 aa, synthetic construct
LGFRRAS

SEQ ID NO: 566 - protein, 7 aa, synthetic construct
LGFRRAS

SEQ ID NO: 567 - protein, 7 aa, synthetic construct
LGFRRAS

SEQ ID NO: 568 - protein, 7 aa, synthetic construct
LGFRRAS

SEQ ID NO: 569 - protein, 7 aa, synthetic construct
LGFRRAS

SEQ ID NO: 570 - protein, 7 aa, synthetic construct
LGFRRAS

SEQ ID NO: 571 - protein, 7 aa, synthetic construct
LGFRRAS

SEQ ID NO: 572 - protein, 7 aa, synthetic construct
LGFRRAS

SEQ ID NO: 573 - protein, 7 aa, synthetic construct
LGFRRAS

SEQ ID NO: 574 - protein, 7 aa, synthetic construct
LGFRRAS

SEQ ID NO: 575 - protein, 7 aa, synthetic construct
LGFRRAS

SEQ ID NO: 576 - protein, 7 aa, synthetic construct
LGFRRAS

SEQ ID NO: 577 - protein, 7 aa, synthetic construct
LGFRRAS

SEQ ID NO: 578 - protein, 7 aa, synthetic construct
LGFRRAS

SEQ ID NO: 579 - protein, 7 aa, synthetic construct
LGFRRAS

SEQ ID NO: 580 - protein, 7 aa, synthetic construct
LGFRRAS

SEQ ID NO: 581 - protein, 7 aa, synthetic construct
LGFRRAS

SEQ ID NO: 582 - protein, 7 aa, synthetic construct
LGFRRAS

SEQ ID NO: 583 - protein, 7 aa, synthetic construct
AGFRRAS

SEQ ID NO: 584 - protein, 7 aa, synthetic construct
KGFRRAS

SEQ ID NO: 585 - protein, 7 aa, synthetic construct
LGLRRAS

SEQ ID NO: 586 - protein, 7 aa, synthetic construct
LGERRAS

SEQ ID NO: 587 - protein, 7 aa, synthetic construct
LGFHRAS

SEQ ID NO: 588 - protein, 7 aa, synthetic construct
LGFRKAS

SEQ ID NO: 589 - protein, 7 aa, synthetic construct
LGFRRHS

SEQ ID NO: 590 - protein, 7 aa, synthetic construct
LGFRRAE

SEQ ID NO: 591 - protein, 7 aa, synthetic construct
LGFRRAK

SEQ ID NO: 592 - protein, 7 aa, synthetic construct
LGFRRAS

SEQ ID NO: 593 - protein, 7 aa, synthetic construct
LGFRRAS

SEQ ID NO: 594 - protein, 7 aa, synthetic construct
LGFRRAS

SEQ ID NO: 595 - protein, 7 aa, synthetic construct
LGFRRAS

SEQ ID NO: 596 - protein, 7 aa, synthetic construct
LGFRRAS

SEQ ID NO: 597 - protein, 7 aa, synthetic construct
LGFRRAS

SEQ ID NO: 598 - protein, 7 aa, synthetic construct
LGFRRAS

SEQ ID NO: 599 - protein, 7 aa, synthetic construct
LGSRRAA

SEQ ID NO: 600 - protein, 7 aa, synthetic construct
LGSRRAA

SEQ ID NO: 601 - protein, 7 aa, synthetic construct
LGSRRAA

SEQ ID NO: 602 - protein, 7 aa, synthetic construct
LGSRRAA

SEQ ID NO: 603 - protein, 7 aa, synthetic construct
LGSRRAA

SEQ ID NO: 604 - protein, 7 aa, synthetic construct
LGSRRAA

SEQ ID NO: 605 - protein, 7 aa, synthetic construct
LGSRRAA

SEQ ID NO: 606 - protein, 7 aa, synthetic construct
LGSRRAA

SEQ ID NO: 607 - protein, 7 aa, synthetic construct
LGSRRAA

SEQ ID NO: 608 - protein, 7 aa, synthetic construct
LGSRRAA

SEQ ID NO: 609 - protein, 7 aa, synthetic construct
LGSRRAA

SEQ ID NO: 610 - protein, 7 aa, synthetic construct
LGSRRAA

SEQ ID NO: 611 - protein, 7 aa, synthetic construct
LGSRRAA

SEQ ID NO: 612 - protein, 7 aa, synthetic construct
LGSRRAA

SEQ ID NO: 613 - protein, 9 aa, synthetic construct
MQALNPPLS

SEQ ID NO: 614 - protein, 9 aa, synthetic construct
MQALNPPLS

SEQ ID NO: 615 - protein, 9 aa, synthetic construct
MQALNPPLS

SEQ ID NO: 616 - protein, 9 aa, synthetic construct
MQALNPPLS

SEQ ID NO: 617 - protein, 9 aa, synthetic construct
MQALNPPLS

SEQ ID NO: 618 - protein, 9 aa, synthetic construct
MQALNPPLS

SEQ ID NO: 619 - protein, 9 aa, synthetic construct
MQALRPPLS

SEQ ID NO: 620 - protein, 9 aa, synthetic construct
MQALNPPLS

SEQ ID NO: 621 - protein, 9 aa, synthetic construct
MQALNPPLS

SEQ ID NO: 622 - protein, 9 aa, synthetic construct
MQALNPPLS

SEQ ID NO: 623 - protein, 9 aa, synthetic construct
MQALNPPLS

SEQ ID NO: 624 - protein, 9 aa, synthetic construct
MQALNPPLS

SEQ ID NO: 625 - protein, 9 aa, synthetic construct
MQALNPPLS

SEQ ID NO: 626 - protein, 9 aa, synthetic construct
MQALNPPLS

SEQ ID NO: 627 - protein, 9 aa, synthetic construct
MQALNPPLS

SEQ ID NO: 628 - protein, 9 aa, synthetic construct
MQALNPPLS

SEQ ID NO: 629 - protein, 9 aa, synthetic construct
MQALNPPLS

SEQ ID NO: 630 - protein, 9 aa, synthetic construct
MQALNPPLS

SEQ ID NO: 631 - protein, 9 aa, synthetic construct
MQALNPPLS

SEQ ID NO: 632 - protein, 9 aa, synthetic construct
MQALNPPLS

SEQ ID NO: 633 - protein, 9 aa, synthetic construct
MQALNPPLS

SEQ ID NO: 634 - protein, 9 aa, synthetic construct
MQALNPPLS

SEQ ID NO: 635 - protein, 9 aa, synthetic construct
MQALNPPLS

SEQ ID NO: 636 - protein, 9 aa, synthetic construct
MQALNPPLS

SEQ ID NO: 637 - protein, 9 aa, synthetic construct
MQALNPPLS

SEQ ID NO: 638 - protein, 9 aa, synthetic construct
MQALNPPLS

SEQ ID NO: 639 - protein, 9 aa, synthetic construct
MQALNPPLS

SEQ ID NO: 640 - protein, 9 aa, synthetic construct
MQALNPPLS

SEQ ID NO: 641 - protein, 9 aa, synthetic construct
MQALNPPLS

SEQ ID NO: 642 - protein, 9 aa, synthetic construct
MQALNPPLS

SEQ ID NO: 643 - protein, 9 aa, synthetic construct
MQALNPPLS

SEQ ID NO: 644 - protein, 9 aa, synthetic construct
MQALNPPLS

SEQ ID NO: 645 - protein, 9 aa, synthetic construct
MQALNPPLS

SEQ ID NO: 646 - protein, 9 aa, synthetic construct
MQALNPPLS

SEQ ID NO: 647 - protein, 9 aa, synthetic construct
MQALNPPLS

SEQ ID NO: 648 - protein, 9 aa, synthetic construct
MQALNPPLS

SEQ ID NO: 649 - protein, 9 aa, synthetic construct
MQALNPPLS

SEQ ID NO: 650 - protein, 9 aa, synthetic construct
MQALNPPLS

SEQ ID NO: 651 - protein, 9 aa, synthetic construct
MQALNPPLS

SEQ ID NO: 652 - protein, 9 aa, synthetic construct
MQALNPPLS

SEQ ID NO: 653 - protein, 9 aa, synthetic construct
MQALNPPLS

SEQ ID NO: 654 - protein, 9 aa, synthetic construct
MQALNPPLS

SEQ ID NO: 655 - protein, 9 aa, synthetic construct
MQALNPPLS

SEQ ID NO: 656 - protein, 9 aa, synthetic construct
MQALNPPLS

SEQ ID NO: 657 - protein, 9 aa, synthetic construct
MQALNPPLS

SEQ ID NO: 658 - protein, 9 aa, synthetic construct
MQALNPPLS

SEQ ID NO: 659 - protein, 9 aa, synthetic construct
MQALNPPLS

SEQ ID NO: 660 - protein, 9 aa, synthetic construct
MQALNPPLS

SEQ ID NO: 661 - protein, 9 aa, synthetic construct
MQALNPPLS

SEQ ID NO: 662 - protein, 9 aa, synthetic construct
MQALNPPLS

SEQ ID NO: 663 - protein, 9 aa, synthetic construct
MQALNPPLS

SEQ ID NO: 664 - protein, 9 aa, synthetic construct
MQALNPPLS

SEQ ID NO: 665 - protein, 9 aa, synthetic construct
MQALNPPLS

SEQ ID NO: 666 - protein, 9 aa, synthetic construct
MQALNPPLS

SEQ ID NO: 667 - protein, 9 aa, synthetic construct
MQALNPPLS

SEQ ID NO: 668 - protein, 9 aa, synthetic construct
MQALNPPLS

SEQ ID NO: 669 - protein, 9 aa, synthetic construct
MQALNPPLS

SEQ ID NO: 670 - protein, 9 aa, synthetic construct
MQALNPPLS

SEQ ID NO: 671 - protein, 9 aa, synthetic construct
MQALNPPLS

SEQ ID NO: 672 - protein, 9 aa, synthetic construct
MQALNPPLS

SEQ ID NO: 673 - protein, 9 aa, synthetic construct
MQALNPPLS

SEQ ID NO: 674 - protein, 9 aa, synthetic construct
MQALNPPLS

SEQ ID NO: 675 - protein, 9 aa, synthetic construct
MQALNPPLS

SEQ ID NO: 676 - protein, 9 aa, synthetic construct
MQALNPPLS

SEQ ID NO: 677 - protein, 9 aa, synthetic construct
MQALNPPLS

SEQ ID NO: 678 - protein, 9 aa, synthetic construct
MQALNPPLS

SEQ ID NO: 679 - protein, 9 aa, synthetic construct
MQALNPPLS

SEQ ID NO: 680 - protein, 9 aa, synthetic construct
MQALNPPLS

SEQ ID NO: 681 - protein, 9 aa, synthetic construct
MQALNPPLS

SEQ ID NO: 682 - protein, 9 aa, synthetic construct
MQALNPPLS

SEQ ID NO: 683 - protein, 9 aa, synthetic construct
MQALNPPLS

SEQ ID NO: 684 - protein, 9 aa, synthetic construct
MQALNPPLS

SEQ ID NO: 685 - protein, 9 aa, synthetic construct
MQALNPPLS

SEQ ID NO: 686 - protein, 9 aa, synthetic construct
MQALNPPLS

SEQ ID NO: 687 - protein, 9 aa, synthetic construct
MQALNPPLS

SEQ ID NO: 688 - protein, 9 aa, synthetic construct
MQALNPPLS

SEQ ID NO: 689 - protein, 9 aa, synthetic construct
MQALNPPLS

SEQ ID NO: 690 - protein, 9 aa, synthetic construct
MQALNPPLS

SEQ ID NO: 691 - protein, 9 aa, synthetic construct
MQALNPPLS

SEQ ID NO: 692 - protein, 9 aa, synthetic construct
AQALNPPLS

SEQ ID NO: 693 - protein, 9 aa, synthetic construct
MAALNPPLS

SEQ ID NO: 694 - protein, 9 aa, synthetic construct
MQAFNPPLS

SEQ ID NO: 695 - protein, 9 aa, synthetic construct
MQAENPPLS

SEQ ID NO: 696 - protein, 9 aa, synthetic construct
MQALNPDLS

SEQ ID NO: 697 - protein, 9 aa, synthetic construct
MQALNPPYS

SEQ ID NO: 698 - protein, 9 aa, synthetic construct
MQALNPPDS

SEQ ID NO: 699 - protein, 9 aa, synthetic construct
MQALRPPLS

SEQ ID NO: 700 - protein, 9 aa, synthetic construct
MQALRPPLS

SEQ ID NO: 701 - protein, 9 aa, synthetic construct
MQALRPPLS

SEQ ID NO: 702 - protein, 9 aa, synthetic construct
MQALRPPLS

SEQ ID NO: 703 - protein, 9 aa, synthetic construct
MQALRPPLS

SEQ ID NO: 704 - protein, 9 aa, synthetic construct
MQALRPPLS

SEQ ID NO: 705 - protein, 9 aa, synthetic construct
MQALRPPLS

SEQ ID NO: 706 - protein, 9 aa, synthetic construct
MQALRPPLS

SEQ ID NO: 707 - protein, 9 aa, synthetic construct
MQALRPPLS

SEQ ID NO: 708 - protein, 9 aa, synthetic construct
MQALRPPLS

SEQ ID NO: 709 - protein, 9 aa, synthetic construct
MQALRPPLS

SEQ ID NO: 710 - protein, 9 aa, synthetic construct
MQALRPPLS

SEQ ID NO: 711 - protein, 9 aa, synthetic construct
MQALRPPLS

SEQ ID NO: 712 - protein, 9 aa, synthetic construct
MQALRPPLS

SEQ ID NO: 713 - protein, 129 aa, synthetic construct; identical to SEQ ID NO: 85

SEQ ID NO: 714 - protein, 129 aa, synthetic construct; identical to SEQ ID NO: 85

SEQ ID NO: 715 - protein, 129 aa, synthetic construct; identical to SEQ ID NO: 85

SEQ ID NO: 716 - protein, 129 aa, synthetic construct; identical to SEQ ID NO: 85

SEQ ID NO: 717 - protein, 129 aa, synthetic construct; identical to SEQ ID NO: 85

SEQ ID NO: 718 - protein, 129 aa, synthetic construct; identical to SEQ ID NO: 85

SEQ ID NO: 719 - protein, 129 aa, synthetic construct; identical to SEQ ID NO: 88

SEQ ID NO: 720 - protein, 129 aa, synthetic construct
EVQVVESGGGEIKPGGSVRLSCTANSFTFDNAWVNWVRQAPGRGLEWVGRLRSDTEGWTKDYAPHLKDRVVISREESGHTVFLEMASLQPQDTGVYYCNTGYWPQSAFLTNRAYAMDVWGRGTTVVVSS

SEQ ID NO: 721 - protein, 129 aa, synthetic construct
EVQVVESGGGEIKPGGSVRLSCTANSFTFSAAWVNWVRQAPGRGLEWVGRLRSDTEGWTKDYAPHLKDRVVISREESGHTVFLEMASLQPQDTGVYYCNTGYWPQSAFLTNRAYAMDVWGRGTTVVVSS

SEQ ID NO: 722 - protein, 129 aa, synthetic construct
EVQVVESGGGEIKPGGSVRLSCTANSFTFSNAEVNWVRQAPGRGLEWVGRLRSDTEGWTKDYAPHLKDRVVISREESGHTVFLEMASLQPQDTGVYYCNTGYWPQSAFLTNRAYAMDVWGRGTTVVVSS

SEQ ID NO: 723 - protein, 129 aa, synthetic construct
EVQVVESGGGEIKPGGSVRLSCTANSFTFSNALVNWVRQAPGRGLEWVGRLRSDTEGWTKDYAPHLKDRVVISREESGHTVFLEMASLQPQDTGVYYCNTGYWPQSAFLTNRAYAMDVWGRGTTVVVSS

SEQ ID NO: 724 - protein, 129 aa, synthetic construct
EVQVVESGGGEIKPGGSVRLSCTANSFTFSNAWVNWVRQAPGRGLEWVGELRSDTEGWTKDYAPHLKDRVVISREESGHTVFLEMASLQPQDTGVYYCNTGYWPQSAFLTNRAYAMDVWGRGTTVVVSS

SEQ ID NO: 725 - protein, 129 aa, synthetic construct
EVQVVESGGGEIKPGGSVRLSCTANSFTFSNAWVNWVRQAPGRGLEWVGRLYSDTEGWTKDYAPHLKDRVVISREESGHTVFLEMASLQPQDTGVYYCNTGYWPQSAFLTNRAYAMDVWGRGTTVVVSS

SEQ ID NO: 726 - protein, 129 aa, synthetic construct
EVQVVESGGGEIKPGGSVRLSCTANSFTFSNAWVNWVRQAPGRGLEWVGRLASDTEGWTKDYAPHLKDRVVISREESGHTVFLEMASLQPQDTGVYYCNTGYWPQSAFLTNRAYAMDVWGRGTTVVVSS

SEQ ID NO: 727 - protein, 129 aa, synthetic construct
EVQVVESGGGEIKPGGSVRLSCTANSFTFSNAWVNWVRQAPGRGLEWVGRLRSKTEGWTKDYAPHLKDRVVISREESGHTVFLEMASLQPQDTGVYYCNTGYWPQSAFLTNRAYAMDVWGRGTTVVVSS

SEQ ID NO: 728 - protein, 129 aa, synthetic construct
EVQVVESGGGEIKPGGSVRLSCTANSFTFSNAWVNWVRQAPGRGLEWVGRLRSYTEGWTKDYAPHLKDRVVISREESGHTVFLEMASLQPQDTGVYYCNTGYWPQSAFLTNRAYAMDVWGRGTTVVVSS

SEQ ID NO: 729 - protein, 129 aa, synthetic construct
EVQVVESGGGEIKPGGSVRLSCTANSFTFSNAWVNWVRQAPGRGLEWVGRLRSDREGWTKDYAPHLKDRVVISREESGHTVFLEMASLQPQDTGVYYCNTGYWPQSAFLTNRAYAMDVWGRGTTVVVSS

SEQ ID NO: 730 - protein, 129 aa, synthetic construct
EVQVVESGGGEIKPGGSVRLSCTANSFTFSNAWVNWVRQAPGRGLEWVGRLRSDEEGWTKDYAPHLKDRVVISREESGHTVFLEMASLQPQDTGVYYCNTGYWPQSAFLTNRAYAMDVWGRGTTVVVSS

SEQ ID NO: 731 - protein, 129 aa, synthetic construct
EVQVVESGGGEIKPGGSVRLSCTANSFTFSNAWVNWVRQAPGRGLEWVGRLRSDYEGWTKDYAPHLKDRVVISREESGHTVFLEMASLQPQDTGVYYCNTGYWPQSAFLTNRAYAMDVWGRGTTVVVSS

SEQ ID NO: 732 - protein, 129 aa, synthetic construct
EVQVVESGGGEIKPGGSVRLSCTANSFTFSNAWVNWVRQAPGRGLEWVGRLRSDTKGWTKDYAPHLKDRVVISREESGHTVFLEMASLQPQDTGVYYCNTGYWPQSAFLTNRAYAMDVWGRGTTVVVSS

SEQ ID NO: 733 - protein, 129 aa, synthetic construct
EVQVVESGGGEIKPGGSVRLSCTANSFTFSNAWVNWVRQAPGRGLEWVGRLRSDTEYWTKDYAPHLKDRVVISREESGHTVFLEMASLQPQDTGVYYCNTGYWPQSAFLTNRAYAMDVWGRGTTVVVSS

SEQ ID NO: 734 - protein, 129 aa, synthetic construct
EVQVVESGGGEIKPGGSVRLSCTANSFTFSNAWVNWVRQAPGRGLEWVGRLRSDTEHWTKDYAPHLKDRVVISREESGHTVFLEMASLQPQDTGVYYCNTGYWPQSAFLTNRAYAMDVWGRGTTVVVSS

SEQ ID NO: 735 - protein, 129 aa, synthetic construct
EVQVVESGGGEIKPGGSVRLSCTANSFTFSNAWVNWVRQAPGRGLEWVGRLRSDTEGSTKDYAPHLKDRVVISREESGHTVFLEMASLQPQDTGVYYCNTGYWPQSAFLTNRAYAMDVWGRGTTVVVSS

SEQ ID NO: 736 - protein, 129 aa, synthetic construct
EVQVVESGGGEIKPGGSVRLSCTANSFTFSNAWVNWVRQAPGRGLEWVGRLRSDTEGWYKDYAPHLKDRVVISREESGHTVFLEMASLQPQDTGVYYCNTGYWPQSAFLTNRAYAMDVWGRGTTVVVSS

SEQ ID NO: 737 - protein, 129 aa, synthetic construct
EVQVVESGGGEIKPGGSVRLSCTANSFTFSNAWVNWVRQAPGRGLEWVGRLRSDTEGWEKDYAPHLKDRVVISREESGHTVFLEMASLQPQDTGVYYCNTGYWPQSAFLTNRAYAMDVWGRGTTVVVSS

SEQ ID NO: 738 - protein, 129 aa, synthetic construct
EVQVVESGGGEIKPGGSVRLSCTANSFTFSNAWVNWVRQAPGRGLEWVGRLRSDTEGWTADYAPHLKDRVVISREESGHTVFLEMASLQPQDTGVYYCNTGYWPQSAFLTNRAYAMDVWGRGTTVVVSS

SEQ ID NO: 739 - protein, 129 aa, synthetic construct
EVQVVESGGGEIKPGGSVRLSCTANSFTFSNAWVNWVRQAPGRGLEWVGRLRSDTEGWTARYAPHLKDRVVISREESGHTVFLEMASLQPQDTGVYYCNTGYWPQSAFLTNRAYAMDVWGRGTTVVVSS

SEQ ID NO: 740 - protein, 129 aa, synthetic construct
EVQVVESGGGEIKPGGSVRLSCTANSFTFSNAWVNWVRQAPGRGLEWVGRLRSDTEGWTKDYAPHLADRVVISREESGHTVFLEMASLQPQDTGVYYCNTGYWPQSAFLTNRAYAMDVWGRGTTVVVSS

SEQ ID NO: 741 - protein, 129 aa, synthetic construct
EVQVVESGGGEIKPGGSVRLSCTANSFTFSNAWVNWVRQAPGRGLEWVGRLRSDTEGWTKDYAPHLYDRVVISREESGHTVFLEMASLQPQDTGVYYCNTGYWPQSAFLTNRAYAMDVWGRGTTVVVSS

SEQ ID NO: 742 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGAWPQSAFLTNRAYAMDVWGRGTTVVVSS

SEQ ID NO: 743 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGRWPQSAFLTNRAYAMDVWGRGTTVVVSS

SEQ ID NO: 744 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYAPQSAFLTNRAYAMDVWGRGTTVVVSS

SEQ ID NO: 745 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYEPQSAFLTNRAYAMDVWGRGTTVVVSS

SEQ ID NO: 746 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPASAFLTNRAYAMDVWGRGTTVVVSS

SEQ ID NO: 747 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPYSAFLTNRAYAMDVWGRGTTVVVSS

SEQ ID NO: 748 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQYAFLTNRAYAMDVWGRGTTVVVSS

SEQ ID NO: 749 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQEAFLTNRAYAMDVWGRGTTVVVSS

SEQ ID NO: 750 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSYFLTNRAYAMDVWGRGTTVVVSS

SEQ ID NO: 751 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSKFLTNRAYAMDVWGRGTTVVVSS

SEQ ID NO: 752 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAALTNRAYAMDVWGRGTTVVVSS

SEQ ID NO: 753 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAQLTNRAYAMDVWGRGTTVVVSS

SEQ ID NO: 754 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFATNRAYAMDVWGRGTTVVVSS

SEQ ID NO: 755 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLENRAYAMDVWGRGTTVVVSS

SEQ ID NO: 756 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLYNRAYAMDVWGRGTTVVVSS

SEQ ID NO: 757 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLTQRAYAMDVWGRGTTVVVSS

SEQ ID NO: 758 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLTARAYAMDVWGRGTTVVVSS

SEQ ID NO: 759 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLTNEAYAMDVWGRGTTVVVSS

SEQ ID NO: 760 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLTNSAYAMDVWGRGTTVVVSS

SEQ ID NO: 761 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLTNRYYAMDVWGRGTTVVVSS

SEQ ID NO: 762 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLTNRNYAMDVWGRGTTVVVSS

SEQ ID NO: 763 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLTNRAAAMDVWGRGTTVVVSS

SEQ ID NO: 764 - protein, 129 aa, synthetic construct
EVQVVESGGGEIKPGGSVRLSCTANSFTFSNAWVNWVRQAPGRGLEWVGRLRSDTEGWTKDYAPHLKDRVVISREESGHTVFLEMASLQPQDTGVYYCNTGYWPQSAFLTNRAKAMDVWGRGTTVVVSS

SEQ ID NO: 765 - protein, 129 aa, synthetic construct; identical to SEQ ID NO: 85

SEQ ID NO: 766 - protein, 129 aa, synthetic construct; identical to SEQ ID NO: 85

SEQ ID NO: 767 - protein, 129 aa, synthetic construct; identical to SEQ ID NO: 85

SEQ ID NO: 768 - protein, 129 aa, synthetic construct; identical to SEQ ID NO: 85

SEQ ID NO: 769 - protein, 129 aa, synthetic construct; identical to SEQ ID NO: 85

SEQ ID NO: 770 - protein, 129 aa, synthetic construct; identical to SEQ ID NO: 85

SEQ ID NO: 771 - protein, 129 aa, synthetic construct; identical to SEQ ID NO: 85

SEQ ID NO: 772 - protein, 129 aa, synthetic construct; identical to SEQ ID NO: 85

SEQ ID NO: 773 - protein, 129 aa, synthetic construct; identical to SEQ ID NO: 85

SEQ ID NO: 774 - protein, 129 aa, synthetic construct; identical to SEQ ID NO: 85

SEQ ID NO: 775 - protein, 129 aa, synthetic construct; identical to SEQ ID NO: 85

SEQ ID NO: 776 - protein, 129 aa, synthetic construct; identical to SEQ ID NO: 85

SEQ ID NO: 777 - protein, 129 aa, synthetic construct; identical to SEQ ID NO: 85

SEQ ID NO: 778 - protein, 129 aa, synthetic construct; identical to SEQ ID NO: 85

SEQ ID NO: 779 - protein, 129 aa, synthetic construct; identical to SEQ ID NO: 85

SEQ ID NO: 780 - protein, 129 aa, synthetic construct; identical to SEQ ID NO: 85

SEQ ID NO: 781 - protein, 129 aa, synthetic construct; identical to SEQ ID NO: 85

SEQ ID NO: 782 - protein, 129 aa, synthetic construct; identical to SEQ ID NO: 85

SEQ ID NO: 783 - protein, 129 aa, synthetic construct; identical to SEQ ID NO: 85

SEQ ID NO: 784 - protein, 129 aa, synthetic construct; identical to SEQ ID NO: 85

SEQ ID NO: 785 - protein, 129 aa, synthetic construct; identical to SEQ ID NO: 85

SEQ ID NO: 786 - protein, 129 aa, synthetic construct; identical to SEQ ID NO: 85

SEQ ID NO: 787 - protein, 129 aa, synthetic construct; identical to SEQ ID NO: 85

SEQ ID NO: 788 - protein, 129 aa, synthetic construct; identical to SEQ ID NO: 85

SEQ ID NO: 789 - protein, 129 aa, synthetic construct; identical to SEQ ID NO: 85

SEQ ID NO: 790 - protein, 129 aa, synthetic construct; identical to SEQ ID NO: 85

SEQ ID NO: 791 - protein, 129 aa, synthetic construct; identical to SEQ ID NO: 85

SEQ ID NO: 792 - protein, 129 aa, synthetic construct; identical to SEQ ID NO: 85

SEQ ID NO: 793 - protein, 129 aa, synthetic construct; identical to SEQ ID NO: 85

SEQ ID NO: 794 - protein, 129 aa, synthetic construct; identical to SEQ ID NO: 85

SEQ ID NO: 795 - protein, 129 aa, synthetic construct; identical to SEQ ID NO: 85

SEQ ID NO: 796 - protein, 129 aa, synthetic construct; identical to SEQ ID NO: 85

SEQ ID NO: 797 - protein, 129 aa, synthetic construct; identical to SEQ ID NO: 85

SEQ ID NO: 798 - protein, 129 aa, synthetic construct; identical to SEQ ID NO: 85

SEQ ID NO: 799 - protein, 129 aa, synthetic construct
EVQVVESGGGVVKPGGSLRLSCTANTFTFNNAWVNWVRQAPGRGLEWVGRLQSDTEHWAKDYAAHMKGRVVISREESGRTVYLEMIGLKVEDTAVYYCNTGYWPQSVFLTNRAYAMDVWGRGTTVIVSS

SEQ ID NO: 800 - protein, 129 aa, synthetic construct
EVQVVESGGGVVKPGGSLRLSCTANTFTFNNAWVNWVRQAPGRGLEWVGRLRSNTEHWAKDYAAHMKGRVVISREESGRTVYLEMIGLKVEDTAVYYCNTGYWPQSVFLTNRAYAMDVWGRGTTVIVSS

SEQ ID NO: 801 - protein, 129 aa, synthetic construct
EVQVVESGGGVVKPGGSLRLSCTANTFTFNNAWVNWVRQAPGRGLEWVGRLRSDQEHWAKDYAAHMKGRVVISREESGRTVYLEMIGLKVEDTAVYYCNTGYWPQSVFLTNRAYAMDVWGRGTTVIVSS

SEQ ID NO: 802 - protein, 129 aa, synthetic construct
EVQVVESGGGVVKPGGSLRLSCTANTFTFNNAWVNWVRQAPGRGLEWVGRLRSDYEHWAKDYAAHMKGRVVISREESGRTVYLEMIGLKVEDTAVYYCNTGYWPQSVFLTNRAYAMDVWGRGTTVIVSS

SEQ ID NO: 803 - protein, 129 aa, synthetic construct
EVQVVESGGGVVKPGGSLRLSCTANTFTFNNAWVNWVRQAPGRGLEWVGRLRSDTQHWAKDYAAHMKGRVVISREESGRTVYLEMIGLKVEDTAVYYCNTGYWPQSVFLTNRAYAMDVWGRGTTVIVSS

SEQ ID NO: 804 - protein, 129 aa, synthetic construct; identical to SEQ ID NO: 88

SEQ ID NO: 805 - protein, 129 aa, synthetic construct; identical to SEQ ID NO: 88

SEQ ID NO: 806 - protein, 129 aa, synthetic construct; identical to SEQ ID NO: 88

SEQ ID NO: 807 - protein, 129 aa, synthetic construct
EVQVVESGGGVVKPGGSLRLSCTANTFTFNNAWVNWVRQAPGRGLEWVGRLRSDTEHWAKDYAAHMKGRVVISREESGRTVYLEMIGLKVEDTAVYYCNTGKWPQSVFLTNRAYAMDVWGRGTTVIVSS

SEQ ID NO: 808 - protein, 129 aa, synthetic construct
EVQVVESGGGVVKPGGSLRLSCTANTFTFNNAWVNWVRQAPGRGLEWVGRLRSDTEHWAKDYAAHMKGRVVISREESGRTVYLEMIGLKVEDTAVYYCNTGYDPQSVFLTNRAYAMDVWGRGTTVIVSS

SEQ ID NO: 809 - protein, 129 aa, synthetic construct
EVQVVESGGGVVKPGGSLRLSCTANTFTFNNAWVNWVRQAPGRGLEWVGRLRSDTEHWAKDYAAHMKGRVVISREESGRTVYLEMIGLKVEDTAVYYCNTGYWPASVFLTNRAYAMDVWGRGTTVIVSS

SEQ ID NO: 810 - protein, 129 aa, synthetic construct
EVQVVESGGGVVKPGGSLRLSCTANTFTFNNAWVNWVRQAPGRGLEWVGRLRSDTEHWAKDYAAHMKGRVVISREESGRTVYLEMIGLKVEDTAVYYCNTGYWPQSVQLTNRAYAMDVWGRGTTVIVSS

SEQ ID NO: 811 - protein, 129 aa, synthetic construct
EVQVVESGGGVVKPGGSLRLSCTANTFTFNNAWVNWVRQAPGRGLEWVGRLRSDTEHWAKDYAAHMKGRVVISREESGRTVYLEMIGLKVEDTAVYYCNTGYWPQSVFLTDRAYAMDVWGRGTTVIVSS

SEQ ID NO: 812 - protein, 129 aa, synthetic construct
EVQVVESGGGVVKPGGSLRLSCTANTFTFNNAWVNWVRQAPGRGLEWVGRLRSDTEHWAKDYAAHMKGRVVISREESGRTVYLEMIGLKVEDTAVYYCNTGYWPQSVFLTNQAYAMDVWGRGTTVIVSS

SEQ ID NO: 813 - protein, 111 aa, synthetic construct
DIVMTQSPLFLSVTPGESASMSCASSESLQRGQYHFLDWYLQKPGQSPQLLIYLGFRRASGVPARFSARASDTHYTLTISRVDTEDVGVYYCMQALNPPLSFGPGTTVDMK

SEQ ID NO: 814 - protein, 111 aa, synthetic construct; identical to SEQ ID NO: 813

SEQ ID NO: 815 - protein, 111 aa, synthetic construct; identical to SEQ ID NO: 813

SEQ ID NO: 816 - protein, 111 aa, synthetic construct; identical to SEQ ID NO: 813

SEQ ID NO: 817 - protein, 111 aa, synthetic construct; identical to SEQ ID NO: 813

SEQ ID NO: 818 - protein, 111 aa, synthetic construct; identical to SEQ ID NO: 813

SEQ ID NO: 819 - protein, 111 aa, synthetic construct; identical to SEQ ID NO: 813

SEQ ID NO: 820 - protein, 111 aa, synthetic construct; identical to SEQ ID NO: 813

SEQ ID NO: 821 - protein, 111 aa, synthetic construct; identical to SEQ ID NO: 813

SEQ ID NO: 822 - protein, 111 aa, synthetic construct; identical to SEQ ID NO: 813

SEQ ID NO: 823 - protein, 111 aa, synthetic construct; identical to SEQ ID NO: 813

SEQ ID NO: 824 - protein, 111 aa, synthetic construct; identical to SEQ ID NO: 813

SEQ ID NO: 825 - protein, 111 aa, synthetic construct; identical to SEQ ID NO: 813

SEQ ID NO: 826 - protein, 111 aa, synthetic construct; identical to SEQ ID NO: 813

SEQ ID NO: 827 - protein, 111 aa, synthetic construct; identical to SEQ ID NO: 813

SEQ ID NO: 828 - protein, 111 aa, synthetic construct; identical to SEQ ID NO: 813

SEQ ID NO: 829 - protein, 111 aa, synthetic construct; identical to SEQ ID NO: 813

SEQ ID NO: 830 - protein, 111 aa, synthetic construct; identical to SEQ ID NO: 813

SEQ ID NO: 831 - protein, 111 aa, synthetic construct; identical to SEQ ID NO: 813

SEQ ID NO: 832 - protein, 111 aa, synthetic construct; identical to SEQ ID NO: 813

SEQ ID NO: 833 - protein, 111 aa, synthetic construct; identical to SEQ ID NO: 813

SEQ ID NO: 834 - protein, 111 aa, synthetic construct; identical to SEQ ID NO: 813

SEQ ID NO: 835 - protein, 111 aa, synthetic construct; identical to SEQ ID NO: 813

SEQ ID NO: 836 - protein, 111 aa, synthetic construct; identical to SEQ ID NO: 813

SEQ ID NO: 837 - protein, 111 aa, synthetic construct; identical to SEQ ID NO: 813

SEQ ID NO: 838 - protein, 111 aa, synthetic construct; identical to SEQ ID NO: 813

SEQ ID NO: 839 - protein, 111 aa, synthetic construct; identical to SEQ ID NO: 813

SEQ ID NO: 840 - protein, 111 aa, synthetic construct; identical to SEQ ID NO: 813

SEQ ID NO: 841 - protein, 111 aa, synthetic construct; identical to SEQ ID NO: 813

SEQ ID NO: 842 - protein, 111 aa, synthetic construct; identical to SEQ ID NO: 813

SEQ ID NO: 843 - protein, 111 aa, synthetic construct; identical to SEQ ID NO: 813

SEQ ID NO: 844 - protein, 111 aa, synthetic construct; identical to SEQ ID NO: 813

SEQ ID NO: 845 - protein, 111 aa, synthetic construct; identical to SEQ ID NO: 813

SEQ ID NO: 846 - protein, 111 aa, synthetic construct; identical to SEQ ID NO: 813

SEQ ID NO: 847 - protein, 111 aa, synthetic construct; identical to SEQ ID NO: 813

SEQ ID NO: 848 - protein, 111 aa, synthetic construct; identical to SEQ ID NO: 813

SEQ ID NO: 849 - protein, 111 aa, synthetic construct; identical to SEQ ID NO: 813

SEQ ID NO: 850 - protein, 111 aa, synthetic construct; identical to SEQ ID NO: 813

SEQ ID NO: 851 - protein, 111 aa, synthetic construct; identical to SEQ ID NO: 813

SEQ ID NO: 852 - protein, 111 aa, synthetic construct; identical to SEQ ID NO: 813

SEQ ID NO: 853 - protein, 111 aa, synthetic construct; identical to SEQ ID NO: 813

SEQ ID NO: 854 - protein, 111 aa, synthetic construct; identical to SEQ ID NO: 813

SEQ ID NO: 855 - protein, 111 aa, synthetic construct; identical to SEQ ID NO: 813

SEQ ID NO: 856 - protein, 111 aa, synthetic construct; identical to SEQ ID NO: 813

SEQ ID NO: 857 - protein, 111 aa, synthetic construct; identical to SEQ ID NO: 813

SEQ ID NO: 858 - protein, 111 aa, synthetic construct; identical to SEQ ID NO: 813

SEQ ID NO: 859 - protein, 111 aa, synthetic construct; identical to SEQ ID NO: 813

SEQ ID NO: 860 - protein, 111 aa, synthetic construct; identical to SEQ ID NO: 813

SEQ ID NO: 861 - protein, 111 aa, synthetic construct; identical to SEQ ID NO: 813

SEQ ID NO: 862 - protein, 111 aa, synthetic construct; identical to SEQ ID NO: 813

SEQ ID NO: 863 - protein, 111 aa, synthetic construct; identical to SEQ ID NO: 813

SEQ ID NO: 864 - protein, 111 aa, synthetic construct; identical to SEQ ID NO: 813

SEQ ID NO: 865 - protein, 111 aa, synthetic construct; identical to SEQ ID NO: 813

SEQ ID NO: 866 - protein, 111 aa, synthetic construct; identical to SEQ ID NO: 813

SEQ ID NO: 867 - protein, 111 aa, synthetic construct; identical to SEQ ID NO: 813

SEQ ID NO: 868 - protein, 111 aa, synthetic construct; identical to SEQ ID NO: 813

SEQ ID NO: 869 - protein, 111 aa, synthetic construct; identical to SEQ ID NO: 813

SEQ ID NO: 870 - protein, 111 aa, synthetic construct; identical to SEQ ID NO: 813

SEQ ID NO: 871 - protein, 111 aa, synthetic construct; identical to SEQ ID NO: 813

SEQ ID NO: 872 - protein, 111 aa, synthetic construct; identical to SEQ ID NO: 813

SEQ ID NO: 873 - protein, 111 aa, synthetic construct; identical to SEQ ID NO: 813

SEQ ID NO: 874 - protein, 111 aa, synthetic construct; identical to SEQ ID NO: 813

SEQ ID NO: 875 - protein, 111 aa, synthetic construct; identical to SEQ ID NO: 813

SEQ ID NO: 876 - protein, 111 aa, synthetic construct; identical to SEQ ID NO: 813

SEQ ID NO: 877 - protein, 111 aa, synthetic construct; identical to SEQ ID NO: 813

SEQ ID NO: 878 - protein, 111 aa, synthetic construct; identical to SEQ ID NO: 813

SEQ ID NO: 879 - protein, 111 aa, synthetic construct; identical to SEQ ID NO: 813

SEQ ID NO: 880 - protein, 111 aa, synthetic construct; identical to SEQ ID NO: 813

SEQ ID NO: 881 - protein, 111 aa, synthetic construct; identical to SEQ ID NO: 813

SEQ ID NO: 882 - protein, 111 aa, synthetic construct; identical to SEQ ID NO: 813

SEQ ID NO: 883 - protein, 111 aa, synthetic construct; identical to SEQ ID NO: 813

SEQ ID NO: 884 - protein, 111 aa, synthetic construct; identical to SEQ ID NO: 813

SEQ ID NO: 885 - protein, 111 aa, synthetic construct; identical to SEQ ID NO: 813

SEQ ID NO: 886 - protein, 111 aa, synthetic construct; identical to SEQ ID NO: 813

SEQ ID NO: 887 - protein, 111 aa, synthetic construct; identical to SEQ ID NO: 813

SEQ ID NO: 888 - protein, 111 aa, synthetic construct; identical to SEQ ID NO: 813

SEQ ID NO: 889 - protein, 111 aa, synthetic construct; identical to SEQ ID NO: 813

SEQ ID NO: 890 - protein, 111 aa, synthetic construct; identical to SEQ ID NO: 813

SEQ ID NO: 891 - protein, 111 aa, synthetic construct; identical to SEQ ID NO: 813

SEQ ID NO: 892 - protein, 111 aa, synthetic construct; identical to SEQ ID NO: 813

SEQ ID NO: 893 - protein, 111 aa, synthetic construct; identical to SEQ ID NO: 813

SEQ ID NO: 894 - protein, 111 aa, synthetic construct; identical to SEQ ID NO: 813

SEQ ID NO: 895 - protein, 111 aa, synthetic construct; identical to SEQ ID NO: 813

SEQ ID NO: 896 - protein, 111 aa, synthetic construct; identical to SEQ ID NO: 813

SEQ ID NO: 897 - protein, 111 aa, synthetic construct; identical to SEQ ID NO: 813

SEQ ID NO: 898 - protein, 111 aa, synthetic construct; identical to SEQ ID NO: 813

SEQ ID NO: 899 - protein, 111 aa, synthetic construct; identical to SEQ ID NO: 813

SEQ ID NO: 900 - protein, 111 aa, synthetic construct; identical to SEQ ID NO: 813

SEQ ID NO: 901 - protein, 111 aa, synthetic construct; identical to SEQ ID NO: 813

SEQ ID NO: 902 - protein, 111 aa, synthetic construct; identical to SEQ ID NO: 813

SEQ ID NO: 903 - protein, 111 aa, synthetic construct; identical to SEQ ID NO: 813

SEQ ID NO: 904 - protein, 111 aa, synthetic construct; identical to SEQ ID NO: 813

SEQ ID NO: 905 - protein, 111 aa, synthetic construct; identical to SEQ ID NO: 813

SEQ ID NO: 906 - protein, 111 aa, synthetic construct; identical to SEQ ID NO: 813

SEQ ID NO: 907 - protein, 111 aa, synthetic construct; identical to SEQ ID NO: 813

SEQ ID NO: 908 - protein, 111 aa, synthetic construct; identical to SEQ ID NO: 813

SEQ ID NO: 909 - protein, 111 aa, synthetic construct; identical to SEQ ID NO: 813

SEQ ID NO: 910 - protein, 111 aa, synthetic construct; identical to SEQ ID NO: 813

SEQ ID NO: 911 - protein, 111 aa, synthetic construct; identical to SEQ ID NO: 813

SEQ ID NO: 912 - protein, 111 aa, synthetic construct; identical to SEQ ID NO: 813

SEQ ID NO: 913 - RNA, 20 nt, synthetic construct
  source 1..20: mol_type = other RNA
  ncRNA 1..20: ncRNA_class = guide_RNA
ggcgccgcaggaggctcccg

SEQ ID NO: 914 - RNA, 20 nt, synthetic construct
  source 1..20: mol_type = other RNA
  ncRNA 1..20: ncRNA_class = guide_RNA
gaggctcccggggcctggtc

SEQ ID NO: 915 - RNA, 20 nt, synthetic construct
  source 1..20: mol_type = other RNA
  ncRNA 1..20: ncRNA_class = guide_RNA
ggcctgcgacgggagaggcg

SEQ ID NO: 916 - RNA, 20 nt, synthetic construct
  source 1..20: mol_type = other RNA
  ncRNA 1..20: ncRNA_class = guide_RNA
gaggccattcggcttcccta

SEQ ID NO: 917 - RNA, 20 nt, synthetic construct
  source 1..20: mol_type = other RNA
gtgtcgtgatgcgtagacgg

SEQ ID NO: 918 - RNA, 20 nt, synthetic construct
  source 1..20: mol_type = other RNA
gtcatcaaggagcattccgt

SEQ ID NO: 919 - protein, 998 aa, Homo sapiens [sequence omitted]

SEQ ID NO: 920 - protein, 530 aa, Homo sapiens
MRRLWGAARKPSGAGWEKEWAEAPQEAPGAWSGRLGPGRSGRKGRAVPGWASWPAHLALAARPARHLGGAGQGPRPLHSGTAPFHSRASGERQRRLEPQLQHESRCRSSTPADAWRAEAALPVRAMGAPWGSPTAAAGGRRGWRRGRGLPWTVCVLAAAGLTCTALITYACWGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER